(19) United States
(12) Patent Application Publication
(10) Pub. No.: US 2022/0267276 A1
(43) Pub. Date: Aug. 25, 2022

(54) NLRP MODULATORS

(71) Applicant: Novartis AG, Basel (CH)

(72) Inventors: Luigi FRANCHI, Ann Arbor, MI (US); Shomir GHOSH, Brookline, MA (US); Gary GLICK, Ann Arbor, MI (US); Jason KATZ, Newton, MA (US); Anthony William OPIPARI, Jr., Dexter, MI (US); William R. ROUSH, Boston, MA (US); Hans Martin SEIDEL, Concord, MA (US); Dong-Ming SHEN, Edison, NJ (US); Shankar VENKATRAMAN, Lansdale, PA (US); David Guenther WINKLER, Arlington, MA (US)

(21) Appl. No.: 17/252,623

(22) PCT Filed: Jul. 2, 2019
(86) PCT No.: PCT/US2019/040400

§ 371 Date: Dec. 15, 2020

Related U.S. Application Data
(60) Provisional application No. 62/812,364, filed on Mar. 1, 2019.
(60) Provisional application No. 62/693,819, filed on Jul. 3, 2018.

(51) Int. Cl. C07D 221/16; C07D 277/36; C07D 417/12; C07D 213/75; C07C 335/42; A61K 45/06

(52) U.S. Cl. CPC C07D 221/16; C07D 277/36; A61K 45/06; C07D 213/75; C07C 335/42; C07D 417/12

(57) ABSTRACT

In one aspect, compounds of Formula AA, or a pharmaceutically acceptable salt thereof, are featured: or a pharmaceutically acceptable salt thereof, wherein the variables shown in Formula A can be as defined anywhere herein, useful to treat connected to the modulation of NRLP3.

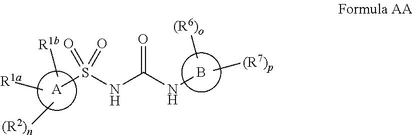

### TECHNICAL FIELD

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) contributes to the pathology and/or symptoms and/or progression of the condition, disease or disorder in a subject (e.g., a human). This disclosure also features compositions as well as other methods of using and making the same.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

NLRP3 can form a complex and has been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary-idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NLRP3.

### SUMMARY

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling).

In some embodiments, provided herein is a compound of Formula AA

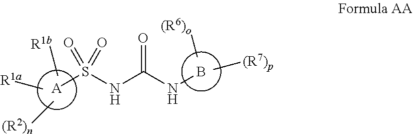

or a pharmaceutically acceptable salt thereof, wherein the variables in Formula AA can be as defined anywhere herein.

This disclosure also features compositions as well as other methods of using and making the same.

An “antagonist” of NLRP3 includes compounds that inhibit the ability of NLRP3 to induce the production of IL-1β and/or IL-18 by directly binding to NLRP3, or by inactivating, destabilizing, altering distribution, of NLRP3 or otherwise.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating (e.g., agonizing, partially agonizing, antagonizing) NLRP3 activity are featured that include contacting NLRP3 with a chemical entity described herein (e.g., a compound described generically or specifically herein or a pharmaceutically acceptable salt thereof or compositions containing the same). Methods include in vitro methods, e.g., contacting a sample that includes one or more cells comprising NLRP3, as well as in vivo methods.

In a further aspect, methods of treatment of a disease in which NLRP3 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In a further aspect, methods of treatment are featured that include administering to a subject a chemical entity described herein (e.g., a compound described generically or specifically herein or a pharmaceutically acceptable salt thereof or compositions containing the same), wherein the chemical entity is administered in an amount effective to treat a disease in which NLRP3 signaling contributes to the pathology and/or symptoms and/or progression of the disease, thereby treating the disease.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis, osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP3 molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP3, or a pharmaceutically acceptable salt and/or hydrate and/or cocrystal thereof) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington; The Science and Practice of Pharmacy, 21st ed.; Lippincott Williams & Wilkins: Philadelphia, Pa., 2005; Handbook of Pharmaceutical Excipients, 6th ed.; Rowe et al, Eds., The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd. ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, saturated or unsaturated, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six-membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocyclic rings include five-membered, six-membered, and seven-membered heterocyclic rings.

The term “cycloalkyl” as used herein includes an nonaromatic cyclic, bicylic, fused, or spire hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to an nonaromatic 5-8 membered monocyclic, 8-12 membered bicyclic, or 11-14 membered tricyclic ring, fused, or spiro system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

The term “aryl” is intended to mean an aromatic ring radical containing 6 to 10 ring carbons. Examples include phenyl and naphthyl.

The term “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl. pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

The term “hydroxy” refers to an OH group.

The term “amino” refers to an NH₂ group.

The term “oxo” refers to O. By way of example, substitution of a CH₂ a group with oxo gives a C═O group.

As used herein, the terms “the ring A” or “A” are used interchangeably to denote

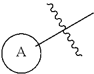

in formula AA, wherein the bond that is shown as being broken by the wavy line [symbol] connects A to the S(O)(NHR³)=N moiety of Formula AA.

As used herein, the terms “the ring B” or “B” are used interchangeably to denote

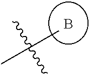

in formula AA wherein the bond that is shown as being broken by the wavy line [symbol] connects B to the NH(CO) group of Formula AA.

[symbol]

As used herein, the term “the substituted ring A” is used to denote

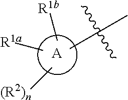

in formula AA, wherein the bond that is shown as being broken by the wavy line [symbol] connects A to the S(O₂)NH moiety of Formula AA.

[symbol]

As used herein, the term “the optionally substituted ring B” is used to denote

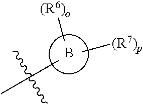

in formula AA, wherein the bond that is shown as being broken by the wavy line [symbol] connects B to the NH(CO) group of Formula AA.

As used herein, the recitation “S(O₂)”, alone or as part of a larger recitation, refers to the group

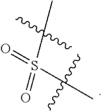 [SMILES: *S(*)(=O)=O]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, by way of example, a compound that is represented as containing the moiety

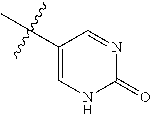

is also intended to include the tautomeric form containing the moiety

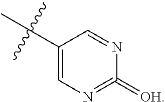

The details of one or more embodiments of the invention are set forth in the accompanying drawings and the description below. Other features and advantages of the invention will be apparent from the description and drawings, and from the claims.

### DETAILED DESCRIPTION

In some embodiments, provided herein is a compound of Formula AA

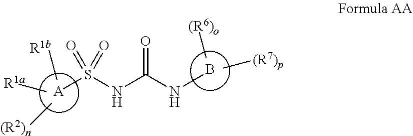

wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is a C₁-C₆ alkyl or —SO₂NR¹¹R¹²;

wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —COR¹³, —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²;

at least one R⁶ is ortho to the bond connecting the B ring to the NH(CO) group of Formula AA; R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH2, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C=NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, C₀NR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R⁶ and R are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₁-C₆ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ and, 5- to 10-membered heteroaryl, NHCOC₆-C₁₀ aryl, NHCO(5 to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₅ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; R¹⁰ is C₁-C₆ alkyl;
   each of R⁸ and R⁹ at each occurrence is independently selected from hydrogen, C₁-C₆ alkyl, (C=NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ and, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy;
   with the proviso that the compound of Formula AA is not a compound selected from the group consisting of:

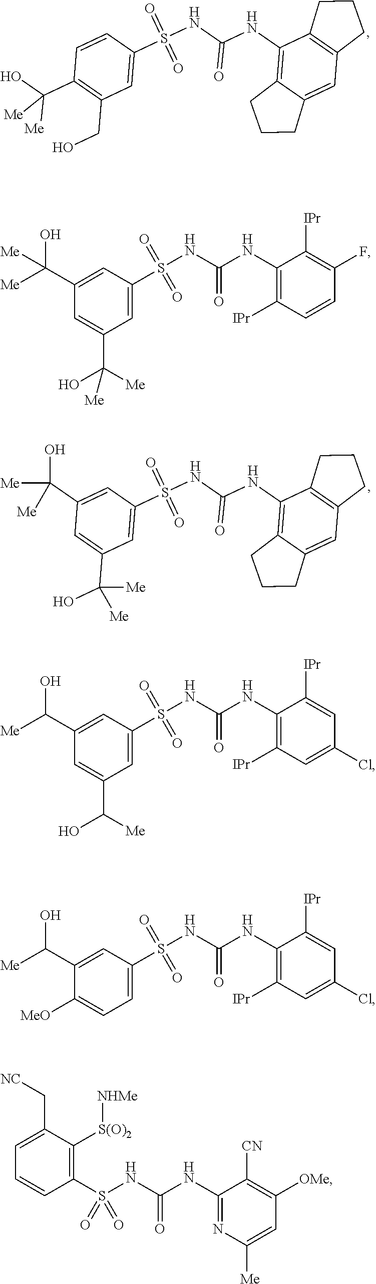

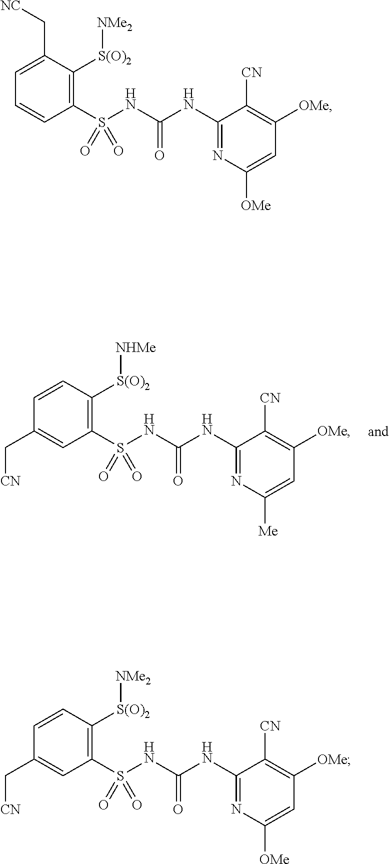

or a pharmaceutically acceptable salt thereof.

Without being bound by theory, it is believed that the presence of the two substituents R^{1a} and R^{1b} result in compounds that cross the intestinal barrier in a limited manner and are therefore result in compounds that are restricted to the gut and provide targeted delivery to the gut. Applicants have surprisingly found that the presence of at least two substituents, and particularly two polar substituents R^{1a} and R^{1b} may provide compounds of formula AA that are poorly absorbed into systemic circulation after oral administration and are therefore restricted to the gut. Without being bound by theory, it is further hypothesized that the gut restricted compounds of the present invention may be used for treatment or prevention or alleviation of symptoms of certain gastrointestinal disorders. It is also hypothesized that the targeting of compounds to the gut may reduce the incidence of side effects due to systemic absorption of compounds.

In some embodiments, provided herein is a compound of Formula AA

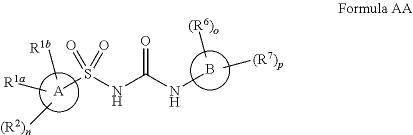

wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is a C₁-C₆ alkyl;
wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —R¹CONR¹¹R¹², —CR¹¹R¹²NR¹¹R¹², and —NR¹¹COR¹²;
   at least one R⁵ is ortho to the bond connecting the B ring to the NH(CO) group of Formula AA; R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C=NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo,
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, C₀NR⁸R⁹, SF₅, SC₁-C₆ alkyl, S(O₂)C₁-C₆ alkyl, C₆-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₁-C₆ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ and, 5- to 10-membered heteroaryl, NHCOC₆-C₁₀ aryl, NHCO(5 to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₅ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, NR¹⁰, COOC₁-C₆ alkyl, C₆-C₁₀ aryl, and CONR⁸R⁹; R¹⁰ is C₁-C₆ alkyl;
   each of R⁸ and R⁹ at each occurrence is independently selected from hydrogen, C₁-C₆ alkyl, (C=NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ and, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy;
   with the proviso that the compound of Formula AA is not a compound selected from the group consisting of:

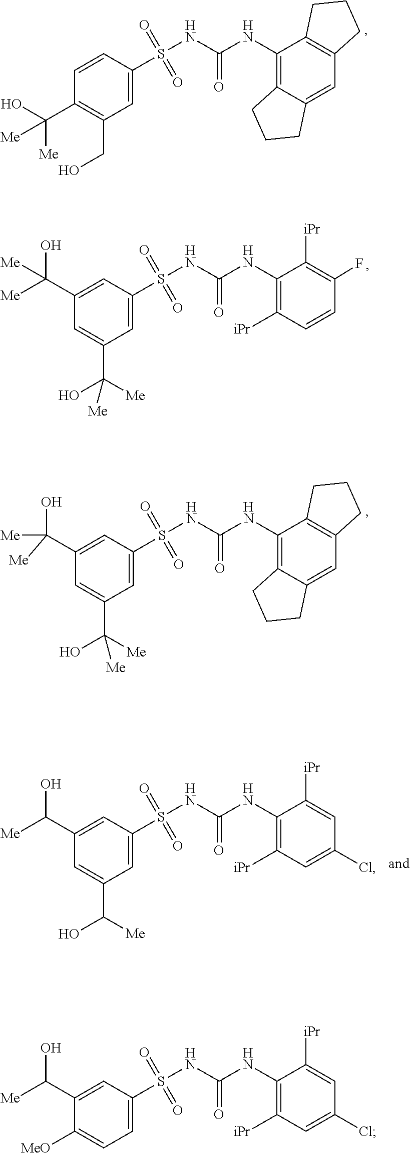

or a pharmaceutically acceptable salt thereof.

In some embodiments, provided herein is a compound of Formula AA

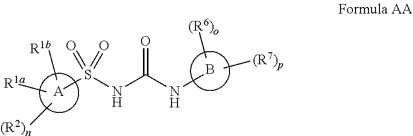

wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is —SO₂NR¹¹R¹²;
R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²:
at least one R⁶ is ortho to the bond connecting the B ring to the NH(CO) group of Formula AA; R² is selected from C₁-C₆ alkyl, C₁-C₆ haloalkyl, C₁-C₆ alkoxy, C₁-C₆ haloalkoxy, halo. CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl,
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R⁶ and R are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl, and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ and, 5- to 10-membered heteroaryl, NHCOC₆-C₁₀ aryl, NHCO(5 to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   R¹⁰ is C₁-C₆ alkyl;
   each of R⁸ and R⁹ at each occurrence is independently selected from hydrogen, C₁-C₆ alkyl, (C=NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy;
   with the proviso that the compound of Formula AA is not a compound selected from the group consisting of;

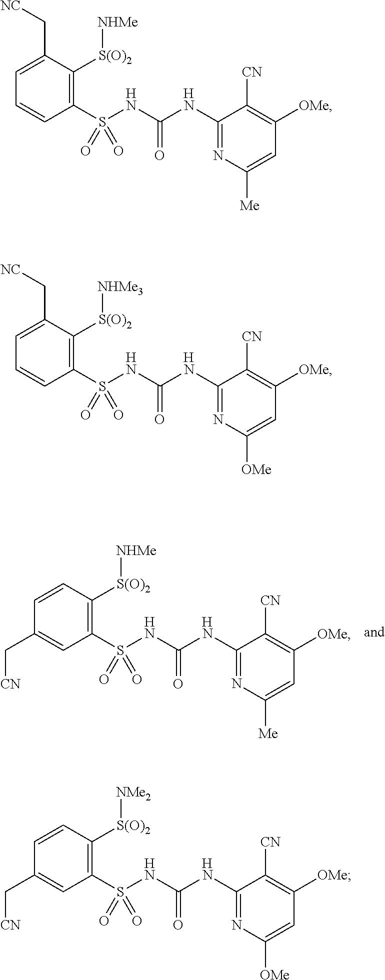

or a pharmaceutically acceptable salt thereof.

In some embodiments, provided herein is a compound of Formula AA

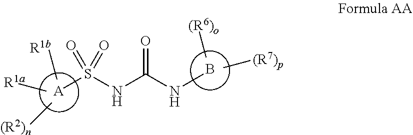

wherein the compound of Formula AA is selected from

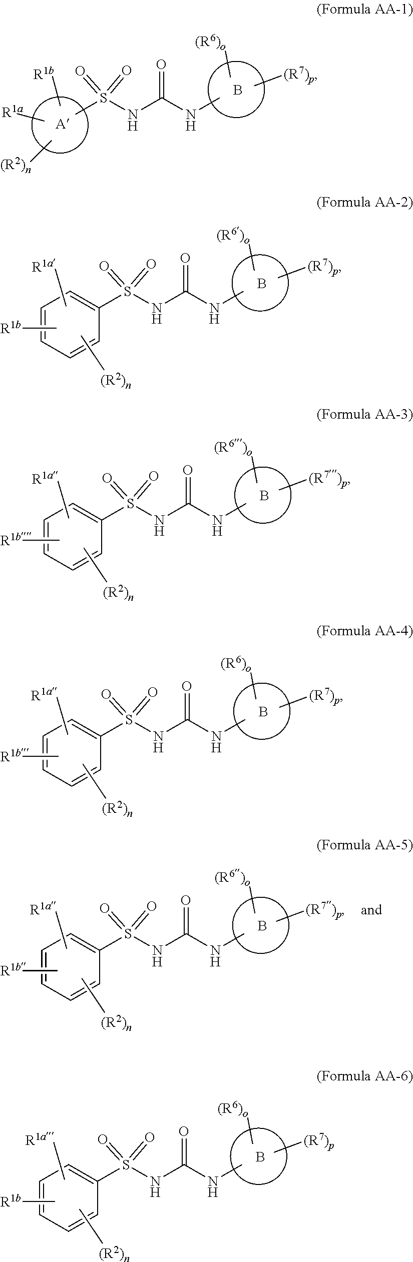

wherein
n=0 or 1;
o=1 or 2;
p=0, 1, 2, or 3;
wherein
A′ is a 5- to 10-membered heteroaryl;
B is a 5- to 10-membered heteroaryl or a C₆-C₁₀ and;
wherein
R^{1a} is a C₁-C₆ alkyl or —SO₂NR¹¹R¹²;
wherein the C₁-C₆ alkyl is substituted with one or more hydroxy or —OSi(R¹³)₃;
   R^{1a′} is —SO₂R¹¹R¹²;
   R^{1a″} is a C₁-C₆ alkyl;
wherein the C₁-C₆ alkyl is substituted with one or more hydroxy,
   R^{1a″} is a C₁-C₆ alkyl;
wherein the C₁-C₆ alkyl is substituted with one or more-OSi(R¹³)₃;
   R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —
   CR¹¹R¹²NR¹¹R¹², and —NR¹¹COR¹²;
   R^{1b″} is —OR¹¹;
   R^{1b′″} is a —SO₂NR¹¹R¹², —SO₂R¹³, —CONR¹¹R¹², —COR¹³; —CO₂R¹³, —NR¹³CONR¹¹R¹²; —
   CR¹¹R¹²CN, —NR¹¹SO₂R¹—NR¹¹CONR¹¹R¹², —CR¹¹R¹²NR¹¹R¹², and —NR¹¹ COR¹²;
   R^{1b′″} is a C₁-C₆ alkyl substituted with one or more hydroxy;
   at least one R⁶ is ortho to the bond connecting the B ring to the NH(CO) group of Formula AA-1 and Formula AA-4;
   at least one R⁶ is ortho to the bond connecting the B ring to the NH(CO) group of Formula AA-2;
   at least one R⁵ is ortho to the bond connecting the B ring to the NH(CO) group of Formula AA-5;
   at least one R^{6′″} is ortho to the bond connecting the B ring to the NH(CO) group of Formula AA-3;
   R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C=NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₀ alkenyl,
   wherein R⁶ and R⁷ are each optionally substituted with one or more substituents independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂.NR⁸R⁹, ═NR¹⁰, COOC₁-C₆ alkyl, C₆-C₁₀ aryl, and CONR⁸R⁹; R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH2, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₆-C₁₀ alkynyl, C₆-C₁₀ aryloxy, and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₅ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alky), C₆-C₁₀ aryl, and CONR⁸R⁹;
   R^{6′″} and R^{7′″} are each independently selected from C₁-C₆ alkyl, C₁-C₆ haloalkyl, C₁-C₆ alkoxy, C₁-C₆ haloalkoxy, F, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH2, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R^{6″} and R^{7″} are each optionally substituted with one or more substituents independently selected from hydroxy, halo, CN, oxo, C₁-C₆ alkyl, C₁-C₆ alkoxy, NR*R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl, and wherein the C₁-C₆ alkyl or C₁-C₆ alkoxy that R⁶ or R^{7″} is substituted with is optionally substituted with one or more hydroxyl, halo, C₁-C₆ aryl or NR⁸R⁹, or wherein R^{6″} or R^{7″} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ and, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R^{6″} and R^{7″} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ and, and CONR⁸R⁹;
   R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R^{6′″} and R^{7′″} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6′″} or R^{7″} is substituted with is optionally substituted with one or more hydroxyl, halo, C₆-C₁₀ aryl or NR⁸R⁹, or wherein R^{6′″} or R^{7′″} is optionally fused to a five- to -seven-membered carbocyclic ring or heterocyclic ring containing one or two heteroatoms independently selected from oxygen, sulfur and nitrogen,
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R^{6′″} and R^{7′″} on adjacent atoms, taken together with the atoms connecting them, independently form at least one C₄, C₆, C₇, or C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   R¹⁰ is C₁-C₆ alkyl;
   each of R⁸ and R⁹ at each occurrence is independently selected from hydrogen, C₁-C₆ alkyl, (C=NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy;
   or a pharmaceutically acceptable salt thereof.

In some embodiments, provided herein is a compound of Formula AA

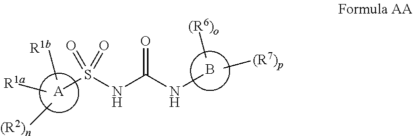

wherein the compound of Formula AA is selected from

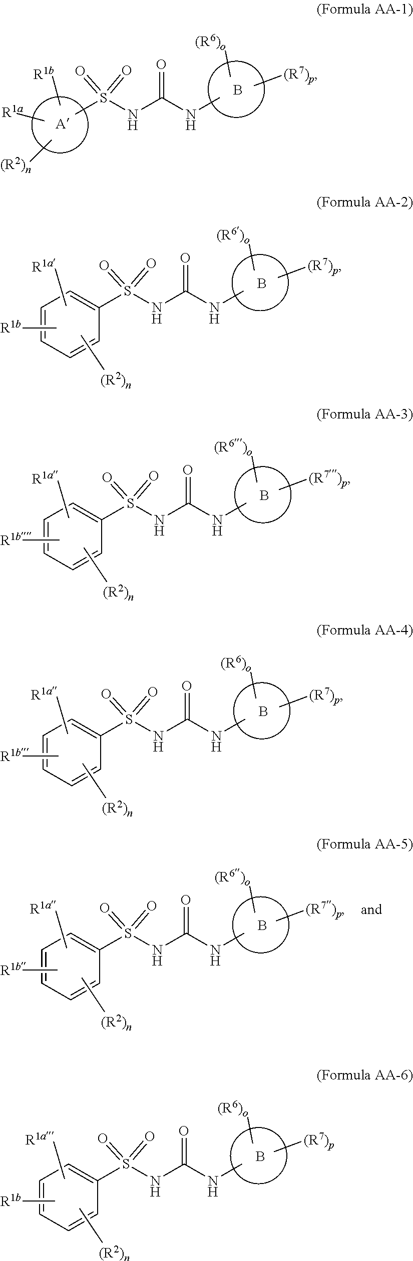

wherein
n=0 or 1;
o=1 or 2;
p=0, 1, 2, or 3;
wherein
A′ is a 5- to 10-membered heteroaryl;
B is a 5- to 10-membered heteroaryl or a C₆-C₁₀ and;
wherein
R^{1a} is a C₁-C₆ alkyl or —SO₂NR¹¹R¹²;
wherein the C₁-C₆ alkyl is substituted with one or more hydroxy or —OSi(R¹³)₃;
   R^{1a′} is —SO₂R¹¹R¹².
   R^{1a″} is a C₁-C₆ alkyl;
wherein the C₁-C₆ alkyl is substituted with one or more hydroxy,
   R^{1a′″} is a C₁-C₆ alkyl;
wherein the C₁-C₆ alkyl is substituted with one or more-OSi(R¹³)₃;
   R^{1b} is a C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²;
   R^{1b″} is —OR¹¹;
   R^{1b′″} is a —SO₂NR¹¹R¹², —SO₂R¹³, —CONR¹¹R¹², —COR¹³; —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²;
   R^{1b″″} is a C₁-C₆ alkyl substituted with one or more hydroxy;
   at least one R^{6′} is ortho to the bond connecting the B ring to the NH(CO) group of Formula AA-1 and Formula AA-4;
   at least one R^{6″} is ortho to the bond connecting the B ring to the NH(CO) group of Formula AA-2;
   at least one R^{6′} is ortho to the bond connecting the B ring to the NH(CO) group of Formula AA-5;
   at least one R^{6′″} is ortho to the bond connecting the B ring to the NH(CO) group of Formula AA-3;
   R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C=NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
   R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₀ alkenyl,
   wherein R⁶ and R⁷ are each optionally substituted with one or more substituents independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
   or at least one pair of R⁶ and R⁷ on adjacent atoms, taken together with the atoms connecting them, independently form at least one C₄-C₅ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH2, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₆-C₁₀ alkynyl, C₆-C₁₀ aryloxy, and S(O₂)C₁-C₆ alkyl; and wherein the C₁-C₆ alkyl or C₁-C₆ alkoxy that R^{6′} or R^{7″} is substituted with is optionally substituted with one or more hydroxyl, halo, C₆-C₁₀ aryl or NR⁸R⁹, or wherein R^{6′} or R^{7′} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alky), C₆-C₁₀ aryl, and CONR⁸R⁹;
   R^{6″} and R^{7″} are each independently selected from C₁-C₆ alkyl, C₁-C₆ haloalkyl, C₁-C₆ alkoxy, C₁-C₆ haloalkoxy, F, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R^{6″} and R^{7″} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl, and wherein the C₁-C₆ alkyl or C₁-C₆ alkoxy that R⁶ or R^{7″} is substituted with is optionally substituted with one or more hydroxyl, halo, C₁-C₆ aryl or NR⁸R⁹, or wherein R^{6″} or R^{7″} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ and, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R^{6″} and R^{7″} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ and, and CONR⁸R⁹;
   R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
   wherein R^{6′″} and R^{7′″} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6′″} or R^{7′″} is substituted with is optionally substituted with one or more hydroxyl, halo, C₆-C₁₀ aryl or NR⁸R⁹, or wherein R^{6′″} or R^{7′″} is optionally fused to a five- to -seven-membered carbocyclic ring or heterocyclic ring containing one or two heteroatoms independently selected from oxygen, sulfur and nitrogen,
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄, C₆, C₇, or C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   R¹⁰ is C₁-C₆ alkyl;
   each of R⁸ and R⁹ at each occurrence is independently selected from hydrogen, C₁-C₆ alkyl, (C=NR¹³)NR¹¹R¹², S(O)₂C₁-C₆ alkyl, S(O₂)NR¹¹R¹², COR¹³, CO₂R¹³ and CONIC R¹ wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or
   R⁸ and R⁹ taken together with the nitrogen they are attached to form a 3- to 7-membered ring optionally containing one or more heteroatoms in addition to the nitrogen they are attached to;
   R¹³ is C₁-C₆ alkyl, C₆-C₁₀ aryl, or 5- to 10-membered heteroaryl;
   each of R¹¹ and R¹² at each occurrence is independently selected from hydrogen and C₁-C₆ alkyl optionally substituted with hydroxy;
   or a pharmaceutically acceptable salt thereof.

In some embodiments the variables shown in the formulae herein are as follows;

The Formula AA

In some embodiments, Formula AA is Formula AA-1

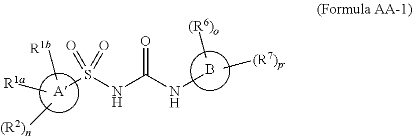

In some embodiments, Formula AA is Formula AA-2

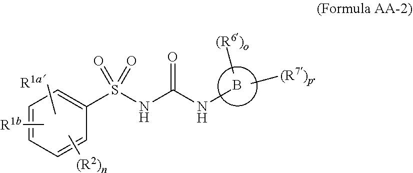

In some embodiments, Formula AA is Formula AA-3

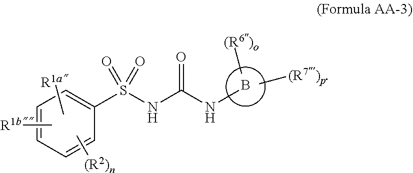

In some embodiments, Formula AA is Formula AA-4

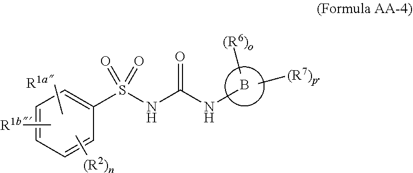

In some embodiments. Formula AA is Formula AA-5

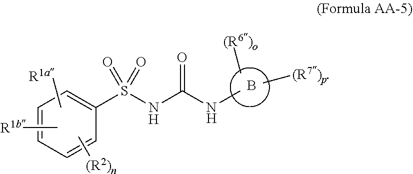

In some embodiments. Formula AA is Formula AA-6

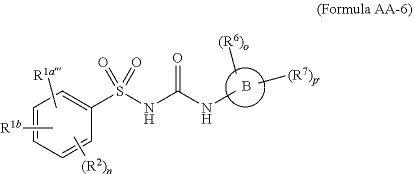

The variable n

In some embodiments n0 or 1. In some embodiments n=0. In some embodiments n=1.

The Ring a and Substitutions on the Ring A

In some embodiments, A is a 5- to 10-membered heteroaryl. In some embodiments, A is a 5- to 6-membered heteroaryl. In some embodiments, A is 5-membered heteroaryl. In some embodiments, A is 6-membered heteroaryl. In some embodiments, A is 10-membered heteroaryl. In some embodiments, A is a monocyclic heteroaryl. In some embodiments, A is a bicyclic heteroaryl. In some embodiments, A is 5-membered heteroaryl including 1-2 (e.g., 1) nitrogen ring members. In some embodiments, A is 5-membered heteroaryl including 1 nitrogen ring member and 1 oxygen ring member. In some embodiments, A is oxazolyl, and n is 0. In some embodiments, A is isoxazolyl, and n is 0. In some embodiments, A is imidazoiyl, and n is 0. In some embodiments, A is imidazoiyl, and n is 1. In some embodiments, A is thiazolyl, and n is 0. In some embodiments, A is a 5- to 6-membered (e.g., 5-membered) heteroaryl containing 1-2 sulfur ring members. In some embodiments, A is a 5-membered heteroaryl containing 1 sulfur ring member. In some embodiments, A is a 5-membered heteroaryl containing a sulfur ring member and one or more nitrogen ring member. In some embodiments, A is a 5-membered heteroaryl containing a sulfur ring member and a nitrogen ring member. In some embodiments, A is a 5-to-10-membered heteroaryl other than pyrazolyl. In some embodiments, A is a 5-to-10-membered heteroaryl other than pyrazolyl (e.g., 3-pyrazolyl), pyrimidinyl, pyridazinyl, pyridyl, triazolyl, and pyrazinyl. In some embodiments, A is selected from the group consisting of: oxazolyl, isoxazolyl, imidazoiyl, thiazolyl, furan, pyridyl, 4-pyrazolyl, isothiazolyl, triazinyl, pyrrolyl, thiadiazolyl, and thiophenyl. In some embodiments, A is selected from the group consisting of: oxazolyl, isoxazolyl, imidazoiyl, thiazolyl, furan, pyridyl, 4-pyrazolyl, isothiazolyl, triazinyl, pyrrolyl, thiadiazolyl, pyrimidinyl, pyridazinyl, pyridyl, triazolyl, pyrazinyl, and thiophenyl. In some embodiments, A is thiazolyl, and n is 0. In some embodiments, A is isothiazolyl, and n is 0.

In some embodiments, the substituted ring A

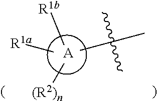

is

In some embodiments, the substituted ring A is

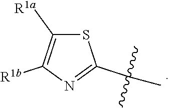

In some embodiments, the substituted ring A is

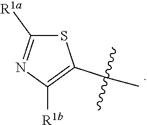

In some embodiments, the substituted ring A is

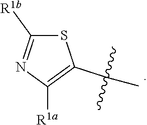

In some embodiments, the substituted ring A is

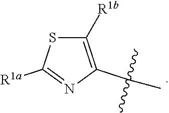

In some embodiments, the substituted ring A is

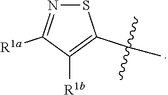

In some embodiments, the substituted ring A is

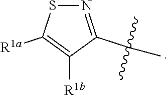

In some embodiments, the substituted ring A is

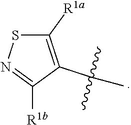

In some embodiments, the substituted ring A is

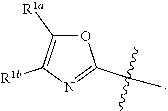

In some embodiments, the substituted ring A is

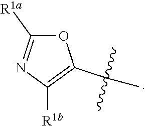

In some embodiments, the substituted ring A is

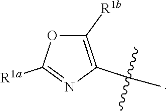

In some embodiments, the substituted ring A is

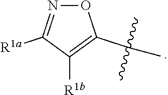

In some embodiments, the substituted ring A is

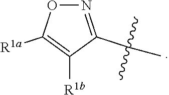

In some embodiments, the substituted ring A is

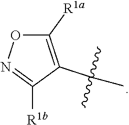

In some embodiments, the substituted ring A is

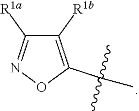

In some embodiments, the substituted ring A is

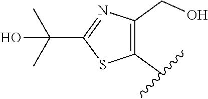 [SMILES: *c1sc(C(C)(C)O)nc1CO]

In some embodiments, the substituted ring A is

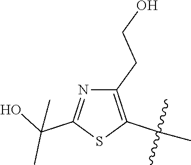 [SMILES: *c1sc(C(C)(C)O)nc1CCO]

In some embodiments, A is C₆-C₁₀ aryl.
In some embodiments, A is

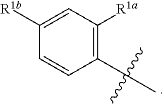

In some embodiments, A is

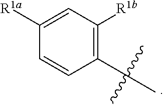

In some embodiments, A is

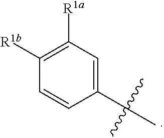

In some embodiments, A is

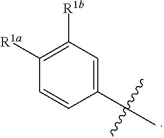

In some embodiments, A is

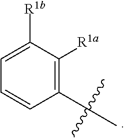

In some embodiments, A is

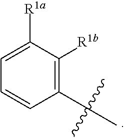

In some embodiments, A is

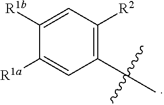

In some embodiments. A is

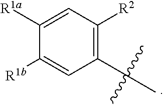

In some embodiments, A is

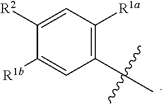

In some embodiments, A is

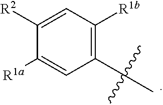

In some embodiments, A is

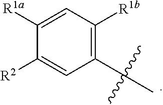

In some embodiments, A is

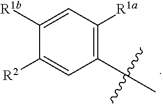

In some embodiments, A is

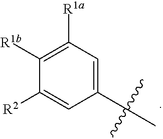

In some embodiments, A is

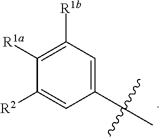

In some embodiments, A is

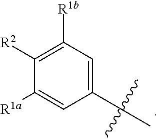

In some embodiments, A is

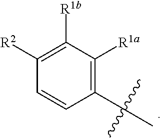

In some embodiments, A is

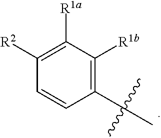

In some embodiments, A is

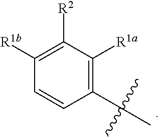

In some embodiments, A is

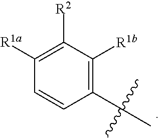

In some embodiments, A is

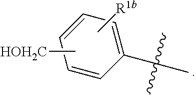

In some embodiments, A is

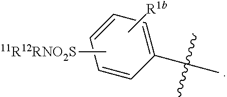

In some embodiments, A is

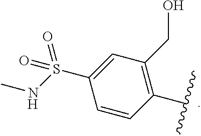

In some embodiments, A is

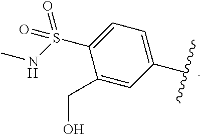

The groups R^{1a}, R^{1b}, R^{1b′}, R^{1b″}, and R^{1b′″}

In some embodiments, R^{1a} is a C₁-C₆ alkyl or —SO₂NR¹¹R¹²;
wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —COR¹³, —CO₂R¹³, —NR¹³CONR¹¹R¹²; —CR¹¹R¹²CN, —NR¹¹SO₂R¹³, —NR¹¹CONR¹², —CR¹¹R¹²NR¹¹R¹², and —NR¹¹COR¹².

In some embodiments, R^{1a} is a C₁-C₆ alkyl or —SO₂NR¹¹R¹²;
wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —COR¹³, —CO₂R¹³, —NR¹³CONR¹¹R¹²; —CR¹¹R¹²CN, —NR¹¹SO₂R¹³, —NR¹¹CONR¹², and —NR¹¹COR¹².

In some embodiments, R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹², and —NR¹¹COR¹².

In some embodiments, one of R^{1a} and R^{1b} is C₁-C₆ alkyl substituted by one hydroxy, and the other one of R^{1a} and R^{1b} is C₁-C₆ alkyl substituted by one hydroxy. In some embodiments, one of R^{1a} and R^{1b} is C₁-C₆ alkyl substituted by two hydroxy, and the other one of R^{1a} and R^{1b} is C₁-C₆ alkyl substituted by one hydroxy. In some embodiments, one of R^{1a} and R^{1b} is C₁-C₅ alkyl substituted by one hydroxy, and the other one of R^{1a} and R^{1b} is C₁-C₅ alkyl substituted by one hydroxy. In some embodiments, one of R^{1a} and R^{1b} is C₁ alkyl substituted by one hydroxy, and the other one of R^{1a} and R^{1b} is C₁ alkyl substituted by one hydroxy. In some embodiments, one of R^{1a} and R^{1b} is C₁ alkyl substituted by one hydroxy, and the other one of R^{1a} and R^{1b} is C₂ alkyl substituted by one hydroxy. In some embodiments, one of R^{1a} and R^{1b} is C₁ alkyl substituted by one hydroxy, and the other one of R^{1a} and R^{1b} is C₃ alkyl substituted by one hydroxy. In some embodiments, one of R^{1a} and R^{1b} is C₁ alkyl substituted by one hydroxy, and the other one of R^{1a} and R^{1b} is C₄ alkyl substituted by one hydroxy. In some embodiments, one of R^{1a} and R^{1b} is C₁ alkyl substituted by one hydroxy, and the other one of R^{1a} and R^{1b} is C₅ alkyl substituted by one hydroxy. In some embodiments, one of R^{1a} and R^{1b} is C₁ alkyl substituted by one hydroxy, and the other one of R^{1a} and R^{1b} is C₆ alkyl substituted by one hydroxy. In some embodiments, one of R^{1a} and R^{1b} is C₁ alkyl substituted by one hydroxy, and the other one of R^{1a} and R^{1b} is C₁ alkyl substituted by one hydroxy. In some embodiments, one of R^{1a} and R^{1b} is C₂ alkyl substituted by one hydroxy, and the other one of R^{1a} and R^{1b} is C₂ alkyl substituted by one hydroxy. In some embodiments, one of R^{1a} and R^{1b} is C₂ alkyl substituted by one hydroxy, and the other one of R^{1a} and R¹⁰ is C₃ alkyl substituted by one hydroxy. In some embodiments, one of R^{1a} and R^{1b} is C₂ alkyl substituted by one hydroxy, and the other one of R^{1a} and R^{1b} is C₄ alkyl substituted by one hydroxy. In some embodiments, one of R^{1a} and R^{1b} is C₂ alkyl substituted by one hydroxy, and the other one of R^{1a} and R^{1b} is C₅ alkyl substituted by one hydroxy. In some embodiments, one of R^{1a} and R^{1b} is C₂ alkyl substituted by one hydroxy, and the other one of R^{1a} and R^{1b} is C₆ alkyl substituted by one hydroxy. In some embodiments, one of R^{1a} and R^{1b} is C₃ alkyl substituted by one hydroxy, and the other one of R^{1a} and R^{1b} is C₁ alkyl substituted by one hydroxy. In some embodiments, one of R^{1a} and R^{1b} is C₃ alkyl substituted by one hydroxy, and the other one of R^{1a} and R^{1b} is C₂ alkyl substituted by one hydroxy. In some embodiments, one of R^{1a} and R^{1b} is C₃ alkyl substituted by one hydroxy, and the other one of R^{1a} and R^{1b} is C₃ alkyl substituted by one hydroxy. In some embodiments, one of R^{1a} and R^{1b} is C₃ alkyl substituted by one hydroxy, and the other one of R^{1a} and R^{1b} is C₄ alkyl substituted by one hydroxy. In some embodiments, one of R^{1a} and R^{1b} is C₃ alkyl substituted by one hydroxy, and the other one of R^{1a} and R^{1b} is C₅ alkyl substituted by one hydroxy. In some embodiments, one of R^{1a} and R^{1b} is C₃ alkyl substituted by one hydroxy, and the other one of R^{1a} and R^{1b} is C₆ alkyl substituted by one hydroxy. In some embodiments, one of R^{1a} and R^{1b} is C₄ alkyl substituted by one hydroxy, and the other one of R^{1a} and R^{1b} is C₁ alkyl substituted by one hydroxy. In some embodiments, one of R^{1a} and R^{1b} is C₄ alkyl substituted by one hydroxy, and the other one of R^{1a} and R^{1b} is C₂ alkyl substituted by one hydroxy. In some embodiments, one of R^{1a} and R^{1b} is C₄ alkyl substituted by one hydroxy, and the other one of R^{1a} and R^{1b} is C₃ alkyl substituted by one hydroxy. In some embodiments, one of R^{1a} and R^{1b} is C₄ alkyl substituted by one hydroxy, and the other one of R^{1a} and R^{1b} is C₄ alkyl substituted by one hydroxy. In some embodiments, one of R^{1a} and R^{1b} is C₄ alkyl substituted by one hydroxy, and the other one of R^{1a} and R^{1b} is C₅ alkyl substituted by one hydroxy. In some embodiments, one of R^{1a} and R^{1b} is C₄ alkyl substituted by one hydroxy, and the other one of R^{1a} and R^{1b} is C₆ alkyl substituted by one hydroxy. In some embodiments, one of R^{1a} and R^{1b} is C₅ alkyl substituted by one hydroxy, and the other one of R^{1a} and R^{1b} is C₁ alkyl substituted by one hydroxy. In some embodiments, one of R^{1a} and R^{1b} is C₅ alkyl substituted by one hydroxy, and the other one of R^{1a} and R^{1b} is C₂ alkyl substituted by one hydroxy. In some embodiments, one of R^{1a} and R^{1b} is C₅ alkyl substituted by one hydroxy, and the other one of R^{1a} and R^{1b} is C₃ alkyl substituted by one hydroxy. In some embodiments, one of R^{1a} and R^{1b} is C₅ alkyl substituted by one hydroxy, and the other one of R^{1a} and R^{1b} is C₄ alkyl substituted by one hydroxy. In some embodiments, one of R^{1a} and R^{1b} is C₅ alkyl substituted by one hydroxy, and the other one of R^{1a} and R^{1b} is C₅ alkyl substituted by one hydroxy. In some embodiments, one of R^{1a} and R^{1b} is C₅ alkyl substituted by one hydroxy, and the other one of R^{1a} and R^{1b} is C₆ alkyl substituted by one hydroxy. One of R^{1a} and R^{1b} is C₆ alkyl substituted by one hydroxy, and the other one of R^{1a} and R^{1b} is C₁ alkyl substituted by one hydroxy. In some embodiments, one of R^{1a} and R^{1b} is C₆ alkyl substituted by one hydroxy, and the other one of R^{1a} and R^{1b} is C₂ alkyl substituted by one hydroxy. In some embodiments, one of R^{1a} and R^{1b} is C₆ alkyl substituted by one hydroxy, and the other one of R^{1a} and R^{1b} is C₃ alkyl substituted by one hydroxy. In some embodiments, one of R^{1a} and R^{1b} is C₆ alkyl substituted by one hydroxy, and the other one of R^{1a} and R^{1b} is C₄ alkyl substituted by one hydroxy. In some embodiments, one of R^{1a} and R^{1b} is C₆ alkyl substituted by one hydroxy, and the other one of R^{1a} and R^{1b} is C₅ alkyl substituted by one hydroxy. In some embodiments, one of R^{1a} and R^{1b} is C₆ alkyl substituted by one hydroxy, and the other one of R^{1a} and R^{1b} is C₆ alkyl substituted by one hydroxy. In some embodiments, one of R^{1a} and R^{1b} is C₁ alkyl substituted by one hydroxy, and the other one of R^{1a} and R^{1b} is C₁ alkyl substituted by two hydroxy. In some embodiments, one of R^{1a} and R^{1b} is C₁ alkyl substituted by one hydroxy, and the other one of R^{1a} and R^{1b} is C₃ alkyl substituted by two hydroxy. In some embodiments, one of R^{1a} and R^{1b} is C₅ alkyl substituted by one hydroxy, and the other one of R^{1a} and R^{1b} is C₄ alkyl substituted by two hydroxy. In some embodiments, one of R^{1a} and R^{1b} is C₁ alkyl substituted by one hydroxy, and the other one of R^{1a} and R^{1b} is C₅ alkyl substituted by two hydroxy. In some embodiments, one of R^{1a} and R^{1b} is C₁ alkyl substituted by one hydroxy, and the other one of R^{1a} and R^{1b} is C₆ alkyl substituted by two hydroxy. In some embodiments, one of R^{1a} and R^{1b} is C₂ alkyl substituted by one hydroxy, and the other one of R^{1a} and R^{1b} is C₂ alkyl substituted by two hydroxy. In some embodiments, one of R^{1a} and R^{1b} is C₂ alkyl substituted by one hydroxy, and the other one of R^{1a} and R^{1b} is C₃ alkyl substituted by two hydroxy. In some embodiments, one of R^{1a} and R^{1b} is C₂ alkyl substituted by one hydroxy, and the other one of R^{1a} and R^{1b} is C₄ alkyl substituted by two hydroxy. In some embodiments, one of R^{1a} and R^{1b} is C₂ alkyl substituted by one hydroxy, and the other one of R^{1a} and R^{1b} is C₅ alkyl substituted by two hydroxy. In some embodiments, one of R^{1a} and R^{1b} is C₂ alkyl substituted by one hydroxy, and the other one of R^{1a} and R^{1b} is C& alkyl substituted by two hydroxy. In some embodiments, one of R^{1a} and R^{1b} is C₃ alkyl substituted by one hydroxy, and the other one of R^{1a} and R^{1b} is C₂ alkyl substituted by two hydroxy. In some embodiments, one of R^{1a} and R^{1b} is C₃ alkyl substituted by one hydroxy, and the other one of R^{1a} and R^{1b} is C₃ alkyl substituted by two hydroxy. In some embodiments, one of R^{1a} and R^{1b} is C₃ alkyl substituted by one hydroxy, and the other one of R^{1a} and R^{1b} is C₄ alkyl substituted by two hydroxy. In some embodiments, one of R^{1a} and R^{1b} is C₃ alkyl substituted by one hydroxy, and the other one of R^{1a} and R^{1b} is C₅ alkyl substituted by two hydroxy. In some embodiments, one of R^{1a} and R^{1b} is C₃ alkyl substituted by one hydroxy, and the other one of R^{1a} and R^{1b} is C₆ alkyl substituted by two hydroxy. In some embodiments, one of R^{1a} and R^{1b} is C₄ alkyl substituted by one hydroxy, and the other one of R^{1a} and R^{1b} is C₂ alkyl substituted by two hydroxy. In some embodiments, one of R^{1a} and R^{1b} is C₄ alkyl substituted by one hydroxy, and the other one of R^{1a} and R^{1b} is €3 alkyl substituted by two hydroxy. In some embodiments, one of R^{1a} and R^{1b} is C₄ alkyl substituted by one hydroxy, and the other one of R^{1a} and R^{1b} is C₄ alkyl substituted by two hydroxy. In some embodiments, one of R^{1a} and R^{1b} is C₄ alkyl substituted by one hydroxy, and the other one of R^{1a} and R^{1b} is C₅ alkyl substituted by two hydroxy. In some embodiments, one of R^{1a} and R^{1b} is C₄ alkyl substituted by one hydroxy, and the other one of R^{1a} and R^{1b} is C₆ alkyl substituted by two hydroxy. In some embodiments, one of R^{1a} and R^{1b} is C₅ alkyl substituted by one hydroxy, and the other one of R^{1a} and R^{1b} is C₂ alkyl substituted by two hydroxy. In some embodiments, one of R^{1a} and R^{1b} is C₅ alkyl substituted by one hydroxy, and the other one of R^{1a} and R^{1b} is C₃ alkyl substituted by two hydroxy. In some embodiments, one of R^{1a} and R^{1b} is C₅ alkyl substituted by one hydroxy, and the other one of R^{1a} and R^{1b} is C₄ alkyl substituted by two hydroxy. In some embodiments, one of R^{1a} and R^{1b} is C₅ alkyl substituted by one hydroxy, and the other one of R^{1a} and R^{1b} is C₅ alkyl substituted by two hydroxy. In some embodiments, one of R^{1a} and R^{1b} is C₅ alkyl substituted by one hydroxy, and the other one of R^{1a} and R^{1b} is C₆ alkyl substituted by two hydroxy. In some embodiments, one of R^{1a} and R^{1b} is C₆ alkyl substituted by one hydroxy, and the other one of R^{1a} and R^{1b} is C₂ alkyl substituted by two hydroxy. In some embodiments, one of R^{1a} and R^{1b} is C₆ alkyl substituted by one hydroxy, and the other one of R^{1a} and R^{1b} is C₃ alkyl substituted by two hydroxy. In some embodiments, one of R^{1a} and R^{1b} is C₆ alkyl substituted by one hydroxy, and the other one of R^{1a} and R^{1b} is C₄ alkyl substituted by two hydroxy. In some embodiments, one of R^{1a} and R^{1b} is C₆ alkyl substituted by one hydroxy, and the other one of R^{1a} and R^{1b} is C₅ alkyl substituted by two one hydroxy. In some embodiments, one of R^{1a} and R^{1b} is C₆ alkyl substituted by one hydroxy, and the other one of R^{1a} and R^{1b} is C₆ alkyl substituted by two hydroxy.

In some embodiments of any of the formulae herein, hydroxyethyl is 1-hydroxyethyl.

In some embodiments of any of the formulae herein, hydroxyethyl is 2-hydroxy ethyl.

In any of the foregoing embodiments, the R^{1a} and/or R^{1b} C₃ alkyl is n-propyl.

In any of the foregoing embodiments, the R^{1a} and/or R^{1b} C₃ alkyl is isopropyl.

In any of the foregoing embodiments, the R^{1a} and/or R^{1b} C₄ alkyl is n-butyl.

In any of the foregoing embodiments, the R^{1a} and/or R^{1b} C₄ alkyl is isobutyl.

In any of the foregoing embodiments, the R^{1a} and/or R^{1b} C₄ alkyl is n-butyl.

In any of the foregoing embodiments, the R^{1a} and/or R^{1b} C₅ alkyl is n-pentyl.

In any of the foregoing embodiments, the R^{1a} and/or R^{1b} C₅ alkyl is 2-methylbutan-2-yl.

In any of the foregoing embodiments, the R^{1a} and/or R^{1b} C₅ alkyl is 2,2-dimethyl propyl.

In any of the foregoing embodiments, the R^{1a} and/or R^{1b} C₅ alkyl is 3-methylbutyl.

In any of the foregoing embodiments, the R^{1a} and/or R^{1b} C₅ alkyl is pentan-2-yl.

In any of the foregoing embodiments, the R^{1a} and/or R^{1b} C₅ alkyl is pentan-3-yl.

In any of the foregoing embodiments, the R^{1a} and/or R^{1b} C₅ alkyl is 3-methylbutan-2-yl.

In any of the foregoing embodiments, the R^{1a} and/or R^{1b} C₅ alkyl is 2-methylbutyl.

In any of the foregoing embodiments, the R^{1a} and/or R^{1b} C₄ alkyl is branched.

In any of the foregoing embodiments, the R^{1a} and/or R^{1b} C₅ alkyl is branched.

In any of the foregoing embodiments, the R^{1a} and/or R^{1b} C₆ alkyl is branched.

In some embodiments, one of R^{1a} and R^{1b} is hydroxymethyl, and the other one of R^{1a} and R^{1b} is hydroxymethyl. In some embodiments, one of R^{1a} and R^{1b} is hydroxymethyl, and the other one of R^{1a} and R^{1b} is hydroxyethyl (e.g., 1-hydroxyethyl or 2-hydroxyethyl). In some embodiments, one of R^{1a} and R^{1b} is hydroxymethyl, and the other one of R^{1a} and R^{1b} is 2-hydroxy-2-propyl. In some embodiments, one of R^{1a} and R^{1b} is hydroxymethyl, and the other one of R^{1a} and R^{1b} is 3-hydroxy-2-propyl. In some embodiments, one of R^{1a} and R^{1b} is hydroxymethyl, and the other one of R^{1a} and R^{1b} is 1-hydroxy-1-propyl. In some embodiments, one of R^{1a} and R^{1b} is hydroxymethyl, and the other one of R^{1a} and R^{1b} is 2-hydroxy-1-propyl. In some embodiments, one of R^{1a} and R^{1b} is hydroxymethyl, and the other one of R^{1a} and R^{1b} is 3-hydroxy-1-propyl. In some embodiments, one of R^{1a} and R^{1b} is hydroxymethyl, and the other one of R^{1a} and R^{1b} is hydroxybutyl (e.g., 4-hydroxy-1-butyl). In some embodiments, one of R^{1a} and R^{1b} is hydroxymethyl, and the other one of R^{1a} and R^{1b} is hydroxypentyl (e.g., 5-hydroxy-1-pentyl). In some embodiments, one of R^{1a} and R^{1b} is hydroxymethyl, and the other one of R^{1a} and R^{1b} is hydroxyhexyl (e.g., 6-hydroxy-1-hexyl). In some embodiments, one of R^{1a} and R^{1b} is hydroxyethyl, and the other one of R^{1a} and R^{1b} is hydroxymethyl. In some embodiments, one of R^{1a} and R^{1b} is hydroxyethyl, and the other one of R^{1a} and R^{1b} is hydroxyethyl. In some embodiments, one of R^{1a} and R^{1b} is hydroxyethyl, and the other one of R^{1a} and R^{1b} is 2-hydroxy-2-propyl. In some embodiments, one of R^{1a} and R^{1b} is hydroxyethyl, and the other one of R^{1a} and R^{1b} is 3-hydroxy-2-propyl. In some embodiments, one of R^{1a} and R^{1b} is hydroxyethyl, and the other one of R^{1a} and R^{1b} is 1-hydroxy-1-propyl. In some embodiments, one of R^{1a} and R^{1b} is hydroxyethyl, and the other one of R^{1a} and R^{1b} is 2-hydroxy-1-propyl. In some embodiments, one of R^{1a} and R^{1b} is hydroxyethyl, and the other one of R^{1a} and R^{1b} is 3-hydroxy-1-propyl. In some embodiments, one of R^{1a} and R^{1b} is hydroxyethyl, and the other one of R^{1a} and R^{1b} is hydroxybutyl. In some embodiments, one of R^{1a} and R^{1b} is hydroxyethyl, and the other one of R^{1a} and R^{1b} is hydroxypentyl. In some embodiments, one of R^{1a} and R^{1b} is hydroxyethyl, and the other one of R^{1a} and R^{1b} is hydroxyhexyl. In some embodiments, one of R^{1a} and R^{1b} is 2-hydroxy-2-propyl, and the other one of R^{1a} and R^{1b} is hydroxymethyl. In some embodiments, one of R^{1a} and R^{1b} is 2-hydroxy-2-propyl, and the other one of R^{1a} and R^{1b} is hydroxyethyl. In some embodiments, one of R^{1a} and R^{1b} is 2-hydroxy-2-propyl, and the other one of R^{1a} and R¹⁰ is 2-hydroxy-2-propyl. In some embodiments, one of R^{1a} and R^{1b} is 2-hydroxy-2-propyl, and the other one of R^{1a} and R^{1b} is 3-hydroxy-2-propyl. In some embodiments, one of R^{1a} and R^{1b} is 2-hydroxy-2-propyl, and the other one of R^{1a} and R^{1b} is 1-hydroxy-1-propyl. In some embodiments, one of R^{1a} and R^{1b} is 2-hydroxy-2-propyl, and the other one of R^{1a} and R¹⁰ is 2-hydroxy-1-propyl. In some embodiments, one of R^{1a} and R^{1b} is 2-hydroxy-2-propyl, and the other one of R^{1a} and R^{1b} is 3-hydroxy-1-propyl. In some embodiments, one of R^{1a} and R^{1b} is 2-hydroxy-2-propyl, and the other one of R^{1a} and R^{1b} is hydroxybutyl. In some embodiments, one of R^{1a} and R^{1b} is 2-hydroxy-2-propyl, and the other one of R^{1a} and R^{1b} is hydroxypentyl. In some embodiments, one of R^{1a} and R^{1b} is 2-hydroxy-2-propyl, and the other one of R^{1a} and R¹⁰ is hydroxyhexyl.

In some embodiments, R^{1a} is C₁-C₆ alkyl substituted by one hydroxy, and R^{1b} is a C₁-C₆ alkyl substituted with one or more hydroxy, —SO₂NR¹¹R¹², —SO₂R¹¹, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², or —NR¹¹COR¹².

In some embodiments, R^{1a} is C₁-C₆ alkyl substituted by one hydroxy, and R^{1b} is a C₁-C₆ alkyl substituted with one or more hydroxyl, —SO₂NR¹¹R¹², —SO₂R¹³, —CONR¹¹R¹², —OR¹¹, —COR¹¹; —NR¹³CONR¹¹R¹²; —CR¹¹R¹²CN, —NR¹¹SO₂R¹³, —NR¹¹CONR¹¹R¹², —CR¹¹R¹²NR¹¹R¹², or —NR¹¹COR¹².

In some embodiments, R^{1a} is C₁-C₆ alkyl substituted by one hydroxy, and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN. In some embodiments, R^{1a} is C₁-C₆ alkyl substituted by one hydroxy, and R^{1b} is a —SO₂NHMe, SO₂NHCH₂CH₂OH, SO₂Me, CONHMe, or OMe.

In some embodiments, R^{1a} is C₁-C₆ alkyl substituted by one hydroxy, and R^{1b} is a —SO₂NHMe or OMe.

In some embodiments, R^{1a} is C₁-C₆ alkyl substituted by one hydroxy, and R^{1b}—SO₂NR¹¹R¹², —SO₂R¹³, —CONR¹¹R¹², —OR¹¹, —COR¹³; —CO₂R¹³, NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a} is C₁-C₆ alkyl substituted by one hydroxy, and R^{1b} is —SO₂NR¹¹R¹².

In some embodiments, R^{1a} is C₁-C₆ alkyl substituted by one hydroxy, and R^{1b} is —SO₂R¹³.

In some embodiments, R^{1a} is C₁-C₆ alkyl substituted by one hydroxy, and R^{1b} is —CONR¹¹R¹².

In some embodiments, R^{1a} is C₁-C₆ alkyl substituted by one hydroxy, and R^{1b} is —OR¹¹.

In some embodiments, R^{1a} is C₁-C₆ alkyl substituted by one hydroxy, and R^{1b} is —COR¹³.

In some embodiments, R^{1a} is C₁-C₆ alkyl substituted by one hydroxy, and R^{1b} is —CO₂R¹³.

In some embodiments, R^{1a} is C₁-C₆ alkyl substituted by one hydroxy, and R^{1b} is —NR¹³CONR¹¹R¹².

In some embodiments, R^{1a} is C₁-C₆ alkyl substituted by one hydroxy, and R^{1b} is —CR¹¹R¹²CN.

In some embodiments, R^{1a} is C₁-C₆ alkyl substituted by one hydroxy, and R^{1b} is —NR¹¹SO₂R¹³.

In some embodiments, R^{1a} is C₁-C₆ alkyl substituted by one hydroxy, and R^{1b} is —NR¹¹CONR¹¹R¹².

In some embodiments, R^{1a} is C₁-C₆ alkyl substituted by one hydroxy, and R^{1b} is —NR¹¹ COR¹².

In some embodiments, R^{1a} is C₁-C₆ alkyl substituted by one hydroxy, and R^{1b} is —CR¹¹R¹²NR¹¹R¹².

In some embodiments, R^{1a} is C₁-C₆ alkyl substituted by one —OSi(R¹³)₃, and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —NR¹¹COR¹².

In some embodiments, R^{1a} is C₁-C₆ alkyl substituted by one —OSi(R¹³)₃, and R^{1b} is a C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², or —NR¹¹COR¹².

In some embodiments, R^{1a} is C₁-C₆ alkyl substituted by one —OSi(R¹³)₃, and R¹⁰ is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a} is C₁-C₆ alkyl substituted by one —OSi(R¹³)₃, and R^{1b} is a —SO₂NHMe, SO₂NHCH₂CH₂OH, SO₂Me, CONHMe, or OMe.

In some embodiments, R^{1a} is C₁-C₆ alkyl substituted by one —OSi(R¹³)₃, and R^{1b} is a —SO₂NHMe or OMe.

In some embodiments, R^{1a} is C₁-C₆ alkyl substituted by one —OSi(R¹³)₃, and R^{1b}—SO₂NR¹¹R¹², —SO₂R¹³, —CONR¹¹R¹², —OR¹¹, —COR¹³, —CO₂R¹³, —NR¹³CONR¹¹R¹², —CR¹¹R¹²CN, —NR¹¹SO₂R¹³, —NR¹¹CONR¹¹R¹², or —NR¹¹COR¹².

In some embodiments, R^{1a} is C₁-C₆ alkyl substituted by one —OSi(R¹³)₃, and R¹⁰ is —SO₂NR¹¹R¹².

In some embodiments, R^{1a} is C₁-C₆ alkyl substituted by one —OSi(R¹³)₃, and R^{1b} is —SO₂R¹³.

In some embodiments, R^{1a} is C₁-C₆ alkyl substituted by one OSi(R¹³)₃, and R^{1b} is —CONR¹¹R¹².

In some embodiments, R^{1a} is C₁-C₆ alkyl substituted by one —OSi(R¹³)₃, and R^{1b} is —OR¹¹.

In some embodiments, R^{1a} is C₁-C₆ alkyl substituted by one —OSi(R¹³)₃, and R^{1b} is —COR¹³.

In some embodiments, R^{1a} is C₁-C₆ alkyl substituted by one —OSi(R¹³)₃, and R^{1b} is —CO₂R¹³.

In some embodiments, R^{1a} is C₁-C₆ alkyl substituted by one —OSi(R¹³)₃, and R^{1b} is —NR¹³CONR¹¹R¹².

In some embodiments, R^{1a} is C₁-C₆ alkyl substituted by one —OSi(R¹³)₃, and R¹⁰ is —CR¹¹R¹²CN.

In some embodiments, R^{1a} is C₁-C₆ alkyl substituted by one —OSi(R¹³)₃, and R^{1b} is —NR¹¹SO₂R¹³.

In some embodiments, R^{1a} is C₁-C₆ alkyl substituted by one —OSi(R¹³)₃, and R^{1b} is —NR¹¹CONR¹¹R¹².

In some embodiments, R^{1a} is C₁-C₆ alkyl substituted by one —OSi(R¹³)₃, and R^{1b} is —NR¹¹COR¹².

In some embodiments, R^{1a} is C₁-C₆ alkyl substituted by one —OSi(R¹³)₃, and R^{1b} is —CR¹¹R¹²NR¹¹R¹².

In any of the foregoing embodiments that include —OSi(R¹³)₃, Si(R¹³)₃ is selected from trimethylsilyl, triethylsilyl, triisopropylsilyl, tert-butyldimethylsilyl, and tert-butyldiphenylsilyl.

In any of the foregoing embodiments that include —OSi(R¹³)₃, Si(R¹³)₃ is selected from tert-butyldimethylsilyl.

In some embodiments, R^{1a} is —SO₂NR¹¹R¹², and R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², or —NR¹¹COR¹².

In some embodiments, R^{1a} is —SO₂NR¹¹R¹², and R^{1b} is a C₁-C₆ alkyl substituted with one or more hydroxyl, —SO₂NR¹¹R¹², —SO₂R¹³, —CONR¹¹R¹², —OR¹¹, —COR¹³, —NR¹³CONR¹¹R¹²; —CR¹¹R¹²CN, —NR¹¹SO₂R¹³, —NR¹¹CONR¹¹R¹², or —NR¹¹ COR¹².

In some embodiments, R^{1a} is —SO₂NR¹¹R¹², and R^{1b} is a —SO₂NR¹¹R¹², —SO₂R¹³, —CONR¹¹R¹², —COR¹³, —CO₂R¹³, —NR¹³CONR¹¹R¹²; or —CR¹¹R¹²CN.

In some embodiments, R^{1a} is —SO₂NR¹¹R¹², and R^{1b} is a —SO₂NHMe, SO₂NHCH₂CH₂OH, SO₂Me, CONHMe, or OMe.

In some embodiments, R^{1a} is —SO₂NR¹¹R¹², and R^{1b} is a —SO₂NHMe or OMe.

In some embodiments, R^{1a} is —SO₂NR¹¹R¹², and R^{1b} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹¹, —NR¹¹ CONR¹¹R¹², or —NR¹¹COR¹².

In some embodiments, R^{1a} is C₁-C₆ alkyl substituted by one hydroxy, and R^{1b} is —SO₂NR¹¹R¹². In some embodiments, R^{1a} is —SO₂NR¹¹R¹², and R^{1b} is —SO₂R¹³. In some embodiments, R^{1a} is —SO₂NR¹¹R¹², and R^{1b} is —CONR¹¹R¹². In some embodiments, R^{1a} is —SO₂NR¹¹R¹², and R^{1b} is —OR¹¹. In some embodiments, R^{1a} is —SO₂NR¹¹R¹², and R^{1b} is —COR¹³. In some embodiments, R^{1a} is —SO₂.NR¹¹R¹², and R^{1b} is —CO₂R¹³. In some embodiments, R^{1a} is —SO₂NR¹¹R¹², and R^{1b} is —NR¹³CONR¹¹R¹². In some embodiments, R^{1a} is —SO₂NR¹¹R¹², and R^{1b} is —CR¹¹R¹²CN. In some embodiments, R^{1a} is —SO₂NR¹¹R¹², and R^{1b} is —NR¹¹SO₂R¹³. In some embodiments, R^{1a} is —SO₂NR¹¹R¹², and R^{1b} is —NR¹¹CONR¹¹R¹². In some embodiments, R^{1a} is —SO₂NR¹¹R¹², and R^{1b} is —NR¹¹COR¹². In some embodiments, R^{1a} is —SO₂NR¹¹R¹², and R^{1b} is —CR¹¹R¹²NR¹¹R¹². In some embodiments, R^{1a} is 2-hydroxy-2-propyl, and R^{1b} is —OMe. In some embodiments, R^{1a} is 2-hydroxy-2-propyl, and R^{1b} is —OH. In some embodiments, R^{1a} is 2-hydroxy-2-propyl, and R^{1b} is —CO₂Me. In some embodiments, R^{1a} is 2-hydroxy-2-propyl, and R^{1b} is hydroxymethyl. In some embodiments, R^{1a} is 2-hydroxy-2-propyl, and R^{1b} is hydroxy ethyl. In some embodiments, R^{1a} is 2-hydroxy-2-propyl, and R^{1b} is 2-hydroxy-2-propyl. In some embodiments, R^{1a} is 2-hydroxy-2-propyl, and R^{1b} is —SO₂NHCH₂CH₂OH. In some embodiments, R^{1a} is 2-hydroxy-2-propyl, and R^{1b} is —SO₂Me. In some embodiments, R^{1a} is 2-hydroxy-2-propyl, and R^{1b} is CONHMe. In some embodiments, R^{1a} is 2-hydroxy-2-propyl, and R^{1b} is cyanomethyl. In some embodiments, R^{1a} is 2-hydroxy-2-propyl, and R^{1b} is dimethylaminomethyl. In some embodiments, R^{1a} is —SO₂NHMe, and R^{1b} is —OMe. In some embodiments, R^{1a} is —SO₂NHMe, and R¹⁰ is —OH. In some embodiments, R^{1a} is —SO₂NHMe, and R^{1b} is —CO₂Me. In some embodiments, R^{1a} is —SO₂NHMe, and R^{1b} is hydroxymethyl. In some embodiments, R^{1a} is —SO₂NHMe, and R¹⁰ is hydroxyethyl. In some embodiments, R^{1a} is —SO₂NHMe, and R¹⁰ is 2-hydroxy-2-propyl. In some embodiments, R^{1a} is —SO₂NHMe, and R^{1b} is —SO₂NHCH₂CH₂OH. In some embodiments, R^{1a} is —SO₂NHMe, and R^{1b} is —SO₂Me. In some embodiments, R^{1a} is —SO₂NHMe, and R^{1b} is CONHMe. In some embodiments, R^{1a} is —SO₂NHMe, and R^{1b} is cyanomethyl. In some embodiments, R^{1a} is —SO₂NHMe, and R^{1b} is dimethylaminomethyl. In some embodiments, R^{1a} is C₁-C₄ alkyl substituted with one —OSi(Me)₂ₜBu, and R^{1b} is —CO₂Me.

In some embodiments, R^{1b} is C₁-C₆ alkyl substituted with one or more hydroxy, —SO₂NR¹¹R¹², —SO₂R¹³, —CONR¹¹R¹², —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹². In some embodiments, R¹⁰ is C₁-C₆ alkyl substituted with one or more hydroxy, —SO₂NR¹¹R¹², —SO₂R¹³, —CONR¹¹R¹², —COR¹³; —CO₂R¹³, —NR¹³CONR¹¹R¹²; —CR¹¹R¹²CN, —NR¹¹SO₂R¹³, —NR¹¹CONR¹¹R¹², and —NR¹¹COR¹². In some embodiments, R^{1b} is C₁-C₆ alkyl substituted with one or more hydroxy, —SO₂NR¹¹R¹², —SO₂R¹³, —CONR¹¹R¹², —COR¹³: —CO₂R¹³, —NR¹³CONR¹¹R¹²; —CR¹¹R¹²CN, —NR¹¹SO₂R¹³, —NR¹¹CONR¹¹R¹², or —NR¹¹COR¹². In some embodiments, R^{1b} is C₁-C₆ alkyl substituted with one or more hydroxy, —SO₂NR¹¹R¹², —SO₂R¹³, —CONR¹¹R¹², —COR¹³, —NR¹³CONR¹¹R¹²; —CR¹¹R¹²CN, —NR¹¹SO₂R¹³, —NR¹¹CONR¹¹R¹², or —NR¹¹COR¹². In some embodiments, R^{1b′} is —SO₂NR¹¹R¹², —SO₂R¹³, —CONR¹¹R¹², —COR¹³, —CO₂R¹³, —NR¹³CONR¹¹R¹², or —CR¹¹R¹²CN. In some embodiments, R^{1b′} is —SO₂NHMe, SO₂NHCH₂CH₂OH, SO₂Me, or CONHMe. In some embodiments, R^{1b} is —SO₂NHMe. In some embodiments, R^{1b} is C₁-C₆ alkyl substituted by one hydroxyl (e.g., 2-hydroxy-2-propyl, hydroxymethyl, or hydroxy ethyl). In some embodiments, R^{1b′} is —SO₂NR¹¹R¹². In some embodiments, R^{1b} is —SO₂R¹³. In some embodiments, R^{1b} is —CONR¹¹R¹². In some embodiments, R^{1b′} is —COR¹². In some embodiments, R^{1b′} is —CO₂R¹³. In some embodiments, R^{1b′} is —NR¹³CONR¹¹R¹². In some embodiments, R^{1b′} is —CR¹¹R¹²CN. In some embodiments, R^{1b′} is NR¹¹SO₂R¹³. In some embodiments, R^{1b′} is —NR¹¹CONR¹¹R¹². In some embodiments, R^{1b′} is —NR¹¹COR¹². In some embodiments, R^{1b′} is —CR¹¹R¹²NR¹¹R¹². In some embodiments, R^{1b″} is —OR¹¹ (e.g., OMe or OH).

In some embodiments, R^{1b′″} is a —SO₂NR¹¹R¹², —SO₂R¹³, —CONR¹¹R¹², —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²; In some embodiments, R^{1b′″} is a —SO₂NR¹¹R¹², —SO₂R¹³, —CONR¹¹R¹², —COR¹³; —CO₂R¹³, —NR¹³CONR¹¹R¹²; —CR¹¹R¹²CN, —NR¹¹SO₂R¹³, —NR¹¹CONR¹¹R¹², and —NR¹¹COR¹². In some embodiments, R^{1b′″} is —SO₂NR¹¹R¹², —SO₂R¹³, —CONR¹¹R¹², —CO₂R¹³, —NR¹³CONR¹¹R¹²; —CR¹¹R¹²CN, —NR¹¹SO₂R¹³, —NR¹¹R¹², or —NR¹¹COR¹². In some embodiments, R^{1b′″} is —SO₂NR¹¹R¹², SO₂R¹³, —CONR¹¹R¹², —COR¹³, —CO₂R¹³, —NR¹³CONR¹¹R¹²; or —CR¹¹R¹²CN. In some embodiments, R^{1b′″} is —SO₂NHMe, SO₂NHCH₂CH₂OH, SO₂Me, or CONHMe. In some embodiments, R^{1b′″} is —SO₂NHMe. In some embodiments, R^{1b′″} is —SO₂NR¹¹R¹². In some embodiments, R^{1b′″} is —SO₂R¹³. In some embodiments, R^{1b′″} is —CONR¹¹R¹². In some embodiments, R^{1b′″} is —COR¹³. In some embodiments, R^{1b′″} is —CO₂R¹³. In some embodiments, R^{1b′″} is —NR¹³CONR¹¹R¹². In some embodiments, R^{1b′″} is —CR¹¹R¹²CN. In some embodiments, R^{1b′″} is —NR¹¹SO₂R¹³. In some embodiments, R^{1b′″} is —NR¹¹CONR¹¹R¹². In some embodiments, R^{1b′″} is —NR¹¹COR¹². In some embodiments, R^{1b′″} is is —CR¹¹R¹²NR¹¹R¹².

In some embodiments, R^{1b″″} is a C₁-C₆ alkyl substituted with one or more hydroxyl (e.g., 2-hydroxy-2-propyl, hydroxymethyl, or hydroxy ethyl).

In some embodiments, R^{1b} is not —CO₂R¹³.

The group R²

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₁-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH2, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo, wherein the 3- to 7-membered heterocycloalkyl, C₆-C₁₀ aryl, and 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo; wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ alkyl, halo, CN, NO₂, COC₁-C₆ alkyl, CO—C₆-C₁₀ and, CO(5- to 10-membered heteroaryl), CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo; wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo; wherein the 3- to 7-membered heterocycloalkyl, C₆-C₁₀ and, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₁-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH2, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently-selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl);
wherein the 3- to 7-membered heterocycloalkyl, C₆-C₁₀ aryl, or 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments,

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH2, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl are each unsubstituted.

In some embodiments,

R² is selected from C₁-C₆ alkyl, halo, CN, COC₁-C₆ alkyl, CO₂C₁-C₆ alkyl, C₆-C₁₀ aryl, S(O)C₁-C₆ alkyl, 5- to 10-membered heteroaryl, and 3- to 7-membered heterocycloalkyl,

wherein the C₁-C₆ alkyl and 3- to 7-membered heterocycloalkyl is optionally substituted with one or more substituents each independently selected from hydroxy and oxo.

In some embodiments, n=1; and

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments, n=1; and,

R² is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl, wherein the C₁-C₆ alkyl and 3- to 7-membered heterocycloalkyl is optionally substituted with one or more substituents each independently selected from hydroxy and oxo.

In some embodiments, n=1; and

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₁-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O)NR¹¹R¹², S(O)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl.

In some embodiments, n=1; and,

R² is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl, wherein the C₁-C₆ alkyl and 3- to 7-membered heterocycloalkyl is optionally substituted with one or more substituents each independently selected from hydroxy and oxo.

Particular Embodiments Wherein n=1;

In some embodiments, one of R^{1a} and R^{1b} is hydroxymethyl, the other one of R^{1a} and R^{1b} is hydroxymethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R¹⁰ is hydroxymethyl, the other one of R^{1a} and R^{1b} is hydroxy ethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 2-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 3-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 1-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 2-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is 3-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxybutyl, and R² is C₁-C₆ alkyl optionally-substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxypentyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxymethyl, the other one of R^{1a} and R^{1b} is hydroxyhexyl, and R² is C₁-C₆ alkyl optionally substituted with one or more hydroxyl (e.g., methyl, isopropyl, 2-hydroxyl-propyl, or 1-hydroxyethyl). In some embodiments, one of R^{1a} and R^{1b} is hydroxyethyl, the other one of R^{1a} and R^{1b} is hydroxymethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R¹⁰ is hydroxyethyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is 2-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is 3-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is 1-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is 2-hydroxy-1-propyl, and R² is C₁-C₆ alkyl optionally substituted with one or more hydroxyl (e.g., methyl, isopropyl, 2-hydroxy-2-propyl, or 1-hydroxy ethyl). In some embodiments, one of R^{1a} and R^{1b} is hydroxy ethyl, the other one of R^{1a} and R^{1b} is 3-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is hydroxybutyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R^{1b} is hydroxypentyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is hydroxyethyl, the other one of R^{1a} and R¹⁰ is hydroxyhexyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 2-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 3-hydroxy-2-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 1-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is 2-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R¹⁰ is 2-hydroxy-2-propyl, the other one of R^{1a} and R^{1b} is 3-hydroxy-1-propyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is hydroxybutyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is hydroxypentyl, and R² is C₁-C₆ alkyl optionally substituted with one or more hydroxyl (e.g., methyl, isopropyl, 2-hydroxy-2-propyl, or 1-hydroxyethyl). In some embodiments, one of R^{1a} and R^{1b} is 2-hydroxy-2-propyl, the other one of R^{1a} and R^{1b} is hydroxyhexyl, and R² is C₁-C₆ alkyl optionally substituted with one or more hydroxyl (e.g., methyl, isopropyl, 2-hydroxyl-propyl, or 1-hydroxyethyl). In some embodiments, one of R^{1a} and R^{1b} is hydroxymethyl, the other one of R^{1a} and R^{1b} is hydroxymethyl, and R² is C₆-C₁₀ aryl (e.g., phenyl). In some embodiments, one of R^{1a} and R^{1b} is hydroxymethyl, the other one of R^{1a} and R^{1b} is hydroxyethyl, and R² is C₆-C₁₀ aryl (e.g., phenyl). In some embodiments, one of R^{1a} and R^{1b} is hydroxymethyl, the other one of R^{1a} and R^{1b} is 2-hydroxy-2-propyl, and R² is C₆-C₁₀ aryl (e.g., phenyl). In some embodiments, one of R^{1a} and R^{1b} is hydroxymethyl, the other one of R^{1a} and R^{1b} is 3-hydroxy-2-propyl, and R² is C₆-C₁₀ aryl (e.g., phenyl). In some embodiments, one of R^{1a} and R^{1b} is hydroxymethyl, the other one of R^{1a} and R^{1b} is 1-hydroxy-1-propyl, and R² is C₆-C₁₀ aryl (e.g., phenyl). In some embodiments, one of R^{1a} and R^{1b} is hydroxymethyl, the other one of R^{1a} and R^{1b} is 2-hydroxy-1-propyl, and R² is C₆-C₁₀ aryl (e.g., phenyl). In some embodiments, one of R^{1a} and R^{1b} is hydroxymethyl, the other one of R^{1a} and R^{1b} is 3-hydroxy-1-propyl, and R² is C₆-C₁₀ aryl (e.g., phenyl). In some embodiments, one of R^{1a} and R^{1b} is hydroxymethyl, the other one of R^{1a} and R^{1b} is hydroxybutyl, and R² is C₆-C₁₀ aryl (e.g., phenyl). In some embodiments, one of R^{1a} and R^{1b} is hydroxymethyl, the other one of R^{1a} and R^{1b} is hydroxypentyl, and R² is C₆-C₁₀ aryl (e.g., phenyl). In some embodiments, one of R^{1a} and R^{1b} is hydroxymethyl, the other one of R^{1a} and R^{1b} is hydroxyhexyl, and R² is C₆-C₁₀ aryl (e.g., phenyl). In some embodiments, one of R^{1a} and R^{1b} is hydroxyethyl, the other one of R^{1a} and R^{1b} is hydroxyethyl, and R² is C₆-C₁₀ aryl (e.g., phenyl). In some embodiments, one of R^{1a} and R^{1b} is hydroxyethyl, the other one of R^{1a} and R^{1b} is 2-hydroxy-2-propyl, and R² is C₆-C₁₀ aryl (e.g., phenyl). In some embodiments, one of R^{1a} and R^{1b} is hydroxyethyl, the other one of R^{1a} and R^{1b} is 3-hydroxy-2-propyl, and R² is C₆-C₁₀ aryl (e.g., phenyl). In some embodiments, one of R^{1a} and R^{1b} is hydroxyethyl, the other one of R^{1a} and R^{1b} is 1-hydroxy-1-propyl, and R² is C₆-C₁₀ aryl (e.g., phenyl). In some embodiments, one of R^{1a} and R¹⁰ is hydroxyethyl, the other one of R^{1a} and R^{1b} is 2-hydroxy-1-propyl, and R² is C₆-C₁₀ aryl (e.g., phenyl). In some embodiments, one of R^{1a} and R^{1b} is hydroxyethyl, the other one of R^{1a} and R^{1b} is 3-hydroxy-1-propyl, and R² is C₆-C₁₀ aryl (e.g., phenyl). In some embodiments, one of R^{1a} and R^{1b} is hydroxyethyl, the other one of R^{1a} and R^{1b} is hydroxybutyl, and R² is C₆-C₁₀ aryl (e.g., phenyl). In some embodiments, one of R^{1a} and R^{1b} is hydroxyethyl, the other one of R^{1a} and R^{1b} is hydroxypentyl, and R² is C₆-C₁₀ aryl (e.g., phenyl). In some embodiments, one of R^{1a} and R^{1b} is hydroxyethyl, the other one of R^{1a} and R^{1b} is hydroxyhexyl, and R² is C₆-C₁₀ aryl (e.g., phenyl). In some embodiments, one of R^{1a} and R^{1b} is 2-hydroxy-2-propyl, the other one of R^{1a} and R¹⁰ is 2-hydroxy-2-propyl, and R² is C₆-C₁₀ aryl (e.g., phenyl). In some embodiments, one of R^{1a} and R^{1b} is 2-hydroxy-2-propyl, the other one of R^{1a} and R^{1b} is 3-hydroxy-2-propyl, and R² is C₆-C₁₀ aryl (e.g., phenyl). In some embodiments, one of R^{1a} and R^{1b} is 2-hydroxy-2-propy 1, the other one of R^{1a} and R^{1b} is 1-hydroxy-1-propyl, and R² is C₆-C₁₀ aryl (e.g., phenyl). In some embodiments, one of R^{1a} and R^{1b} is 2-hydroxy-2-propyl, the other one of R^{1a} and R^{1b} is 2-hydroxy-1-propyl, and R² is C₆-C₁₀ aryl. (e.g., phenyl). In some embodiments, one of R^{1a} and R^{1b} is 2-hydroxy-2-propyl, the other one of R^{1a} and R^{1b} is 3-hydroxy-1-propyl, and R² is C₆-C₁₀ aryl (e.g., phenyl). In some embodiments, one of R^{1a} and R^{1b} is 2-hydroxy-2-propyl, the other one of R^{1a} and R^{1b} is hydroxybutyl, and R² is C₆-C₁₀ aryl (e.g., phenyl). In some embodiments, one of R^{1a} and R^{1b} is 2-hydroxy-2-propyl, the other one of R^{1a} and R^{1b} is hydroxypentyl, and R² is C₆-C₁₀ aryl (e.g., phenyl). In some embodiments, one of R^{1a} and R^{1b} is 2-hydroxy-2-propyl, the other one of R^{1a} and R^{1b} is hydroxyhexyl, and R² is C₆-C₁₀ aryl (e.g., phenyl).

In some embodiments, one of R^{1a} and R^{1b} is hydroxymethyl, the other one of R^{1a} and R^{1b} is hydroxymethyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is hydroxyethyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is 2-hydroxy-2-propyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is 3-hydroxy-2-propyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is 1-hydroxy-1-propyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is 2-hydroxy-1-propyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is 3-hydroxy-1-propyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is hydroxybutyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is hydroxypentyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is hydroxyhexyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is hydroxyethyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is 2-hydroxy-2-propyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is 3-hydroxy-2-propyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is 1-hydroxy-1-propyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is 2-hydroxy-1-propyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is 3-hydroxy-1-propyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is hydroxybutyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is hydroxypentyl, and R² is 5- to 10-membered heteroaryl (e.g., pyridyl or pyrazolyl).

In some embodiments, one of R^{1a} and R^{1b} is hydroxyethyl, the other one of R^{1a} and R^{1b} is hydroxyhexyl, and R² is 5- to 10-membered heteroaryl (e.g., pyridyl or pyrazolyl).

In some embodiments, one of R^{1a} and R^{1b} is 2-hydroxy-2-propyl, the other one of R^{1a} and R^{1b} is 2-hydroxy-2-propyl, and R² is 5- to 10-membered heteroaryl (e.g., pyridyl or pyrazolyl).

In some embodiments, one of R^{1a} and R^{1b} is 2-hydroxy-2-propyl, the other one of R^{1a} and R^{1b} is 3-hydroxy-2-propyl, and R² is 5- to 10-membered heteroaryl (e.g., pyridyl or pyrazolyl).

In some embodiments, one of R^{1a} and R^{1b} is 2-hydroxy-2-propyl, the other one of R^{1a} and R^{1b} is 1-hydroxy-1-propyl, and R² is 5- to 10-membered heteroaryl (e.g., pyridyl or pyrazolyl).

In some embodiments, one of R^{1a} and R^{1b} is 2-hydroxy-2-propyl, the other one of R^{1a} and R^{1b} is 2-hydroxy-1-propyl, and R² is 5- to 10-membered heteroaryl (e.g., pyridyl or pyrazolyl),

In some embodiments, one of R^{1a} and R^{1b} is 2-hydroxy-2-propyl, the other one of R^{1a} and R^{1b} is 3-hydroxy-1-propyl, and R² is 5- to 10-membered heteroaryl (e.g., pyridyl or pyrazolyl).

In some embodiments, one of R^{1a} and R^{1b} is 2-hydroxy-2-propyl, the other one of R^{1a} and R^{1b} is hydroxybutyl, and R² is 5- to 10-membered heteroaryl (e.g., pyridyl or pyrazolyl).

In some embodiments, one of R^{1a} and R^{1b} is 2-hydroxy-2-propyl, the other one of R^{1a} and R^{1b} is hydroxypentyl, and R² is 5- to 10-membered heteroaryl (e.g., pyridyl or pyrazolyl).

In some embodiments, one of R^{1a} and R^{1b} is 2-hydroxy-2-propyl, the other one of R^{1a} and R^{1b} is hydroxyhexyl, and R² is 5- to 10-membered heteroaryl (e.g., pyridyl or pyrazolyl).

In some embodiments, one of R^{1a} and R^{1b} is hydroxymethyl, the other one of R^{1a} and R^{1b} is hydroxymethyl, and R² is SC₁-C₆ alkyl. In some embodiments, one of R^{1a} and R¹⁰ is hydroxymethyl, the other one of R^{1a} and R^{1b} is hydroxyethyl, and R² is SC₁-C₆ alkyl. In some embodiments, one of R^{1a} and R^{1b} is hydroxymethyl, the other one of R^{1a} and R^{1b} is 2-hydroxy-2-propyl, and R² is SC₁-C₆ alkyl. In some embodiments, one of R^{1a} and R^{1b} is hydroxymethyl, the other one of R^{1a} and R^{1b} is 3-hydroxy-2-propyl, and R² is SC₁-C₆ alkyl. In some embodiments, one of R^{1a} and R¹⁰ is hydroxymethyl, the other one of R^{1a} and R^{1b} is 1-hydroxy-1-propyl, and R² is SC₁-C₆ alkyl. In some embodiments, one of R^{1a} and R^{1b} is hydroxymethyl, the other one of R^{1a} and R^{1b} is 2-hydroxy-1-propyl, and R² is SC₁-C₆ alkyl. In some embodiments, one of R^{1a} and R^{1b} is hydroxymethyl, the other one of R^{1a} and R^{1b} is 3-hydroxy-1-propyl, and R² is SC₁-C₆ alkyl. In some embodiments, one of R^{1a} and R^{1b} is hydroxymethyl, the other one of R^{1a} and R^{1b} is hydroxybutyl, and R² is SC₁-C₆ alkyl. In some embodiments, one of R^{1a} and R^{1b} is hydroxymethyl, the other one of R^{1a} and R^{1b} is hydroxypentyl, and R² is SC₁-C₆ alkyl. In some embodiments, one of R^{1a} and R^{1b} is hydroxymethyl, the other one of R^{1a} and R^{1b} is hydroxyhexyl, and R² is SC₁-C₆ alkyl. In some embodiments, one of R^{1a} and R^{1b} is hydroxyethyl, the other one of R^{1a} and R^{1b} is hydroxyethyl, and R² is SC₁-C₆ alkyl. In some embodiments, one of R^{1a} and R^{1b} is hydroxyethyl, the other one of R^{1a} and R^{1b} is 2-hydroxy-2-propyl, and R² is SC₁-C₆ alkyl. In some embodiments, one of R^{1a} and R^{1b} is hydroxyethyl, the other one of R^{1a} and R^{1b} is 3-hydroxy-2-propyl, and R² is SC₁-C₆ alkyl. In some embodiments, one of R^{1a} and R^{1b} is hydroxyethyl, the other one of R^{1a} and R^{1b} is 1-hydroxy-1-propyl, and R² is SC₁-C₆ alkyl. In some embodiments, one of R^{1a} and R^{1b} is hydroxyethyl, the other one of R^{1a} and R^{1b} is 2-hydroxy-1-propyl, and R² is SC₁-C₆ alkyl. In some embodiments, one of R^{1a} and R^{1b} is hydroxyethyl, the other one of R^{1a} and R^{1b} is 3-hydroxy-1-propyl, and R² is SC₁-C₆ alkyl. In some embodiments, one of R^{1a} and R^{1b} is hydroxyethyl, the other one of R^{1a} and R^{1b} is hydroxybutyl, and R² is SC₁-C₆ alkyl. In some embodiments, one of R^{1a} and R^{1b} is hydroxyethyl, the other one of R^{1a} and R^{1b} is hydroxypentyl, and R² is SC₁-C₆ alkyl. In some embodiments, one of R^{1a} and R^{1b} is hydroxyethyl, the other one of R^{1a} and R^{1b} is hydroxy hexyl, and R² is SC₁-C₆ alkyl. In some embodiments, one of R^{1a} and R^{1b} is 2-hydroxy-2-propyl, the other one of R^{1a} and R^{1b} is 2-hydroxy-2-propyl, and R² is SC₁-C₆ alkyl. In some embodiments, one of R^{1a} and R^{1b} is 2-hydroxy-2-propyl, the other one of R^{1a} and R^{1b} is 3-hydroxy-2-propyl, and R² is SC₁-C₆ alkyl. In some embodiments, one of R^{1a} and R^{1b} is 2-hydroxy-2-propyl, the other one of R^{1a} and R^{1b} is 1-hydroxy-1-propyl, and R² is SC₁-C₆ alkyl. In some embodiments, one of R^{1a} and R^{1b} is 2-hydroxy-2-propyl, the other one of R^{1a} and R^{1b} is 2-hydroxy-1-propyl, and R² is SC₁-C₆ alkyl. In some embodiments, one of R^{1a} and R^{1b} is 2-hydroxy-2-propyl, the other one of R^{1a} and R^{1b} is 3-hydroxy-1-propyl, and R² is SC₁-C₆ alkyl. In some embodiments, one of R^{1a} and R^{1b} is 2-hydroxy-2-propyl, the other one of R^{1a} and R^{1b} is hydroxybutyl, and R² is SC₁-C₆ alkyl. In some embodiments, one of R^{1a} and R^{1b} is 2-hydroxy-2-propyl, the other one of R^{1a} and R^{1b} is hydroxypentyl, and R² is SC₁-C₆ alkyl. In some embodiments, one of R^{1a} and R^{1b} is 2-hydroxy-2-propyl, the other one of R^{1a} and R^{1b} is hydroxyhexyl, and R² is SC₁-C₆ alkyl. In some embodiments, one of R^{1a} and R^{1b} is hydroxymethyl, the other one of R^{1a} and R^{1b} is hydroxymethyl, and R² is S(O₂)C₁-C₆ alkyl (e.g., S(O₂)CH₃). In some embodiments, one of R^{1a} and R¹⁰ is hydroxymethyl, the other one of R^{1a} and R^{1b} is hydroxyethyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 2-hydroxy-2-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 3-hydroxy-2-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 1-hydroxy-1-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 2-hydroxy-1-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is 3-hydroxy-1-propyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxybutyl, and R² is S(O₂)C₁-C₆ alkyl (e.g., S(O₂)CH₃). In some embodiments, one of R^{1a} and R¹⁰ is hydroxymethyl, the other one of R^{1a} and R^{1b} is hydroxypentyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxyhexyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxyethyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 2-hydroxy-2-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 3-hydroxy-2-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 1-hydroxy-1-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 2-hydroxy-1-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is 3-hydroxy-1-propyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxybutyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxypentyl, and R² is S(O₂)C₁-C₆ alkyl (e.g., S(O₂)CH₃). In some embodiments, one of R^{1a} and R^{1b} is hydroxyethyl, the other one of R^{1a} and R^{1b} is hydroxyhexyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 2-hydroxy-2-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 3-hydroxy-2-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 1-hydroxy-1-propyl, and R² is S(O₂)C₁-C₆ alkyl (e.g., S(O₂)CH₃). In some embodiments, one of R^{1a} and R¹⁰ is 2-hydroxy-2-propyl, the other one of R^{1a} and R^{1b} is 2-hydroxy-1-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is 3-hydroxy-1-propyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is hydroxybutyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R¹⁰ is hydroxypentyl, and R² is S(O₂)C₁-C₆ alkyl (e.g., S(O₂)CH₃). In some embodiments, one of R^{1a} and R^{1b} is 2-hydroxy-2-propyl, the other one of R^{1a} and R^{1b} is hydroxyhexyl, and R² is S(O₂)C₁-C₆ alkyl (e.g., S(O₂)CH₃). In some embodiments, one of R^{1a} and R^{1b} is hydroxymethyl, the other one of R^{1a} and R^{1b} is hydroxymethyl, and R² is halo (e.g., fluoro or chloro). In some embodiments, one of R^{1a} and R^{1b} is hydroxymethyl, the other one of R^{1a} and R^{1b} is hydroxyethyl, and R² is halo (e.g., fluoro or chloro). In some embodiments, one of R^{1a} and R^{1b} is hydroxymethyl, the other one of R^{1a} and R^{1b} is 2-hydroxy-2-propyl, and R² is halo (e.g., fluoro or chloro). In some embodiments, one of R^{1a} and R^{1b} is hydroxymethyl, the other one of R^{1a} and R^{1b} is 3-hydroxy-2-propyl, and R² is halo (e.g., fluoro or chloro). In some embodiments, one of R^{1a} and R^{1b} is hydroxymethyl, the other one of R^{1a} and R^{1b} is 1-hydroxy-1-propyl, and R² is halo (e.g., fluoro or chloro). In some embodiments, one of R^{1a} and R^{1b} is hydroxymethyl, the other one of R^{1a} and R^{1b} is 2-hydroxy-1-propyl, and R² is halo (e.g., fluoro or chloro). In some embodiments, one of R^{1a} and R^{1b} is hydroxymethyl, the other one of R^{1a} and R¹⁰ is 3-hydroxy-1-propyl, and R² is halo (e.g., fluoro or chloro). In some embodiments, one of R^{1a} and R^{1b} is hydroxymethyl, the other one of R^{1a} and R^{1b} is hydroxybutyl, and R² is halo (e.g., fluoro or chloro). In some embodiments, one of R^{1a} and R^{1b} is hydroxymethyl, the other one of R^{1a} and R^{1b} is hydroxypentyl, and R² is halo (e.g., fluoro or chloro). In some embodiments, one of R^{1a} and R^{1b} is hydroxymethyl, the other one of R^{1a} and R^{1b} is hydroxyhexyl, and R² is halo (e.g., fluoro or chloro). In some embodiments, one of R^{1a} and R^{1b} is hydroxyethyl, the other one of R^{1a} and R^{1b} is hydroxyethyl, and R² is halo (e.g., fluoro or chloro). In some embodiments, one of R^{1a} and R¹⁰ is hydroxyethyl, the other one of R^{1a} and R^{1b} is 2-hydroxy-2-propyl, and R² is halo (e.g., fluoro or chloro). In some embodiments, one of R^{1a} and R¹⁰ is hydroxyethyl, the other one of R^{1a} and R^{1b} is 3-hydroxy-2-propyl, and R² is halo (e.g., fluoro or chloro). In some embodiments, one of R^{1a} and R^{1b} is hydroxyethyl, the other one of R^{1a} and R^{1b} is 1-hydroxy-1-propyl, and R² is halo (e.g., fluoro or chloro). In some embodiments, one of R^{1a} and R^{1b} is hydroxyethyl, the other one of R^{1a} and R^{1b} is 2-hydroxy-1-propyl, and R² is halo (e.g., fluoro or chloro). In some embodiments, one of R^{1a} and R^{1b} is hydroxyethyl, the other one of R^{1a} and R^{1b} is 3-hydroxy-1-propyl, and R² is halo (e.g., fluoro or chloro). In some embodiments, one of R^{1a} and R^{1b} is hydroxyethyl, the other one of R^{1a} and R^{1b} is hydroxybutyl, and R² is halo (e.g., fluoro or chloro). In some embodiments, one of R^{1a} and R^{1b} is hydroxyethyl, the other one of R^{1a} and R^{1b} is hydroxypentyl, and R² is halo (e.g., fluoro or chloro). In some embodiments, one of R^{1a} and R^{1b} is hydroxyethyl, the other one of R^{1a} and R^{1b} is hydroxyhexyl, and R² is halo (e.g., fluoro or chloro). In some embodiments, one of R^{1a} and R^{1b} is 2-hydroxy-2-propyl, the other one of R^{1a} and R^{1b} is 2-hydroxy-2-propyl, and R² is halo (e.g., fluoro or chloro).

In some embodiments, one of R^{1a} and R^{1b} is 2-hydroxy-2-propyl, the other one of R^{1a} and R^{1b} is 3-hydroxy-2-propyl, and R² is halo (e.g., fluoro or chloro). In some embodiments, one of R^{1a} and R^{1b} is 2-hydroxy-2-propyl, the other one of R^{1a} and R^{1b} is 1-hydroxy-1-propyl, and R² is halo (e.g., fluoro or chloro). In some embodiments, one of R^{1a} and R^{1b} is 2-hydroxy-2-propyl, the other one of R^{1a} and R^{1b} is 2-hydroxy-1-propyl, and R² is halo (e.g., fluoro or chloro). In some embodiments, one of R^{1a} and R^{1b} is 2-hydroxy-2-propyl, the other one of R^{1a} and R^{1b} is 3-hydroxy-1-propyl, and R² is halo (e.g., fluoro or chloro). In some embodiments, one of R^{1a} and R^{1b} is 2-hydroxy-2-propyl, the other one of R^{1a} and R^{1b} is hydroxy butyl, and R² is halo (e.g., fluoro or chloro). In some embodiments, one of R^{1a} and R^{1b} is 2-hydroxy-2-propyl, the other one of R^{1a} and R^{1b} is hydroxypentyl, and R² is halo (e.g., fluoro or chloro). In some embodiments, one of R^{1a} and R¹⁰ is 2-hydroxy-2-propyl, the other one of R^{1a} and R^{1b} is hydroxyhexyl, and R² is halo (e.g., fluoro or chloro).

In some embodiments, one of R^{1a} and R^{1b} is hydroxymethyl, the other one of R^{1a} and R^{1b} is hydroxymethyl, and R² is C₃-C₇ cycloalkyl optionally substituted with one or more hydroxy (e.g, 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is hydroxyethyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is 2-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is 3-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is 1-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is 2-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is 3-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is hydroxybutyl, and R² is C₃-C₇ cycloalkyl optionally substituted with one or more hydroxy (e.g, 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is hydroxypentyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is hydroxyhexyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxy ethyl, the other one of R^{1a} and R^{1b} is hydroxyethyl, and R² is C₃-C₇ cycloalkyl optionally substituted with one or more hydroxy (e.g. 1-hydroxy-1-cyclopropyl, l-hydroxy-1-cyclobutyl, 1-hydroxy-1-cyclopentyl, or 1-hydroxy-1-cyclohexyl).

In some embodiments, one of R^{1a} and R^{1b} is hydroxyethyl, the other one of R^{1a} and R^{1b} is 2-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl),

In some embodiments, one of R^{1a} and R^{1b} is hydroxyethyl, the other one of R^{1a} and R^{1b} is 3-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxyethyl, the other one of R^{1a} and R^{1b} is 1-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxyethyl, the other one of R^{1a} and R^{1b} is 2-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxyethyl, the other one of R^{1a} and R^{1b} is 3-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxyethyl, the other one of R^{1a} and R^{1b} is hydroxybutyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxyethyl, the other one of R^{1a} and R^{1b} is hydroxypentyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxyethyl, the other one of R^{1a} and R^{1b} is hydroxyhexyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is 2-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is 3-hydroxy-2-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is 1-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is 2-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is 3-hydroxy-1-propyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is hydroxybutyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is hydroxypentyl, and R² is C₃-C₇ cycloalkyl optionally substituted with one or more hydroxy (e.g. 1-hydroxy-1-cyclopropyl, l-hydroxy-1-cyclobutyl, 1-hydroxy-1-cyclopentyl, or 1-hydroxy-1-cyclohexyl).

In some embodiments, one of R^{1a} and R^{1b} is 2-hydroxy-2-propyl, the other one of R^{1a} and R^{1b} is hydroxyhexyl, and R² is C₃-C₇ cycloalkyl optionally substituted with one or more hydroxy (e.g. 1-hydroxy-1-cyclopropyl, 1-hydroxy-1-cyclobutyl, 1-hydroxy-1-cyclopentyl, or 1-hydroxy-1-cyclohexyl).

In some embodiments, one of R^{1a} and R^{1b} is hydroxymethyl, the other one of R^{1a} and R^{1b} is hydroxymethyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is hydroxyethyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is 2-hydroxy-2-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is 3-hydroxy-2-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is 1-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is 2-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is 3-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is hydroxybutyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is hydroxypentyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is hydroxyhexyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxy ethyl, the other one of R^{1a} and R^{1b} is hydroxyethyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is 2-hydroxy-2-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is 3-hydroxy-2-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is 1-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is 2-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is 3-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is hydroxybutyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is hydroxypentyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxyethyl, the other one of R^{1a} and R^{1b} is hydroxyhexyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is 2-hydroxy-2-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is 3-hydroxy-2-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is 1-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is 2-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is 3-hydroxy-1-propyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is hydroxybutyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is hydroxypentyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is 2-hydroxy-2-propyl, the other one of R^{1a} and R^{1b} is hydroxyhexyl, and R² is 3- to 7-membered heterocycloalkyl optionally substituted with one or more hydroxy (e.g., morpholinyl or 1,3-dioxolan-2-yl).

In some embodiments, one of R^{1a} and R^{1b} is hydroxymethyl, the other one of R^{1a} and R^{1b} is hydroxymethyl, and R² is COCH₃. In some embodiments, one of R^{1a} and R^{1b} is hydroxymethyl, the other one of R^{1a} and R^{1b} is hydroxy ethyl, and R² is COCH₃. In some embodiments, one of R^{1a} and R^{1b} is hydroxymethyl, the other one of R^{1a} and R^{1b} is 2-hydroxy-2-propyl, and R² is COCH₃. In some embodiments, one of R^{1a} and R^{1b} is hydroxymethyl, the other one of R^{1a} and R^{1b} is 3-hydroxy-2-propyl, and R² is COCH₃. In some embodiments, one of R^{1a} and R^{1b} is hydroxymethyl, the other one of R^{1a} and R^{1b} is 1-hydroxy-1-propyl, and R² is COCH₃. In some embodiments, one of R^{1a} and R^{1b} is hydroxymethyl, the other one of R^{1a} and R^{1b} is 2-hydroxy-1-propyl, and R² is COCH₃. In some embodiments, one of R^{1a} and R¹⁰ is hydroxymethyl, the other one of R^{1a} and R^{1b} is 3-hydroxy-1-propyl, and R² is COCH₃. In some embodiments, one of R^{1a} and R¹⁰ is hydroxymethyl, the other one of R^{1a} and R^{1b} is hydroxybutyl, and R² is COCH₃. In some embodiments, one of R^{1a} and R^{1b} is hydroxymethyl, the other one of R^{1a} and R^{1b} is hydroxypentyl, and R² is COCH₃. In some embodiments, one of R^{1a} and R^{1b} is hydroxymethyl, the other one of R^{1a} and R^{1b} is hydroxyhexyl, and R² is COCH₃. In some embodiments, one of R^{1a} and R^{1b} is hydroxy ethyl, the other one of R^{1a} and R^{1b} is hydroxy ethyl, and R² is COCH₃. In some embodiments, one of R^{1a} and R^{1b} is hydroxy ethyl, the other one of R^{1a} and R^{1b} is 2-hydroxy-2-propyl, and R² is COCH₃. In some embodiments, one of R^{1a} and R¹⁰ is hydroxyethyl, the other one of R^{1a} and R^{1b} is 3-hydroxy-2-propyl, and R² is COCH₃. In some embodiments, one of R^{1a} and R^{1b} is hydroxyethyl, the other one of R^{1a} and R^{1b} is 1-hydroxy-1-propyl, and R² is COCH₃. In some embodiments, one of R^{1a} and R^{1b} is hydroxyethyl, the other one of R^{1a} and R^{1b} is 2-hydroxy-1-propyl, and R² is COCH₃. In some embodiments, one of R^{1a} and R^{1b} is hydroxyethyl, the other one of R^{1a} and R^{1b} is 3-hydroxy-1-propyl, and R² is COCH₃. In some embodiments, one of R^{1a} and R^{1b} is hydroxyethyl, the other one of R^{1a} and R^{1b} is hydroxybutyl, and R² is COCH₃. In some embodiments, one of R^{1a} and R¹⁰ is hydroxyethyl, the other one of R^{1a} and R^{1b} is hydroxypentyl, and R² is COCH₃. In some embodiments, one of R^{1a} and R^{1b} is hydroxyethyl, the other one of R^{1a} and R^{1b} is hydroxyhexyl, and R² is COCH₃. In some embodiments, one of R^{1a} and R^{1b} is 2-hydroxy-2-propyl, the other one of R^{1a} and R^{1b} is 2-hydroxy-2-propyl, and R² is COCH₃; In some embodiments, one of R^{1a} and R^{1b} is 2-hydroxy-2-propyl, the other one of R^{1a} and R¹⁰ is 3-hydroxy-2-propyl, and R² is COCH₃. In some embodiments, one of R^{1a} and R^{1b} is 2-hydroxy-2-propyl, the other one of R^{1a} and R^{1b} is 1-hydroxy-1-propyl, and R² is COCH₃. In some embodiments, one of R^{1a} and R¹⁰ is 2-hydroxy-2-propyl, the other one of R^{1a} and R^{1b} is 2-hydroxy-1-propyl, and R² is COCH₃. In some embodiments, one of R^{1a} and R^{1b} is 2-hydroxy-2-propyl, the other one of R^{1a} and R^{1b} is 3-hydroxy-1-propyl, and R² is COCH₃. In some embodiments, one of R^{1a} and R^{1b} is 2-hydroxy-2-propyl, the other one of R^{1a} and R^{1b} is hydroxybutyl, and R² is COCH₃. In some embodiments, one of R^{1a} and R^{1b} is 2-hydroxy-2-propyl, the other one of R^{1a} and R^{1b} is hydroxypentyl, and R² is COCH₃; In some embodiments, one of R^{1a} and R^{1b} is 2-hydroxy-2-propyl, the other one of R^{1a} and R¹⁰ is hydroxyhexyl, and R² is COCH₃.

In some embodiments, one of R^{1a} and R^{1b} is hydroxymethyl, the other one of R^{1a} and R^{1b} is hydroxymethyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is hydroxyethyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is 2-hydroxy-2-propyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is 3-hydroxy-2-propyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is 1-hydroxy-1-propyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is 2-hydroxy-1-propyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is 3-hydroxy-1-propyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is hydroxybutyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is hydroxypentyl, and R² is C₁-C₆ alkyl optionally substituted with one or more C₁-C₆ alkoxy.

In some embodiments, one of R^{1a} and R^{1b} is hydroxymethyl, the other one of R^{1a} and R^{1b} is hydroxyhexyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is hydroxyethyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is 2-hydroxy-2-propyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is 3-hydroxy-2-propyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is 1-hydroxy-1-propyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is 2-hydroxy-1-propyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is 3-hydroxy-1-propyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is hydroxybutyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is hydroxypentyl, and R² is C₁-C₆ alkyl optionally substituted with one or more C₁-C₆ alkoxy.

In some embodiments, one of R^{1a} and R^{1b} is hydroxyethyl, the other one of R^{1a} and R^{1b} is hydroxyhexyl, and R² is C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is 2-hydroxy-2-propyl, and R² C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is 3-hydroxy-2-propyl, and R² C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is 1-hydroxy-1-propyl, and R² C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is 2-hydroxy-1-propyl, and R² is C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is 3-hydroxy-1-propyl, and R² is C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is hydroxybutyl, and R² is C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is hydroxypentyl, and R² is C₁-C₆ alkyl optionally substituted with one or more C₁-C₆ alkoxy.

In some embodiments, one of R^{1a} and R^{1b} is 2-hydroxy-2-propyl, the other one of R^{1a} and R^{1b} is hydroxyhexyl, and R² is C₁-C₆ alkyl optionally substituted with one or more C₁-C₆ alkoxy.

In some embodiments, R^{1a} is different from R^{1b}. In some embodiments, R^{1a} is the same as R^{1b}. In some embodiments, R^{1a} and R² are different. In some embodiments, R^{1b} and R² are different. In some embodiments, R^{1a} is the same as R^{1b}, and R^{1a} is different from R². In some embodiments, R^{1a} is different from R^{1b}, and one of R^{1a} and R^{1b} is the same as R². In some embodiments, R^{1a} is different from R^{1b}, and both R^{1a} and R^{1b} are different from R². In some embodiments, R² comprises a carbonyl group. In some embodiments, R² comprises 1 or 2 (e.g., 1) nitrogen atoms.

In some embodiments, R² comprises 1 or 2 (e.g., 1) oxygen atoms. In some embodiments, R² comprises a sulfur atom. In some embodiments, R² comprises a carbonyl group. In some embodiments, R² comprises a sulfur atom. In some embodiments, R^{1a} is ortho to R^{1b}. In some embodiments, R^{1a} is meta to R^{1b}. In some embodiments, R^{1a} is para to R^{1b}.

The Variables o and p

In some embodiments, o=1 or 2. In some embodiments, o=T. In some embodiments, o=2. In some embodiments, p=Q, 1, 2, or 3. In some embodiments, p=0. In some embodiments, p=1. In some embodiments, p=2. In some embodiments, o=1 and p=0. In some embodiments, o=2 and p=0. In some embodiments, o=1 and p=1. In some embodiments, o=T and p=2. In some embodiments, o=2 and p=1. In some embodiments, o=2 and p=2. In some embodiments, o=2 and p=3.

The Ring B and Substitutions on the Ring B

In some embodiments, B is a 5- to 10-membered monocyclic or bicyclic heteroaryl or a C₆-C₁₀ monocyclic or bicyclic aryl, such as phenyl. In some embodiments, B is a 5- to 6-membered monocyclic heteroaryl or a C₆ monocyclic aryl. In some embodiments, B is a 5- to 10-membered monocyclic or bicyclic heteroaryl. In some embodiments, B is a C₆-C₁₀ monocyclic or bicyclic aryl. In some embodiments, B is phenyl substituted with 1 or 2 R⁶ and optionally substituted with 1, 2, or 3 R⁷. In some embodiments, B is pyridyl substituted with 1 or 2 R⁶ and optionally substituted with 1, 2, or 3 R⁷. In some embodiments, B is phenyl, o is 1 or 2, and p is 0, 1, 2 or 3. In some embodiments, B is pyridyl, o is 1 or 2, and p is 0, 1, 2 or 3. In some embodiments, B is phenyl, o is 1 or 2, and p is 0. In some embodiments, B is pyridyl, o is 1 or 2, and p is 0. In some embodiments, B is phenyl, o is 1 or 2, and p is 1. In some embodiments, B is pyridyl, o is 1 or 2, and p is 1. In some embodiments, B is phenyl, o is 1, and p is 0, 1, 2 or 3. In some embodiments, B is phenyl, o is 2, and p is 0, 1, 2 or 3. In some embodiments, B is pyridyl, o is 1, and p is 0, 1, 2 or 3. In some embodiments, B is pyridyl, o is 2, and p is 0, 1, 2 or 3. In some embodiments, B is phenyl, o is 1, and p is 0 or 1. In some embodiments, B is phenyl, o is 2, and p is 0 or 1. In some embodiments, B is pyridyl, o is 1, and p is 0 or 1. In some embodiments, B is pyridyl, o is 2, and p is 0 or 1.

In some embodiments, B is one of the rings disclosed hereinbelow, substituted as disclosed hereinbelow, wherein in each case the bond that is shown as being broken by the wavy line [symbol] connects B to the NH(CO) group of Formula AA.

In some embodiments, the optionally substituted ring B

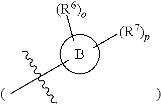

is

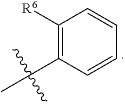

In some embodiments, the optionally substituted ring B

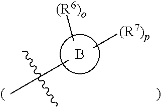

is

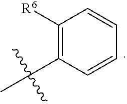

In some embodiments, the optionally substituted ring B is

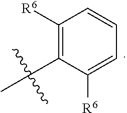

In some embodiments, the optionally substituted ring B is

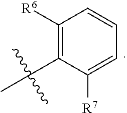

In some embodiments, the optionally substituted ring B is

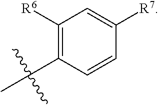

In some embodiments, the optionally substituted ring B is

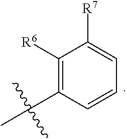

In some embodiments, the optionally substituted ring B is

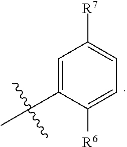

In some embodiments, the optionally substituted ring B is

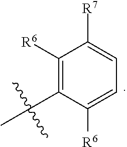

In some embodiments, the optionally substituted ring B is

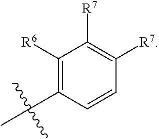

In some embodiments, the optionally substituted ring B is

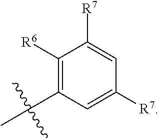

In some embodiments, the optionally substituted ring B is

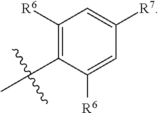

In some embodiments, the optionally substituted ring B is

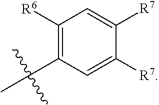

In some embodiments, the optionally substituted ring B is

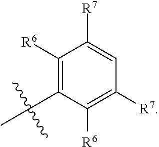

In some embodiments, the optionally substituted ring B is

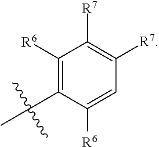

In some embodiments, the optionally substituted ring B is

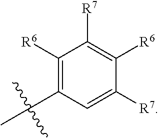

In some embodiments, the optionally substituted ring B is

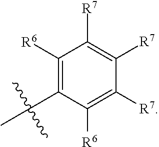

In some embodiments, the optionally substituted ring B is

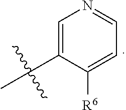

In some embodiments, the optionally substituted ring B is

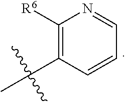

In some embodiments, the optionally substituted ring B is

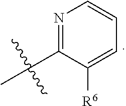

In some embodiments, the optionally substituted ring B is

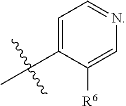

In some embodiments, the optionally substituted ring B is

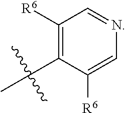

The Groups R⁶, R^{6′}, R^{6″}, R⁷, R^{7′}, and R^{7″}

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and a C₂-C₆ alkenyl,

wherein R⁶ and R⁷ are each optionally substituted with one or more substituents independently selected from

hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ and, NHCO(5- to 10-membered heteroaryl), NHCO(3- to 7-membered heterocycloalkyl), NHCOC₂-C₆ alkynyl,

C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo. CCG alkyl, and OC₁-C₆ alkyl;

or at least one pair of R⁶ and R⁷ on adjacent atoms, taken together with the atoms connecting them, independently form at least one C₄-C₅ carbocyclic ring or at least one 5-to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₁-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ and, 5- to 10-membered heteroaryl, NHCOC₆-C₁₀ aryl, NHCO(5 to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ and, and CONR⁸R⁹.

In some embodiments,

R⁶ and R⁷ are each independently selected from C₁-C₆ alkyl, halo, CN, NO₂, COC₁-C₆ alkyl, CO₂C₁-C₆, alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)-. CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₅ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ and, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ and, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₁-C₆ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl are each unsubstituted;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ and, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from Q, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, at least one pair of R⁶ and R⁷ on adjacent atoms, taken together with the atoms connecting them, independently form at least one C₄-C₈ carbocyclic ring or at least one 5-to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R⁶ and R⁷ are each independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

In some embodiments, R⁶ is CN. In some embodiments, R⁶ is C₁-C₆ alkyl substituted with hydroxyl (e.g., hydroxymethyl, hydroxyethyl, or 2-hydroxy-2-propyl. In some embodiments, R⁶ is C₁-C₆ alkyl substituted with C₁-C₆ alkoxy (e.g., methoxymethyl) In some embodiments, R⁶ is imidazolyl. In some embodiments, R⁶ is pyrazolyl. In some embodiments, R⁶ is pyrrolyl. In some embodiments, R⁶ is thiazolyl. In some embodiments, R⁶ is isothiazolyl. In some embodiments, R⁶ is oxazolyl. In some embodiments, R⁶ is isoxazolyl. In some embodiments, R⁶ is pyridyl. In some embodiments, R⁶ is pyrimidinyl. In some embodiments, R⁷ is CN. In some embodiments, R⁷ is C₁-C₆ alkyl substituted with hydroxyl (e.g., hydroxymethyl, hydroxyethyl, or 2-hydroxy-2-propyl. In some embodiments, R⁷ is C₁-C₆ alkyl substituted with C₁-C₆ alkoxy (e.g., methoxymethyl) In some embodiments, R⁷ is imidazolyl. In some embodiments, R⁷ is pyrazolyl. In some embodiments, R⁷ is pyrrolyl. In some embodiments, R⁷ is thiazolyl. In some embodiments, R⁷ is isothiazolyl. In some embodiments, R⁷ is oxazolyl. In some embodiments, R⁷ is isoxazolyl. In some embodiments, R⁷ is pyridyl. In some embodiments, R⁷ is pyrimidinyl.

In some embodiments, o=1; p=0; and

R⁶ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ and, OCO(5- to 10-membered heteroaryl), OCO(3- to 7-membered heterocycloalkyl), C₆-C₁₀ and, 5-to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently-selected from hydroxy, halo, CN, oxo, C₁-C₆ alkyl, C₆-C₁₀ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=0; and

R⁶ is selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl, wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀) aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4 to 6-membered heterocycloalkyl), NHCOC₁-C₆ alkyl, NHCOC₆-C₁₀ aryl, NHCO(5- to 10-membered heteroaryl), NHCO(4 to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo.

In some embodiments, o=1 or 2, p=1, 2, or 3; and

one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3, and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R taken together with the atoms connecting them independently form a C₄-C₈ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

Particular Embodiments Wherein o=1; p=0:

In some embodiments, R⁶ is C₁-C₆ alkyl. In some embodiments, R⁶ is isopropyl. In some embodiments, R⁶ is ethyl. In some embodiments, R⁶ is methyl. In some embodiments, R⁶ is C₁-C₆ alkyl substituted with one or more halo. In some embodiments, R⁶ is trifluoromethyl. In some embodiments, R⁶ is trifluoromethoxy. In some embodiments, R⁶ is C₃-C₇ cycloalkyl. In some embodiments, R⁶ is cyclopropyl. In some embodiments, R⁶ is halo. In some embodiments, R⁶ is chloro. In some embodiments, R⁶ is fluoro. In some embodiments, R⁶ is cyano. In some embodiments, R⁶ is attached to a carbon of an aryl ring B. In some embodiments, R⁶ is attached to a carbon of a heteroaryl ring B. In some embodiments, R⁶ is attached to a nitrogen of a heteroaryl ring B.

Particular Embodiments Wherein o=1 or 2; p=1, 2, Or 3:

In some embodiments, at least one R⁶ is C₁-C₆ alkyl, and at least one R⁷ is C₁-C₆ alkyl optionally substituted with one or more halo. In some embodiments, at least one R⁶ is C₁-C₆ alkyl and at least one R⁷ is C₁-C₆ alkyl. In some embodiments, at least one R⁶ is isopropyl and at least one R⁷ is methyl. In some embodiments, at least one R^{b} is isopropyl and at least one R⁷ is isopropyl. In some embodiments, o=1; p=1; R⁶ is isopropyl; and R⁷ is isopropyl. In some embodiments, at least one R⁶ is C₁-C₆ alkyl, and at least one R⁷ is C₁-C₆ alkyl substituted with one or more halo. In some embodiments, at least one R⁶ is isopropyl and at least one R⁷ is trifluoromethyl. In some embodiments, at least one R⁶ is C₁-C₆ alkyl, and at least one R⁷ is C₃-C₇ cycloalkyl. In some embodiments, at least one R⁶ is isopropyl and at least one R⁷ is cyclopropyl. In some embodiments, o=1; p=1; R⁶ is isopropyl; and R⁷ is cyclopropyl. In some embodiments, at least one R⁶ is C₁-C₆ alkyl, and at least one R⁷ is halo. In some embodiments, at least one R⁶ is isopropyl and at least one R⁷ is halo. In some embodiments, at least one R⁶ is isopropyl and at least one R⁷ is chloro. In some embodiments, at least one R^{b} is isopropyl and at least one R⁷ is fluoro. In some embodiments, o=1; p=1; R⁶ is isopropyl; and R⁷ is chloro. In some embodiments, o=2; p=1; at least one R⁶ is isopropyl; and R⁷ is chloro. In some embodiments, o=1; p=1; R⁶ is isopropyl; and R⁷ is fluoro. In some embodiments, o=2; p=1; at least one R⁶ is isopropyl; and R⁷ is fluoro. In some embodiments, o=2; p=2; at least one R⁶ is isopropyl; and at least one R⁷ is fluoro. In some embodiments, o=2; p=2; at least one R⁶ is isopropyl; one R⁷ is fluoro; and the other R⁷ is cyano. In some embodiments, o=2; p=3; at least one R⁶ is isopropyl; two R⁷ are fluoro; and one R⁷ is chloro. In some embodiments, o=2; p=1; at least one R⁶ is ethyl; and R⁷ is fluoro. In some embodiments, o=2; p=1; one R⁶ is isopropyl; the other R⁶ is trifluoromethyl; and R⁷ is chloro. In some embodiments, at least, one R⁶ is C₁-C₆ alkyl, and at least one R⁷ is cyano. In some embodiments, at least one R⁶ is isopropyl and at least one R⁷ is cyano. In some embodiments, o=1; p=1; R⁶ is isopropyl; and R⁷ is cyano. In some embodiments, o=2; p=1; at least one R⁶ is isopropyl; and R⁷ is cyano. In some embodiments, at least one R⁶ is C₃-C₇ cycloalkyl, and at least one R⁷ is C₃-C₇ cycloalkyl. In some embodiments, at least one R⁶ is cyclopropyl, and at least one R⁷ is cyclopropyl. In some embodiments, at least one R⁶ is C₃-C₇ cycloalkyl, and at least one R⁷ is halo. In some embodiments, at least one R⁶ is cyclopropyl and at least one R⁷ is halo. In some embodiments, at least one R⁶ is cyclopropyl and at least one R⁷ is chloro. In some embodiments, at least one R⁶ is cyclopropyl and at least one R⁷ is fluoro. In some embodiments, o=1; p=1; R⁶ is cyclopropyl; and R⁷ is chloro. In some embodiments, o=1; p=1; R⁶ is cyclopropyl; and R⁷ is fluoro. In some embodiments, at least one R⁶ is C₁-C₆ alkyl, and at least one R is C₁-C₆ alkoxy optionally substituted with one or more halo. In some embodiments, at least one R⁶ is isopropyl, and at least one R⁷ is C₁-C₆ alkoxy. In some embodiments, at least one R⁶ is isopropyl, and at least one R⁷ is methoxy. In some embodiments, o=1; p=1; R^{b} is isopropyl, and R⁷ is methoxy. In some embodiments, o=2; p=1; at least one R⁶ is isopropyl, and R⁷ is methoxy. In some embodiments, at least one R⁷ is C₁-C₆ alkyl, and at least one R⁷ is C₁-C₆ alkoxy substituted with one or more halo. In some embodiments, at least one R⁶ is isopropyl, and at least one R⁷ is trifluoromethoxy. In some embodiments, at least one R⁶ is isopropyl, and at least one R⁷ is difluoromethoxy. In some embodiments, at least one R⁶ is halo, and at least one R⁷ is C₁-C₆ haloalkyl optionally substituted with hydroxy. In some embodiments, o=1; p=1; R⁶ is chloro, and R⁷ is trifluoromethyl. In some embodiments, at least one R⁶ is halo, and at least one R⁷ is C₁-C₆ haloalkoxy. In some embodiments, at least one R⁶ is chloro, and at least one R⁷ is trifluoromethoxy. In some embodiments, o=1; p=1; R^{b} is chloro, and R⁷ is trifluoromethoxy. In some embodiments, at least one R⁶ is C₁-C₆ alkoxy; and at least one R⁷ is halo. In some embodiments, o=1; p=2; R⁶ is C₁-C₆ alkoxy; and at least one R⁷ is chloro.

In some embodiments, at least one R⁷ is C₁-C₆ alkyl, and at least one R⁶ is C₁-C₆ alkyl optionally substituted with one or more halo. In some embodiments, at least one R⁷ is isopropyl and at least one R⁶ is methyl. In some embodiments, at least one R⁷ is C₁-C₆ alkyl, and at least one R⁶ is C₁-C₆ alkyl substituted with one or more halo. In some embodiments, at least one R⁷ is isopropyl and at least one R⁶ is trifluoromethyl. In some embodiments, at least one R⁷ is C₁-C₆ alkyl, and at least one R⁶ is C₃-C₇ cycloalkyl. In some embodiments, at least one R⁷ is isopropyl and at least one R⁶ is cyclopropyl. In some embodiments, o=1; p=1; R⁷ is isopropyl; and R⁶ is cyclopropyl. In some embodiments, at least one R⁷ is C₁-C₆ alkyl, and at least one R⁶ is halo. In some embodiments, at least one R⁷ is isopropyl and at least one R⁶ is halo. In some embodiments, at least one R⁷ is isopropyl and at least one R⁶ is chloro. In some embodiments, at least one R⁷ is isopropyl and at least one R⁶ is fluoro. In some embodiments, o=1; p=1; R is isopropyl, and R⁶ is chloro. In some embodiments, o=2, p=1; R⁷ is isopropyl; and at least one R⁶ is chloro. In some embodiments, o=1; p=1; R⁷ is isopropyl; and R⁶ is fluoro. In some embodiments, o=2; p=1; R⁷ is isopropyl, and at least one R⁶ is fluoro. In some embodiments, o==2; p=2; at least one R⁷ is isopropyl; and at least one R⁶ is fluoro. In some embodiments, o=2; p=2; at least one R⁷ is isopropyl, one R⁶ is fluoro; and the other R⁶ is cyano; In some embodiments, o=2; p=1; R⁷ is ethyl; and at least one R⁶ is fluoro. In some embodiments, o=1; p=2; one R⁷ is isopropyl; the other R⁷ is trifluoromethyl, and R⁶ is chloro. In some embodiments, at least one R⁷ is C₁-C₆ alkyl, and at least one R⁶ is cyano. In some embodiments, at least one R⁷ is isopropyl and at least one R⁶ is cyano. In some embodiments, o=1; p=1; R⁷ is isopropyl; and R⁶ is cyano. In some embodiments, o=2, p=1; R⁷ is isopropyl; and at least one R⁶ is cyano. In some embodiments, at least one R is C₃-C₇ cycloalkyl, and at least one R⁶ is C₃-C₇ cycloalkyl. In some embodiments, at least one R⁷ is cyclopropyl, and at least one R⁶ is cyclopropyl. In some embodiments, at least one R⁷ is C₃-C₇ cycloalkyl, and at least one R⁵ is halo. In some embodiments, at least one R⁷ is cyclopropyl and at least one R⁶ is halo. In some embodiments, at least one R⁷ is cyclopropyl and at least one R⁶ is chloro. In some embodiments, at least one R⁷ is cyclopropyl and at least one R⁵ is fluoro. In some embodiments, o=1; p=1; R⁷ is cyclopropyl; and R⁶ is chloro. In some embodiments, o=1; p=1; R is cyclopropyl; and R⁶ is fluoro. In some embodiments, at least one R⁷ is C₁-C₆ alkyl, and at least one R⁶ is C₁-C₆ alkoxy optionally substituted with one or more halo. In some embodiments, at least one R⁷ is isopropyl, and at least one R⁷ is C₁-C₆ alkoxy. In some embodiments, at least one R⁷ is isopropyl, and at least one R⁶ is methoxy. In some embodiments, o=1; p=1; R⁷ is isopropyl, and R⁶ is methoxy. In some embodiments, o=2; p=1; R⁷ is isopropyl, and at least one R⁵ is methoxy. In some embodiments, at least one R⁷ is C₁-C₆ alkyl, and at least one R⁶ is C₁-C₆ alkoxy substituted with one or more halo. In some embodiments, at least one R⁷ is isopropyl, and at least one R⁶ is trifluoromethoxy. In some embodiments, at least one R⁷ is halo, and at least one R⁶ is C₁-C₆ haloalkyl optionally substituted with one or more hydroxy. In some embodiments, o=1; p=1; R⁶ is chloro, and R⁶ is trifluoromethyl. In some embodiments, at least one R⁷ is halo, and at least one R⁶ is C₃-C₆ haloalkoxy. In some embodiments, at least one R is chloro, and at least one R⁶ is trifluoromethoxy. In some embodiments, o=1; p=1; R⁷ is chloro, and R⁶ is trifluoromethoxy. In some embodiments, at least one R⁷ is C₁-C₆ alkoxy; and at least one R⁶ is halo. In some embodiments, o=1; p=2; at least one R⁷ is C₁-C₆ alkoxy; and R⁶ is chloro. In some embodiments, R⁶ and R⁷ are each attached to a carbon of an aryl ring B. In some embodiments, R⁶ and R⁷ are each attached to a carbon of a heteroaryl ring B. In some embodiments, R⁶ is attached to a carbon and R⁷ is attached to a nitrogen of a heteroaryl ring B. In some embodiments, R⁷ is attached to a carbon and R⁶ is attached to a nitrogen of a heteroaryl ring B.

In some embodiments, one R⁶ and one R⁷ are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₅ aliphatic carbocyclic ring.

In some embodiments, R⁶ and R⁷ are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₅ aliphatic carbocyclic ring.

In some embodiments, R⁶ and R⁷ are on adjacent atoms, and taken together with the atoms connecting them, form a C₆ aromatic carbocyclic ring.

In some embodiments, R⁶ and R⁷ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 5-membered aliphatic heterocyclic ring containing 1 or 2 heteroatoms independently selected from O, N, and S.

In some embodiments, R⁶ and R⁷ are on adjacent atoms, and taken together with the atoms connecting them, form a 5-membered heteroaromatic ring containing 1 or 2 heteroatoms independently selected from O, N, and S.

In some embodiments, R⁶ and R⁷ are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 6-membered aliphatic heterocyclic ring containing 1 or 2 heteroatoms independently selected from O, N, and S,

In some embodiments, R⁶ and R⁷ are on adjacent atoms, and taken together with the atoms connecting them, form a 6-membered heteroaromatic ring containing 1 or 2 heteroatoms independently selected from O, N, and S.

In some embodiments, one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S,

wherein the ring is fused to the B ring at the 2- and 3-positions relative to the bond connecting the B ring to the NH(CO)group.

In some embodiments, o=2; p=2 or 3; and two pairs, each of one R⁶ and one R⁷, are on adjacent atoms, and each pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶ and one R⁷, are on adjacent atoms, and each pair of one R⁶ and one R⁷ taken together with the atoms connecting them form a C₅ aliphatic carbocyclic ring. In some embodiments, o=2; p=2 or 3; and two pairs, each of one R⁶ and one R⁷, are on adjacent atoms, and each pair of one R⁶ and one R⁷ taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶ and one R⁷, are on adjacent atoms, and each pair of one R⁶ and one R⁷ taken together with the atoms connecting them form a C₆ aliphatic carbocyclic ring. In some embodiments, o=2; p=2 or 3; and two pairs, each of one R⁶ and one R⁷, are on adjacent atoms, and each pair of one R⁶ and one R⁷ taken together with the atoms connecting them form a C₆ aromatic carbocyclic ring. In some embodiments, o=2; p=2 or 3; and two pairs, each of one R⁶ and one R⁷, are on adjacent atoms, and each pair of one R⁶ and one R⁷ taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2, p=2 or 3; and two pairs, each of one R⁶ and one R⁷, are on adjacent atoms, and each pair of one R⁶ and one R⁷ taken together with the atoms connecting them form a 5-membered aliphatic heterocyclic ring containing 1 or 2 heteroatoms independently selected from O, N, and S. In some embodiments, o=2; p=2 or 3; and two pairs, each of one R⁶ and one R⁷, are on adjacent atoms, and each pair of one R⁶ and one R⁷ taken together with the atoms connecting them form a 5-membered heteroaromatic ring containing 1 or 2 heteroatoms independently selected from O, N, and S. In some embodiments, o=2; p=2 or 3; and two pairs, each of one R⁶ and one R⁷, are on adjacent atoms, and each pair of one R⁶ and one R⁷ taken together with the atoms connecting them form a 6-membered heterocyclic ring containing 1 or 2 heteroatoms independently selected from Q, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2 or 3; and two pairs, each of one R⁶ and one R⁷, are on adjacent atoms, and each pair of one R⁶ and one R⁷ taken together with the atoms connecting them form a 6-membered aliphatic heterocyclic ring containing 1 or 2 heteroatoms independently selected from O, N, and S. In some embodiments, o=2; p=2 or 3; and two pairs, each of one R⁶ and one R⁷, are on adjacent atoms, and each pair of one R⁶ and one R⁷ taken together with the atoms connecting them form a 6-membered heteroaromatic ring containing 1 or 2 heteroatoms independently selected from O, N, and S; In some embodiments, o=2; p=2 or 3, and two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein one of the two rings is fused to the B ring at the 2- and 3-positions relative to the bond connecting the B ring to the NH(CO)group, and the other of the two rings is fused to the B ring at the 5- and 6-positions relative to the bond connecting the B ring to the NH(CO) group. In some embodiments, o=2; p=2; and two pairs, each of one R⁶ and one R⁷, are on adjacent atoms, and each pair of one R⁶ and one R⁷ taken together with the atoms connecting them form a C₅ aliphatic carbocyclic ring. In some embodiments, o=2; p=3; and two pairs, each of one R⁶ and one R⁷, are on adjacent atoms, and each pair of one R⁶ and one R⁷ taken together with the atoms connecting them form a C₅ aliphatic carbocyclic ring, and one R⁷ is halo (e.g., Cl or F). In some embodiments, o=2; p=3; and two pairs, each of one R⁶ and one R⁷, are on adjacent atoms, and each pair of one R⁶ and one R⁷ taken together with the atoms connecting them form a C₅ aliphatic carbocyclic ring; and one R⁷ is CN.

In some embodiments, one R⁷ is pyrazolyl and is para to the bond connecting the B ring to the NH(CO) group of Formula AA. In some embodiments, one R⁷ is 3-pyrazolyl and is para to the bond connecting the B ring to the NH(CO) group of Formula AA. In some embodiments, one R⁷ is 4-pyrazoyl and is para to the bond connecting the B ring to the NH(CO) group of Formula AA. In some embodiments, one R⁷ is 5-pyrazolyl and is para to the bond connecting the B ring to the NH(CO) group of Formula AA. In some embodiments, one R⁷ is thiazolyl and is para to the bond connecting the B ring to the NH(CO) group of Formula AA. In some embodiments, one R⁷ is 4-thiazolyl and is para to the bond connecting the B ring to the NH(CO) group of Formula AA. In some embodiments, one R⁷ is 5-thiazoyl and is para to the bond connecting the B ring to the NH(CO) group of Formula AA; In some embodiments, one R⁷ is furyl and is para to the bond connecting the B ring to the NH(CO) group of Formula AA. In some embodiments, one R⁷ is 2-furyl and is para to the bond connecting the B ring to the NH(CO) group of Formula AA. In some embodiments, one R⁷ is thiophenyl and is para to the bond connecting the B ring to the NH(CO) group of Formula AA. In some embodiments, one R⁷ is 2-thiophenyl and is para to the bond connecting the B ring to the NH(CO) group of Formula AA. In some embodiments, one R⁷ is phenyl and is para to the bond connecting the B ring to the NH(CO) group of Formula AA. In some embodiments, one R⁷ is cycloalkenyl (e.g., cyclopentenyl, e.g., 1-cyclopentenyl) and is para to the bond connecting the B ring to the NH(CO) group of Formula AA. In some embodiments, one R⁷ is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the NH(CO) group of Formula AA. In some embodiments, one R⁷ is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the NH(CO) group of Formula AA. In some embodiments, one R⁷ is phenyl optionally substituted with one or more C₆-C₁₀ aryloxy (e.g., phenoxy) and is para to the bond connecting the B ring to the NH(CO) group of Formula AA. In some embodiments, one R⁷ is phenyl optionally substituted with one or more CN and is para to the bond connecting the B ring to the NH(CO) group of Formula AA. In some embodiments, one R⁷ is phenyl optionally substituted with one or more halo (e.g., F, Cl) and is para to the bond connecting the B ring to the NH(CO) group of Formula AA and is para to the bond connecting the B ring to the NH(CO) group of Formula AA. In some embodiments, one R⁷ is phenyl optionally substituted with one or more COOC₁-C₆ alkyl (e.g., CO₂ₜ-Bu) and is para to the bond connecting the B ring to the NH(CO) group of Formula AA. In some embodiments, one R⁷ is phenyl optionally substituted with one or more S(O₂)C₁-C₆ alkyl (e.g., S(O₂)methyl) and is para to the bond connecting the B ring to the NH(CO) group of Formula AA. In some embodiments, one R⁷ is phenyl optionally substituted with one or more 3-to 7-membered heterocycloalkyl (e.g., morpholinyl) and is para to the bond connecting the B ring to the NH(CO) group of Formula AA. In some embodiments, one R⁷ is phenyl optionally substituted with one or more CONR⁸R⁹ (e.g., unsubstituted amido) and is para to the bond connecting the B ring to the NH(CO) group of Formula AA; In some embodiments, one R⁷ is phenyl optionally substituted with one or more C₁-C₆ alkyl (e.g., methyl or propyl, e.g., 2-propyl) and with one or more halo (e.g., F, Cl) and is para to the bond connecting the B ring to the NH(CO) group of Formula AA and is para to the bond connecting the B ring to the NH(CO) group of Formula AA.

In some embodiments, R⁶ and R⁷ are each attached to a carbon of an and ring B. In some embodiments, R⁶ and R⁷ are each attached to a carbon of a heteroaryl ring B. In some embodiments, R⁶ is attached to a carbon and R⁷ is attached to a nitrogen of a heteroaryl ring B. In some embodiments, R⁷ is attached to a carbon and R⁶ is attached to a nitrogen of a heteroaryl ring B.
In some embodiments, the optionally substituted ring B is

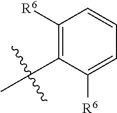

R⁶ and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, =NR¹⁰, COOC₁-C₆ alkyl, CONR⁸R⁹, 4- to 6-membered heterocycloalkyl, C₆-C₁₀ aryl, 5-to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
In some embodiments, the optionally substituted ring B is

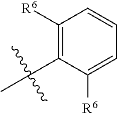

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.
In some embodiments, the optionally substituted ring B is

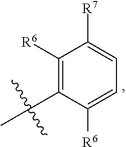

wherein each R^{b} is independently selected from C₁-C₆alkyl, C₃-C₇cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from Q, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

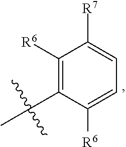

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and
In some embodiments, the optionally substituted ring B is

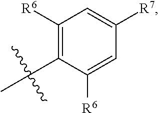

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl. OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
In some embodiments, the optionally substituted ring B is

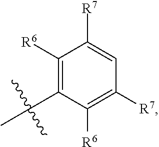

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

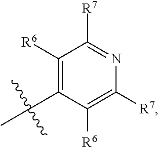

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ and, and CONR⁸R⁹.
In some embodiments, the optionally substituted ring B is

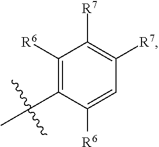

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form a C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, =NR¹⁰, COOC₁-C₆ alkyl, C₆-C₁₀ and, and CONR⁸R⁹.
In some embodiments, the optionally substituted ring B is

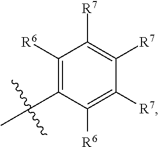

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ and, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, =NR¹⁰, COOC₁-C₆ alkyl, C₆-C₁₀ aryl, and CONR⁸R⁹.

In some embodiments, the optionally substituted ring B

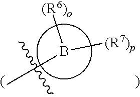

is other than

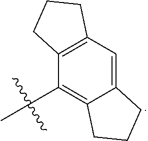 [SMILES: *c1c2c(cc3c1CCC3)CCC2]

The Groups R^{6′} and R^{7′}

R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and C₂-C₆ alkenyl,

wherein R^{6′} and R^{7′} are each optionally substituted with one or more substituents independently selected from

hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6′} and R^{7′} on adjacent atoms, taken together with the atoms connecting them, independently form at least one C₄-C₈ carbocyclic ring or at least one 5-to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂.NR⁸R⁹, ═NR¹⁰, COOC₁-C₆ alkyl, C₆-C₁₀ aryl, and CONR⁸R⁹.

In some embodiments,

R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ and, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6′} and R^{7′} are each independently selected from C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆, alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,

or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

In some embodiments,

R^{6′} and R^{7′} are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ and, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ and, 5- to 10-membered heteroaryl, NHCOC₆-C₁₀ and, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₃-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₃-C₆ alkoxy, NR—R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5 to 10-membered heteroaryl) and NHCO(3- to 7-membered heterocycloalkyl) are unsubstituted;
   or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

In some embodiments,

R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl are each unsubstituted;

or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R⁶ is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₆-C₁₀ alkynyl;
and R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5-to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R^{6′} and R^{7′} are each independently selected from C₁-C₆ alkyl, C₂-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo, or at least one pair of R^{6′} and R^{7′} on adjacent atoms, taken together with the atoms connecting them, independently form at least one C₄-C₈ carbocyclic ring, wherein the carbocyclic ring is optionally independently substituted with one or more hydroxy or oxo.

In some embodiments, R^{6′} and R^{7′} are each independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

In some embodiments, R^{6′} is CN. In some embodiments, R^{6′} is C₁-C₆ alkyl substituted with hydroxyl (e.g., hydroxymethyl, hydroxyethyl, or 2-hydroxy-2-propyl). In some embodiments, R^{6′} is C₁-C₆ alkyl substituted with C₁-C₆ alkoxy (e.g., methoxymethyl) In some embodiments, R^{6′} is imidazolyl. In some embodiments, R^{6′} is pyrazolyl. In some embodiments, R^{6′} is pyrrolyl. In some embodiments, R^{6′} is thiazolyl. In some embodiments, R^{6′} is isothiazolyl. In some embodiments, R^{6′} is oxazolyl. In some embodiments, R^{6′} is isoxazolyl. In some embodiments, R⁶ is pyridyl. In some embodiments, R⁶ is pyrimidinyl. In some embodiments, R⁷ is CN. In some embodiments, R^{7′} is C₁-C₆ alkyl substituted with hydroxyl (e.g., hydroxymethyl, hydroxyethyl, or 2-hydroxy-2-propyl. In some embodiments, R^{7′} is C₁-C₆ alkyl substituted with C₁-C₆ alkoxy (e.g., methoxymethyl) In some embodiments, R^{7′} is imidazolyl. In some embodiments, R^{7′} is pyrazolyl. In some embodiments, R^{7′} is pyrrolyl. In some embodiments, R^{7′} is thiazolyl. In some embodiments, R^{7′} is isothiazolyl. In some embodiments, R^{7′} is oxazolyl. In some embodiments, R^{7′} is isoxazolyl. In some embodiments, R^{7′} is pyridyl. In some embodiments, R^{7′} is pyrimidinyl.

In some embodiments, o=1; p=0; and

R^{6′} is selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5-to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₃-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₃-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5 to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=1; and

R^{6′} is selected from C₁-C₆ alkyl, C₂-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl, wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R^{6′} is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R^{6′} and R^{7′}, taken together with the atoms connecting them, independently form C₄-C₇ carbocyclic ring or at least one 5-to-7-membered heterocyclic ring containing 1 or 2 heteroatoms independently selected from Q, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3, and

each R^{6′} is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ and, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R^{6′} and R^{7′} on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂.NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

R^{6′} and R^{7′} are each independently selected from C₁-C₆ alkyl, C₂-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R^{6′} and R^{7′} on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R^{6′} and R^{7′} are each independently selected from C₁-C₆ alkyl, C₂-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R^{6′} and one R^{7′} are on adjacent atoms, and taken together with the atoms connecting them, form a C₄-C₈ carbocyclic-ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R^{6′} and one R^{7′} are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹,

In some embodiments, o=1 or 2, p=1, 2, or 3; and

one R^{6′} and one R^{7′} are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R^{6′} and one R^{7′}, are on adjacent atoms, and each pair of one R^{6′} and one R^{7′} taken together with the atoms connecting them independently form a C₄-C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6′} and one R^{7′}, are on adjacent atoms, and each pair of one R^{6′} and one R^{7′} taken together with the atoms connecting them independently form a C₆ carbocyclic ring or a 5-to 6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3, and

two pairs, each of one R^{6′} and one R^{7′}, are on adjacent atoms, and each pair of one R^{6′} and one R^{7′} taken together with the atoms connecting them independently form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹,

In some embodiments, o=2; p=2 or 3; and

two pairs, each of one R^{6′} and one R^{7′}, are on adjacent atoms, and each pair of one R^{6′} and one R^{7′} taken together with the atoms connecting them form a C₅ aliphatic carbocyclic ring.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a C₆ aliphatic carbocyclic ring.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a C₆ aromatic carbocyclic ring.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R^{6′} and one R⁷, are on adjacent atoms, and each pair of one R^{6′} and one R^{7′} taken together with the atoms connecting them form a 5-membered heteroaromatic ring containing 1 or 2 heteroatoms independently selected from O, N, and S.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a 6-membered aliphatic heterocyclic ring containing 1 or 2 heteroatoms independently selected from O, N, and S.

In some embodiments, o=2; p=2 or 3; and

two pairs, each of one R^{6′} and one R^{7′}, are on adjacent atoms, and each pair of one R^{6′} and one R^{7′} taken together with the atoms connecting them form a 6-membered heteroaromatic ring containing 1 or 2 heteroatoms independently selected from O, N, and S.

In some embodiments, o=2; p=2 or 3; and

two pairs, each of one R^{6′} and one R^{7′}, are on adjacent atoms, and each pair of one R^{6′} and one R^{7′} taken together with the atoms connecting them independently form a C₄-C₈ carbocyclic ring or a 5- to 8-membered heterocyclic ring containing 1 or 2 heteroatoms independently selected from O, N, and S,

wherein one of the two rings is fused to the R ring at the 2- and 3-positions relative to the bond connecting the B ring to the NH(CO)group, and the other of the two rings is fused to the B ring at the 5- and 6-positions relative to the bond connecting the B ring to the NH(CO) group.

In some embodiments, o=2; p=2; and

two pairs, each of one R^{6′} and one R^{7′}, are on adjacent atoms, and each pair of one R^{6′} and one R^{7′} taken together with the atoms connecting them form a C₅ aliphatic carbocyclic ring.

In some embodiments, o=2, p=3; and

two pairs, each of one R^{6′} and one R^{7′}, are on adjacent atoms, and each pair of one R^{6′} and one R^{7′} taken together with the atoms connecting them form a C₅ aliphatic carbocyclic ring; and one R^{7′} is halo (e.g., Cl or F).

In some embodiments, o=2; p=3; and

two pairs, each of one R^{6′} and one R⁷, are on adjacent atoms, and each pair of one R^{b′} and one R^{7′} taken together with the atoms connecting them form a C₅ aliphatic carbocyclic ring; and one R^{7′} is CN.

In some embodiments, one R^{7′} is pyrazolyl and is para to the bond connecting the B ring to the NH(CO) group of Formula AA; In some embodiments, one R⁷ is 3-pyrazolyl and is para to the bond connecting the B ring to the NH(CO) group of Formula AA. In some embodiments, one R^{7′} is 4-pyrazolyl and is para to the bond connecting the B ring to the NH(CO) group of Formula AA. In some embodiments, one R^{7′} is 5-pyrazolyl and is para to the bond connecting the B ring to the NH(CO) group of Formula AA. In some embodiments, one R^{7′} is thiazolyl and is para to the bond connecting the B ring to the NH(CO) group of Formula AA. In some embodiments, one R^{7′} is 4-thiazolyl and is para to the bond connecting the B ring to the NH(CO) group of Formula AA. In some embodiments, one R^{7′} is 5-thiazolyl and is para to the bond connecting the B ring to the NH(Co) group of Formula AA. In some embodiments, one R^{7′} is fund and is para to the bond connecting the B ring to the NH(CO) group of Formula AA. In some embodiments, one R⁷ is 2-furyl and is para to the bond connecting the B ring to the NH(CO) group of Formula AA. In some embodiments, one R^{7′} is thiophenyl and is para to the bond connecting the B ring to the NH(CO) group of Formula AA. In some embodiments, one R^{7′} is 2-thiophenyl and is para to the bond connecting the B ring to the NH(CO) group of Formula AA. In some embodiments, one R⁷ is phenyl and is para to the bond connecting the B ring to the NH(CO) group of Formula AA. In some embodiments, one R^{7′} is cycloalkenyl (e.g., cyclopentenyl, e.g., 1-cyclopentenyl) and is para to the bond connecting the B ring to the NH(CO) group of Formula AA. In some embodiments, one R^{7′} is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the NH(CO) group of Formula AA. In some embodiments, one R^{7′} is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the NH(CO) group of Formula AA. In some embodiments, one R^{7′} is phenyl optionally substituted with one or more C₆-C₁₀ aryloxy (e.g., phenoxy) and is para to the bond connecting the B ring to the NH(CO) group of Formula AA; In some embodiments, one R^{7′} is phenyl optionally substituted with one or more CN and is para to the bond connecting the B ring to the NH(CO) group of Formula AA; In some embodiments, one R^{7′} is phenyl optionally substituted with one or more halo (e.g., F, Cl) and is para to the bond connecting the B ring to the NH(CO) group of Formula AA and is para to the bond connecting the B ring to the NH(CO) group of Formula AA. In some embodiments, one R⁷ is phenyl optionally substituted with one or more COOC₁-C₆ alkyl (e.g., CO₂ₜ-Bu) and is para to the bond connecting the B ring to the NH(CO) group of Formula AA; In some embodiments, one R⁷ is phenyl optionally substituted with one or more S(O₂)C₁-C₆ alkyl (e.g., S(O₂)methyl) and is para to the bond connecting the B ring to the NH(CO) group of Formula AA. In some embodiments, one R^{7′} is phenyl optionally substituted with one or more 3- to 7-membered heterocycloalkyl (e.g., morpholinyl) and is para to the bond connecting the B ring to the NH(CO) group of Formula AA. In some embodiments, one R^{7′} is phenyl optionally substituted with one or more CONR⁸R⁹ (e.g., unsubstituted aminocarbonyl) and is para to the bond connecting the B ring to the NH(CO) group of Formula AA. In some embodiments, one R^{7′} is phenyl optionally substituted with one or more C₁-C₆ alkyl (e.g., methyl or propyl, e.g., 2-propyl) and with one or more halo (e.g., F, Cl) and is para to the bond connecting the B ring to the NH(CO) group of Formula AA.

In some embodiments, R^{6′} and R^{7′} are each attached to a carbon of an aryl ring B. In some embodiments, R^{6′} and R^{7′} are each attached to a carbon of a heteroaryl ring B. In some embodiments, R^{6′} is attached to a carbon and R^{7′} is attached to a nitrogen of a heteroaryl ring B. In some embodiments, R^{7′} is attached to a carbon and R^{6′} is attached to a nitrogen of a heteroaryl ring B.
In some embodiments, the optionally substituted ring B is

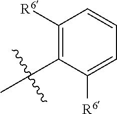

and each R^{6′} is independently selected from the group consisting of: C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, =NR¹⁰, COOC₁-C₆ alkyl, CONR⁸R⁹, 4- to 6-membered heterocycloalkyl, C₆-C₁₀ aryl, 5-to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
In some embodiments, the optionally substituted ring B is

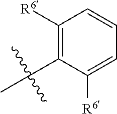

and each R^{6′} is independently selected from the group consisting of: C₁-C₆ alkyl, C₃-C₇ cycloalkyl, C₁-C₆ haloalkyl, C₂-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.
In some embodiments, the optionally substituted ring B is

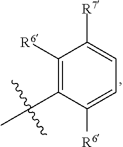

wherein each R^{6′} is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₆-C₁₀ alkynyl;
wherein R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R^{6′} and R^{7′}, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

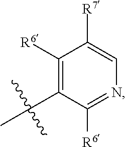

wherein each R^{6′} is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6′} and R^{7′}, taken together with the atoms connecting them, independently form C₄-C₇ carbocyclic ring or at least one 5-to-7-membered heterocyclic ring containing 1 or 2 heteroatoms independently selected from Q, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

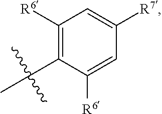

wherein each R^{6′} is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4 to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
In some embodiments, the optionally substituted ring B is

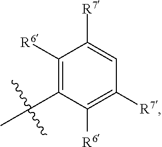

wherein each R⁵ is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R^{6′} and R^{7′} on adjacent atoms, taken together with the atoms connecting them, independently form at least one C₄-C₇ carbocyclic ring or at least one 5-to-7-membered heterocyclic ring containing 1 or 2 heteroatoms independently selected from Q, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

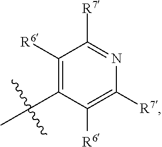

wherein each R⁶ is independently selected from C₁-C₆alkyl, C₃-C₇cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R^{6′} and R^{7′} on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

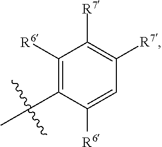

wherein each R⁶ is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R^{6′} and R^{7′}, taken together with the atoms connecting them, independently form a C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

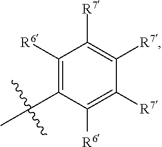

wherein each R^{6′} is independently selected from C₁-C₆ alkyl, C₃-C₇ cycloalkyl, C₁-C₆ haloalkyl, C₂-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5 to 10-membered heteroaryl), NHCO(4- to 6-membered heterocycloalkyl), and NHCOC₂-C₆ alkynyl;
wherein each R^{7′} is independently selected from C₁-C₆ alkyl, C₁-C₆ haloalkyl, C₂-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, =NR¹⁰, COOC₁-C₆ alkyl, C₆-C₁₀ and, and CONR⁸R⁹;

The Groups R^{6″} and R^{7″}

In some embodiments,

R^{6″} and R^{7″} are each independently selected from C₁-C₆ alkyl, C₁-C₆ haloalkyl, C₁-C₆ alkoxy, C₁-C₆ haloalkoxy, F, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,

wherein R^{6″} and R^{7″} are each optionally substituted with one or more substituents independently selected from

hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,

C₆-C₁₀ aryloxy, and S(O₂)C₁-C₆ alkyl; and wherein the C₁-C₆ alkyl or C₁-C₆ alkoxy that R^{6″} or R^{7″} is substituted with is optionally substituted with one or more hydroxyl, halo, C₆-C₁₀ aryl or NR⁸R⁹, or wherein R^{6″} or R^{7″} is optionally fused to a five- to -seven-membered carbocyclic ring or heterocyclic ring containing one or two heteroatoms independently selected from oxygen, sulfur and nitrogen,
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,

or at least one pair of R^{6″} and R^{7″} on adjacent atoms, taken together with the atoms connecting them, independently form at least one C₄-C₈ carbocyclic ring or at least one 5-to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂.NR⁸R⁹, ═NR¹⁰, COOC₁-C₆ alkyl, C₆-C₁₀ aryl, and CONR⁸R⁹.

In some embodiments,

R^{6″} and R^{7″} are each independently selected from C₁-C₆ alkyl, C₁-C₆ haloalkyl, C₁-C₆ alkoxy, C₁-C₆ haloalkoxy, F, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, C₀NR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6′} and R^{7′} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{b″} and R^{7″} are each independently selected from C₁-C₆ haloalkyl, C₁-C₆ alkoxy, C₁-C₆ haloalkoxy, F, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ and, 5- to 10-membered heteroaryl, OCOC₁-C₆ alkyl, OCOC₆-C₁₀ aryl, OCO(5- to 10-membered heteroaryl), OCO(3 to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷″ on adjacent atoms, taken together with the atoms connecting them, independently form at least one C₄-C₅ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6″} and R^{7″} are each independently selected from C₁-C₆ alkyl, F, Br, I, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ and, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl,
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6″} and R^{7″} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6″} and R^{7″} are each independently selected from C₁-C₆ alkyl, C₁-C₆ haloalkyl, C₁-C₆ alkoxy, C₁-C₆ haloalkoxy, F, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3 to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted,

or at least one pair of R^{7″} and R^{7″} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

In some embodiments,

R^{6″} and R^{7″} are each independently selected from C₁-C₆ alkyl, C₁-C₆ haloalkyl, C₁-C₆ alkoxy, C₁-C₆ haloalkoxy, F, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl are each unsubstituted;

or at least one pair of R^{6″} and R^{7″} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R^{7″} is independently selected from C₁-C₆ alkyl, C₁-C₆ haloalkyl, C₁-C₆ alkoxy, C₁-C₆ haloalkoxy, F, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ and, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6″} and R^{7″}, taken together with the atoms connecting them, independently form C₄-C₇ carbocyclic ring or at least one 5-to-7-membered heterocyclic ring containing 1 or 2 heteroatoms independently selected from Q, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹,

In some embodiments,

R^{7″} and R^{7″} are each independently selected from C₁-C₆ alkyl, C₁-C₆ alkoxy, F, Br, I, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo, or at least one pair of R^{7″} and R^{7″} on adjacent atoms, taken together with the atoms connecting them, independently form at least one C₄-C₈ carbocyclic ring, wherein the carbocyclic ring is optionally independently substituted with one or more hydroxy or oxo.

In some embodiments, R^{6″} and R^{7″} are each independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

In some embodiments, R⁶ is CN. In some embodiments, R^{6″} is C₁-C₆ alkyl substituted with hydroxyl (e.g., hydroxymethyl, hydroxyethyl, or 2-hydroxy-2-propyl. In some embodiments, R^{6″} is C₁-C₆ alkyl substituted with C₁-C₆ alkoxy (e.g., methoxymethyl) In some embodiments, R^{6″} is imidazolyl. In some embodiments, R^{6″} is pyrazoyl. In some embodiments, R^{6″} is pyrrolyl. In some embodiments, R^{6″} is thiazolyl. In some embodiments, R^{6″} is isothiazolyl. In some embodiments, R^{6″} is oxazolyl; In some embodiments, R^{6″} is isoxazolyl. In some embodiments, R^{6″} is pyridyl. In some embodiments, R^{6″} is pyrimidinyl. In some embodiments, R^{7″} is CN. In some embodiments, R^{7″} is C₁-C₆ alkyl substituted with hydroxyl (e.g., hydroxymethyl, hydroxyethyl, or 2-hydroxy-2-propyl. In some embodiments, R^{7″} is C₁-C₆ alkyl substituted with C₁-C₆ alkoxy (e.g., methoxymethyl) In some embodiments, R^{7″} is imidazolyl. In some embodiments, R^{7″} is pyrazolyl. In some embodiments, R^{7″} is pyrrolyl. In some embodiments, R^{7″} is thiazolyl. In some embodiments, R^{7″} is isothiazolyl. In some embodiments, R^{7″} is oxazolyl. In some embodiments, R^{7″} is isoxazolyl. In some embodiments, R^{7″} is pyridyl. In some embodiments, R^{7″} is pyrimidinyl.

In some embodiments, o=1; p=0; and

R^{6″} is selected from C₁-C₆ alkyl, C₁-C₆ haloalkyl, C₁-C₆ alkoxy, C₁-C₆ haloalkoxy, F, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5-to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=1; and

R^{6″} and R^{7″} are each independently selected from C₁-C₆ alkyl, C₁-C₆ alkoxy, F, Br, I, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo, or R^{6″} and R^{7″} on adjacent atoms, taken together with the atoms connecting them, independently form at least one CVCs carbocyclic ring, wherein the carbocyclic ring is optionally independently substituted with one or more hydroxy or oxo.

In some embodiments, o=1 or 2; p=1, 2, or 3; and

R^{6″} and R^{7″} are each independently selected from C₁-C₆ alkyl, C₁-C₆ haloalkyl, C₁-C₆ alkoxy, C₁-C₆ haloalkoxy, F, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2, p=1; and

each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₁-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5 to 10-membered heteroaryl), NHCO(4- to 6-membered heterocycloalkyl), and NHCOC₆-C₁₀ alkynyl;
and R^{7″} is independently selected from C₁-C₆ alkyl, C₁-C₆ haloalkyl, C₁-C₆ alkoxy, C₁-C₆ haloalkoxy, F, Br, I, CN, COC₁-C₆ alkyl, CO₂C₁-C₆ alkyl, CO₂C₃-C₆ cycloalkyl, OCOC₁-C₆ alkyl, OCOC₆-C₁₀ and, OCO(5- to 10-membered heteroaryl), OCO(3- to 7-membered heterocycloalkyl), C₆-C₁₀ aryl, 5-to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6″} and R^{7″}, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl; CONR⁸R⁹, and 4- to 6-membered heterocycloalkyl,

wherein the C₃-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₃-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7″} is independently selected from C₁-C₆ alkyl, C₁-C₆ haloalkyl, C₁-C₆ alkoxy, C₁-C₆ haloalkoxy, F, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R^{7″} and R^{7″} on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

In some embodiments, o=1 or 2; p=1, 2, or 3; and

R^{6″} and R^{7″} are each independently selected from C₁-C₆ alkyl, C₁-C₆ alkoxy, F, Br, I, NO₂, COC₁-C₆ alkyl, CO₂G-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo, or at least one pair of R^{7″} and R^{7″} on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2, p=1, 2, or 3; and

R^{6″} and R^{7″} are each independently selected from C₁-C₆ alkyl, C₁-C₆ alkoxy, F, Br, I, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R^{6″} and one R^{7″} are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and one R^{6″} and one R^{7″} are on adjacent atoms, and taken together with the atoms connecting them, form a C₆ carbocyclic ring or a 5-to 6-membered heterocyclic ring containing 1 or 2 heteroatoms independently selected from Q, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R^{6″} and one R^{7″} are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R^{6″} and one R^{7″}, are on adjacent atoms, and each pair of one R^{6″} and one R^{7″} taken together with the atoms connecting them independently form a C₄-C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6″} and one R^{7″}, are on adjacent atoms, and each pair of one R^{6″} and one R^{7″} taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, NR¹⁰. COOC₁-C₆ alkyl, C₆-C₁₀ aryl, and CONR⁸R⁹.

In some embodiments, o=2, p=2 or 3; and

two pairs, each of one R^{6″} and one R^{7″}, are on adjacent atoms, and each pair of one R^{6″} and one R^{7″} taken together with the atoms connecting them independently form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

Particular Embodiments Wherein o=1; p=0:

In some embodiments, R^{6″} is C₁-C₆ alkyl. In some embodiments, R^{6″} is isopropyl. In some embodiments, R^{6″} is ethyl. In some embodiments, R^{6″} is methyl. In some embodiments, R^{6″} is C₁-C₆ alkyl substituted with one or more halo. In some embodiments, R^{6″} is trifluoromethyl. In some embodiments, R^{6″} is trifluoromethoxy. In some embodiments, R^{6″} is C₃-C₇ cycloalkyl. In some embodiments, R^{6″} is cyclopropyl. In some embodiments, R^{6″} is bromo. In some embodiments, R^{6″} is fluoro. In some embodiments, R^{6″} is cyano. In some embodiments, R^{6″} is attached to a carbon of an aryl ring B. In some embodiments, R^{6″} is attached to a carbon of a heteroaryl ring B. In some embodiments, R⁶ is attached to a nitrogen of a heteroaryl ring B.

Particular Embodiments Wherein o=1 or 2; p=1, 2, or 3:

In some embodiments, at least one R^{6″} is C₁-C₆ alkyl, and at least one R^{7″} is C₁-C₆ alkyl optionally substituted with one or more halo. In some embodiments, at least one R^{6″} is C₁-C₆ alkyl and at least one R^{7″} is C₁-C₆ alkyl. In some embodiments, at least one R^{6″} is isopropyl and at least one R^{7″} is methyl. In some embodiments, at least one R^{6″} is isopropyl and at least one R^{7″} is isopropyl. In some embodiments. o=1; p=1; R^{6″} is isopropyl; and R^{7″} is isopropyl. In some embodiments, at least one R^{6″} is C₁-C₆ alkyl, and at least one R^{7″} is C₁-C₆ alkyl substituted with one or more halo. In some embodiments, at least one R^{6″} is isopropyl and at least one R^{7″} is trifluoromethyl. In some embodiments, at least one R^{6″} is C₁-C₆ alkyl, and at least one R^{7″} is C₃-C₇ cycloalkyl. In some embodiments, at least one R^{6″} is isopropyl and at least one R^{7″} is cyclopropyl. In some embodiments, o=1; p=1; R^{6″} is isopropyl, and R^{7″} is cyclopropyl. In some embodiments, at least one R^{6″} is C₁-C₆ alkyl, and at least one R^{7″} is fluoro. In some embodiments, at least one R^{6″} is isopropyl and at least one R^{7″} is fluoro. In some embodiments, at least one R^{6″} is isopropyl and at least one R^{7″} is bromo. In some embodiments, o=1; p=1; R^{6″} is isopropyl; and R^{7″} is bromo. In some embodiments, o=2; p=1; at least one R^{7″} is isopropyl; and R⁷ is bromo. In some embodiments, o=1, p=1, R^{6″} is isopropyl; and R^{7″} is bromo. In some embodiments, o=2; p=1; at least one R^{6″} is isopropyl; and R^{7″} is bromo. In some embodiments, o=2; p=2; at least one R^{6″} is isopropyl; and at least one R^{7″} is bromo. In some embodiments, o=2; p=2; at least one R^{6″} is isopropyl; one R^{7″} is fluoro; and the other R^{7″} is cyano. In some embodiments, o=2; p=3; at least one R^{6″} is isopropyl; two R^{7″} are fluoro; and one R^{7″} is bromo. In some embodiments, o=2; p=1; at least one R^{6″} is ethyl; and R^{7″} is fluoro. In some embodiments, o=2; p=1; one R^{6″} is isopropyl; the other R^{6″} is trifluoromethyl; and R^{7″} is fluoro. In some embodiments, at least one R^{6″} is C₁-C₆ alkyl, and at least one R^{7″} is cyano. In some embodiments, at least one R^{6″} is isopropyl and at least one R^{7″} is cyano. In some embodiments, o=1; p=1; R^{6″} is isopropyl, and R^{7″} is cyano. In some embodiments, o=2, p=1, at least one R^{6″} is isopropyl; and R^{7″} is cyano. In some embodiments, at least one R^{6″} is C₃-C₇ cycloalkyl, and at least one R^{7″} is C₃-C₇ cycloalkyl. In some embodiments, at least one R^{6″} is cyclopropyl, and at least one R^{7″} is cyclopropyl. In some embodiments, at least one R^{6″} is C₃-C₇ cycloalkyl, and at least one R^{7″} is fluoro, boromo or iodo. In some embodiments, at least one R^{6″} is cyclopropyl and at least one R^{7″} is fluoro, boromo or iodo. In some embodiments, at least one R^{6″} is cyclopropyl and at least one R^{7″} is bromo. In some embodiments, at least one R^{6″} is cyclopropyl and at least one R^{7″} is fluoro. In some embodiments, o=1, p=1; R^{6″} is cyclopropyl; and R^{7″} is bromo. In some embodiments, o=1; p=1; R^{6″} is cyclopropyl; and R^{7″} is fluoro. In some embodiments, at least one R^{6″} is C₁-C₆ alkyl, and at least one R^{7″} is C₁-C₆ alkoxy optionally substituted with one or more halo. In some embodiments, at least one R^{6″} is isopropyl, and at least one R^{7″} is C₁-C₆ alkoxy. In some embodiments, at least one R^{6″} is isopropyl, and at least one R^{7″} is methoxy. In some embodiments, o=1; p=1; R^{6″} is isopropyl, and R^{7″} is methoxy. In some embodiments, 0=2; p=1; at least one R⁶ is isopropyl, and R^{7″} is methoxy. In some embodiments, at least one R^{6′} is C₁-C₆ alkyl, and at least one R⁷ is C₁-C₆ alkoxy substituted with one or more halo. In some embodiments, at least one R^{6″} is isopropyl, and at least one R^{7″} is trifluoromethoxy. In some embodiments, at least one R^{6″} is isopropyl, and at least one R^{7″} is difluoromethoxy. In some embodiments, at least one R^{6″} is fluoro, and at least one R^{7″} is C₁-C₆ haloalkyl optionally substituted with hydroxy. In some embodiments, o=1; p=1; R⁶ is fluoro, and R^{7″} is trifluoromethyl. In some embodiments, at least one R^{6″} is fluoro, and at least one R^{7″} is C₁-C₆ haloalkoxy. In some embodiments, at least one R^{6″} is fluoro, and at least one R^{7″} is trifluoromethoxy. In some embodiments, o=1; p=1; R^{6″} is fluoro, and R^{7″} is trifluoromethoxy. In some embodiments, at least one R⁶ is C₁-C₆ alkoxy; and at least one R^{7″} is fluoro. In some embodiments, o=1; p=2; R^{6″} is C₁-C₆ alkoxy; and at least one R^{7″} is fluoro.

In some embodiments, at least one R^{7″} is C₁-C₆ alkyl, and at least one R^{6″} is C₁-C₆ alkyl optionally substituted with one or more halo. In some embodiments, at least one R⁷ is isopropyl and at least one R^{6″} is methyl. In some embodiments, at least one R⁷ is C₁-C₆ alkyl, and at least one R^{6″} is C₁-C₆ alkyl substituted with one or more halo. In some embodiments, at least one R^{7″} is isopropyl and at least one R^{6″} is trifluoromethyl. In some embodiments, at least one R^{7″} is C₁-C₆ alkyl, and at least one R^{6″} is C₃-C₇ cycloalkyl. In some embodiments, at least one R^{7″} is isopropyl and at least one R^{6″} is cyclopropyl. In some embodiments, o=1; p=1; R^{7″} is isopropyl; and R^{6″} is cyclopropyl. In some embodiments, at least one R^{7″} is C₁-C₆ alkyl, and at least one R^{6″} is fluoro. In some embodiments, at least one R⁷ is isopropyl and at least one R^{6″} is bromo. In some embodiments, at least one R^{7″} is isopropyl and at least one R^{6″} is fluoro. In some embodiments, o=1; p=1; R^{7″} is isopropyl; and R^{6″} is bromo. In some embodiments, o=2; p=1; R^{7″} is isopropyl; and at least one R^{6″} is bromo. In some embodiments, o=1; p=1; R^{7″} is isopropyl; and R^{6″} is fluoro. In some embodiments, o=2; p=1; R^{7′} is isopropyl; and at least one R^{6″} is fluoro. In some embodiments, o=2; p=2; at least one R^{7″} is isopropyl; and at least one R^{6″} is fluoro. In some embodiments, o=2; p=2; at least one R^{7″} is isopropyl; one R^{6″} is fluoro; and the other R^{6″} is cyano. In some embodiments, o=2; p=1; R^{7″} is ethyl; and at least one R^{6″} is fluoro. In some embodiments, o=1; p=2; one R^{7′} is isopropyl; the other R^{7″} is trifluoromethyl; and R^{6″} is bromo. In some embodiments, at least one R^{7″} is C₁-C₆ alkyl, and at least one R^{6″} is cyano. In some embodiments, at least one R^{7′} is isopropyl and at least one R^{6″} is cyano. In some embodiments, o=4; p=4; R^{7″} is isopropyl; and R^{6″} is cyano. In some embodiments, o=2; p=1; R^{7″} is isopropyl; and at least one R^{6″} is cyano. In some embodiments, at least one R^{7″} is C₃-C₇ cycloalkyl, and at least one R^{6″} is C₃-C₇ cycloalkyl. In some embodiments, at least one R^{7″} is cyclopropyl, and at least one R^{6″} is cyclopropyl. In some embodiments, at least one R^{7″} is C₃-C₇ cycloalkyl, and at least one R^{6″} is fluoro. In some embodiments, at least one R^{7″} is cyclopropyl and at least one R^{6″} is bromo. In some embodiments, at least one R^{7″} is cyclopropyl and at least one R^{6″} is fluoro. In some embodiments, 0=4; p=4; R^{7″} is cyclopropyl; and R^{6″} is fluoro. In some embodiments, at least one R^{7″} is C₁-C₆ alkyl, and at least one R^{6″} is C₁-C₆ alkoxy optionally substituted with one or more halo. In some embodiments, at least one R^{7″} is isopropyl, and at least one R^{6″} is C₁-C₆ alkoxy. In some embodiments, at least one R^{7″} is isopropyl, and at least one R^{6″} is methoxy. In some embodiments, o=1; p=1; R^{7″} is isopropyl, and R^{7″} is methoxy. In some embodiments, o=2; p=1; R^{7″} is isopropyl, and at least one R^{6″} is methoxy. In some embodiments, at least one R^{7″} is C₁-C₆ alkyl, and at least one R^{6″} is C₁-C₆ alkoxy substituted with one or more halo. In some embodiments, at least one R^{7″} is isopropyl, and at least one R^{6″} is trifluoromethoxy. In some embodiments, at least one R^{7″} is fluoro, and at least one R^{6″} is C₁-C₆ haloalkyl optionally substituted with one or more hydroxy. In some embodiments, o=1; p=1; R^{7″} is fluoro, and R^{6″} is trifluoromethyl. In some embodiments, at least one R^{7″} is fluoro, and at least one R^{6″} is C₁-C₆ haloalkoxy. In some embodiments, at least one R^{7′} is fluoro, and at least one R^{6″} is trifluoromethoxy. In some embodiments, o=1; p=1; R^{7″} is fluoro, and R^{6″} is trifluoromethoxy. In some embodiments, at least one R⁷ is C₁-C₆ alkoxy; and at least one R^{6″} is bromo. In some embodiments, o=1; p=2; at least one R^{7″} is C₁-C₆ alkoxy; and R^{6″} is bromo.

In some embodiments, R^{6″} and R^{7″} are each attached to a carbon of an aryl ring B. In some embodiments, R^{6″} and R^{7′} are each attached to a carbon of a heteroaryl ring B. In some embodiments, R^{6″} is attached to a carbon and R^{7″} is attached to a nitrogen of a heteroaryl ring B.

In some embodiments, R^{7″} is attached to a carbon and R^{6″} is attached to a nitrogen of a heteroaryl ring B.

In some embodiments, one R^{6″} and one R^{7″} are on adjacent atoms, and taken together with the atoms connecting them, form a C₅ carbocyclic ring optionally substituted with one or more substituents independently selected from hydroxy, F, Br, I, oxo, C₁-C₆ alkyl, C₁-C₆ alkoxy, NR⁸R⁹, ═NR¹⁰, COOC₁-C₆ alkyl, C₆-C₁₀ and, and CONR⁸R⁹.

In some embodiments, R^{6″} and R^{7″} are on adjacent atoms, and taken together with the atoms connecting them, form a C₅ aliphatic carbocyclic ring.

In some embodiments, R^{6″} and R^{7″} are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R^{6″} and R^{7″} are on adjacent atoms, and taken together with the atoms connecting them, form a C₆ aliphatic carbocyclic ring.

In some embodiments, R^{6″} and R^{7″} are on adjacent atoms, and taken together with the atoms connecting them, form a C₆ aromatic carbocyclic ring.

In some embodiments, R^{6″} and R^{7″} are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, F, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R^{6″} and R^{7″} are on adjacent atoms, and taken together with the atoms connecting them, form a 5-membered aliphatic heterocyclic ring containing 1 or 2 heteroatoms independently selected from O, N, and S.

In some embodiments, R^{6″} and R^{7″} are on adjacent atoms, and taken together with the atoms connecting them, form a 5-membered heteroaromatic ring containing 1 or 2 heteroatoms independently selected from O, N, and S.

In some embodiments, R^{6″} and R^{7′} are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, F, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R^{6″} and R^{7″} are on adjacent atoms, and taken together with the atoms connecting them, form a 6-membered aliphatic heterocyclic ring containing 1 or 2 heteroatoms independently selected from O, N, and S,

In some embodiments, R^{6″} and R^{7″} are on adjacent atoms, and taken together with the atoms connecting them, form a 6-membered heteroaromatic ring containing 1 or 2 heteroatoms independently selected from O, N, and S.

In some embodiments, one R^{6″} and one R^{7″} are on adjacent atoms, and taken together with the atoms connecting them, form a C₄-C₈ carbocyclic ring or a 5- to 8-membered heterocyclic ring containing 1 or 2 heteroatoms independently selected from O, N, and S,

wherein the ring is fused to the B ring at the 2- and 3-positions relative to the bond connecting the B ring to the NH(CO)group.

In some embodiments, o=2; p=2 or 3; and

two pairs, each of one R^{6″} and one R^{7″}, are on adjacent atoms, and each pair of one R^{6″} and one R^{7″} taken together with the atoms connecting them form a C₅ carbocyclic ring optionally independently substituted with one or more substituents independently selected from hydroxy, F, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3, and

two pairs, each of one R^{6″} and one R^{7″}, are on adjacent atoms, and each pair of one R^{6″} and one R^{7″} taken together with the atoms connecting them form a C₅ aliphatic carbocyclic ring.

In some embodiments, o=2; p=2 or 3, and

two pairs, each of one R^{6″} and one R^{7″}, are on adjacent atoms, and each pair of one R^{6″} and one R^{7″} taken together with the atoms connecting them form a C₆ carbocyclic ring optionally independently substituted with one or more substituents independently selected from hydroxy, F, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6″} and one R^{7″}, are on adjacent atoms, and each pair of one R^{6″} and one R^{7″} taken together with the atoms connecting them form a C₆ aliphatic carbocyclic ring,

In some embodiments, o=2; p=2 or 3; and

two pairs, each of one R^{6″} and one R^{7″}, are on adjacent atoms, and each pair of one R^{6″} and one R^{7″} taken together with the atoms connecting them form a C₆ aromatic carbocyclic ring.

In some embodiments, o=2; p=2 or 3; and

two pairs, each of one R^{6″} and one R^{7″}, are on adjacent atoms, and each pair of one R^{6″} and one R^{7″} taken together with the atoms connecting them form a 5-membered heterocyclic ring containing 1 or 2 heteroatoms independently selected from O, N, and S, optionally substituted with one or more substituents independently selected from hydroxy, F, Br, I, oxo, C₁-C₆ alkyl, C₁-C₆ alkoxy, NR⁸R⁹, —NR¹⁰, COOC₁-C₆ alkyl, C₆-C₁₀ aryl, and CONR⁸R⁹.

In some embodiments, o=2; p=2 or 3, and

two pairs, each of one R^{6″} and one R^{7″}, are on adjacent atoms, and each pair of one R^{6″} and one R^{7″} taken together with the atoms connecting them form a 5-membered aliphatic heterocyclic ring containing 1 or 2 heteroatoms independently selected from O, N, and S.

In some embodiments, o=2; p=2 or 3, and

two pairs, each of one R^{6″} and one R^{7″}, are on adjacent atoms, and each pair of one R^{6′} and one R^{7″} taken together with the atoms connecting them form a 5-membered heteroaromatic ring containing 1 or 2 heteroatoms independently selected from O, N, and S.

In some embodiments, o=2; p=2 or 3; and

two pairs, each of one R^{6′} and one R^{7″} are on adjacent atoms, and each pair of one R^{6″} and one R^{7″} taken together with the atoms connecting them form a 6-membered heterocyclic ring containing 1 or 2 heteroatoms independently selected from O, N, and S, optionally substituted with one or more substituents independently selected from hydroxy, F, Br, I, oxo, C₁-C₆ alkyl, C₁-C₆ alkoxy, NR⁸R⁹, —NR¹⁰, COOC₁-C₆ alkyl, C₆-C₁₀ aryl, and CONR⁸R⁹.

In some embodiments, o=2; p=2 or 3; and

two pairs, each of one R^{6″} and one R^{7″}, are on adjacent atoms, and each pair of one R^{6″} and one R^{7″} taken together with the atoms connecting them form a 6-membered aliphatic heterocyclic ring containing 1 or 2 heteroatoms independently selected from O, N, and S.

In some embodiments, o=2; p=2 or 3, and

two pairs, each of one R^{6″} and one R^{7″}, are on adjacent atoms, and each pair of one R^{6″} and one R^{7″} taken together with the atoms connecting them form a 6-membered heteroaromatic ring containing 1 or 2 heteroatoms independently selected from O, N, and S.

In some embodiments, o=2; p=2 or 3; and

two pairs, each of one R^{6″} and one R^{7″}, are on adjacent atoms, and each pair of one R^{6′} and one R^{7″} taken together with the atoms connecting them independently form a C₄-C₈ carbocyclic ring or a 5- to 8-membered heterocyclic ring containing 1 or 2 heteroatoms independently selected from O, N, and S,

wherein one of the two rings is fused to the B ring at the 2- and 3-positions relative to the bond connecting the B ring to the NH(CO)group, and the other of the two rings is fused to the B ring at the 5- and 6-positions relative to the bond connecting the B ring to the NH(CO) group.

In some embodiments, o=2; p=2; and

two pairs, each of one R^{6′} and one R^{7″} are on adjacent atoms, and each pair of one R^{6″} and one R^{7′} taken together with the atoms connecting them form a C₅ aliphatic carbocyclic ring.

In some embodiments, o=2, p=3; and

two pairs, each of one R^{6′} and one R⁷″, are on adjacent atoms, and each pair of one R^{6″} and one R^{7″} taken together with the atoms connecting them form a C₅ aliphatic carbocyclic ring, and one R^{7″} is Br or F.

In some embodiments, o=2; p=3, and

two pairs, each of one R⁶ and one R⁷, are on adjacent atoms, and each pair of one R^{6″} and one R^{7″} taken together with the atoms connecting them form a C₅ aliphatic carbocyclic ring; and one R^{7″} is CN.

In some embodiments, one R^{7″} is pyrazolyl and is para to the bond connecting the B ring to the NH(CO) group of Formula AA. In some embodiments, one R^{7″} is 3-pyrazolyl and is para to the bond connecting the B ring to the NH(CO) group of Formula AA. In some embodiments, one R^{7″} is 4-pyrazolyl and is para to the bond connecting the B ring to the NH(CO) group of Formula AA. In some embodiments, one R⁷ is 5-pyrazolyl and is para to the bond connecting the B ring to the NH(CO) group of Formula AA. In some embodiments, one R⁷ is thiazolyl and is para to the bond connecting the B ring to the NH(CO) group of Formula AA. In some embodiments, one R^{7″} is 4-thiazolyl and is para to the bond connecting the B ring to the NH(CO) group of Formula AA. In some embodiments, one R^{7″} is 5-thiazolyl and is para to the bond connecting the B ring to the NH(CO) group of Formula AA; In some embodiments, one R^{7″} is furyl and is para to the bond connecting the B ring to the NH(CO) group of Formula AA. In some embodiments, one R^{7″} is 2-furyl and is para to the bond connecting the B ring to the NH(CO) group of Formula AA. In some embodiments, one R^{7″} is thiophenyl and is para to the bond connecting the B ring to the NH(CO) group of Formula AA; In some embodiments, one R^{7″} is 2-thiophenyl and is para to the bond connecting the B ring to the NH(CO) group of Formula AA. In some embodiments, one R^{7″} is phenyl and is para to the bond connecting the B ring to the NH(CO) group of Formula AA; In some embodiments, one R^{7″} is cycloalkenyl (e.g., cyclopentenyl, e.g., 1-cyclopentenyl) and is para to the bond connecting the B ring to the NH(CO) group of Formula AA; In some embodiments, one R^{7″} is phenyl optionally substituted with one or more C₁-C₆ alkyl (e.g., methyl or propyl, e.g., 2-propyl) optionally substituted with one or more hydroxyl, NR⁸R⁹ (e.g., dimethyl amino), or C₆-C₁₀ aryl (e.g., phenyl, naphthyl, or methylenedioxyphenyl) and is para to the bond connecting the B ring to the NH(CO) group of Formula AA. In some embodiments, one R^{7″} is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the NH(CO) group of Formula AA. In some embodiments, one R^{7″} is phenyl optionally substituted with one or more C₆-C₁₀ aryloxy (e.g., phenoxy) and is para to the bond connecting the B ring to the NH(CO) group of Formula AA. In some embodiments, one R^{7″} is phenyl optionally substituted with one or more CN and is para to the bond connecting the B ring to the NH(CO) group of Formula AA. In some embodiments, one R^{7″} is phenyl optionally substituted with one or more F and is para to the bond connecting the B ring to the NH(Co) group of Formula AA. In some embodiments, one R^{7″} is phenyl optionally substituted with one or more COOC₁-C₆ alkyl (e.g., CO₂ₜ-Bu) and is para to the bond connecting the B ring to the NH(Co) group of Formula AA. In some embodiments, one R^{7″} is phenyl optionally substituted with one or more S(O₂)C₁-C₆ alkyl (e.g., S(O₂)methyl) and is para to the bond connecting the B ring to the NH(CO) group of Formula AA. In some embodiments, one R^{7″} is phenyl optionally substituted with one or more 3- to 7-membered heterocycloalkyl (e.g., morpholinyl) and is para to the bond connecting the B ring to the NH(Co) group of Formula AA. In some embodiments, one R^{7″} is phenyl optionally substituted with one or more CONR⁸R⁹ (e.g., unsubstituted amido) and is para to the bond connecting the B ring to the NH(CO) group of Formula AA. In some embodiments, one R^{7″} is phenyl optionally substituted with one or more C₁-C₆ alkyl (e.g., methyl or propyl, e.g., 2-propyl) and with one or more F and is para to the bond connecting the B ring to the NH(CO) group of Formula AA.
In some embodiments, the optionally substituted ring B is

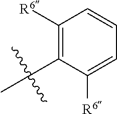

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, F, Br, I, C₁-C₆ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, =NR¹⁰, COOC₁-C₆ alkyl, CONR⁸R⁹, 4- to 6-membered heterocycloalkyl, C₆-C₁₀ aryl, 5-to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
In some embodiments, the optionally substituted ring B is

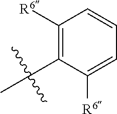

and each R^{6″} is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, F, Br, I, or oxo.
In some embodiments, the optionally substituted ring B is

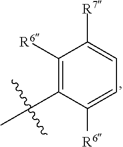

wherein each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7″} is independently selected from C₁-C₆ alkyl, C₁-C₆ haloalkyl, C₁-C₆ alkoxy, C₁-C₆ haloalkoxy, F, Br, I, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6″} and R^{7″}, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹,

In some embodiments, the optionally substituted ring B is

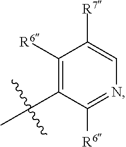

wherein each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl. OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7″} is independently selected from C₁-C₆ alkyl, C₁-C₆ haloalkyl, C₁-C₆ alkoxy, C₁-C₆ haloalkoxy, F, Br, I, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy,

or R^{6″} and R^{7″}, taken together with the atoms connecting them, independently form C₄-C₇ carbocyclic ring or at least one 5-to 7-membered heterocyclic ring containing 1 or 2 heteroatoms independently selected from O, N, and S, wherein the carbocyclic ring or heterocyclic ring is optionally independently substituted with one or more substituents independently selected from hydroxy, F, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

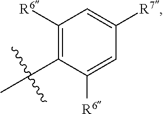

wherein each R^{6′} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7″} is independently selected from C₁-C₆alkyl, C₁-C₆ haloalkyl, C₁-C₆ alkoxy, C₁-C₆ haloalkoxy, F, Br, I, COC₁-C₆ alkyl, CO₂C₁-C₆ alkyl, CO₂C₃-C₀ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
In some embodiments, the optionally substituted ring B is

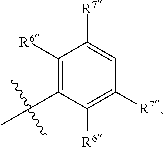

wherein each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ and, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₆-C₁₀ alkynyl;
wherein each R^{7″} is independently selected from C₁-C₆ alkyl, C₁-C₆ haloalkyl, C₁-C₆ alkoxy, C₁-C₆ haloalkoxy, F, Br, I, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶″ and R⁷″ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

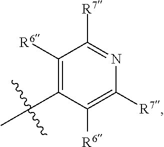

wherein each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5 to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, F, Br, I, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R^{6″} and R^{7″} on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₅-C₁₀ aryl, and CONR⁸R⁹.
In some embodiments, the optionally substituted ring B

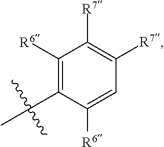

wherein each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, F, Br, I, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form a C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, —NR¹⁰, COOC₁-C₆ alkyl, C₆-C₁₀ aryl, and CONR⁸R⁹.
In some embodiments, the optionally substituted ring B is

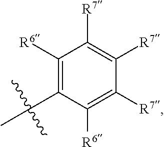

wherein each R^{6″} is independently selected from C₁-C₆ alkyl, C₃-C₇ cycloalkyl, C₁-C₆ haloalkyl, C₁-C₆ alkoxy, C₁-C₆ haloalkoxy, F, Br, I, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, F, Br, I, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R^{6″} and R^{7″} on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, =NR¹⁰, COOC₁-C₆ alkyl, C₆-C₁₀ and, and CONR⁸R⁹.

In some embodiments,

R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,

wherein R^{6′″} and R^{7′″} are each optionally substituted with one or more substituents independently selected from

hydroxy, halo, CN, oxo, C₁-C₆ alkyl, C₁-C₆ alkoxy, NR⁸R⁹, —NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,

C₆-C₁₀ aryloxy, and S(O₂)C₁-C₆ alkyl; and wherein the C₁-C₆ alkyl or C₁-C₆ alkoxy that R^{6′″} or R^{7′″} is substituted with is optionally substituted with one or more hydroxyl, halo, C₆-C₁₀ aryl or NR⁸R⁹, or wherein R^{6′″} or R^{7′″} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6′″} and R^{7′″} on adjacent atoms, taken together with the atoms connecting them, independently form at least one C₄, C₆, C₇, or C₅ carbocyclic ring or at least one 5-to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, —NR¹⁰, COOC₁-C₆ alkyl, C₆-C₁₀ aryl, and CONR⁸R⁹.

In some embodiments,

R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ and, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6′″} and R^{7′″} on adjacent atoms, taken together with the atoms connecting them, independently form at least one C₄, C₆, C₇, or C₅ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, NR¹⁰, COOC₁-C₆ alkyl, C₆-C₁₀ and, and CONR⁸R⁹.

In some embodiments,

R^{6′″} and R^{7′″} are each independently selected from C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ and, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl; or at least one pair of R⁶ and R⁷ on adjacent atoms, taken together with the atoms connecting them, independently form at least one C₄, C₆, C₇, or C₅ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6′″} and R^{7′″} are each independently selected from C₁-C₆ alkyl, Br, I, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3 to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl is optionally-substituted with one or more substituents each independently selected from hydroxy, halo, CN, oxo, C₁-C₆ alkyl, C₁-C₆ alkoxy, NR⁸R⁹, NR¹⁹, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl,
wherein the 3- to 7-membered heterocycloalkyl, C₆-C₁₀ aryl, 5- to 10-membered heteroaryl, NHCOC₆-C₁₀ aryl, NHCO(5 to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R^{6′″} and R^{7′″} on adjacent atoms, taken together with the atoms connecting them, independently form at least one C₄, C₆, C₇, or C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;

or at least one pair of R^{6′″} and R^{7′″} on adjacent atoms, taken together with the atoms connecting them, independently form at least one C₄, C₆, C₇, or C₈ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ and, and CONR⁸R⁹.

In some embodiments,

R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl are each unsubstituted;

or at least one pair of R^{6′″} and R^{7′″} on adjacent atoms, taken together with the atoms connecting them, independently form at least one C₄, C₆, C₇, or C₈ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R^{6′″} is independently selected from C₁-C₆ alkyl, C₃-C₇cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₆-C₁₀ alkynyl;
and R^{7′″} is independently selected from C₁-C₆ alkyl, C₁-C₆ haloalkyl, C₁-C₆, alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5-to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6′″} and R^{7′″}, taken together with the atoms connecting them, independently form C₄, C₆, or C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R^{6′″} and R^{7′″} are each independently selected from C₁-C₆ alkyl, C₁-C₆ alkoxy, Br, I, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo, or at least one pair of R^{6′″} and R^{7′″} on adjacent atoms, taken together with the atoms connecting them, independently form at least one C₄, C₆, C₇, or C₈ carbocyclic ring, wherein the carbocyclic ring is optionally independently substituted with one or more hydroxy or oxo.

In some embodiments, R^{6′″} and R^{7′″} are each independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

In some embodiments, R^{6′″} is CN. In some embodiments, R^{6′″} is C₁-C₆ alkyl substituted with hydroxyl (e.g., hydroxymethyl, hydroxyethyl, or 2-hydroxy-2-propyl. In some embodiments, R^{6′″} is C₁-C₆ alkyl substituted with C₁-C₆ alkoxy (e.g., methoxymethyl) In some embodiments, R^{6′″} is imidazolyl. In some embodiments, R^{6′″} is pyrazolyl. In some embodiments, R^{6′″} is pyrrolyl. In some embodiments, R^{6′″} is thiazolyl. In some embodiments, R^{6′″} is isothiazolyl. In some embodiments, R^{6′″} is oxazolyl. In some embodiments, R^{6′″} is isoxazolyl. In some embodiments, R^{6′″} is pyridyl. In some embodiments, R^{6′″} is pyrimidinyl. In some embodiments, R^{7′″} is CN. In some embodiments, R^{7′″} is C₁-C₆ alkyl substituted with hydroxyl (e.g., hydroxymethyl, hydroxyethyl, or 2-hydroxy-2-propyl. In some embodiments, R^{7′″} is C₁-C₆ alkyl substituted with C₁-C₆ alkoxy (e.g., methoxymethyl) In some embodiments, R^{7′″} is imidazolyl. In some embodiments, R⁷ is pyrazolyl. In some embodiments, R^{7′″} is pyrrolyl. In some embodiments, R^{7″} is thiazolyl. In some embodiments, R^{7″} is isothiazolyl. In some embodiments, R⁷ is oxazolyl. In some embodiments, R⁷ is isoxazolyl. In some embodiments, R^{7″} is pyridyl. In some embodiments, R^{7″} is pyrimidinyl.

In some embodiments, o=1; p=0; and

R^{6′″} is selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ and, 5-to 10-membered heteroaryl, NIB, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₆-C₁₀ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=0; and

R^{6′″} is selected from C₁-C₆ alkyl, C₁-C₆ alkoxy, Br, I, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl, wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3 to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R^{6′″} is independently selected from C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R^{7′″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6′″} and R^{7′″}, taken together with the atoms connecting them, independently form C₄, C₆, or C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2, p=2 or 3; and

each R^{6′″} is independently selected from C₁-C₆ alkyl, C₃-C₇cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5 to 10-membered heteroaryl), NHCO(4- to 6-membered heterocycloalkyl), and NHCOC₆-C₁₀ alkynyl;
wherein each R^{7′″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R^{6′″} and R^{7′″} on adjacent atoms, taken together with the atoms connecting them, independently form at least one C₄, C₆, or C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ and, and CONR⁸R⁹.

In some embodiments, o=1 or 2; p=1, 2, or 3; and

R^{6′″} and R^{7′″} are each independently selected from C₁-C₆ alkyl, C₁-C₆ alkoxy, Br, I, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo, or at least one pair of R^{6′″} and R^{7′″} on adjacent atoms, taken together with the atoms connecting them, independently form at least one C₄, C₆, C₇, or C₅ carbocyclic ring, wherein the carbocyclic ring is optionally independently substituted with one or more hydroxy or oxo.

In some embodiments, o=1 or 2, p=1, 2, or 3; and R^{6′″} and R^{7′″} are each independently selected from C₁-C₆ alkyl, C₁-C₆ alkoxy, Br, I, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R^{6′″} and one R^{7′″} are on adjacent atoms, and taken together with the atoms connecting them, form a C₄, C₆, C₇, or C₈ carbocyclic ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R^{6′″} and one R^{7′″} are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2, p=1, 2, or 3; and

one R^{6′″} and one R^{7′″} are on adjacent atoms, and taken together with the atoms connecting them, form a C₄, C₆, C₇, or C₅ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R^{6′″} and one R^{7′″}, are on adjacent, atoms, and each pair of one R^{6′″} and one R^{7′″} taken together with the atoms connecting them independently form a C₄, C₆, C₇, or C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6′″} and one R^{7′″}, are on adjacent atoms, and each pair of one R^{6′″} and one R^{7′″} taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, NR¹⁰, COOC₁-C₆ alkyl, C₆-C₁₀ aryl, and CONR⁸R⁹.

In some embodiments, o=2; p=2 or 3; and

two pairs, each of one R^{6′″} and one R^{7′″}, are on adjacent atoms, and each pair of one R^{6′″} and one R^{7′″} taken together with the atoms connecting them independently form a C₄, C₆, C₇, or C₈ carbocyclic ring or a 5- to 8-membered heterocyclic ring containing 1 or 2 heteroatoms independently selected from O, N, and S, wherein the carbocyclic ring or heterocyclic ring is unsubstituted.

Particular Embodiments Wherein o=1; p=0:

In some embodiments, R^{6′″} is C₁-C₆ alkyl. In some embodiments, R^{6′″} is isopropyl. In some embodiments, R^{6′″} is ethyl. In some embodiments, R^{6′″} is methyl. In some embodiments, R^{6′″} is C₁-C₆ alkyl substituted with one or more halo. In some embodiments, R^{6′″} is trifluoromethyl. In some embodiments, R^{6′″} is trifluoromethoxy. In some embodiments, R^{6′″} is C₃-C₇ cycloalkyl. In some embodiments, R^{6′″} is cyclopropyl. In some embodiments, R^{6′″} is Br. In some embodiments, R^{6′″} is 1. In some embodiments, R^{6′″} is cyano. In some embodiments, R^{6′″} is attached to a carbon of an and ring B. In some embodiments, R^{6′″} is attached to a carbon of a heteroaryl ring B. In some embodiments, R^{6′″} is attached to a nitrogen of a heteroaryl ring B.

Particular Embodiments Wherein o=1 or 2; p=1, 2, or 3:

In some embodiments, at least one R^{6′″} is C₁-C₆ alkyl, and at least one R^{7′″} is C₁-C₆ alkyl optionally substituted with one or more halo. In some embodiments, at least one R^{6′″} is C₁-C₆ alkyl and at least one R^{7′″} is C₁-C₆ alkyl. In some embodiments, at least one R^{6′″} is isopropyl and at least one R^{7′″} is methyl. In some embodiments, at least one R⁶ is isopropyl and at least one R^{7′″} is isopropyl. In some embodiments, o=1; p=1; R^{6′″} is isopropyl; and R^{7′″} is isopropyl. In some embodiments, at least one R^{6′″} is C₁-C₆ alkyl, and at least one R^{7′″} is C₁-C₆ alkyl substituted with one or more halo. In some embodiments, at least one R^{6′″} is isopropyl and at least one R^{7′″} is trifluoromethyl. In some embodiments, at least one R^{6′″} is C₁-C₆ alkyl, and at least one R^{7′″} is C₃-C₇ cycloalkyl. In some embodiments, at least one R^{6′″} is isopropyl and at least one R^{7′″} is cyclopropyl. In some embodiments, o=1; p=1; R^{6′″} is isopropyl; and R^{7′″} is cyclopropyl. In some embodiments, at least one R^{6″} is C₁-C₆ alkyl, and at least one R⁷ is Br or I. In some embodiments, at least one R^{6′″} is isopropyl and at least one R^{7′″} is Br. In some embodiments, at least one R⁶ is isopropyl and at least one R^{7′″} is 1. In some embodiments, o=1; p=1; R^{6′″} is isopropyl; and R^{7′″} is Br. In some embodiments, o=2; p=1; at least one R^{6′″} is isopropyl, and R⁷ is Br. In some embodiments, o=1; p=1; R⁶ is isopropyl; and R⁷ is 1. In some embodiments, o=2; p=1; at least one R^{6′″} is isopropyl; and R^{7′″} is 1. In some embodiments, o=2; p=2; at least one R^{6′″} is isopropyl, and at least one R^{7′″} is 1. In some embodiments, o=2; p=2; at least one R^{6′″} is isopropyl; one R^{7′″} is Br; and the other R^{7′″} is cyano. In some embodiments, o=2; p=3; at least one R^{6′″} is isopropyl; two R^{7′″} are fluoro; and one R^{7′″} is Br. In some embodiments, o==2; p=1; at least one R^{b} is ethyl, and R^{7′} is Br. In some embodiments, o=2; p=1; one R^{6′″} is isopropyl; the other R^{6′″} is trifluoromethyl; and R^{7′″} is Br. In some embodiments, at least one R^{6′″} is C₁-C₆ alkyl, and at least one R^{7′″} is cyano. In some embodiments, at least one R^{6′″} is isopropyl and at least one R^{7′″} is cyano. In some embodiments, o=1; p=1; R^{6′″} is isopropyl; and R^{7′″} is cyano. In some embodiments, o=2; p=1; at least one R^{6′″} is isopropyl; and R^{7′″} is cyano. In some embodiments, at least one R^{6′″} is C₃-C₇ cycloalkyl, and at least one R^{7′″} is C₃-C₇ cycloalkyl. In some embodiments, at least, one R^{6′″} is cyclopropyl, and at least one R^{7′″} is cyclopropyl. In some embodiments, at least one R^{6′″} is C₃-C₇ cycloalkyl, and at least one R^{7′″} is Br or I. In some embodiments, at least one R^{6′″} is cyclopropyl and at least one R^{7′″} is Br. In some embodiments, at least one R^{6′″} is cyclopropyl and at least one R^{7′″} is 1. In some embodiments, o=1; p=1; R^{6′″} is cyclopropyl; and R⁷ is Br. In some embodiments, o=1; p=1; R^{6′″} is cyclopropyl; and R^{7′″} is 1. In some embodiments, at least one R^{6′″} is C₁-C₆ alkyl, and at least one R^{7′″} is C₁-C₆ alkoxy optionally substituted with one or more halo. In some embodiments, at least one R^{6′″} is isopropyl, and at least one R^{7′″} is C₁-C₆ alkoxy. In some embodiments, at least one R^{6′″} is isopropyl, and at least one R^{7′″} is methoxy. In some embodiments, o=1; p=1; R^{6′″} is isopropyl, and R⁷ is methoxy. In some embodiments, o=2; p=1; at least one R^{6′″} is isopropyl, and R^{7′″} is methoxy. In some embodiments, at least one R^{6′″} is C₁-C₆ alkyl, and at least one R^{7′″} is C₁-C₆ alkoxy substituted with one or more halo. In some embodiments, at least one R^{6′″} is isopropyl, and at least one R^{7′″} is trifluoromethoxy. In some embodiments, at least one R^{6′″} is isopropyl, and at least one R^{7′″} is difluoromethoxy. In some embodiments, at least one R^{6′″} is Br or I, and at least one R^{7′″} is C₁-C₆ haloalkyl optionally substituted with hydroxy. In some embodiments, o=1; p=1; R^{6′″} is Br, and R⁷ is trifluoromethyl. In some embodiments, at least, one R^{6′″} is Br, and at least one R^{7′″} is C₁-C₆ haloalkoxy. In some embodiments, at least one R^{6′″} is Br, and at least one R^{7′″} is trifluoromethoxy. In some embodiments, o=1; p=1, R^{6′″} is Br, and R^{7′″} is trifluoromethoxy. In some embodiments, at least one R^{6′″} is C₁-C₆ alkoxy; and at least one R^{7′″} is Br. In some embodiments, o=1, p=2, R^{6′″} is C₁-C₆ alkoxy; and at least one R^{7′″} is Br.

In some embodiments, at least one R^{7′″} is C₁-C₆ alkyl, and at least one R^{6′″} is C₁-C₆ alkyl optionally substituted with one or more halo. In some embodiments, at least one R^{7′″} is isopropyl and at least one R^{6′″} is methyl. In some embodiments, at least one R^{7′″} is C₁-C₆ alkyl, and at least one R^{6′″} is C₁-C₆ alkyl substituted with one or more halo. In some embodiments, at least one R^{7′″} is isopropyl and at least one R^{6′″} is trifluoromethyl. In some embodiments, at least one R^{7′″} is C₁-C₆ alkyl, and at least, one R^{6′″} is C₃-C₇ cycloalkyl. In some embodiments, at least, one R^{7′″} is isopropyl and at least one R^{6′″} is cyclopropyl. In some embodiments, o=1; p=1; R^{7′″} is isopropyl; and R^{6′″} is cyclopropyl. In some embodiments, at least one R^{7′″} is C₁-C₆ alkyl, and at least one R^{6′″} is Br or I. In some embodiments, at least one R^{7′″} is isopropyl and at least one R^{6′″} is Br. In some embodiments, at least one R^{7′″} is isopropyl and at least one R^{6′″} is Br. In some embodiments, at least one R^{7′} is isopropyl and at least one R^{6′″} is Br. In some embodiments, o=1; p=1; R^{7′″} is isopropyl, and R^{6′″} is Br; In some embodiments, o=2; p=1; R is isopropyl; and at least one R^{6′″} is Br. In some embodiments, o=1; p=1; R^{7′″} is isopropyl; and R^{6′″} is 1. In some embodiments, o=2; p=1; R^{7′″} is isopropyl, and at least one R^{6′″} is 1. In some embodiments, o=2; p=2; at least one R^{7′″} is isopropyl; and at least one R^{6′″} is 1. In some embodiments, o=2; p=2; at least one R^{7′″} is isopropyl; one R^{6′″} is Br; and the other R^{6′″} is cyano. In some embodiments, o=2, p=1, R^{7′} is ethyl; and at least one R^{6′″} is Br. In some embodiments, o=1; p=2; one R^{7′″} is isopropyl; the other R^{7′″} is trifluoromethyl; and R^{6′″} is Br. In some embodiments, at least one R⁷ is C₁-C₆ alkyl, and at least one R^{6′″} is cyano. In some embodiments, at least one R^{7′} is isopropyl and at least one R^{6′″} is cyano. In some embodiments, o=1; p=1; R^{7′″} is isopropyl, and R^{6′″} is cyano. In some embodiments, o=2; p=1; R^{7′″} is isopropyl; and at least one R^{6′″} is cyano. In some embodiments, at least one R^{7′} is C₃-C₇ cycloalkyl, and at least one R^{6′″} is C₃-C₇ cycloalkyl. In some embodiments, at least one R^{7′″} is cyclopropyl, and at least one R^{6′″} is cyclopropyl. In some embodiments, at least one R is C₃-C₇ cycloalkyl, and at least one R^{6′″} is Br or I. In some embodiments, at least one R^{7′″} is cyclopropyl and at least one R^{6′″} is Br or I. In some embodiments, at least one R^{7′″} is cyclopropyl and at least one R^{6′″} is Br. In some embodiments, at least one R^{7′″} is cyclopropyl and at least one R^{6′″} is 1. In some embodiments, o=1; p=1; R^{7′″} is cyclopropyl; and R^{6′″} is Br. In some embodiments, o=1; p=1; R^{7′″} is cyclopropyl; and R^{6′″} is 1. In some embodiments, at least one R^{7′″} is C₁-C₆ alkyl, and at least one R^{7′″} is C₁-C₆ alkoxy optionally substituted with one or more Br or I. In some embodiments, at least one R^{7′″} is isopropyl, and at least one R^{6′″} is C₁-C₆ alkoxy. In some embodiments, at least one R^{7′″} is isopropyl, and at least one R^{6′″} is methoxy.

In some embodiments, o=1; p=1; R^{7′″} is isopropyl, and R^{6′″} is methoxy. In some embodiments, o=2; p=1; R^{7′″} is isopropyl, and at least one R^{6′″} is methoxy. In some embodiments, at least one R^{7′″} is C₁-C₆ alkyl, and at least one R^{6′″} is C₁-C₆ alkoxy substituted with one or more Br or I. In some embodiments, at least one R^{7′″} is isopropyl, and at least one R^{6′″} is trifluoromethoxy. In some embodiments, at least one R^{7′″} is Br or I, and at least one R^{6′″} is C₁-C₆ haloalkyl optionally substituted with one or more hydroxy. In some embodiments, o=1; p=1; R^{6′″} is Br, and R^{6′″} is trifluoromethyl. In some embodiments, at least one R^{7′″} is Br or I, and at least one R^{6′″} is C₁-C₆ haloalkoxy. In some embodiments, at least one R^{7′″} is Br. and at least one R^{6′″} is trifluoromethoxy. In some embodiments, o=1; p=1; R⁷ is Br, and R^{6′″} is trifluoromethoxy. In some embodiments, at least one R^{7′″} is C₁-C₆ alkoxy; and at least one R^{6′″} is Br or I. In some embodiments, o=1; p=2; at least one R^{7′″} is C₁-C₆ alkoxy, and R^{6′″} is Br.

In some embodiments, R^{6′″} and R^{7′″} are each attached to a carbon of an aryl ring B. In some embodiments, R^{6′″} and R^{7′″} are each attached to a carbon of a heteroaryl ring B. In some embodiments, R^{6′″} is attached to a carbon and R^{7′″} is attached to a nitrogen of a heteroaryl ring B. In some embodiments, R^{7′″} is attached to a carbon and R^{6′″} is attached to a nitrogen of a heteroaryl ring B.

In some embodiments, one R^{6′″} and one R^{7′″} are on adjacent atoms, and taken together with the atoms connecting them, form a C₄ carbocyclic ring optionally substituted with one or more substituents independently selected from hydroxy. Br, I, oxo, C₁-C₆ alkyl, C₁-C₆ alkoxy. NR⁸R⁹, =NR¹⁰, COOC₁-C₆ alkyl, C₆-C₁₀ aryl, and CONR⁸R⁹.

In some embodiments, R^{6′″} and R^{7′″} are on adjacent atoms, and taken together with the atoms connecting them, form a C₅ aliphatic carbocyclic ring.

In some embodiments, R^{6′} and R^{7′″} are on adjacent atoms, and taken together with the atoms connecting them, form a C₆ carbocyclic ring optionally substituted with one or more substituents independently selected from hydroxy, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R^{6′″} and R^{7′″} are on adjacent atoms, and taken together with the atoms connecting them, form a C₆ aliphatic carbocyclic ring.

In some embodiments, R^{6′″} and R^{7′″} are on adjacent atoms, and taken together with the atoms connecting them, form a C₆ aromatic carbocyclic ring.

In some embodiments, R^{6′″} and R⁷ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R^{6′″} and R^{7′″} are on adjacent atoms, and taken together with the atoms connecting them, form a 5-membered aliphatic heterocyclic ring containing 1 or 2 heteroatoms independently selected from O, N, and S,

In some embodiments, R^{6′″} and R^{7′″} are on adjacent atoms, and taken together with the atoms connecting them, form a 5-membered heteroaromatic ring containing 1 or 2 heteroatoms independently selected from O, N, and S.

In some embodiments, R^{6′″} and R^{7′″} are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R^{6′″} and R^{7′″} are on adjacent atoms, and taken together with the atoms connecting them, form a 6-membered aliphatic heterocyclic ring containing 1 or 2 heteroatoms independently selected from O, N, and S.

In some embodiments, R^{6′″} and R^{7′″} are on adjacent atoms, and taken together with the atoms connecting them, form a 6-membered heteroaromatic ring containing 1 or 2 heteroatoms independently selected from O, N, and S.

In some embodiments, one R^{6′″} and one R^{7′″} are on adjacent atoms, and taken together with the atoms connecting them, form a C₄, C₆, C₇, or C₈ carbocyclic ring or a 5- to 8-membered heterocyclic ring containing 1 or 2 heteroatoms independently selected from O, N, and S, wherein the ring is fused to the B ring at the 2- and 3-positions relative to the bond connecting the B ring to the NH(Co)group.

In some embodiments, o=2; p=2 or 3, and

two pairs, each of one R^{6′″} and one R^{7′″}, are on adjacent atoms, and each pair of one R^{6′″} and one R^{7′″} taken together with the atoms connecting them form a C₄ carbocyclic ring optionally independently substituted with one or more substituents independently selected from hydroxy, Br, I, oxo, C₁-C₆ alkyl, C₁-C₆ alkoxy, NR⁸R⁹, ═NR¹⁰, COOC₁-C₆ alkyl, C₆-C₁₀ and, and CONR⁸R⁹.

In some embodiments, o=2; p=2 or 3; and

two pairs, each of one R^{6′″} and one R^{7′″}, are on adjacent atoms, and each pair of one R^{6′″} and one R^{7′″} taken together with the atoms connecting them form a C₄ aliphatic carbocyclic ring.

In some embodiments, o=2; p=2 or 3; and

two pairs, each of one R^{6′″} and one R^{7′″}, are on adjacent atoms, and each pair of one R^{6′″} and one R^{7′″} taken together with the atoms connecting them form a C₆ carbocyclic ring optionally independently substituted with one or more substituents independently selected from hydroxy, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R^{6′″} and one R^{7′″}, are on adjacent atoms, and each pair of one R^{6′} and one R^{7′″} taken together with the atoms connecting them form a C₆ aliphatic carbocyclic ring.

In some embodiments, o=2; p=2 or 3, and

two pairs, each of one R^{6′″} and one R^{7′″}, are on adjacent atoms, and each pair of one R^{6′″} and one R^{7′″} taken together with the atoms connecting them form a C₆ aromatic carbocyclic ring.

In some embodiments, o=2; p=2 or 3; and

two pairs, each of one R^{6′″} and one R^{7′″} are on adjacent atoms, and each pair of one R^{6″} and one R^{7′″} taken together with the atoms connecting them form a 5-membered heterocyclic ring containing 1 or 2 heteroatoms independently selected from O, N, and S, optionally substituted with one or more substituents independently selected from hydroxy, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3, and

two pairs, each of one R^{6′″} and one R^{7′″}, are on adjacent atoms, and each pair of one R^{6′″} and one R^{7′″} taken together with the atoms connecting them form a 5-membered aliphatic heterocyclic ring containing 1 or 2 heteroatoms independently selected from O, N, and S.

In some embodiments, o=2; p=2 or 3, and

two pairs, each of one R^{6′″} and one R^{7′″}, are on adjacent atoms, and each pair of one R^{6′″} and one R^{7′″} taken together with the atoms connecting them form a 5-membered heteroaromatic ring containing 1 or 2 heteroatoms independently selected from O, N, and S.

In some embodiments, o=2; p=2 or 3; and

two pairs, each of one R^{6′″} and one R^{7′″}, are on adjacent atoms, and each pair of one R^{6′″} and one R^{7′″} taken together with the atoms connecting them form a 6-membered heterocyclic ring containing 1 or 2 heteroatoms independently selected from O, N, and S, optionally substituted with one or more substituents independently selected from hydroxy, Br, I, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2, p=2 or 3; and

two pairs, each of one R^{6′″} and one R^{7′″}, are on adjacent atoms, and each pair of one R^{6′″} and one R^{7′″} taken together with the atoms connecting them form a 6-membered aliphatic heterocyclic ring containing 1 or 2 heteroatoms independently selected from Q, N, and S.

In some embodiments, o=2; p=2 or 3; and

two pairs, each of one R^{6′″} and one R^{7′″}, are on adjacent atoms, and each pair of one R^{6′} and one R^{7′″} taken together with the atoms connecting them form a 6-membered heteroaromatic ring containing 1 or 2 heteroatoms independently selected from O, N, and S.

In some embodiments, o=2; p=2 or 3; and

two pairs, each of one R^{6′″} and one R^{7′″}, are on adjacent, atoms, and each pair of one R⁶ and one R^{7′″} taken together with the atoms connecting them independently form a C₄, C₆, C₇, or C₈ carbocyclic ring or a 5- to 8-membered heterocyclic ring containing 1 or 2 heteroatoms independently selected from O, N, and S,

wherein one of the two rings is fused to the B ring at the 2- and 3-positions relative to the bond connecting the B ring to the NH(Co)group, and the other of the two rings is fused to the B ring at the 5- and 6-positions relative to the bond connecting the B ring to the NH(CO) group.

In some embodiments, o=2; p=2; and

two pairs, each of one R^{6′″} and one R^{7′″}, are on adjacent atoms, and each pair of one R^{6′″} and one R^{7′″} taken together with the atoms connecting them form a C₄ aliphatic carbocyclic ring.

In some embodiments, o=2; p=3; and

two pairs, each of one R^{6′″} and one R^{7′″}, are on adjacent atoms, and each pair of one R^{6″} and one R^{7″} taken together with the atoms connecting them form a C₄ aliphatic carbocyclic ring; and one R^{7′″} is Br or I

In some embodiments, o=2; p=3; and

two pairs, each of one R^{6′″} and one R^{7′″}, are on adjacent atoms, and each pair of one R^{6′} and one R^{7′″} taken together with the atoms connecting them form a C₄ aliphatic carbocyclic ring, and one R^{7′″} is CN.

In some embodiments, one R⁷ is pyrazolyl and is para to the bond connecting the B ring to the NH(CO) group of Formula AA. In some embodiments, one R^{7″} is 3-pyrazolyl and is para to the bond connecting the B ring to the NH(CO) group of Formula AA. In some embodiments, one R^{7′″} is 4-pyrazolyl and is para to the bond connecting the B ring to the NH(CO) group of Formula AA. In some embodiments, one R^{7′″} is 5-pyrazolyl and is para to the bond connecting the B ring to the NH(CO) group of Formula AA. In some embodiments, one R^{7′″} is thiazolyl and is para to the bond connecting the B ring to the NH(CO) group of Formula AA. In some embodiments, one R^{7′″} is 4-thiazolyl and is para to the bond connecting the B ring to the NH(CO) group of Formula AA. In some embodiments, one R^{7′″} is 5-thiazolyl and is para to the bond connecting the B ring to the NH(CO) group of Formula AA. In some embodiments, one R^{7′″} is fund and is para to the bond connecting the B ring to the NH(CO) group of Formula AA. In some embodiments, one R^{7′″} is 2-furyl and is para to the bond connecting the B ring to the NH(CO) group of Formula AA. In some embodiments, one R^{7′″} is thiophenyl and is para to the bond connecting the B ring to the NH(CO) group of Formula AA. In some embodiments, one R^{7′″} is 2-thiophenyl and is para to the bond connecting the B ring to the NH(CO) group of Formula AA; In some embodiments, one R^{7′″} is phenyl and is para to the bond connecting the B ring to the NH(CO) group of Formula AA. In some embodiments, one R^{7′″} is cycloalkenyl (e.g., cyclopentenyl, e.g., 1-cyclopentenyl) and is para to the bond connecting the B ring to the NH(CO) group of Formula AA; In some embodiments, one R^{7′″} is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the NH(CO) group of Formula AA. In some embodiments, one R^{7′″} is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl) and is para to the bond connecting the B ring to the NH(CO) group of Formula AA. In some embodiments, one R^{7′″} is phenyl optionally substituted with one or more C₆-C₁₀ aryloxy (e.g., phenoxy) and is para to the bond connecting the B ring to the NH(CO) group of Formula AA. In some embodiments, one R^{7″} is phenyl optionally substituted with one or more CN and is para to the bond connecting the B ring to the NH(CO) group of Formula AA. In some embodiments, one R^{7′″} is phenyl optionally substituted with one or more halo (e.g., F, Cl) and is para to the bond connecting the B ring to the NH(CO) group of Formula AA and is para to the bond connecting the B ring to the NH(CO) group of Formula AA. In some embodiments, one R^{7′″} is phenyl optionally substituted with one or more COOC₁-C₆ alkyl (e.g., CO₂ₜ-Bu) and is para to the bond connecting the B ring to the NH(CO) group of Formula AA. In some embodiments, one R^{7′″} is phenyl optionally substituted with one or more S(O₂)C₁-C₆ alkyl (e.g., S(O₂)methyl) and is para to the bond connecting the B ring to the NH(CO) group of Formula AA. In some embodiments, one R⁷ is phenyl optionally substituted with one or more 3- to 7-membered heterocycloalkyl (e.g., morpholinyl) and is para to the bond connecting the B ring to the NH(CO) group of Formula AA. In some embodiments, one R^{7″} is phenyl optionally substituted with one or more CONR⁸R⁹ (e.g., unsubstituted amido) and is para to the bond connecting the B ring to the NH(CO) group of Formula AA. In some embodiments, one R^{7″} is phenyl optionally substituted with one or more C₁-C₆ alkyl (e.g., methyl or propyl, e.g., 2-propyl) and with one or more Br or I and is para to the bond connecting the B ring to the NH(Co) group of Formula AA and is para to the bond connecting the B ring to the NH(CO) group of Formula AA,

In some embodiments, R^{6′″} and R^{7′″} are each attached to a carbon of an aryl ring B. In some embodiments, R^{6′″} and R^{7′″} are each attached to a carbon of a heteroaryl ring B. In some embodiments, R^{6′″} is attached to a carbon and R^{7′″} is attached to a nitrogen of a heteroaryl ring B; In some embodiments, R^{7′″} is attached to a carbon and R^{6′″} is attached to a nitrogen of a heteroaryl ring B.
In some embodiments, the optionally substituted ring B is

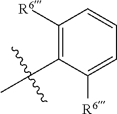

R^{6′″} and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 1.0-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, =NR¹⁰, COOC₁-C₆ alkyl, CONR⁸R⁹, 4- to 6-membered heterocycloalkyl, C₆-C₁₀ aryl, 5-to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
In some embodiments, the optionally substituted ring B is

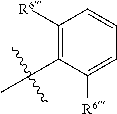

and each R^{6′″} is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.
In some embodiments, the optionally substituted ling B is

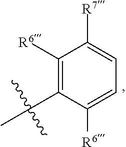

wherein each R^{6′″} is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, Br, I, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7′″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6′″} and R^{7′″}, taken together with the atoms connecting them, independently form C₄, C₆, C₇, or C₈ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

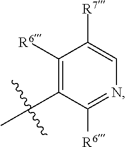

wherein each R^{6′″} is independently selected from C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7′″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R^{6′″} and R^{7′″}, taken together with the atoms connecting them, independently form C₄, C₆, C₇, or C₈ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

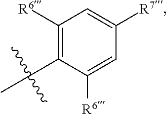

wherein each R^{6′″} is independently selected from C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R^{7′″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
In some embodiments, the optionally substituted ring B is

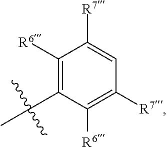

wherein each R^{6′″} is independently selected from C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7′″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ and, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R^{6′″} and R^{7′″} on adjacent atoms, taken together with the atoms connecting them, independently form at least one C₄, C₆, C₇, or C₈ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

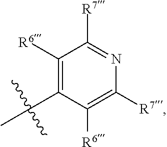

wherein each R^{6′″} is independently selected from C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7′″} is independently selected from C₁-C₆ alkyl, G-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R^{6′″} and R^{7′″} on adjacent atoms, taken together with the atoms connecting them, independently form at least one C₄, C₆, or C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the optionally substituted ring B is

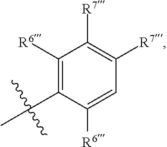

wherein each R^{6′″} is independently selected from C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R^{7″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R^{6′″} and R^{7′″}, taken together with the atoms connecting them, independently form a C₄, C₆, or C₁ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the optionally substituted ring B is

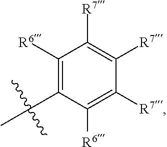

wherein each R^{6′″} is independently selected from C₁-C₆ alkyl, C₃-C₇ cycloalkyl, C₁-C₆ haloalkyl, C₁-C₆ alkoxy, C₁-C₆ haloalkoxy, Br, I, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₆-C₁₀ alkynyl;
wherein each R^{7′″} is independently selected from C₁-C₆ alkyl, C₁-C₆ haloalkyl, C₁-C₆ alkoxy, C₁-C₆ haloalkoxy, Br, I, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R^{6′″} and R^{7′″} on adjacent atoms, taken together with the atoms connecting them, independently form at least one C₄, C₆, or C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

The group R¹⁰

In some embodiments, R¹⁰ is C₁-C₆ alkyl. In some embodiments, R¹⁰ is methyl. In some embodiments, R¹⁰ is ethyl.

The groups R⁸ and R⁹

In some embodiments, each of R⁸ and R⁹ at each occurrence is independently selected from hydrogen, C₁-C₆ alkyl, (C=NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ and, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to.

In some embodiments, each of R⁸ and R⁹ at each occurrence is hydrogen; In some embodiments, each R⁸ at each occurrence is hydrogen and each R⁹ at each occurrence is C₁-C₆ alkyl. In some embodiments, each R⁸ at each occurrence is hydrogen and each R⁹ at each occurrence is methyl. In some embodiments, each R⁸ at each occurrence is hydrogen and each R⁹ at each occurrence is ethyl. In some embodiments, each of R⁸ and R⁹ at each occurrence is methyl. In some embodiments, each of R⁸ and R⁹ at each occurrence is ethyl. In some embodiments, R⁸ and R⁹ taken together with the nitrogen they are attached to form a 3-membered ring. In some embodiments, R⁸ and R⁹ taken together with the nitrogen they are attached to form a 4-membered ring. In some embodiments, R⁸ and R⁹ taken together with the nitrogen they are attached to form a 5-membered ring. In some embodiments, R⁸ and R⁹ taken together with the nitrogen they are attached to form a 6-membered ring optionally containing one or more oxygen atoms in addition to the nitrogen they are attached to. In some embodiments, R⁸ and R⁹ taken together with the nitrogen they are attached to form a 6-membered ring optionally containing one or more nitrogen atoms in addition to the nitrogen they are attached to. In some embodiments, R⁸ and R⁹ taken together with the nitrogen they are attached to form a 7-membered ring.

The group R¹³

In some embodiments, R¹³ is C₁-C₆ alkyl. In some embodiments, R¹³ is methyl. In some embodiments, R¹³ is ethyl. In some embodiments, R¹³ is C₆-C₁₀ aryl. In some embodiments, R¹³ is phenyl. In some embodiments, R¹³ is 5- to 10-membered heteroaryl.

The Groups R¹¹ and R¹²

In some embodiments, each of R¹¹ and R¹² at each occurrence is independently selected from hydrogen and C₁-C₆ alkyl optionally substituted with hydroxy. In some embodiments, each of R¹¹ and R¹² at each occurrence is independently selected from hydrogen and unsubstituted C₁-C₆ alkyl. In some embodiments, each of R¹¹ and R¹² at each occurrence is hydrogen; In some embodiments, each R^{u} at each occurrence is hydrogen and each R¹² at each occurrence is CCG alkyl. In some embodiments, each R¹¹ at each occurrence is hydrogen and each R¹² at each occurrence is methyl. In some embodiments, each R¹¹ at each occurrence is hydrogen and each R¹² at each occurrence is ethyl. In some embodiments, each R¹¹ at each occurrence is hydrogen and each R¹² at each occurrence is hydroxy ethyl. In some embodiments, each of R¹¹ and R¹² at each occurrence is methyl. In some embodiments, each of R¹¹ and R¹² at each occurrence is ethyl.

In some embodiments of the compound of formula AA,

the substituted ring A is

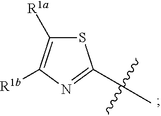

and R^{1a} and R^{1b} are one of the following combinations:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxy pentyl; R^{1a} is hydroxymethyl, and R^{1b} is hydroxyhexyl; R^{1a} is hydroxyethyl, and R^{1b} is hydroxymethyl; R^{1a} is hydroxyethyl, and R¹⁰ is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl, R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R¹⁰ is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl, R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl, R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl, R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,
the substituted ring A is

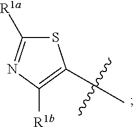

and R^{1a} and R^{1b} are one of the following combinations:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R¹⁰ is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R¹⁰ is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl, R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R¹⁰ is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R¹⁰ is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl, R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is

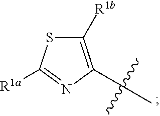

and R^{1a} and R^{1b} are one of the following combinations: R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl, R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R¹⁰ is 3-hydroxy-2-propyl; R^{1a} is 2-hydroxy-2-propyl, and R^{1b} is 1-hydroxy-1-propyl, R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl, R^{1a} is 2-hydroxy-2-propyl, and R^{1b} is hydroxypentyl, R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl, R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl, R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl, R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R¹⁰ is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is;

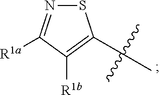

and R^{1a} and R^{1b} are one of the following combinations:

R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl, R^{1a} is hydroxymethyl, and R^{1b} is 2-hydroxy-1-propyl; R^{1a} is hydroxymethyl, and R¹⁰ is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl, R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R¹⁰ is hydroxyhexyl, R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R¹⁰ is 3-hydroxy-2-propyl; R^{1a} is 2-hydroxy-2-propyl, and R^{1b} is 1-hydroxy-1-propyl; R^{1a} is 2-hydroxy-2-propyl, and R¹⁰ is 2-hydroxy-1-propyl; R^{1a} is 2-hydroxy-2-propyl, and R^{1b} is 3-hydroxy-1-propyl, R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R¹⁰ is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl, R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl, R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is R^{1b},

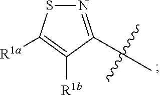

and R^{1a} and R^{1b} are one of the following combinations:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R¹⁰ is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R¹⁰ is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl, R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxy pentyl; R^{1b} is hydroxymethyl, and R^{1a} is hydroxyhexyl; R^{1b} is hydroxyethyl, and R^{1a} is hydroxymethyl, R^{1b} is hydroxyethyl, and R^{1a} is hydroxyethyl; R^{1b} is hydroxyethyl. and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R¹⁰ is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propy 1, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl, R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is R^{1b};

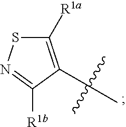

and R^{1a} and R^{1b} are one of the following combinations:
R^{1a} is hydroxymethyl, and R^{1b} is hydroxymethyl, R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl, R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl, R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R¹⁰ is 3-hydroxy-2-propyl; R^{1a} is 2-hydroxy-2-propyl, and R^{1b} is 1-hydroxy-1-propyl, R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl, R^{1a} is 2-hydroxy-2-propyl, and R^{1b} is hydroxypentyl, R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl, R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl, R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl, R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R¹⁰ is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; and R^{1b} is 2-hydroxy-2-propyl, and R^{1a} is hydroxyhexyl.

In some embodiments of the compound of formula AA,

the substituted ring A is

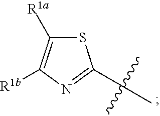

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂R¹¹R¹², and R^{1b} is —NR¹¹COR¹².

In some embodiments of the compound of formula AA,

the substituted ring A is N⁵;

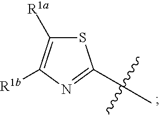

R^{1a} and R^{1b} are one of the following combinations:

In some embodiments, R^{1a} is 2-hydroxy-2-propyl, and R¹⁰ is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxy ethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂.NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

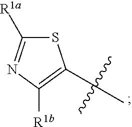

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R¹⁰ is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R¹⁰ is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂.NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is SO₂NR¹¹R¹², and R^{1b} is —OR¹¹; R^{1a} is —SO₂.NR¹¹R¹², and R^{1b} is —COR¹³; R^{1a} is —SO₂NR¹¹R¹², and R^{1b} is —CO₂R¹³; R^{1a} is —SO₂.NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

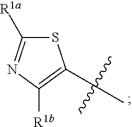

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propy 1, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

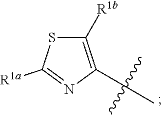

R^{1a} and R^{1b} are one of the following combinations:

R^{1a} is C₁-C₆ alkyl substituted with one or more hydroxy, and R^{1b} is —SO₂.NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)3, and R^{1b} is NR¹³CONR¹¹R¹²; R^{1a} is C₁-C₆ alkyl substituted with one or more —OSi(R¹³)3, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³, R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹²

In some embodiments of the compound of formula AA,

the substituted ring A is

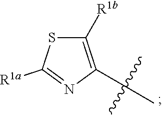

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is cyanomethyl; and In some embodiments, R^{1a} is —SO₂NHMe, and R^{1b} is dimethylaminomethyl; In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

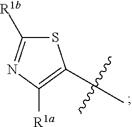

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R^{1J})₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1a} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹ COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

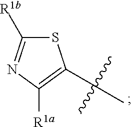

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxy ethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

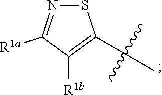

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³, R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹³R¹²CN; R^{1a} is C₁-C₆ alkyl substituted with one or more hydroxy, and R^{1b} is —NR¹¹SO₂R¹³, R^{1a} is C₁-C₆ alkyl substituted with one or more hydroxy, and R^{1b} is —NR¹¹CONR^{1J}R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹¹; R^{1a} is C₁-C₆ alkyl substituted with one or more —OSi(R¹³)₃, and R¹⁰ is —SO₂R¹³; R^{1a} is C₁-C₆ alkyl substituted with one or more —OSi(R^{1J})₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂.NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂.NR¹¹R¹², and R^{1b} is —COR¹³; R^{1a} is —SO₂NR¹¹R¹², and R^{1b} is —CO₂R¹³; R^{1a} is —SO₂.NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA, the substituted ring A is

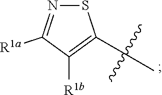

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1a} is —SO₂NHCH₂CH₂OH; In some embodiments, R^{1a} is —SO₂NHMe, and R^{1b} is —SO₂NHMe; In some embodiments, R^{1a} is —SO₂NHMe, and R^{1a} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

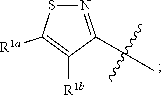

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R¹⁰ is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂N¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³, R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is R^{1b};

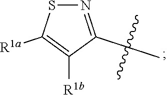

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R¹⁰ is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1a} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

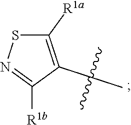

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1a} is —NR¹¹SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹N¹²R¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more OSi(R¹³)₃, and R^{1b} is —CO₂R¹³; R^{1a} is C₃-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R¹⁰ is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

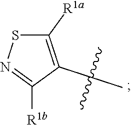

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments. R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is cyanomethyl; and In some embodiments, R^{1a} is SO₂NHMe, and R^{1b} is dimethylaminomethyl; In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

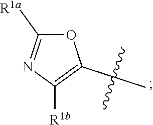

R^{1a} and R^{1b} are one of the following combinations: In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂NHMe, and R^{1b} is —C₀₂Me; In some embodiments, R^{1a} is —SO₂NHMe, and R^{1b} is hydroxymethyl; In some embodiments, R^{1a} is —SO₂NHMe, and R¹⁰ is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂.NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

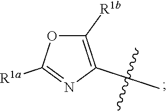

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹, R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂₋NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂₋NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

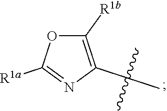

R^{1a} and R^{1b} are one of the following combinations: In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

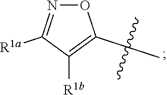

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR^{1J}R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R^{1J})₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³, R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³, R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

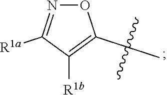

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R¹⁰ is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is R^{1b};

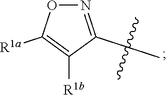

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R¹⁰ is —NR¹¹CONR¹¹R¹²; R^{1a} is C₁-C₆ alkyl substituted with one or more —OSi(R¹³)₃, and R^{1b} is —NR¹¹ COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

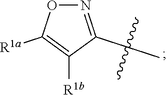

R^{1a} and R^{1b} are one of the following combinations:

In some embodiments, R^{1a} is 2-hydroxy-2-propyl, and R¹⁰ is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

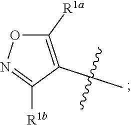

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R¹⁰ is —NR¹¹SO₂R¹³; R^{1a} is C₁-C₆ alkyl substituted with one or more hydroxy, and R^{1b} is NR¹¹CONR¹¹R¹²; R^{1a} is C₁-C₆ alkyl substituted with one or more hydroxy, and R^{1b} is —NR¹″COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R¹⁰ is —SO₂R¹³; R^{1a} is C₁-C₆ alkyl substituted with one or more —OSi(R^{1J})₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂.NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is;

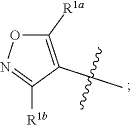

R^{1a} and R^{1b} are one of the following combinations:

In some embodiments, R^{1a} is 2-hydroxy-2-propyl, and R¹⁰ is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

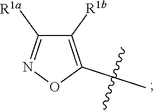

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R¹⁰ is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³, R^{1a} is —SO₂NR¹¹R¹², and R^{1b} is —CO₂R¹³; R^{1a} is —SO₂NR¹¹R¹², and R^{1b} is —NR¹¹CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is O;

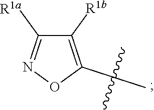

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R¹⁰ is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R¹⁰ is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

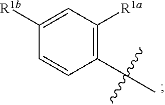

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R¹⁰ is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

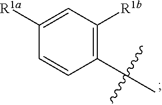

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R¹⁰ is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R¹⁰ is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³, R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂.NR¹¹R¹², and R^{1b} is —NR¹¹COR¹².

In some embodiments of the compound of formula AA,

the substituted ring A is

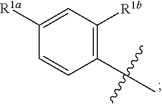

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SC₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SC₂NHMe, and R^{1b} is —OMe; In some embodiments, R^{1a} is —SO₂NHMe, and R¹⁰ is —OH; In some embodiments, R^{1a} is —SC₂NHMe, and R^{1b} is —CO₂Me; In some embodiments, R^{1a} is —SO₂NHMe, and R^{1b} is hydroxymethyl; In some embodiments, R^{1a} is —SC₂NHMe, and R^{1a} is hydroxy ethyl; In some embodiments, R^{1a} is —SO₂NHMe, and R^{1b} is 2-hydroxy-2-propyl; In some embodiments, R^{1a} is —SC₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

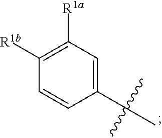

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹³R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR^{1J}R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R¹⁰ is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂.NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂.NR¹¹R¹², and R^{1b} is —COR¹³; R^{1a} is —SO₂NR¹¹R¹², and R^{1b} is —CO₂R¹³; R^{1a} is —SO₂.NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

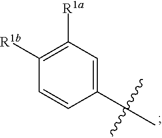

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

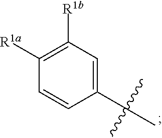

R^{1a} and R^{1b} are one of the following combinations;

In some embodiments, R^{1a} is 2-hydroxy-2-propyl, and R¹⁰ is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is;

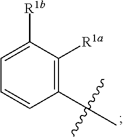

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹³R¹²CN; R^{1a} is C₁-C₆ alkyl substituted with one or more hydroxy, and R^{1b} is —NR¹¹SO₂R¹³, R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R¹⁰ is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂.NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

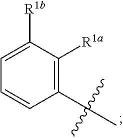

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂₋NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂.NHMe, and R^{1b} is —CO₂₋Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂.NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is;

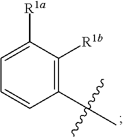

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹, R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂₋NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂₋NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is;

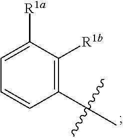

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

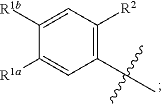

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹³R¹²CN; R^{1a} is C₁-C₆ alkyl substituted with one or more hydroxy, and R^{1b} is —NR¹¹SO₂R¹³, R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R¹⁰ is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹, R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is;

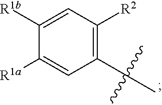

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is;

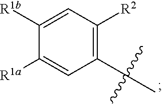

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹, R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³, R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹², R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

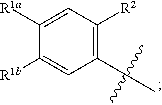

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

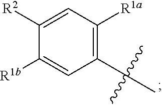

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³, R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R¹⁰ is —NR¹¹CONR¹¹R¹²; R^{1a} is C₁-C₆ alkyl substituted with one or more —OSi(R¹³)₃, and R^{1b} is —NR¹¹ COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

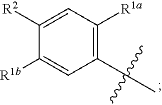

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

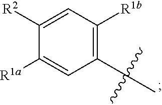

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is (u-CA alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹³R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R¹⁰ is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹¹; R^{1a} is —SO₂NR¹¹N¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹ COR¹².

In some embodiments of the compound of formula AA,

the substituted ring A is

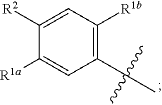

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R¹⁰ is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

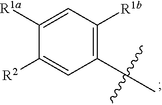

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³, R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R¹⁰ is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R¹⁰ is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³, R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂.NR¹¹R¹², and R^{1b} is —NR¹¹COR¹².

In some embodiments of the compound of formula AA,

the substituted ring A is

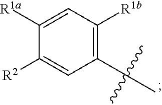

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxy ethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

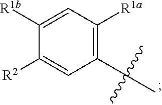

R^{1a} and R^{1b} are one of the following combinations:

R^{1a} is C₁-C₆ alkyl substituted with one or more hydroxy, and R^{1b} is —SO₂NR^{1I}R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is (u-CA alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³, R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R¹⁰ is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R¹⁰ is —CR¹¹R¹³CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more OSi(R¹³)₃, and R¹⁰ is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR^{1b}R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹ COR¹².

In some embodiments of the compound of formula AA,

the substituted ring A is

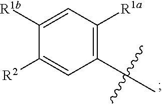

R^{1a} and R^{1b} are one of the following combinations: In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1a} is cyanomethyl; In some embodiments, R^{1a} is 2-hydroxy-2-propyl, and R^{1b} is dimethylaminomethyl; In some embodiments, R^{1a} is —SO₂NHMe, and R¹⁰ is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe. and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R*° is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

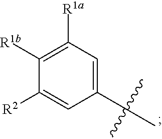

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₂, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

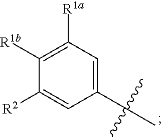

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SC₂NHMe, and R^{1b} is CONHMe; In some embodiments, R^{1a} is —SO₂NHMe, and R^{1b} is dimethylaminomethyl; In some embodiments, R^{1a} is —SC₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

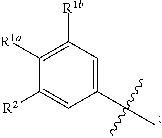

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³, R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R^{1J})₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³, R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

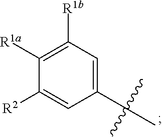

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxy ethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

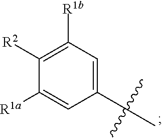

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R^{1J})₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R¹⁰ is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R¹⁰ is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

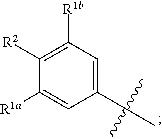

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SO₂NHMe, and R^{1b} is —CO₂Me; In some embodiments, R^{1a} is —SO₂.NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

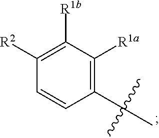

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆

alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR^{1J}R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R^{1J})₃, and R^{1b} is —CONR¹¹R¹²; R^{1a} is C₁-C₆ alkyl substituted with one or more —OSi(R^{1J})₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³, R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

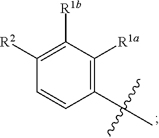

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R¹⁰ is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂.NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

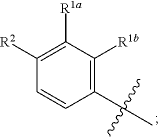

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R¹⁰ is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³, R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂₋NR¹¹R¹², and R^{1b} is —NR¹¹COR¹².

In some embodiments of the compound of formula AA,

the substituted ring A is

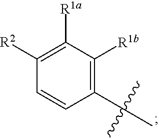

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxy ethyl; In some embodiments, R^{1a} is 2-hydroxy-2-propyl, and R^{1b} is 2-hydroxy-2-propyl; In some embodiments, R^{1a} is 2-hydroxy-2-propyl, and R¹⁰ is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SC₂NHMe, and R^{1b} is —OMe; In some embodiments, R^{1a} is —SO₂NHMe, and R^{1b} is —OH; In some embodiments, R^{1a} is —SO₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SO₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R¹⁰ is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₆ alkyl substituted with one —OSi(Me)₂ₜBu, and R^{1b} is —CO₂Me.

In some embodiments of the compound of formula AA,

the substituted ring A is

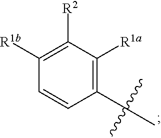

R^{1a} and R^{1b} are one of the following combinations:

R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R⁰; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R^{1b} is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R^{1b} is —SO₂R¹³; R^{1a} is C₁-C₆ alkyl substituted with one or more —OSi(R¹³)₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is —SO₂NR¹¹R¹², and R^{1b} is —OR¹¹; R^{1a} is —SO₂NR¹¹R¹², and R^{1b} is —COR¹³; R^{1a} is —SO₂NR¹¹R¹², and R^{1b} is —CO₂R¹³; R^{1a} is —SO₂NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is

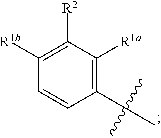

R^{1a} and R^{1b} are one of the following combinations:

In some embodiments, R^{1a} is 2-hydroxy-2-propyl, and R¹⁰ is —OMe; In some embodiments, R^{1a} is 2-hydroxy-2-propyl, and R^{1b} is —OH; In some embodiments, R^{1a} is 2-hydroxy-2-propyl, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SO₂NHMe, and R^{1b} is —OMe; In some embodiments, R^{1a} is —SO₂NHMe, and R¹⁰ is —OH; In some embodiments, R^{1a} is —SC₂NHMe, and R^{1b} is —CO₂Me; In some embodiments, R^{1a} is —SO₂NHMe, and R^{1b} is hydroxymethyl; In some embodiments, R^{1a} is —SO₂NHMe, and R^{1b} is hydroxyethyl; In some embodiments, R^{1a} is —SC₂NHMe, and R^{1b} is 2-hydroxy-2-propyl; In some embodiments, R^{1a} is —SO₂NHMe, and R^{1b} is —SO₂NHCH₂CH₂OH; In some embodiments, R^{1a} is —SO₂NHMe, and R^{1b} is —SO₂Me; In some embodiments, R^{1a} is —SO₂NHMe, and R^{1b} is CONHMe; In some embodiments, R^{1a} is —SO₂NHMe, and R^{1b} is dimethylaminomethyl; In some embodiments, R^{1a} is —SO₂NHMe, and R^{1b} is cyanomethyl; and In some embodiments, R^{1a} is C₁-C₄ alkyl substituted with one —OSi(Me)₂ₜBu, and R^{1b} is —CO₂Me.

In some embodiments of the compound of formula AA,

the substituted ring A is

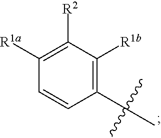

R^{1a} and R^{1b} are one of the following combinations: R^{1a} is C₁-C₆ alkyl substituted with one or more hydroxy, and R^{1b} is —SO₂NR¹¹R¹²; R^{1a} is C₁-C₆ alkyl substituted with one or more hydroxy, and R^{1b} is —SO₂R¹³; R^{1a} is C₁-C₆ alkyl substituted with one or more hydroxy, and R^{1b} is —CONR¹¹R¹²; R^{1a} is C₁-C₆ alkyl substituted with one or more hydroxy, and R^{1b} is —OR¹¹; R^{1a} is C₁-C₆ alkyl substituted with one or more hydroxy, and R^{1b} is —COR¹³; R^{1a} is C₁-C₆ alkyl substituted with one or more hydroxy, and R^{1b} is —CO₂R¹³; R^{1a} is C₁-C₆ alkyl substituted with one or more hydroxy, and R^{1b} is —NR¹³CONR¹¹R¹²; R^{1a} is C₁-C₆ alkyl substituted with one or more hydroxy, and R^{1b} is —CR¹¹R¹²CN; R^{1a} is C₁-C₆ alkyl substituted with one or more hydroxy, and R¹⁰ is —NR¹¹SO₂R¹³; R^{1a} is C₁-C₆ alkyl substituted with one or more hydroxy, and R^{1b} is —NR¹¹CONR¹¹R¹²; R^{1a} is C₁-C₆ alkyl substituted with one or more hydroxy, and R^{1b} is —NR¹¹COR¹²; R^{1a} is C₁-C₆ alkyl substituted with one or more hydroxy, and R^{1b} is —CR¹¹R¹²NR¹¹R¹²; R^{1a} is C₁-C₆ alkyl substituted with one or more —OSi(R¹³)₃, and R^{1b} is —SO₂NR¹¹R¹²; R^{1a} is C₁-C₆ alkyl substituted with one or more —OSi(R¹³)₃, and R¹⁰ is —SO₂R¹³; R^{1a} is C₁-C₆ alkyl substituted with one or more —OSi(R^{1J})₃, and R^{1b} is —CONR¹¹R¹²; R^{1a} is C₁-C₆ alkyl substituted with one or more —OSi(R¹³)₃, and R^{1b} is —OR¹¹; R^{1a} is C₁-C₆ alkyl substituted with one or more —OSi(R¹³)₃, and R^{1b} is —COR¹³; R^{1a} is C₁-C₆ alkyl substituted with one or more —OSi(R¹³)₃, and R^{1b} is —CO₂R¹³; R^{1a} is C₁-C₆ alkyl substituted with one or more —OSi(R¹³)₃, and R^{1b} is —NR¹³CONR¹¹R¹²; R^{1a} is C₁-C₆ alkyl substituted with one or more —OSi(R¹³)₃, and R^{1b} is —CR¹¹R¹²CN; R^{1a} is C₁-C₆ alkyl substituted with one or more —OSi(R¹³)₃, and R^{1b} is —NR¹¹SO₂R¹³; R^{1a} is C₁-C₆ alkyl substituted with one or more —OSi(R¹³)₃, and R^{1b} is —NR¹¹CONR¹¹R¹²; R^{1a} is C₁-C₆ alkyl substituted with one or more —OSi(R¹³)₃, and R^{1b} is —NR¹¹COR¹²; R^{1a} is C₁-C₆ alkyl substituted with one or more —OSi(R¹³)₃, and R^{1b} is —CR¹¹R¹²NR¹¹R¹²; R^{1a} is —SO₂.NR¹¹R¹², and R^{1b} is —SO₂NR¹¹R¹²; R^{1a} is —SO₂NR¹¹R¹², and R^{1b} is —SO₂R¹³; R^{1a} is —SO₂NR¹¹R¹², and R^{1b} is —CONR¹¹R¹²; R^{1a} is SO₂NR¹¹R¹², and R^{1b} is —OR¹¹; R^{1a} is —SO₂.NR¹¹R¹², and R^{1b} is —COR¹³; R^{1a} is —SO₂NR¹¹R¹², and R^{1b} is —CO₂R¹³; R^{1a} is —SO₂.NR¹¹R¹², and R^{1b} is —NR¹³CONR¹¹R¹²; R^{1a} is —SO₂NR¹¹R¹², and R^{1b} is —CR¹¹R¹²CN; R^{1a} is —SO₂NR¹¹R¹², and R^{1b} is —NR¹¹SO₂R¹³; R^{1a} is —SO₂NR¹¹R¹², and R^{1b} is —CR¹¹R¹²NR¹¹R¹²; R^{1a} is —SO₂NR¹¹R¹², and R^{1b} is —NR¹¹CONR¹¹R¹²; and R^{1a} is —SO₂NR¹¹R¹², and R^{1b} is —NR¹¹COR¹².

In some embodiments of the compound of formula AA,

the substituted ring A is;

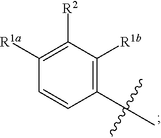

R^{1a} and R^{1b} are one of the following combinations:

In some embodiments, R^{1a} is 2-hydroxy-2-propyl, and R^{1b} is —OMe; In some embodiments, R^{1a} is 2-hydroxy-2-propyl, and R^{1b} is —OH; In some embodiments, R^{1a} is 2-hydroxy-2-propy 1, and R^{1b} is —CO₂Me; In some embodiments, R^{1a} is 2-hydroxy-2-propyl, and R^{1b} is hydroxymethyl; In some embodiments, R^{1a} is 2-hydroxy-2-propyl, and R^{1b} is hydroxyethyl; In some embodiments, R^{1a} is 2-hydroxy-2-propyl, and R^{1b} is 2-hydroxy-2-propyl; In some embodiments, R^{1a} is 2-hydroxy-2-propyl, and R^{1b} is —SO₂NHCH₂CH₂OH; In some embodiments, R^{1a} is 2-hydroxy-2-propyl, and R^{1b} is —SO₂Me; In some embodiments, R^{1a} is 2-hydroxy-2-propyl, and R^{1b} is CONHMe; In some embodiments, R^{1a} is 2-hydroxy-2-propyl, and R^{1b} is cyanomethyl; In some embodiments, R^{1a} is 2-hydroxy-2-propyl, and R^{1b} is dimethylaminomethyl; In some embodiments, R^{1a} is —SC₂NHMe, and R^{1b} is —OMe; In some embodiments, R^{1a} is —SO₂NHMe, and R^{1b} is —OH; In some embodiments, R^{1a} is —SC₂NHMe, and R^{1b} is —CO₂Me; In some embodiments, R^{1a} is —SC₂NHMe, and R^{1b} is hydroxymethyl; In some embodiments, R^{1a} is —SO₂NHMe, and R^{1b} is hydroxyethyl; In some embodiments, R^{1a} is —SC₂NHMe, and R^{1b} is 2-hydroxy-2-propyl; In some embodiments, R^{1a} is —SC₂NHMe, and R^{1b} is —SO₂NHCH₂CH₂OH; In some embodiments, R^{1a} is —SC₂NHMe, and R^{1b} is —SC₂Me; In some embodiments, R^{1a} is —SC₂NHMe, and R^{1b} is CONHMe; In some embodiments, R^{1a} is —SC₂NHMe, and R^{1b} is cyanomethyl; and In some embodiments, R^{1a} is —SO₂NHMe, and R¹⁰ is dimethylaminomethyl; In some embodiments, R^{1a} is C₁-C₄ alkyl substituted with one —OSi(Me)₂ₜBu, and R^{1b} is —CO₂Me.

In some embodiments of the compound of formula AA,

the optionally substituted ring B is

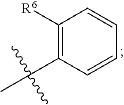

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

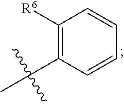

and R⁶ is selected from:
isopropyl, ethyl, methyl, trifluoromethyl, trifluoromethoxy, cyclopropyl, chloro, and fluoro.

In some embodiments of the compound of formula AA,

the optionally substituted ring B is

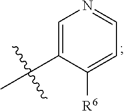

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

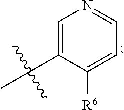

and R⁶ is selected from:
isopropyl, ethyl, methyl, trifluoromethyl, trifluoromethoxy, cyclopropyl, chloro, and fluoro.

In some embodiments of the compound of formula AA,

the optionally substituted ring B is

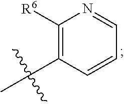

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

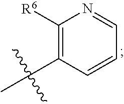

and R⁶ is selected from:
isopropyl, ethyl, methyl, trifluoromethyl, trifluoromethoxy, cyclopropyl, chloro, and fluoro.

In some embodiments of the compound of formula AA,

the optionally substituted ring B

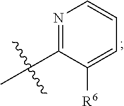

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

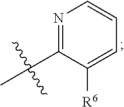

and R is selected from:
isopropyl, ethyl, methyl, trifluoromethyl, trifluoromethoxy, cyclopropyl, chloro, and fluoro.

In some embodiments of the compound of formula AA,

the optionally substituted ring B is

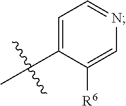

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the optionally substituted ring B is

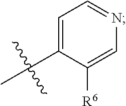

and R⁶ is selected from:
isopropyl, ethyl, methyl, trifluoromethyl, trifluoromethoxy, cyclopropyl, chloro, and fluoro.

In some embodiments, of the compound of formula AA,

the optionally substituted ring B is

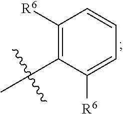

and the two R⁶ are one of the following combinations:
One R⁶ is C₁-C₆ alkyl, and the other R⁶ is C₁-C₆ alkyl optionally substituted with one or more halo; One R⁶ is C₁-C₆ alkyl and the other R^{b} is C₁-C₆ alkyl, One R^{b} is C₁-C₆ alkyl, and the other R⁶ is C₁-C₆ alkyl substituted with one or more halo; One R⁶ is C₁-C₆ alkyl, and the other R^{b} is C₃-C₇ cycloalkyl; One R^{b} is C₁-C₆ alkyl, and the other R⁶ is halo; One R⁶ is C₁-C₆ alkyl, and the other R⁶ is cyano; One R⁶ is C₃-C₇ cycloalkyl, and the other R⁶ is C₃-C₇ cycloalkyl; One R⁶ is C₃-C₇ cycloalkyl, and the other R⁶ is halo; One R⁶ is cyclopropyl and the other R⁶ is halo; One R⁶ is C₁-C₆ alkyl, and the other R⁶ is C₁-C₆ alkoxy optionally substituted with one or more halo; One R⁶ is C₁-C₆ alkyl, and the other R⁶ is C₁-C₆ alkoxy; One R⁶ is C₁-C₆ alkyl, and the other R⁶ is C₁-C₆ alkoxy substituted with one or more halo, One R⁶ is halo, and the other R⁶ is C₁-C₆ haloalkyl; One R⁶ is halo, and the other R⁶ is C₁-C₆ haloalkoxy; One R⁶ is C₁-C₆ alkoxy; and the other R⁶ is halo; One R⁶ is C₁-C₆ alkoxy; and the other R⁶ is chloro.

In some embodiments, of the compound of formula AA,

the optionally substituted ring B is

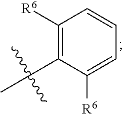

and the two R⁶ are one of the following combinations:
One R⁶ is isopropyl; and the other R⁶ is methyl, One R^{b} is isopropyl, and the other R⁶ is 77-propyl; One R⁶ is isopropyl; and the other R⁶ is isopropyl; One R⁶ is isopropyl; and the other R⁶ is trifluoromethyl; One R⁶ is isopropyl; and the other R⁶ is cyclopropyl; One R⁶ is isopropyl; and the other R⁶ is chloro; One R⁶ is isopropyl; and the other R⁶ is fluoro; One R⁶ is ethyl; and the other R⁶ is fluoro; One R⁶ is isopropyl; and the other R⁶ is cyano; One R⁶ is cyclopropyl; and the other R⁶ is cyclopropyl; One R⁶ is cyclopropyl, and the other R⁶ is chloro; One R⁶ is cyclopropyl; and the other R⁶ is fluoro; One R⁶ is isopropyl; and the other R⁶ is methoxy; One R⁶ is isopropyl; and the other R⁶ is methoxy; or One R⁶ is isopropyl; and the other R⁶ is trifluoromethoxy.

In some embodiments, of the compound of formula AA,

the optionally substituted ring B is

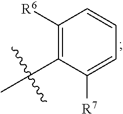

and R⁶ and R⁷ are one of the following combinations:
R⁶ is isopropyl; and R⁷ is methyl; R⁶ is isopropyl; and R⁷ is isopropyl; R⁶ is isopropyl; and R⁷ is trifluoromethyl; R⁶ is isopropyl; and R is cyclopropyl; R⁶ is isopropyl; and R⁷ is chloro; R⁶ is isopropyl; and R⁷ is fluoro; R⁶ is ethyl; and R⁷ is fluoro, R⁶ is isopropyl; and R⁷ is cyano; R⁶ is cyclopropyl; and R⁷ is cyclopropyl; R⁶ is cyclopropyl; and R⁷ is chloro; R⁶ is cyclopropyl; and R⁶ is fluoro; R⁶ is isopropyl; and R⁶ is methoxy; R⁶ is isopropyl; and R⁷ is trifluoromethoxy; R^{b} is chloro; and R⁷ is trifluoromethyl, R⁶ is chloro; and R⁷ is trifluoromethoxy; R⁷ is isopropyl; and R⁶ is methyl; R⁶ is isopropyl; and R⁶ is trifluoromethyl, R⁷ is isopropyl, and R⁶ is cyclopropyl; R⁷ is isopropyl; and R⁶ is chloro; R⁷ is ethyl; and R⁶ is fluoro; R⁶ is isopropyl; and R⁶ is cyano; R⁷ is cyclopropyl; and R⁶ is cyclopropyl; R⁶ is cyclopropyl, and R^{b} is chloro; R⁷ is cyclopropyl; and R⁶ is fluoro; R⁷ is isopropyl; and R⁶ is methoxy; R⁷ is isopropyl; and R⁶ is trifluoromethoxy; R is chloro; and R⁶ is trifluoromethyl; or R⁶ is chloro; and R⁶ is trifluoromethoxy.

In some embodiments, of the compound of formula AA,

the optionally substituted ring B is

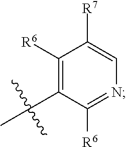

and R⁶ and R⁷ are one of the following combinations:

each R^{b} is independently C₁-C₆ alkyl, and R⁷ is C₃-C₆ alkyl optionally substituted with one or more halo; each R⁶ is independently C₁-C₆ alkyl and R⁷ is C₁-C₆ alkyl; each R⁶ is independently C₃-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R is C₃-C₇ cycloalkyl; each R⁶ is independently C₃-C₇ cycloalkyl, and R is halo; each R^{b} is independently cyclopropyl, and R⁷ is halo, each R⁶ is independently C₁-C₆ alkyl, and R is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy, and R is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R^{b} is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo, R⁷ is C₁-C₆ alkyl and each R⁶ is independently halo; R⁷ is C₁-C₆ alkyl, and R⁶ is cyano; R⁷ is C₃-C₆ cycloalkyl, and each R⁶ is independently C₃-C₇ cycloalkyl; R⁷ is C₃-C₇ cycloalkyl, and each R^{b} is independently halo, R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₃-C₆ alkoxy; R⁷ is C₃-C₆ alkyl, and each R⁶ is independently C₁-C₆ alkoxy substituted with one or more halo; R⁷ is halo, and each R⁶ is independently C₃-C₆ haloalkyl; R⁷ is halo, and each R⁶ is independently C₁-C₆ haloalkoxy; R⁷ is C₃-C₆ alkoxy; and each R⁶ is independently halo; or R⁷ is C₃-C₆ alkoxy; and R⁶ is chloro.

In some embodiments, of the compound of formula AA,

the optionally substituted ring B is

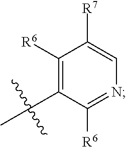

and R⁶ and R⁷ are one of the following combinations:
each R⁶ is isopropyl; and R⁷ is methyl; each R⁶ is isopropyl; and R⁷ is isopropyl; each R⁶ is isopropyl, and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl, and R⁷ is cyclopropyl; each R⁶ is cyclopropyl; and R⁷ is chloro; each R⁵ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl, and each R⁶ is methyl; R⁷ is isopropyl; and each R⁶ is trifluoromethyl, R is isopropyl, and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl, R⁷ is chloro, and each R⁶ is trifluoromethoxy; or one R⁶ is isopropyl; the other R⁶ is trifluoromethyl; and R⁷ is chloro.

In some embodiments, of the compound of formula AA,

the optionally substituted ring B is

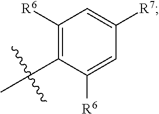

and R⁶ and R⁷ are one of the following combinations:
each R⁶ is independently C₁-C₆ alkyl, and R⁷ is C₁-C₆ alkyl optionally substituted with one or more halo, each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo, each R⁶ is independently cyclopropyl and R⁷ is halo, each R⁶ is independently C₁-C₆ alkyl, and R⁷ is C₁-C₆ alkoxy optionally substituted with one or more halo, each R⁶ is independently C₁-C₆ alkyl, and R⁷ is C₁-C₆ alkoxy, each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy, each R⁶ is independently C₁-C₆ alkoxy, and R is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R^{b} is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo, R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl, R⁷ is C₃-C₇ cycloalkyl, and each R⁷ is independently halo, R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₃-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; or R⁷ is C₁-C₆ alkoxy; and R⁶ is chloro.

In some embodiments, of the compound of formula AA,

the optionally substituted ring B is

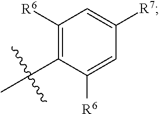

and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl, and R⁷ is cyclopropyl; each R⁶ is isopropyl; and R⁷ is chloro; each R⁶ is isopropyl, and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl, and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R^{b} is chloro; and R⁷ is trifluoromethyl, each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro, R⁷ is isopropyl, and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; or one R⁶ is isopropyl; the other R⁶ is trifluoromethyl, and R⁷ is chloro.

In some embodiments, of the compound of formula AA,

the optionally substituted ring B is

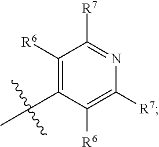

and R⁶ and R⁷ are one of the following combinations:
each R⁶ is independently C₁-C₆ alkyl, and each R⁷ is independently C₁-C₆ alkyl optionally substituted with one or more halo; each R⁶ is independently C₁-C₆ alkyl and each R⁷ is independently C₁-C₆ alkyl, each R⁶ is independently C₁-C₆ alkyl, and each R⁷ is independently C₁-C₆ alkyl substituted with one or more halo; each R⁵ is independently C₁-C₆ alkyl, and each R is independently C₃-C₇ cycloalkyl; each R⁶ is independently C₁-C₆ alkyl, and each R⁷ is independently halo; each R⁶ is independently C₁-C₆ alkyl, and R⁷ is cyano; each R⁶ is independently C₃-C₇ cycloalkyl, and each R⁷ is independently C₃-C₇ cycloalkyl; each R⁶ is independently C₃-C₇ cycloalkyl, and each R⁷ is independently halo; each R⁷ is independently cyclopropyl and each R⁷ is independently halo; each R⁶ is independently C₁-C₆ alkyl, and each R⁷ is independently C₁-C₆ alkoxy optionally substituted with one or more halo; each R⁶ is independently C₁-C₆ alkyl, and each R⁷ is independently C₁-C₆ alkoxy; each R⁶ is independently C₁-C₆ alkyl, and each R⁷ is independently C₁-C₆ alkoxy substituted with one or more halo; each R⁶ is independently halo, and each R⁷ is independently C₁-C₆ haloalkyl; each R⁶ is independently halo, and each R⁷ is independently C₁-C₆ haloalkoxy; each R⁶ is independently C₁-C₆ alkoxy, and each R is independently halo; each R⁶ is independently C₁-C₆ alkoxy; and R⁷ is chloro; each R⁷ is independently C₁-C₆ alkyl, and each R⁶ is independently C₁-C₆ alkyl optionally substituted with one or more halo; each R⁷ is independently C₁-C₆ alkyl, and each R⁶ is independently C₁-C₆ alkyl substituted with one or more halo; each R⁷ is independently C₁-C₆ alkyl, and each R⁶ is independently C₃-C₇ cycloalkyl; each R⁷ is independently C₁-C₆ alkyl, and each R⁶ is independently halo; each R⁷ is independently C₁-C₆ alkyl and each R⁶ is independently halo; each R⁷ is independently C₁-C₆ alkyl, and R⁶ is cyano; each R is independently C₃-C₇ cycloalkyl, and each R⁶ is independently C₃-C₇ cycloalkyl; each R⁷ is independently C₃-C₇ cycloalkyl, and each R⁵ is independently halo, each R⁷ is independently C₃-C₇ cycloalkyl and each R⁶ is independently halo; each R⁷ is independently C₁-C₆ alkyl, and each R⁶ is independently C₁-C₆ alkoxy optionally substituted with one or more halo; each R⁷ is independently C₁-C₆ alkyl, and each R⁶ is independently C₁-C₆ alkoxy; each R⁷ is independently C₁-C₆ alkyl, and each R⁶ is independently C₁-C₆ alkoxy substituted with one or more halo; each R⁷ is independently halo, and each R⁶ is independently C₁-C₆ haloalkyl; each R⁷ is independently halo, and each R⁶ is independently C₁-C₆ haloalkoxy; each R⁷ is independently C₁-C₆ alkoxy; and each R⁵ is independently halo; each R⁷ is independently C₁-C₆ alkoxy; and R⁶ is chloro, or two pairs, each of one R⁶ and one R⁷, are on adjacent atoms, and each pair of one R⁶ and one R⁷ taken together with the atoms connecting them form a C₁-C₆ aliphatic carbocyclic ring.

In some embodiments, of the compound of formula AA,

the optionally substituted ring B is

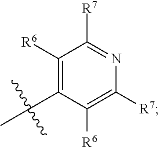

and R⁶ and R⁷ are one of the following combinations:
each R⁶ is isopropyl; and each R⁷ is methyl; each R⁶ is isopropyl; and each R⁷ is isopropyl; each R⁶ is isopropyl; and each R⁷ is trifluoromethyl; each R⁶ is isopropyl; and each R⁷ is cyclopropyl; each R⁶ is isopropyl; and each R⁷ is chloro; each R⁶ is isopropyl; and each R⁷ is fluoro; each R⁶ is ethyl; and each R⁷ is fluoro; each R⁶ is isopropyl, and each R⁷ is cyano; each R⁶ is cyclopropyl; and each R⁷ is cyclopropyl; each R⁶ is cyclopropyl; and each R⁷ is chloro; each R⁶ is cyclopropyl; and each R⁷ is fluoro; each R⁶ is isopropyl; and each R⁷ is methoxy; each R⁶ is isopropyl; and each R⁷ is trifluoromethoxy; each R⁶ is chloro; and each R⁷ is trifluoromethyl; each R⁶ is chloro; and each R⁷ is trifluoromethoxy; each R⁷ is isopropyl; and each R⁶ is methyl; each R⁷ is isopropyl; and each R⁶ is trifluoromethyl; each R⁷ is isopropyl; and each R⁶ is cyclopropyl; each R⁷ is isopropyl; and each R⁶ is chloro; each R⁷ is ethyl; and each R⁶ is fluoro; each R⁷ is isopropyl; and each R⁶ is cyano, each R⁷ is cyclopropyl; and each R⁶ is cyclopropyl; each R⁷ is cyclopropyl; and each R⁶ is chloro; each R⁷ is cyclopropyl; and each R⁶ is fluoro, each R⁷ is isopropyl, and each R⁶ is methoxy; each R⁷ is isopropyl; and each R⁶ is trifluoromethoxy; each R⁷ is chloro; and each R⁶ is trifluoromethyl; each R⁷ is chloro; and each R⁶ is trifluoromethoxy; one R⁶ is isopropyl; the other R⁶ is trifluoromethyl; and each R⁷ is chloro; each R⁶ is isopropyl; one R⁷ is fluoro; and the other R⁷ is cyano, or two pairs, each of one R⁶ and one R⁷, are on adjacent atoms, and each pair of one R⁶ and one R⁷ taken together with the atoms connecting them form a C₅ aliphatic carbocyclic ring.

In some embodiments, of the compound of formula AA,

the optionally substituted ring B is

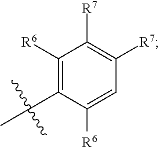

and R⁶ and R⁷ are one of the following combinations:
each R⁶ is independently C₁-C₆ alkyl, and each R⁷ is independently C₁-C₆ alkyl optionally substituted with one or more halo; each R⁶ is independently C₁-C₆ alkyl and each R⁷ is independently C₁-C₆ alkyl; each R⁶ is independently C₁-C₆ alkyl, and each R⁷ is independently C₁-C₆ alkyl substituted with one or more halo; each R⁶ is independently C₁-C₆ alkyl, and each R⁷ is independently C₃-C₇ cycloalkyl; each R⁶ is independently C₁-C₆ alkyl, and each R⁷ is independently halo; each R⁶ is independently C₁-C₆ alkyl, and R⁷ is cyano; each R⁶ is independently C₃-C₇ cycloalkyl, and each R⁷ is independently C₃-C₇ cycloalkyl; each R⁶ is independently C₃-C₇ cycloalkyl, and each R⁷ is independently halo, each R⁶ is independently cyclopropyl and each R⁷ is independently halo; each R⁵ is independently C₁-C₆ alkyl, and each R⁷ is independently C₁-C₆ alkoxy optionally substituted with one or more halo, each R⁶ is independently C₁-C₆ alkyl, and each R⁷ is independently C₁-C₆ alkoxy; each R⁶ is independently C₁-C₆ alkyl, and each R⁷ is independently C₁-C₆ alkoxy substituted with one or more halo; each R⁶ is independently halo, and each R⁷ is independently C₁-C₆ haloalkyl; each R⁶ is independently halo, and each R⁷ is independently C₁-C₆ haloalkoxy; each R⁶ is independently C₁-C₆ alkoxy; and each R⁷ is independently halo, each R⁶ is independently C₁-C₆ alkoxy; and R⁷ is chloro; each R⁷ is independently C₁-C₆ alkyl, and each R⁶ is independently C₁-C₆ alkyl optionally substituted with one or more halo; each R⁷ is independently C₁-C₆ alkyl, and each R⁶ is independently C₁-C₆ alkyl substituted with one or more halo; each R⁷ is independently C₁-C₆ alkyl, and each R⁶ is independently C₃-C₇ cycloalkyl, each R⁷ is independently C₁-C₆ alkyl, and each R^{b} is independently halo; each R is independently C₁-C₆ alkyl and each R⁶ is independently halo; each R⁷ is independently C₁-C₆ alkyl, and R⁶ is cyano; each R⁷ is independently C₃-C₇ cycloalkyl, and each R⁶ is independently C₃-C₇ cycloalkyl; each R is independently C₃-C₇ cycloalkyl, and each R⁶ is independently halo; each R⁷ is independently C₃-C₇ cycloalkyl and each R⁶ is independently halo; each R⁷ is independently C₁-C₆ alkyl, and each R⁶ is independently C₁-C₆ alkoxy optionally substituted with one or more halo; each R⁷ is independently C₁-C₆ alkyl, and each R^{b} is independently C₁-C₆ alkoxy, each R⁷ is independently C₁-C₆ alkyl, and each R⁶ is independently C₁-C₆ alkoxy substituted with one or more halo; each R⁷ is independently halo, and each R⁶ is independently C₁-C₆ haloalkyl; each R⁷ is independently halo, and each R⁶ is independently C₁-C₆ haloalkoxy; each R⁷ is independently C₁-C₆ alkoxy; and each R⁶ is independently halo; or each R⁷ is independently C₁-C₆ alkoxy, and R⁶ is chloro.

In some embodiments, of the compound of formula AA,

the optionally substituted ring B is

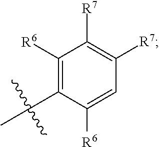

and R⁶ and R⁷ are one of the following combinations:
each R⁶ is isopropyl, and each R⁷ is methyl; each R⁶ is isopropyl; and each R⁷ is isopropyl; each R⁶ is isopropyl; and each R⁷ is trifluoromethyl; each R⁶ is isopropyl; and each R⁷ is cyclopropyl; each R⁶ is isopropyl; and each R⁷ is chloro; each R⁶ is isopropyl; and each R⁷ is fluoro; each R⁶ is ethyl; and each R is fluoro; each R⁶ is isopropyl; and each R⁷ is cyano; each R⁶ is cyclopropyl; and each R⁷ is cyclopropyl; each R⁶ is cyclopropyl; and each R⁷ is chloro; each R⁶ is cyclopropyl; and each R is fluoro; each R⁶ is isopropyl; and each R⁷ is methoxy; each R⁶ is isopropyl; and each R⁷ is trifluoromethoxy; each R⁶ is chloro; and each R⁷ is trifluoromethyl; each R⁶ is chloro, and each R⁷ is trifluoromethoxy, each R⁷ is isopropyl; and each R⁶ is methyl; each R⁷ is isopropyl; and each R⁶ is trifluoromethyl; each R⁷ is isopropyl, and each R⁶ is cyclopropyl; each R⁷ is isopropyl; and each R⁶ is chloro; each R⁷ is ethyl; and each R⁶ is fluoro; each R⁷ is isopropyl; and each R⁶ is cyano; each R⁷ is cyclopropyl; and each R⁶ is cyclopropyl; each R⁷ is cyclopropyl; and each R⁶ is chloro; each R⁷ is cyclopropyl; and each R⁶ is fluoro; each R⁷ is isopropyl; and each R⁶ is methoxy; each R⁷ is isopropyl, and each R⁶ is trifluoromethoxy; each R⁷ is chloro, and each R⁶ is trifluoromethyl, each R⁷ is chloro; and each R⁶ is trifluoromethoxy, one R^{b} is isopropyl, the other R⁶ is trifluoromethyl; and R⁷ is chloro; or R⁶ is isopropyl; one R⁷ is fluoro; and the other R⁷ is cyano.

In some embodiments, of the compound of formula AA,

the optionally substituted ring B is

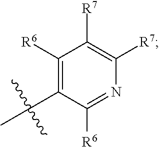

and R⁶ and R⁷ are one of the following combinations:
each R^{b} is independently C₁-C₆ alkyl, and each R⁷ is independently C₁-C₆ alkyl optionally substituted with one or more halo; each R⁶ is independently C₁-C₆ alkyl and each R is independently C₁-C₆ alkyl, each R⁶ is independently C₁-C₆ alkyl, and each R⁷ is independently C₁-C₆ alkyl substituted with one or more halo; each R⁶ is independently C₁-C₆ alkyl, and each R is independently C₃-C₇ cycloalkyl; each R⁶ is independently C₁-C₆ alkyl, and each R⁷ is independently halo; each R⁶ is independently C₁-C₆ alkyl, and R⁷ is cyano; each R⁶ is independently C₃-C₇ cycloalkyl, and each R⁷ is independently C₃-C₇ cycloalkyl; each R⁶ is independently C₃-C₇ cycloalkyl, and each R⁷ is independently halo; each R⁶ is independently cyclopropyl and each R⁷ is independently halo, each R⁶ is independently C₁-C₆ alkyl, and each R⁷ is independently C₁-C₆ alkoxy optionally substituted with one or more halo; each R⁶ is independently C₁-C₆ alkyl, and each R⁷ is independently C₁-C₆ alkoxy; each R⁶ is independently C₁-C₆ alkyl, and each R⁷ is independently C₁-C₆ alkoxy substituted with one or more halo; each R⁶ is independently halo, and each R⁷ is independently C₁-C₆ haloalkyl; each R⁶ is independently halo, and each R⁷ is independently C₁-C₆ haloalkoxy; each R⁶ is independently C₁-C₆ alkoxy; and each R is independently halo; each R⁶ is independently C₁-C₆ alkoxy; and R⁷ is chloro; each R⁷ is independently C₁-C₆ alkyl, and each R^{b} is independently C₁-C₆ alkyl optionally substituted with one or more halo; each R⁷ is independently C₁-C₆ alkyl, and each R⁶ is independently C₁-C₆ alkyl substituted with one or more halo; each R⁷ is independently C₁-C₆ alkyl, and each R^{b} is independently C₃-C₇ cycloalkyl; each R⁷ is independently C₁-C₆ alkyl, and each R⁶ is independently halo; each R⁷ is independently C₁-C₆ alkyl and each R⁶ is independently halo; each R⁷ is independently C₁-C₆ alkyl, and R⁶ is cyano; each R⁷ is independently C₃-C₇ cycloalkyl, and each R⁶ is independently C₃-C₇ cycloalkyl; each R⁷ is independently C₃-C₇ cycloalkyl, and each R⁶ is independently halo, each R⁷ is independently C₃-C₇ cycloalkyl and each R⁶ is independently halo; each R⁷ is independently C₁-C₆ alkyl, and each R⁶ is independently C₁-C₆ alkoxy optionally substituted with one or more halo; each R⁷ is independently C₁-C₆ alkyl, and each R⁶ is independently C₁-C₆ alkoxy; each R⁷ is independently C₁-C₆ alkyl, and each R⁶ is independently C₁-C₆ alkoxy substituted with one or more halo, each R⁷ is independently halo, and each R⁶ is independently C₁-C₆ haloalkyl; each R⁷ is independently halo, and each R⁶ is independently C₁-C₆ haloalkoxy; each R⁷ is independently C₁-C₆ alkoxy; and each R⁶ is independently halo; or each R⁷ is independently C₁-C₆ alkoxy; and R⁶ is chloro.

In some embodiments, of the compound of formula AA,

the optionally substituted ring B is

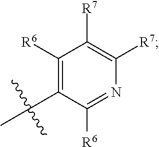

and R⁶ and R⁷ are one of the following combinations:
each R⁶ is isopropyl; and each R⁷ is methyl; each R⁶ is isopropyl; and each R⁷ is isopropyl; each R^{b} is isopropyl, and each R⁷ is trifluoromethyl; each R⁶ is isopropyl; and each R⁷ is cyclopropyl; each R⁶ is isopropyl; and each R⁷ is chloro; each R⁶ is isopropyl; and each R⁷ is fluoro; each R⁶ is ethyl; and each R⁷ is fluoro; each R⁶ is isopropyl; and each R⁷ is cyano; each R⁶ is cyclopropyl; and each R⁷ is cyclopropyl; each R⁶ is cyclopropyl; and each R⁷ is chloro; each R⁶ is cyclopropyl, and each R⁷ is fluoro; each R⁶ is isopropyl; and each R⁷ is methoxy; each R⁶ is isopropyl; and each R is trifluoromethoxy; each R⁶ is chloro; and each R⁷ is trifluoromethyl; each R⁶ is chloro; and each R⁷ is trifluoromethoxy; each R⁷ is isopropyl, and each R^{b} is methyl; each R⁷ is isopropyl; and each R⁶ is trifluoromethyl; each R⁷ is isopropyl; and each R⁶ is cyclopropyl; each R⁷ is isopropyl; and each R⁶ is chloro; each R⁷ is ethyl, and each R^{b} is fluoro; each R⁷ is isopropyl, and each R⁶ is cyano; each R⁷ is cyclopropyl; and each R⁵ is cyclopropyl; each R⁷ is cyclopropyl; and each R⁶ is chloro; each R⁷ is cyclopropyl; and each R⁶ is fluoro; each R⁷ is isopropyl; and each R⁶ is methoxy; each R⁷ is isopropyl; and each R⁶ is trifluoromethoxy; each R⁷ is chloro; and each R⁶ is trifluoromethyl, each R⁷ is chloro; and each R⁶ is trifluoromethoxy, one R⁶ is isopropyl, the other R⁶ is trifluoromethyl, and each R⁷ is chloro; or each R⁶ is isopropyl; one R⁷ is fluoro; and the other R⁷ is cyano.

In some embodiments, of the compound of formula AA,

the optionally substituted ring Bis R⁶;

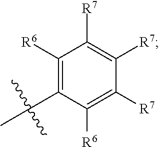

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C³-C₆ alkyl, and R⁷ is cyano; each R⁶ is independently C₃-C₇ cycloalkyl, and R⁷ is C₃-C₇ cycloalkyl; each R^{b} is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R^{b} is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro, R⁷ is C₁-C₆ alkyl, and each R⁶ is independently C₁-C₆ alkyl optionally substituted with one or more halo; R is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo, R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl, R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R^{b} is independently C₁-C₆ haloalkyl; R is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; two pairs, each of one R⁶ and one R⁷, are on adjacent atoms, and each pair of one R⁶ and one R⁷ taken together with the atoms connecting them form a C₄-C₈ aliphatic carbocyclic ring; and one R⁷ is halo, or two pairs, each of one R⁶ and one R⁷, are on adjacent atoms, and each pair of one R⁶ and one R⁷ taken together with the atoms connecting them form a C₄-C₈ aliphatic carbocyclic ring, and one R⁷ is cyano.

In some embodiments, of the compound of formula AA,

the optionally substituted ring B is

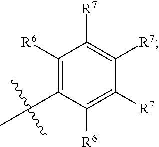

and R⁶ and R⁷ are one of the following combinations:
each R⁶ is isopropyl; and each R⁷ is methyl; each R⁶ is isopropyl; and each R⁷ is isopropyl; each R⁶ is isopropyl; and each R⁷ is trifluoromethyl; each R⁶ is isopropyl; and each R is cyclopropyl; each R⁶ is isopropyl; and each R⁷ is chloro; each R⁶ is isopropyl; and each R⁷ is fluoro; each R⁶ is ethyl; and each R⁷ is fluoro; each R⁶ is isopropyl, and each R⁷ is cyano; each R⁶ is cyclopropyl; and each R⁷ is cyclopropyl; each R⁶ is cyclopropyl; and each R⁷ is chloro; each R⁶ is cyclopropyl; and each R⁷ is fluoro; each R⁶ is isopropyl; and each R⁷ is methoxy; each R⁶ is isopropyl; and each R⁷ is trifluoromethoxy; each R⁶ is chloro; and each R⁷ is trifluoromethyl; each R⁶ is chloro; and each R⁷ is trifluoromethoxy; each R⁷ is isopropyl; and each R⁶ is methyl; each R⁷ is isopropyl; and each R⁶ is trifluoromethyl; each R⁷ is isopropyl; and each R⁶ is cyclopropyl; each R⁷ is isopropyl; and each R⁶ is chloro; each R⁷ is ethyl; and each R⁶ is fluoro; each R⁷ is isopropyl; and each R⁶ is cyano, each R⁷ is cyclopropyl; and each R⁶ is cyclopropyl; each R⁷ is cyclopropyl; and each R⁶ is chloro; each R⁷ is cyclopropyl; and each R⁶ is fluoro, each R⁷ is isopropyl, and each R⁶ is methoxy; each R⁷ is isopropyl; and each R⁶ is trifluoromethoxy; each R⁷ is chloro; and each R⁶ is trifluoromethyl; each R⁷ is chloro; and each R⁶ is trifluoromethoxy; each R^{b} is isopropyl, two R⁷ are fluoro; and one R⁷ is chloro; two pairs, each of one R⁶ and one R⁷, are on adjacent atoms, and each pair of one R⁶ and one R⁷ taken together with the atoms connecting them form a C₅ aliphatic carbocyclic ring; and one R⁷ is chloro; or two pairs, each of one R⁶ and one R⁷, are on adjacent atoms, and each pair of one R⁶ and one R⁷ taken together with the atoms connecting them form a C₅ aliphatic carbocyclic ring; and one R⁷ is fluoro.
In some embodiments, the compound of formula AA is a compound of Formula Va

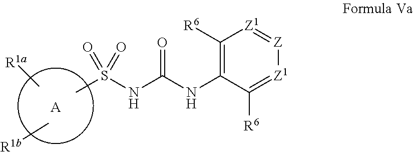

wherein

A is thiazolyl;

R^{1a} is a C₁-C₆ alkyl substituted with one or more hydroxy or —OSi(R¹³)₃;

R^{1b} is a C₁-C₆ alkyl substituted with one or more hydroxy;

Z is N, CH, or CR⁷;

each R⁶ is independently hydrogen, C₁-C₆ alkoxy, halo, C₁-C₆ haloalkyl, C₃-C₇ cycloalkyl, or C₁-C₆ alkyl optionally substituted with hydroxy;

each Z¹ is independently N, CH or CR⁷, each R⁷ is independently hydrogen, C₁-C₆ alkoxy, halo, C₁-C₆ haloalkyl, CN, C₁-C₆ haloalkoxy, C₃-C₇ cycloalkyl, or C₁-C₆ alkyl optionally substituted with hydroxy;

or at least one pair of R⁶ and R⁷ in adjacent positions, taken together with the carbon atoms connecting them, form a four-membered to seven-membered carbocyclic or heterocyclic ring containing 1 or 2 heteroatoms independently selected from O, N, and S; and

wherein the four-membered to seven-membered carbocyclic or heterocyclic ring is optionally independently substituted with one or more substituents selected from H, F, C₁-C₆ alkyl, C₁-C₆ alkoxy, NR⁸R⁹, oxo, and ═NR¹⁰.

In some embodiments of the compound of formula Va, A is 2-thiazolyl. In some embodiments of the compound of formula Va, A is 4-thiazolyl. In some embodiments of the compound of formula Va, A is 5-thiazolyl.

In some embodiments, the compound of Formula Va is a compound of Formula Va-i:

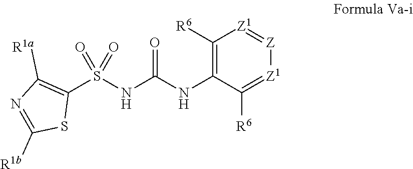

In some embodiments, the compound of Formula Va is a compound of Formula Va-ii:

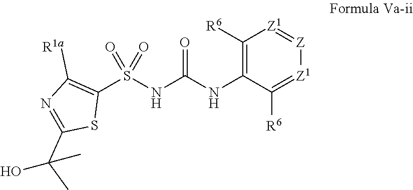

In some embodiments, the compound of Formula Va is a compound of Formula Va-iii:

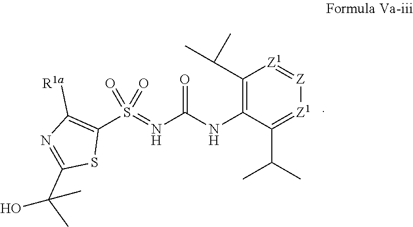

In some embodiments, the compound of Formula Va is a compound of Formula Va-iv:

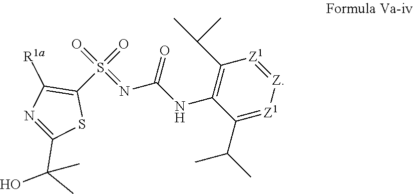

wherein Z¹ is CH or CR⁷; and R^{1a} is an unbranched C₁-C₆ alkyl substituted with one hydroxy.

In some embodiments of the compound of formula Va, Va-i, Va-ii, and Va-iii, Z is N. In some embodiments of the compound of formula Va, Va-i, Va-ii, Va-iii, and Va-iv, Z is CR⁷. In some embodiments of the compound of formula Va, Va-i, Va-ii, Va-iii, and Va-iv, Z is CH.

In some embodiments of the compound of formula Va, Va-i, Va-ii, Va-iii, and Va-vi, R^{1a} is hydroxymethyl. In some embodiments of the compound of formula Va, Va-i, Va-ii, Va-iii, and Va-vi, R^{1a} is hydroxyethyl. In some embodiments of the compound of formula Va, Va-i, Va-ii, Va-iii, and Va-vi, R^{1a} is 3-hydroxy-1-propyl. In some embodiments of the compound of formula Va, Va-i, Va-ii, and Va-iii, R^{1a} is 2-hydroxy-2-propyl. In some embodiments of the compound of formula Va, Va-i, Va-ii, and Va-iii, R^{1a} is 3-hydroxy-2-propyl. In some embodiments of the compound of formula Va, Va-i, Va-ii, and Va-iii, R^{1a} is 1-hydroxy-1-propyl. In some embodiments of the compound of formula Va, Va-i, Va-ii, and Va-iii, R^{1a} is 2-hydroxy-1-propyl. In some embodiments of the compound of formula Va, Va-i, Va-ii, and Va-iii, R^{1a} is hydroxybutyl. In some embodiments of the compound of formula Va, Va-i, Va-ii, and Va-iii, R^{1a} is hydroxypentyl. In some embodiments of the compound of formula Va, Va-i, Va-ii, and Va-iii, R^{1a} is hydroxyhexyl. In some embodiments of the compound of formula Va, Va-i, Va-ii, and Va-iii, R^{1a} is an unbranched C₁-C₆ alkyl substituted with one hydroxy. In some embodiments of the compound of formula Va, Va-i, Va-ii, and Va-iii, R^{1a} is a branched C₁-C₆ alkyl substituted with one hydroxy.

In some embodiments of the compound of formula Va and Va-i, R^{1b} is hydroxymethyl. In some embodiments of the compound of formula Va and Va-i, R^{1b} is hydroxyethyl. In some embodiments of the compound of formula Va and Va-i, R^{1b} is 2-hydroxy-2-propyl. In some embodiments of the compound of formula Va and Va-i, R^{1b} is 3-hydroxy-2-propyl. In some embodiments of the compound of formula Va and Va-i, R^{1b} is 1-hydroxy-1-propyl. In some embodiments of the compound of formula Va and Va-i, R^{1b} is 2-hydroxy-1-propyl. In some embodiments of the compound of formula Va and Va-i, R^{1b} is hydroxybutyl. In some embodiments of the compound of formula Va and Va-i, R^{1b} is hydroxypentyl. In some embodiments of the compound of formula Va and Va-i, R^{1b} is hydroxyhexyl. In some embodiments of the compound of formula Va and Va-i, R^{1b} is an unbranched C₁-C₆ alkyl substituted with one hydroxy. In some embodiments of the compound of formula Va and Va-i, R^{1b} is a branched C₁-C₆ alkyl substituted with one hydroxy.

In some embodiments of the compound of formula Va, Va-i, Va-ii, Va-iii, and Va-iv, each Z¹ is CH. In some embodiments of the compound of formula Va, Va-i, Va-ii, Va-iii, and Va-iv, one Z¹ is CH and the other Z¹ is CR⁷. In some embodiments of the compound of formula Va, Va-i, Va-ii, Va-iii, and Va-iv, each Z¹ is CR⁷. In some embodiments of the compound of formula Va, Va-i, Va-ii, Va-iii, and Va-iv, Z is CR⁷ wherein R⁷ is CN. In some embodiments of the compound of formula Va, Va-i, Va-ii, Va-iii, and Va-iv, Z is CR⁷ wherein R⁷ is halo (e.g., F). In some embodiments of the compound of formula Va, Va-i, Va-ii, Va-iii, and Va-iv, Z is CR wherein R⁷ is CO₂C₁-C₆ alkyl. In some embodiments of the compound of formula Va, Va-i, Va-ii, Va-iii, and Va-iv, Z is CR⁷ wherein R is CONR¹¹R¹²; In some embodiments of the compound of formula Va, Va-i, Va-ii, Va-iii, and Va-iv, Z is CR⁷ wherein R⁷ is C₁-C₆ alkyl. In some embodiments of the compound of formula Va, Va-i, Va-ii, Va-iii, and Va-iv, Z is CR⁷ wherein R⁷ is C₁-C₆ alkoxy; In some embodiments of the compound of formula Va, Va-i, Va-ii, Va-iii, and Va-iv, Z is CR⁷ wherein R⁷ is C₁-C₆ haloalkyl. In some embodiments of the compound of formula Va, Va-i, Va-ii, Va-iii, and Va-iv, each R⁷ that is meta to the NH(CO) group is hydrogen. In some embodiments of the compound of formula Va, Va-i, Va-ii, Va-iii, and Va-iv, each R⁷ that is meta to the NH(CO) group is C₁-C₆ alkoxy. In some embodiments of the compound of formula Va, Va-i, Va-ii, Va-iii, and Va-iv, each R⁷ that is meta to the NH(CO) group is halo. In some embodiments of the compound of formula Va, Va-i, Va-ii, Va-iii, and Va-iv, each R⁷ that is meta to the NH(CO) group is fluoro. In some embodiments of the compound of formula Va, Va-i, Va-ii, Va-iii, and Va-iv, each R that is meta to the NH(Co) group is C₁-C₆ haloalkyl. In some embodiments of the compound of formula Va, Va-i, Va-ii, Va-iii, and Va-iv, each R⁷ that is meta to the NH(CO) group is CN. In some embodiments of the compound of formula Va, Va-i, Va-ii, Va-iii, and Va-iv, each R⁷ that is meta to the NH(CO) group is C₃-C₇ cycloalkyl. In some embodiments of the compound of formula Va, Va-i, Va-ii, Va-iii, and Va-iv, each R⁷ that is meta to the NH(CO) group is C₁-C₆ alkyl optionally substituted with hydroxyl. In some embodiments of the compound of formula Va, Va-i, Va-ii, Va-iii, and Va-iv, each R⁷ that is meta to the NH(CO) group is unsubstituted C₁-C₆ alkyl. In some embodiments of the compound of formula Va, Va-i, Va-ii, Va-iii, and Va-iv, one R⁷ that is meta to the NH(CO) group is hydrogen and is different from the other R⁷ that is meta to the NH(CO) group. In some embodiments of the compound of formula Va, Va-i, Va-ii, Va-iii, and Va-iv, one R⁷ that is meta to the NH(CO) group is C₁-C₆ alkoxy and is different from the other R⁷ that is meta to the NH(CO) group. In some embodiments of the compound of formula Va. Va-i, Va-ii, Va-iii, and Va-iv, one R⁷ that is meta to the NH(CO) group is halo and is different from the other R that is meta to the NH(CO) group. In some embodiments of the compound of formula Va, Va-i, Va-ii, Va-iii, and Va-iv, one R⁷ that is meta to the NH(CO) group is C₁-C₆ haloalkyl and is different from the other R⁷ that is meta to the NH(CO) group. In some embodiments of the compound of formula Va, Va-i, Va-ii, Va-iii, and Va-iv, one R⁷ that is meta to the NH(CO) group is CN and is different from the other R⁷ that is meta to the NH(CO) group. In some embodiments of the compound of formula Va, Va-i, Va-ii, Va-iii, and Va-iv, one R⁷ that is meta to the NH(Co) group is C₃-C₇ cycloalkyl and is different from the other R⁷ that is meta to the NH(CO) group. In some embodiments of the compound of formula Va, Va-i, Va-ii, Va-iii, and Va-iv, one R⁷ that is meta to the NH(CO) group is C₁-C₆ alkyl optionally substituted with hydroxyl and is different from the other R⁷ that is meta to the NH(CO) group. In some embodiments of the compound of formula Va, Va-i, Va-ii, Va-iii, and Va-iv, one R⁷ that is meta to the NH(CO) group is unsubstituted C₁-C₆ alkyl and is different from the other R⁷ that is meta to the NH(CO) group.

In some embodiments of the compound of formula Va, Va-i, and Va-ii, each R⁶ is C₁-C₆ alkoxy. In some embodiments of the compound of formula Va, Va-i, and Va-ii, each R⁶ is halo. In some embodiments of the compound of formula Va, Va-i, and Va-ii, each R⁶ is C₁-C₆ haloalkyl. In some embodiments of the compound of formula Va, Va-i, and Va-ii, each R⁶ is CN. In some embodiments of the compound of formula Va, Va-i, and Va-ii, each R⁶ is C₃-C₇ cycloalkyl. In some embodiments of the compound of formula Va, Va-i, and Va-ii, each R⁵ is C₁-C₆ alkyl optionally substituted with hydroxyl (e.g., 2-hydroxy-2-propyl). In some embodiments of the compound of formula Va, Va-i, and Va-ii, each R⁶ is unsubstituted C₁-C₆ alkyl. In some embodiments of the compound of formula Va, Va-i, and Va-ii, one R⁶ is hydrogen and is different from the other R^{b}. In some embodiments of the compound of formula Va, Va-i, and Va-ii, one R⁶ is C₁-C₆ alkoxy and is different from the other R⁶. In some embodiments of the compound of formula Va, Va-i, and Va-ii, one R^{b} is halo and is different from the other R⁶; In some embodiments of the compound of formula Va, Va-i, and Va-ii, one R⁶ is C₁-C₆ haloalkyl and is different from the other R⁶. In some embodiments of the compound of formula Va, Va-i, and Va-ii, one R⁶ is CN and is different from the other R⁶. In some embodiments of the compound of formula Va, Va-i, and Va-ii, one R⁶ is C₃-C₇ cycloalkyl and is different from the other R⁶. In some embodiments of the compound of formula Va, Va-i, and Va-ii, one R⁶ is C₁-C₆ alkyl optionally substituted with hydroxyl and is different from the other R⁶. In some embodiments of the compound of formula Va, Va-i, and Va-ii, one R⁶ is unsubstituted C₁-C₆ alkyl and is different from the other R^{b}.

In some embodiments of the compound of formula Va, Va-i, and Va-ii, at least one pair of R⁶ and If in adjacent, positions, taken together with the carbon atoms connecting them, form a four-membered to seven-membered carbocyclic or heterocyclic ring containing 1 or 2 heteroatoms independently selected from O, N, and S;

wherein the four-membered to seven-membered carbocyclic or heterocyclic ring is optionally independently substituted with one or more substituents selected from F, C₁-C₆ alkyl, C₁-C₆ alkoxy, NR⁸R⁹, oxo, and ═NR¹⁰.

In some more embodiments of the compound of formula Va, Va-i, and Va-ii, the optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, the optionally independently substituted four-membered to seven-membered carbocyclic or heterocyclic ring is an optionally independently substituted four-membered carbocyclic ring.

In some embodiments of the compound of formula Va, Va-i, and Va-ii, both pairs of R⁶ and R in adjacent positions, taken together with the carbon atoms connecting them, each form a four-membered to seven-membered carbocyclic or heterocyclic ring containing 1 or 2 heteroatoms independently selected from O, N, and S;

wherein each four-membered to seven-membered carbocyclic or heterocyclic ring is optionally independently substituted with one or more substituents selected from F, C₁-C₆ alkyl, C₁-C₆ alkoxy, NR⁸R⁹, oxo, and ═NR¹⁰,

In some more embodiments of the compound of formula Va, Va-i, and Va-ii, each optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, one optionally independently substituted four-membered to seven-membered carbocyclic or heterocyclic ring is an optionally independently substituted four-membered carbocyclic ring, and the other optionally independently substituted four-membered to seven-membered carbocyclic or heterocyclic ring is an optionally independently substituted five-membered carbocyclic ring optionally substituted with one or more F or methyl.

In some more embodiments of the compound of formula Va, Va-i, and Va-ii, one optionally independently substituted four-membered to seven-membered carbocyclic or heterocyclic ring is an optionally independently substituted five-membered carbocyclic ring, and the other optionally independently substituted four-membered to seven-membered carbocyclic or heterocyclic ring is an optionally independently substituted five-membered carbocyclic ring optionally substituted with one or more F or methyl.

Non-Limiting Combinations of Substituted Ring A and Optionally Substituted Ring B

In some embodiments, the compound of formula AA is a compound wherein the substituted ring

A is

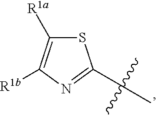

the optionally optionally substituted ring B is

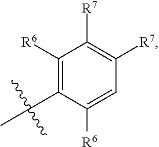

and wherein:
R^{1a} is hydroxymethyl, and R^{1b} is hydroxymethyl; R^{1a} is hydroxymethyl, and R¹⁰ is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is I-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl. and R^{1b} is 3-hydroxy-1-propyl; R^{1a} is hydroxyethyl. and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R¹⁰ is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2 propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl, R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl, R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations;
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl, each R⁶ is independently C₁-C₆ alkyl, and R⁷ is C₁-C₆ alkyl substituted with one or more halo; each R⁵ is independently C₁-C₆ alkyl, and R is C₃-C₇ cycloalkyl; each R^{b} is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl, each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy, and R is chloro; R⁷ is C₃-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁵ is independently halo; R⁷ is C₁-C₆ alkyl and each R⁶ is independently halo, R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R⁷ is C₃-C₇ cycloalkyl and each R^{b} is independently halo, R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy, R⁷ is C₁-C₆ alkoxy, and each R⁶ is independently halo; R⁷ is C₁-C₆ alkoxy; and R⁶ is chloro; R⁵ and R⁷ on adjacent atoms taken together with the atoms connecting them form a C₅ aliphatic carbocyclic ring.

In some embodiments, the compound of formula AA is a compound wherein the substituted ring A is

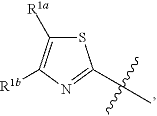

the optionally optionally substituted ring B is

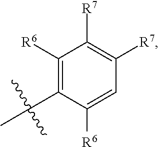

and wherein:
R^{1a} is hydroxymethyl, and R^{1b} is hydroxymethyl; R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl, R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl, R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R¹⁰ is hydroxyhexyl, R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R¹⁰ is 3-hydroxy-2-propyl; R^{1a} is 2-hydroxy-2-propyl, and R^{1b} is 1-hydroxy-1-propyl; R^{1a} is 2-hydroxy-2-propyl, and R¹⁰ is 2-hydroxy-1-propyl; R^{1a} is 2-hydroxy-2-propyl, and R^{1b} is 3-hydroxy-1-propyl, R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl, R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl, R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is isopropyl; and R⁷ is methyl; each R⁶ is isopropyl; and R is isopropyl; each R⁶ is isopropyl, and R⁷ is trifluoromethyl; each R⁶ is isopropyl; and R⁷ is cyclopropyl; each R^{b} is isopropyl; and R⁷ is chloro; each R⁶ is isopropyl; and R⁷ is fluoro; each R⁶ is ethyl; and R⁷ is fluoro; each R⁶ is isopropyl; and R⁷ is eyano; each R⁶ is cyclopropyl; and R⁷ is cyclopropyl; each R⁶ is cyclopropyl; and R⁷ is chloro; each R⁶ is cyclopropyl; and R is fluoro; each R⁶ is isopropyl; and R⁷ is methoxy; each R⁶ is isopropyl; and R⁷ is trifluoromethoxy, each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl, and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring A is

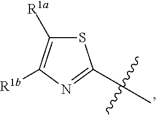

the optionally optionally substituted ring B is

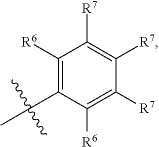

and wherein:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R¹⁰ is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxy ethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl, R^{1a} is hydroxyethyl, and R^{1b} is hydroxypentyl; R^{1a} is hydroxyethyl, and R¹⁰ is hydroxyhexyl; R^{1a} is 2-hydroxy-2-propyl, and R¹⁰ is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl, R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R¹⁰ is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl, R^{1b} is hydroxyethyl, and R^{1a} is 2-hydroxy-2-propyl, R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propy 1, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R^{b} is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R is C₁-C₆ haloalkoxy; each R⁶ is independently C₁-C₆ alkoxy; and R⁷ is halo; each R⁶ is independently C₁-C₆ alkoxy, and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl, R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁵ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy, R⁷ is C₁-C₆ alkoxy, and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring A is

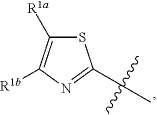

the optionally optionally substituted ring B is

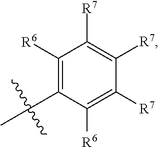

and wherein:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R¹⁰ is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl, R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R¹⁰ is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl, R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propy 1, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl, R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is isopropyl; and R⁷ is methyl; each R⁶ is isopropyl; and R⁷ is isopropyl; each R⁶ is isopropyl, and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl, R is isopropyl, and each R^{b} is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl, R⁷ is chloro, and each R⁶ is trifluoromethoxy; one R⁶ is isopropyl; the other R⁶ is trifluoromethyl; and R⁷ is chloro, R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring A is

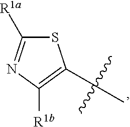

the optionally optionally substituted ring B is

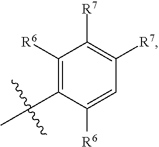

and wherein: R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R¹⁰ is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl, R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R¹⁰ is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl, R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propy 1, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl, R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁵ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R^{b} is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R is C₁-C₆ haloalkoxy; each R⁶ is independently C₁-C₆ alkoxy; and R⁷ is halo; each R⁶ is independently C₁-C₆ alkoxy, and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl, R⁷ is C₃-C₇ cycloalkyl, and each R⁶ is independently halo; R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy, and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring A is

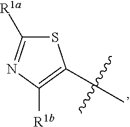

the optionally optionally substituted ring B is

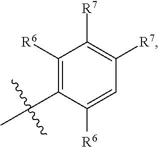

and wherein:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl, R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl, R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R¹⁰ is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R¹⁰ is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl, R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl; and R⁷ is cyclopropyl; each R⁶ is isopropyl; and R⁷ is chloro; each R⁶ is isopropyl, and R⁷ is fluoro; each R⁶ is ethyl; and R⁷ is fluoro; each R⁶ is isopropyl; and R⁷ is eyano; each R⁶ is cyclopropyl; and R⁷ is cyclopropyl; each R⁶ is cyclopropyl, and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R^{b} is chloro; and R⁷ is trifluoromethyl; each R⁶ is chloro; and R⁷ is trifluoromethoxy; R⁷ is isopropyl; and each R⁶ is methyl; R is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R is isopropyl; and each R⁶ is trifluoromethoxy; R⁷ is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring A is

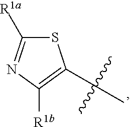

the optionally optionally substituted ring B is

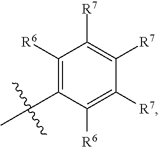

and wherein:
R^{1a} is hydroxymethyl, and R^{1b} is hydroxymethyl, R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl, R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl, R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R¹⁰ is hydroxyhexyl, R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R¹⁰ is 3-hydroxy-2-propyl; R^{1a} is 2-hydroxy-2-propyl, and R^{1b} is 1-hydroxy-1-propyl; R^{1a} is 2-hydroxy-2-propyl, and R¹⁰ is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R¹⁰ is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl, R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl, R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R is C₁-C₆ haloalkoxy, each R⁶ is independently C₁-C₆ alkoxy, and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo, R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R^{b} is independently halo; R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₃-C₆ alkoxy; R⁷ is C₃-C₆ alkyl, and each R⁶ is independently C₁-C₆ alkoxy substituted with one or more halo; R⁷ is halo, and each R⁶ is independently C₃-C₆ haloalkyl; R is halo, and each R⁶ is independently C₁-C₆ haloalkoxy; R⁷ is C₁-C₆ alkoxy; and each R⁶ is independently halo; R⁷ is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring A is

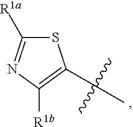

the optionally optionally substituted ring B is

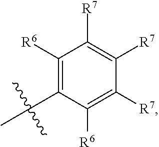

and wherein:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R¹⁰ is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R¹⁰ is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxy ethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R¹⁰ is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl, R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl, R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl, R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is isopropyl; and R⁷ is methyl; each R⁶ is isopropyl; and R⁷ is isopropyl; each R⁶ is isopropyl, and R⁷ is trifluoromethyl; each R⁶ is isopropyl; and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is eyano; each R⁶ is cyclopropyl; and R⁷ is cyclopropyl; each R⁶ is cyclopropyl; and R⁷ is chloro; each R⁵ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R⁶ is chloro; and R⁷ is trifluoromethoxy; R⁷ is isopropyl; and each R⁶ is methyl; R⁷ is isopropyl; and each R⁶ is trifluoromethyl, R is isopropyl, and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R is chloro; and each R⁶ is trifluoromethyl; R⁷ is chloro; and each R⁶ is trifluoromethoxy; one R⁶ is isopropyl; the other R⁶ is trifluoromethyl; and R⁷ is chloro, R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring A is

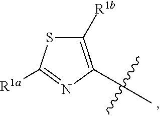

the optionally optionally substituted ring B is

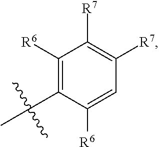

and wherein:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R¹⁰ is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R¹⁰ is hydroxymethyl; R^{1a} is 2-hydroxyl-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl, R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R¹⁰ is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxy pentyl; R^{1b} is hydroxymethyl, and R^{1a} is hydroxyhexyl; R^{1b} is hydroxyethyl, and R^{1a} is hydroxymethyl, R^{1b} is hydroxyethyl, and R^{1a} is hydroxyethyl; R^{1b} is hydroxyethyl. and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R¹⁰ is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propy 1, and R^{1a} is 3-hydroxy-2-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl, R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is independently C₁-C₆ alkyl, and R⁷ is C₁-C₆ alkyl optionally substituted with one or more halo; each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R is C₃-C₇ cycloalkyl; each R^{b} is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl, each R⁶ is independently C₃-C₇ cycloalkyl, and R⁷ is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R is C₁-C₆ haloalkoxy; each R⁶ is independently C₁-C₆ alkoxy; and R⁷ is halo; each R⁶ is independently C₁-C₆ alkoxy, and R is chloro; R⁷ is C₃-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R⁷ is C₃-C₇ cycloalkyl, and each R⁶ is independently C₃-C₇ cycloalkyl; R⁷ is C₃-C₇ cycloalkyl, and each R⁶ is independently halo; R is C₃-C₇ cycloalkyl and each R⁶ is independently halo, R⁷ is C₁-C₆ alkyl, and each R⁶ is independently C₁-C₆ alkoxy optionally substituted with one or more halo, R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R⁷ is C₁-C₆ alkoxy; and each R⁶ is independently halo; R is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring A is

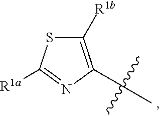

the optionally optionally substituted ring B is

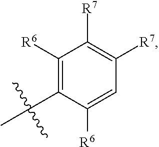

and wherein:
R^{1a} is hydroxymethyl, and R^{1b} is hydroxymethyl; R^{1a} is hydroxymethyl, and R^{1b} is hydroxyethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1b} is hydroxyhexyl; R^{1a} is 2-hydroxy-2-propyl, and R¹⁰ is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl; R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl; R^{1a} is 2-hydroxy-2-propyl, and R^{1b} is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxy ethyl; R^{1b} is hydroxymethyl, and R^{1a} is 2-hydroxy-2-propyl; R^{1b} is hydroxymethyl, and R^{1a} is 3-hydroxy-2-propyl, R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R¹⁰ is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl, R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl, R^{1b} is 2-hydroxy-2-propyl, and R^{1a} is hydroxypentyl; R^{1b} is 2-hydroxy-2-propyl, and R^{1a} is hydroxyhexyl;

and R⁶ and R are one of the following combinations;
each R⁶ is isopropyl; and R⁷ is methyl; each R⁶ is isopropyl; and R⁷ is isopropyl; each R⁶ is isopropyl; and R⁷ is trifluoromethyl; each R⁶ is isopropyl, and R⁷ is cyclopropyl; each R⁶ is isopropyl; and R⁷ is chloro; each R⁶ is isopropyl; and R⁷ is fluoro; each R⁶ is ethyl; and R⁷ is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and R⁷ is fluoro; each R⁶ is isopropyl; and R⁷ is methoxy; each R⁶ is isopropyl; and R⁷ is trifluoromethoxy; each R⁶ is chloro; and R⁷ is trifluoromethyl, each R⁶ is chloro; and R⁷ is trifluoromethoxy; R⁷ is isopropyl; and each R⁶ is methyl; R⁷ is isopropyl; and each R⁶ is trifluoromethyl; R⁷ is isopropyl; and each R⁶ is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R is cyclopropyl; and each R⁶ is fluoro; R⁷ is isopropyl; and each R⁶ is methoxy; R⁷ is isopropyl; and each R^{b} is trifluoromethoxy; R⁷ is chloro, and each R^{b} is trifluoromethyl; R⁷ is chloro; and each R⁶ is trifluoromethoxy; one R⁶ is isopropyl; the other R⁶ is trifluoromethyl; and R⁷ is chloro; R⁶ and R⁷ on adjacent atoms taken together with the atoms connecting them form a C₅ aliphatic carbocyclic ring; and one R⁶ is fluoro, chloro, or cyano.

In some embodiments, the compound of formula AA is a compound wherein the substituted ring A is

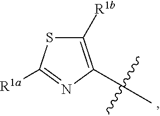

the optionally optionally substituted ring B is

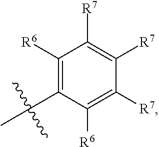

and wherein:
R^{1a} is hydroxymethyl, and R^{1b} is hydroxymethyl, R^{1a} is hydroxymethyl, and R^{1b} is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R¹⁰ is hydroxyhexyl, R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R¹⁰ is 3-hydroxy-2-propyl; R^{1a} is 2-hydroxy-2-propyl, and R^{1b} is 1-hydroxy-1-propyl, R^{1a} is 2-hydroxy-2-propyl, and R^{1b} is 2-hydroxy-1-propyl; R^{1a} is 2-hydroxy-2-propyl, and R^{1b} is 3-hydroxy-1-propyl; R^{1a} is 2-hydroxy-2-propyl, and R^{1b} is hydroxybutyl, R^{1a} is 2-hydroxy-2-propyl, and R^{1b} is hydroxypentyl, R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl, R^{1b} is hydroxyethyl, and R^{1a} is hydroxymethyl; R^{1b} is hydroxyethyl, and R^{1a} is hydroxyethyl; R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl, R^{1b} is hydroxyethyl, and R^{1a} is 2-hydroxy-1-propyl; R^{1b} is hydroxyethyl, and R^{1a} is 3-hydroxy-1-propyl; R^{1b} is hydroxyethyl, and R^{1a} is hydroxybutyl; R¹⁰ is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2hydroxy 2-propyl, and R^{1a} is hydroxyhexyl;

and R⁶ and R are one of the following combinations:
each R⁶ is independently C₁-C₆ alkyl, and R⁷ is C₁-C₆ alkyl optionally substituted with one or more halo, each R⁶ is independently C₁-C₆ alkyl and R⁷ is C₁-C₆ alkyl; each R⁶ is independently C₁-C₆ alkyl, and R⁷ is C₁-C₆ alkyl substituted with one or more halo; each R⁶ is independently C₁-C₆ alkyl, and R⁷ is C₃-C₇ cycloalkyl; each R⁶ is independently C₁-C₆ alkyl, and R⁷ is halo; each R⁶ is independently C₁-C₆ alkyl, and R⁷ is cyano; each R⁶ is independently C₃-C₇ cycloalkyl, and R⁷ is C₃-C₇ cycloalkyl; each R^{b} is independently C₃-C₇ cycloalkyl, and R is halo; each R⁶ is independently cyclopropyl and R⁷ is halo; each R⁶ is independently C₁-C₆ alkyl, and R⁷ is C₁-C₆ alkoxy optionally substituted with one or more halo; each R⁶ is independently C₁-C₆ alkyl, and R⁷ is C₁-C₆ alkoxy; each R⁶ is independently C₁-C₆ alkyl, and R⁷ is C₁-C₆ alkoxy substituted with one or more halo; each R⁶ is independently halo, and R⁷ is C₁-C₆ haloalkyl; each R⁶ is independently halo, and R⁷ is C₁-C₆ haloalkoxy; each R⁶ is independently C₁-C₆ alkoxy; and R⁷ is halo; each R⁶ is independently C₁-C₆ alkoxy; and R⁷ is chloro; R⁷ is C₁-C₆ alkyl, and each R⁶ is independently C₁-C₆ alkyl optionally substituted with one or more halo; R⁷ is C₁-C₆ alkyl, and each R⁶ is independently C₁-C₆ alkyl substituted with one or more halo; R⁷ is C₁-C₆ alkyl, and each R⁶ is independently C₃-C₇ cycloalkyl; R⁷ is C₁-C₆ alkyl, and each R⁶ is independently halo; R⁷ is C₁-C₆ alkyl and each R⁶ is independently halo; R⁷ is C₁-C₆ alkyl, and R⁶ is cyano; R is C₃-C₇ cycloalkyl, and each R⁶ is independently C₃-C₇ cycloalkyl; R⁷ is C₃-C₇ cycloalkyl, and each R⁶ is independently halo, R⁷ is C₃-C₇ cycloalkyl and each R⁶ is independently halo; R⁷ is C₁-C₆ alkyl, and each R⁶ is independently C₁-C₆ alkoxy optionally substituted with one or more halo; R⁷ is C₁-C₆ alkyl, and each R⁶ is independently C₁-C₆ alkoxy; R⁷ is C₁-C₆ alkyl, and each R⁶ is independently C₁-C₆ alkoxy substituted with one or more halo; R⁷ is halo, and each R⁶ is independently C₁-C₆ haloalkyl; R⁷ is halo, and each R⁶ is independently C₁-C₆ haloalkoxy; R is C₁-C₆ alkoxy; and each R⁶ is independently halo; R is C₁-C₆ alkoxy; and R⁶ is chloro; R⁶ and R⁷ on adjacent atoms taken together with the atoms connecting them form a C₅ aliphatic carbocyclic ring.

In some embodiments, the compound of formula AA is a compound wherein the substituted ring A is

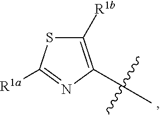

the optionally optionally substituted ring B is

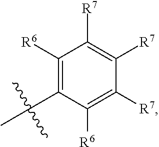

and wherein:
R^{1a} is hydroxymethyl, and R^{1b} is hydroxymethyl, R^{1a} is hydroxymethyl, and R¹⁰ is hydroxy ethyl; R^{1a} is hydroxymethyl, and R^{1b} is 2-hydroxy-2-propyl; R^{1a} is hydroxymethyl, and R^{1b} is 3-hydroxy-2-propyl; R^{1a} is hydroxymethyl, and R^{1b} is 1-hydroxy-1-propyl; R^{1a} is hydroxymethyl, and R^{1b} is 2-hydroxy-1-propyl; R^{1a} is hydroxymethyl, and R^{1b} is 3-hydroxy-1-propyl; R^{1a} is hydroxymethyl, and R^{1b} is hydroxybutyl; R^{1a} is hydroxymethyl, and R^{1b} is hydroxypentyl; R^{1a} is hydroxymethyl, and R^{1b} is hydroxyhexyl; R^{1a} is hydroxyethyl, and R^{1b} is hydroxymethyl; R^{1a} is hydroxyethyl, and R^{1b} is hydroxyethyl; R^{1a} is hydroxyethyl, and R^{1b} is 2-hydroxy-2-propyl; R^{1a} is hydroxyethyl, and R^{1b} is 3-hydroxy-2-propyl; R^{1a} is hydroxyethyl, and R^{1b} is 1-hydroxy-1-propyl; R^{1a} is hydroxyethyl, and R^{1b} is 2-hydroxy-1-propyl; R^{1a} is hydroxyethyl, and R^{1b} is 3-hydroxy-1-propyl; R^{1a} is hydroxyethyl, and R^{1b} is hydroxybutyl; R^{1a} is hydroxyethyl, and R^{1b} is hydroxypentyl; R^{1a} is hydroxyethyl, and R^{1a} is hydroxyhexyl; R^{1a} is 2-hydroxy-2-propyl, and R^{1b} is hydroxymethyl; R^{1a} is 2-hydroxy-2-propyl, and R^{1b} is hydroxyethyl; R^{1a} is 2-hydroxy-2-propyl, and R^{1b} is 2-hydroxy-2-propyl; R^{1a} is 2-hydroxy-2-propyl, and R^{1b} is 3-hydroxy-2-propyl; R^{1a} is 2-hydroxy-2-propyl, and R^{1b} is 1-hydroxy-1-propyl: R^{1a} is 2-hydroxy-2-propyl, and R^{1b} is 2-hydroxy-1-propyl; R^{1a} is 2-hydroxy-2-propyl, and R^{1b} is 3-hydroxy-1-propyl, R^{1a} is 2-hydroxy-2-propyl, and R^{1b} is hydroxybutyl; R^{1a} is 2-hydroxy-2-propyl, and R¹⁰ is hydroxypentyl; R^{1a} is 2-hydroxy-2-propyl, and R^{1b} is hydroxyhexyl; R^{1b} is hydroxymethyl, and R^{1a} is hydroxymethyl; R^{1b} is hydroxymethyl, and R^{1a} is hydroxyethyl; R^{1b} is hydroxymethyl, and R^{1a} is 2-hydroxy-2-propyl; R^{1b} is hydroxymethyl, and R^{1a} is 3-hydroxy-2-propyl; R^{1b} is hydroxymethyl, and R^{1a} is 1-hydroxy-1-propyl; R^{1b} is hydroxymethyl, and R^{1a} is 2-hydroxy-1-propyl; R^{1b} is hydroxymethyl, and R^{1a} is 3-hydroxy-1-propyl; R^{1b} is hydroxymethyl, and R^{1a} is hydroxybutyl; R^{1b} is hydroxymethyl, and R^{1a} is hydroxypentyl; R^{1b} is hydroxymethyl, and R^{1a} is hydroxyhexyl; R^{1b} is hydroxyethyl, and R^{1a} is hydroxymethyl; R^{1b} is hydroxyethyl, and R^{1a} is hydroxyethyl, R^{1b} is hydroxyethyl, and R^{1a} is 2-hydroxy-2-propyl; R^{1b} is hydroxyethyl, and R^{1a} is 3-hydroxy-2-propyl; R^{1b} is hydroxyethyl, and R^{1a} is 1-hydroxy-1-propyl; R^{1b} is hydroxyethyl, and R^{1a} is 2-hydroxy-J-propyl, R^{1b} is hydroxyethyl, and R^{1a} is 3-hydroxy-1-propyl; R^{1b} is hydroxyethyl, and R^{1a} is hydroxybutyl; R^{1b} is hydroxyethyl, and R^{1a} is hydroxypentyl; R^{1b} is hydroxyethyl, and R^{1a} is hydroxyhexyl; R^{1b} is 2-hydroxy-2-propyl, and R^{1a} is hydroxymethyl; R^{1b} is 2-hydroxy-2-propyl, and R^{1a} is hydroxyethyl; R^{1b} is 2-hydroxy-2-propyl, and R^{1a} is 2-hydroxy-2-propyl; R^{1b} is 2-hydroxy-2-propyl, and R^{1a} is 3-hydroxyl-propyl; R^{1b} is 2-hydroxy-2-propyl, and R^{1a} is 1-hydroxy-1-propyl; R^{1b} is 2-hydroxy-2-propyl, and R^{1a} is 2-hydroxy-1-propyl; R^{1b} is 2-hydroxy-2-propyl, and R^{1a} is 3-hydroxy-1-propyl; R^{1b} is 2-hydroxy-2-propyl, and R^{1a} is hydroxybutyl; R^{1b} is 2-hydroxy-2-propyl, and R^{1a} is hydroxypentyl; R^{1b} is 2-hydroxy-2-propyl, and R^{1a} is hydroxyhexyl;

and R⁶ and R⁷ are one of the following combinations:
each R⁶ is isopropyl; and R⁷ is methyl; each R⁶ is isopropyl; and R is isopropyl; each R⁶ is isopropyl; and R⁷ is trifluoromethyl; each R⁶ is isopropyl; and R⁷ is cyclopropyl; each R^{b} is isopropyl; and R⁷ is chloro; each R⁶ is isopropyl; and R⁷ is fluoro; each R^{b} is ethyl; and R is fluoro; each R⁶ is isopropyl; and R⁷ is cyano; each R⁶ is cyclopropyl; and R⁷ is cyclopropyl; each R⁶ is cyclopropyl; and R⁷ is chloro; each R⁶ is cyclopropyl; and If is fluoro; each R⁶ is isopropyl; and R⁷ is methoxy; each R⁶ is isopropyl; and R⁷ is trifluoromethoxy; each R⁶ is chloro; and R⁷ is trifluoromethyl; each R^{b} is chloro; and R⁷ is trifluoromethoxy; R⁷ is isopropyl; and each R⁶ is methyl; R⁷ is isopropyl; and each R⁶ is trifluoromethyl; IV is isopropyl; and each IV is cyclopropyl; R⁷ is isopropyl; and each R⁶ is chloro; R⁷ is ethyl; and each R⁶ is fluoro; R⁷ is isopropyl; and each R⁶ is cyano; R⁷ is cyclopropyl; and each R⁶ is cyclopropyl; R⁷ is cyclopropyl; and each R⁶ is chloro; R⁷ is cyclopropyl; and each R⁶ is fluoro; R⁷ is isopropyl; and each R⁶ is methoxy; R⁷ is isopropyl; and each R⁶ is trifluoromethoxy; R⁷ is chloro; and each R⁶ is trifluoromethyl, R⁷ is chloro, and each R⁶ is trifluoromethoxy; one R⁶ is isopropyl; the other R⁵ is trifluoromethyl; and R⁷ is chloro, R⁶ and R⁷ on adjacent atoms taken together with the atoms connecting them form a C₅ aliphatic carbocyclic ring; and one R⁶ is fluoro, chloro, or cyano.

### Additional Features of the Embodiments Herein

In some embodiments, the compound of Formula AA is not a compound selected from the group consisting of:

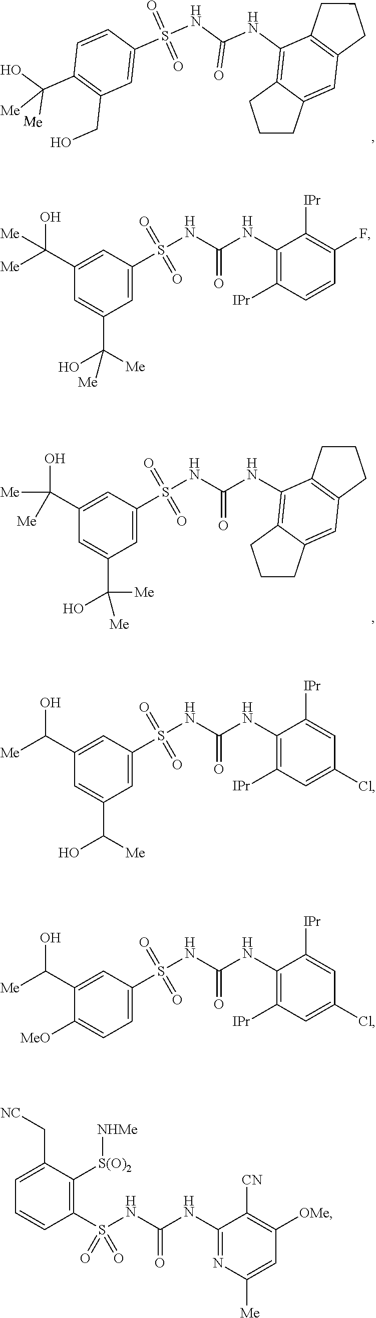

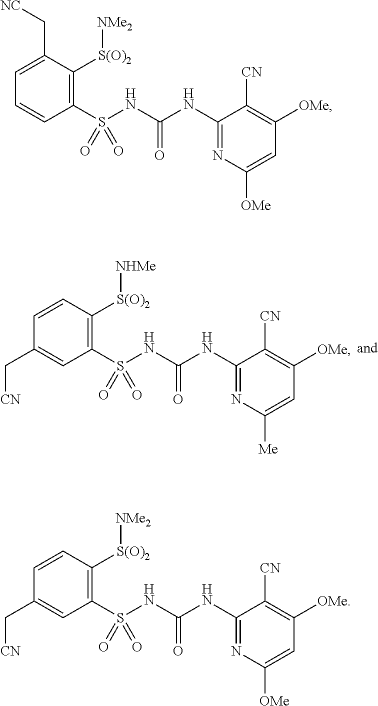

In some embodiments, the compound of Formula AA is not a compound selected from the group consisting of:

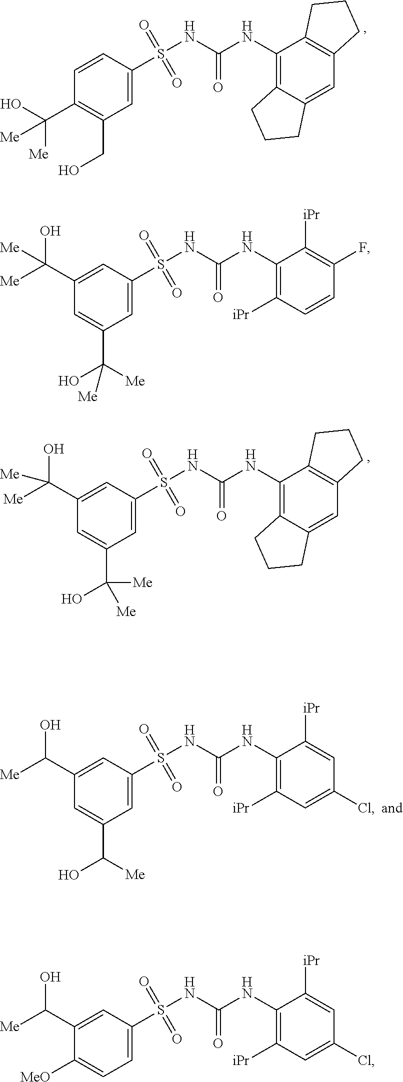 [SMILES: CC(C)(O)c1ccc(S(=O)(=O)NC(=O)Nc2c3c(cc4c2CCC4)CCC3)cc1CO ; CC(C)c1ccc(F)c(C(C)C)c1NC(=O)NS(=O)(=O)c1cc(C(C)(C)O)cc(C(C)(C)O)c1 ; CC(C)(O)c1cc(C(C)(C)O)cc(S(=O)(=O)NC(=O)Nc2c3c(cc4c2CCC4)CCC3)c1 ; CC(C)c1cc(Cl)cc(C(C)C)c1NC(=O)NS(=O)(=O)c1cc(C(C)O)cc(C(C)O)c1 ; "and" ; COc1ccc(S(=O)(=O)NC(=O)Nc2c(C(C)C)cc(Cl)cc2C(C)C)cc1C(C)O]

In some embodiments, the compound of Formula AA is not a compound selected from the group consisting of:

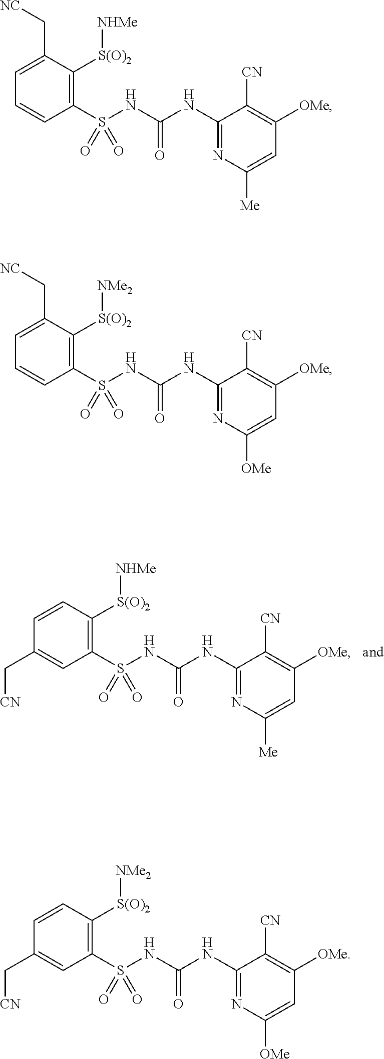

In some embodiments, if one or more R⁶ is CN and/or if one or more R⁷ is CN, then the one or more CN is not ortho to the bond connecting the B ring to the NH(CO) group of Formula AA.

In some embodiments of any of the formulae herein, R^{1b} is not —CO₂R¹³.

In some embodiments the compound of any of the formulae herein is not a compound disclosed in any of Examples 1-150 of patent publication WO2001/019390.

In some embodiments the compound of any of the formulae herein is not a compound disclosed in any of Examples 1-130 of patent publication WO 98/32733.

In some embodiments the compound of any of the formulae herein is not a compound disclosed in any of the Examples at [00123] of patent publication WO2016/131098,

In some embodiments the compound of any of the formulae herein is not a compound disclosed in DK 2006/00313,

In some embodiments the compound of any of the formulae herein is not a compound disclosed in U.S. Pat. No. 4,927,453.

In some embodiments the compound of any of the formulae herein is not a compound disclosed in EP 03/18620.

In some embodiments the compound of any of the formulae herein is not a compound disclosed in EP 02/05348.

In some embodiments the compound of any of the formulae herein is not a compound disclosed in WO2019034686, WO2019034688, WO2019034690, WO2019034692, WO2019034693, WO2019034696, and/or WO2019034697.

Unless otherwise indicated, when a disclosed compound is named or depicted by a structure without specifying the stereochemistry and has one or more chiral centers, it is understood to represent all possible stereoisomers of the compound.

It is understood that the combination of variables in the formulae herein is such that the compounds are stable.

In some embodiments, provided herein is a compound that is selected from the group consisting of the compounds in Table 1A:

TABLE 1A

| Compound | Structure | m/z |
|---|---|---|
| 101 | 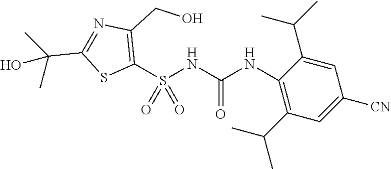 [SMILES: CC(C)c1cc(C#N)cc(C(C)C)c1NC(=O)NS(=O)(=O)c1sc(C(C)(C)O)nc1CO] | 481.2 |
| 102 | 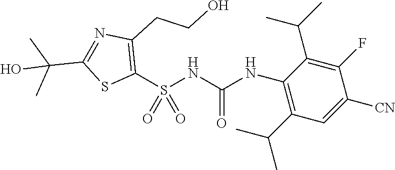 [SMILES: CC(C)c1cc(C#N)c(F)c(C(C)C)c1NC(=O)NS(=O)(=O)c1sc(C(C)(C)O)nc1CCO] | 512.2 |
| 103 | 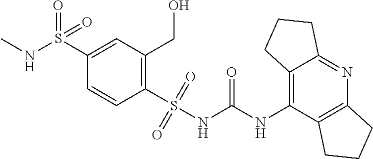 [SMILES: CNS(=O)(=O)c1ccc(S(=O)(=O)NC(=O)Nc2c3c(nc4c2CCC4)CCC3)c(CO)c1] | 480 |
| 104 | 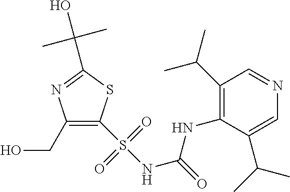 [SMILES: CC(C)c1cncc(C(C)C)c1NC(=O)NS(=O)(=O)c1sc(C(C)(C)O)nc1CO] | 456.2 |
| 105 | 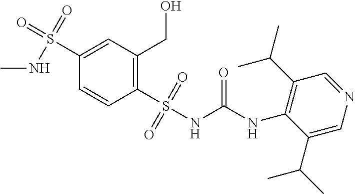 [SMILES: CNS(=O)(=O)c1ccc(S(=O)(=O)NC(=O)Nc2c(C(C)C)cncc2C(C)C)c(CO)c1] | 484.1 |
| 106 | 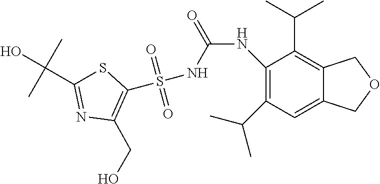 [SMILES: CC(C)c1cc2c(c(C(C)C)c1NC(=O)NS(=O)(=O)c1sc(C(C)(C)O)nc1CO)COC2] | 497.2 |
| 107 | 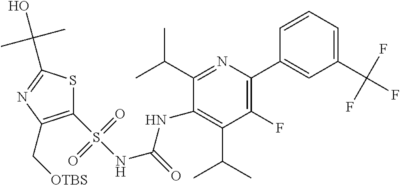 [SMILES: CC(C)c1nc(-c2cccc(C(F)(F)F)c2)c(F)c(C(C)C)c1NC(=O)NS(=O)(=O)c1sc(C(C)(C)O)nc1CO[Si](C)(C)C(C)(C)C] | 732.0 |
| 108 | 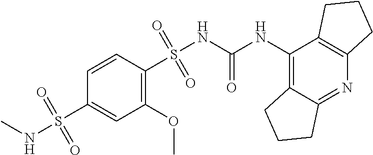 [SMILES: CNS(=O)(=O)c1ccc(S(=O)(=O)NC(=O)Nc2c3c(nc4c2CCC4)CCC3)c(OC)c1] | 480.1 |
| 109 | 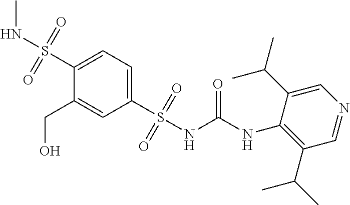 [SMILES: CNS(=O)(=O)c1ccc(S(=O)(=O)NC(=O)Nc2c(C(C)C)cncc2C(C)C)cc1CO] | 485.2 |
| 110 | 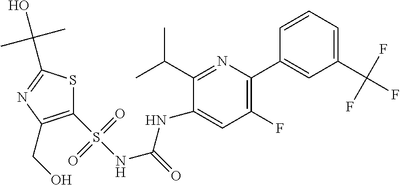 [SMILES: CC(C)c1nc(-c2cccc(C(F)(F)F)c2)c(F)cc1NC(=O)NS(=O)(=O)c1sc(C(C)(C)O)nc1CO] | 618 |
| 111 | 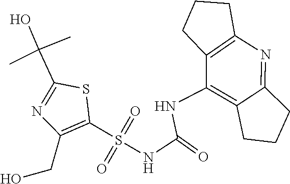 [SMILES: CC(C)(O)c1nc(CO)c(S(=O)(=O)NC(=O)Nc2c3c(nc4c2CCC4)CCC3)s1] | 452.12 |
| 112 | 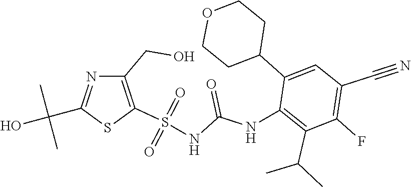 [SMILES: CC(C)c1c(F)c(C#N)cc(C2CCOCC2)c1NC(=O)NS(=O)(=O)c1sc(C(C)(C)O)nc1CO] | 541.1 |
| 113 | 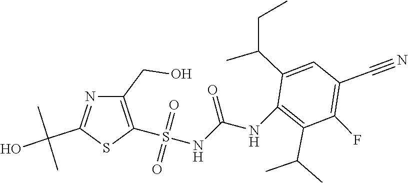 [SMILES: CCC(C)c1cc(C#N)c(F)c(C(C)C)c1NC(=O)NS(=O)(=O)c1sc(C(C)(C)O)nc1CO] | 513.2 |
| 114 | 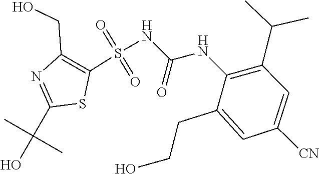 [SMILES: CC(C)c1cc(C#N)cc(CCO)c1NC(=O)NS(=O)(=O)c1sc(C(C)(C)O)nc1CO] | 483.1 |
| 115 | 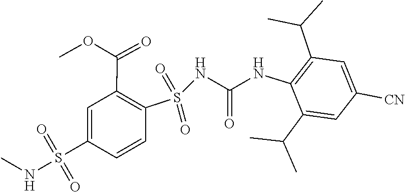 [SMILES: CNS(=O)(=O)c1ccc(S(=O)(=O)NC(=O)Nc2c(C(C)C)cc(C#N)cc2C(C)C)c(C(=O)OC)c1] | 537 |
| 116 | 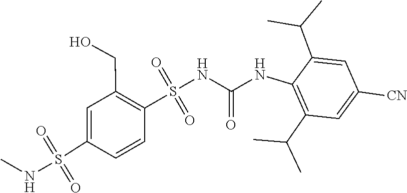 [SMILES: CNS(=O)(=O)c1ccc(S(=O)(=O)NC(=O)Nc2c(C(C)C)cc(C#N)cc2C(C)C)c(CO)c1] | 508.1 |
| 117 | 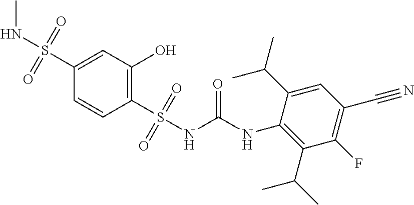 [SMILES: CNS(=O)(=O)c1ccc(S(=O)(=O)NC(=O)Nc2c(C(C)C)cc(C#N)c(F)c2C(C)C)c(O)c1] | 511.1 |
| 118 | 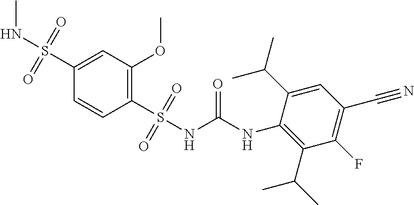 [SMILES: CNS(=O)(=O)c1ccc(S(=O)(=O)NC(=O)Nc2c(C(C)C)cc(C#N)c(F)c2C(C)C)c(OC)c1] | 527.1 |
| 119 | 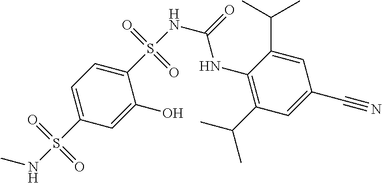 [SMILES: CNS(=O)(=O)c1ccc(S(=O)(=O)NC(=O)Nc2c(C(C)C)cc(C#N)cc2C(C)C)c(O)c1] | 493.05 |
| 120 | 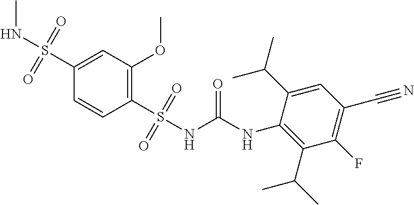 [SMILES: CNS(=O)(=O)c1ccc(S(=O)(=O)NC(=O)Nc2c(C(C)C)cc(C#N)c(F)c2C(C)C)c(OC)c1] | 525.1 |
| 121 | 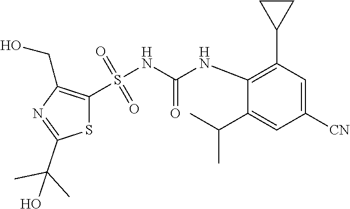 [SMILES: CC(C)c1cc(C#N)cc(C2CC2)c1NC(=O)NS(=O)(=O)c1sc(C(C)(C)O)nc1CO] | 479.1 |
| 122 | 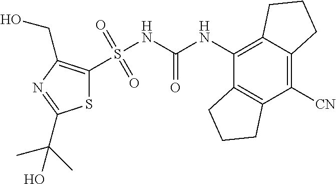 [SMILES: CC(C)(O)c1nc(CO)c(S(=O)(=O)NC(=O)Nc2c3c(c(C#N)c4c2CCC4)CCC3)s1] | 477.1 |
| 123 | 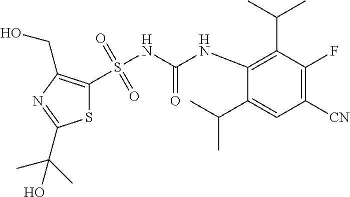 [SMILES: CC(C)c1cc(C#N)c(F)c(C(C)C)c1NC(=O)NS(=O)(=O)c1sc(C(C)(C)O)nc1CO] | 499.2 |
| 124 | 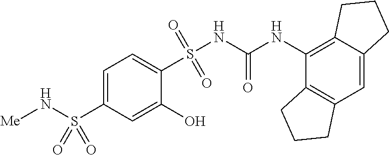 [SMILES: CNS(=O)(=O)c1ccc(S(=O)(=O)NC(=O)Nc2c3c(cc4c2CCC4)CCC3)c(O)c1] | 465.1 |

and pharmaceutically acceptable salts thereof.

In some embodiments, provided herein is a compound that is selected from the group consisting of the compounds in Table 1B:

TABLE 1B

| Compound | Structure |
|---|---|
| 201 | 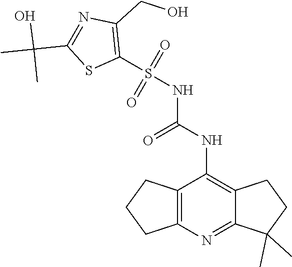 [SMILES: CC(C)(O)c1nc(CO)c(S(=O)(=O)NC(=O)Nc2c3c(nc4c2CCC4(C)C)CCC3)s1] |
| 202 | 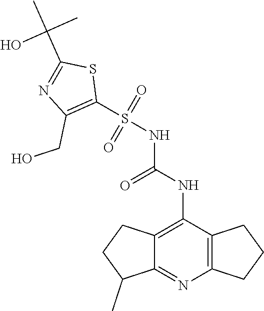 [SMILES: CC1CCc2c1nc1c(c2NC(=O)NS(=O)(=O)c2sc(C(C)(C)O)nc2CO)CCC1] |
| 203 | 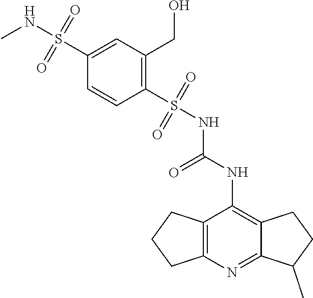 [SMILES: CNS(=O)(=O)c1ccc(S(=O)(=O)NC(=O)Nc2c3c(nc4c2CCC4C)CCC3)c(CO)c1] |
| 204 | 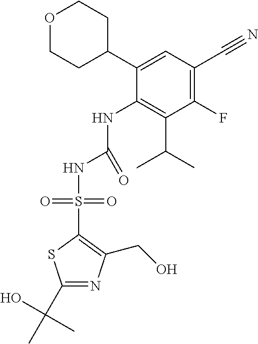 [SMILES: CC(C)c1c(F)c(C#N)cc(C2CCOCC2)c1NC(=O)NS(=O)(=O)c1sc(C(C)(C)O)nc1CO] |

and pharmaceutically acceptable salts thereof.

In some embodiments, provided herein is a compound that is selected from the group consisting of the compounds in Table 1C:

TABLE 1C

| Compound | Structure |
|---|---|
| 301 | 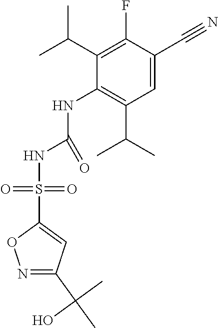 [SMILES: CC(C)c1cc(C#N)c(F)c(C(C)C)c1NC(=O)NS(=O)(=O)c1cc(C(C)(C)O)no1] |
| 302 | 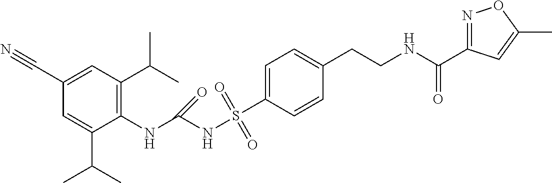 [SMILES: Cc1cc(C(=O)NCCc2ccc(S(=O)(=O)NC(=O)Nc3c(C(C)C)cc(C#N)cc3C(C)C)cc2)no1] |
| 303 | 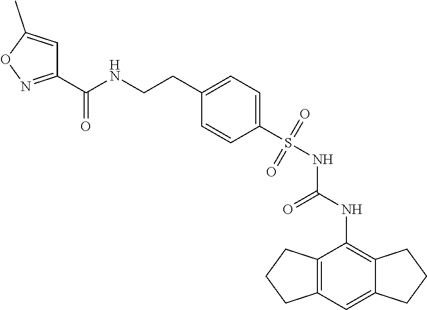 [SMILES: Cc1cc(C(=O)NCCc2ccc(S(=O)(=O)NC(=O)Nc3c4c(cc5c3CCC5)CCC4)cc2)no1] |
| 304 | 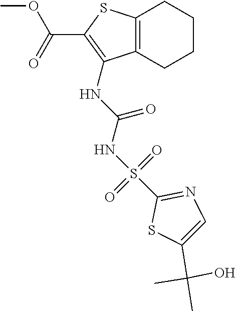 [SMILES: COC(=O)c1sc2c(c1NC(=O)NS(=O)(=O)c1ncc(C(C)(C)O)s1)CCCC2] |
| 305 | 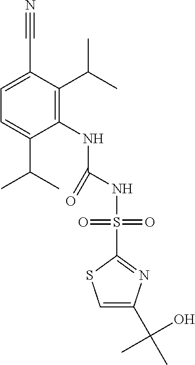 [SMILES: CC(C)c1ccc(C#N)c(C(C)C)c1NC(=O)NS(=O)(=O)c1nc(C(C)(C)O)cs1] |
| 306 | 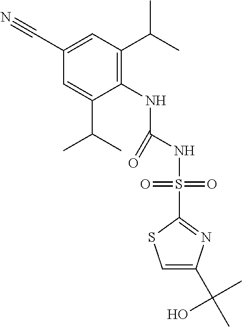 [SMILES: CC(C)c1cc(C#N)cc(C(C)C)c1NC(=O)NS(=O)(=O)c1nc(C(C)(C)O)cs1] |
| 307 | 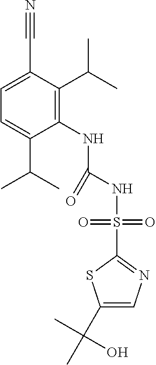 [SMILES: CC(C)c1ccc(C#N)c(C(C)C)c1NC(=O)NS(=O)(=O)c1ncc(C(C)(C)O)s1] |
| 308 | 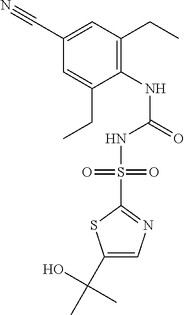 [SMILES: CCc1cc(C#N)cc(CC)c1NC(=O)NS(=O)(=O)c1ncc(C(C)(C)O)s1] |
| 309 | 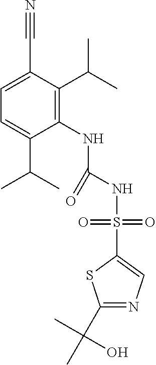 [SMILES: CC(C)c1ccc(C#N)c(C(C)C)c1NC(=O)NS(=O)(=O)c1cnc(C(C)(C)O)s1] |
| 310 | 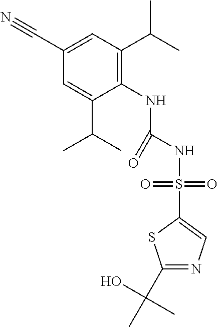 [SMILES: CC(C)c1cc(C#N)cc(C(C)C)c1NC(=O)NS(=O)(=O)c1cnc(C(C)(C)O)s1] |
| 311 | 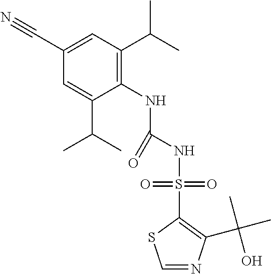 [SMILES: CC(C)c1cc(C#N)cc(C(C)C)c1NC(=O)NS(=O)(=O)c1scnc1C(C)(C)O] |
| 312 | 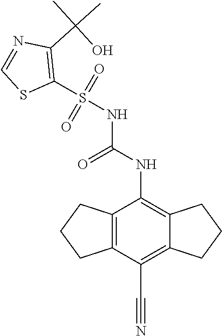 [SMILES: CC(C)(O)c1ncsc1S(=O)(=O)NC(=O)Nc1c2c(c(C#N)c3c1CCC3)CCC2] |
| 313 | 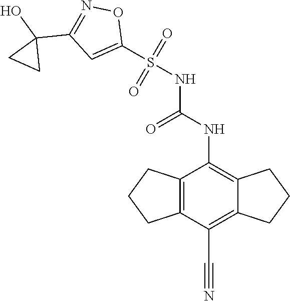 [SMILES: N#Cc1c2c(c(NC(=O)NS(=O)(=O)c3cc(C4(O)CC4)no3)c3c1CCC3)CCC2] |
| 314 | 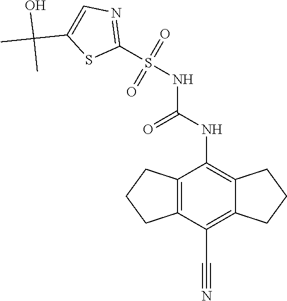 [SMILES: CC(C)(O)c1cnc(S(=O)(=O)NC(=O)Nc2c3c(c(C#N)c4c2CCC4)CCC3)s1] |
| 315 | 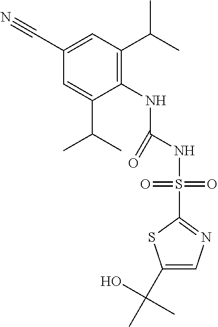 [SMILES: CC(C)c1cc(C#N)cc(C(C)C)c1NC(=O)NS(=O)(=O)c1ncc(C(C)(C)O)s1] |
| 316 | 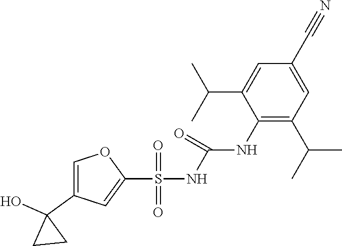 [SMILES: CC(C)c1cc(C#N)cc(C(C)C)c1NC(=O)NS(=O)(=O)c1cc(C2(O)CC2)co1] |
| 317 | 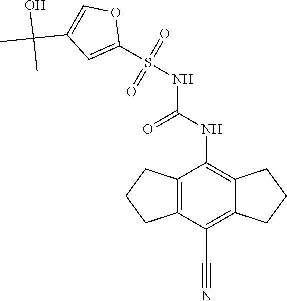 [SMILES: CC(C)(O)c1coc(S(=O)(=O)NC(=O)Nc2c3c(c(C#N)c4c2CCC4)CCC3)c1] |
| 318 | 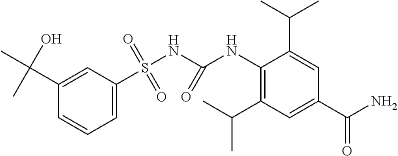 [SMILES: CC(C)c1cc(C(N)=O)cc(C(C)C)c1NC(=O)NS(=O)(=O)c1cccc(C(C)(C)O)c1] |
| 319 | 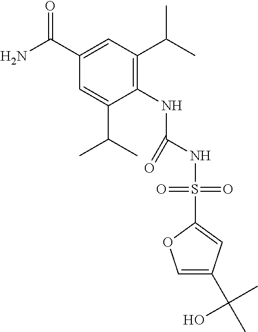 [SMILES: CC(C)c1cc(C(N)=O)cc(C(C)C)c1NC(=O)NS(=O)(=O)c1cc(C(C)(C)O)co1] |
| 320 | 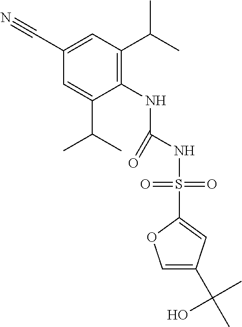 [SMILES: CC(C)c1cc(C#N)cc(C(C)C)c1NC(=O)NS(=O)(=O)c1cc(C(C)(C)O)co1] |
| 321 | 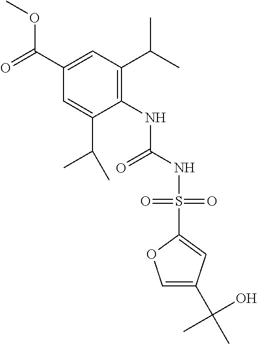 [SMILES: COC(=O)c1cc(C(C)C)c(NC(=O)NS(=O)(=O)c2cc(C(C)(C)O)co2)c(C(C)C)c1] |
| 322 | 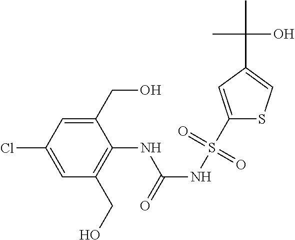 [SMILES: CC(C)(O)c1csc(S(=O)(=O)NC(=O)Nc2c(CO)cc(Cl)cc2CO)c1] |
| 323 | 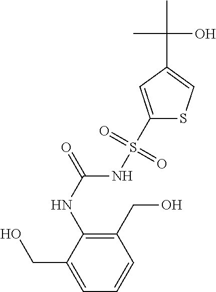 [SMILES: CC(C)(O)c1csc(S(=O)(=O)NC(=O)Nc2c(CO)cccc2CO)c1] |
| 324 | 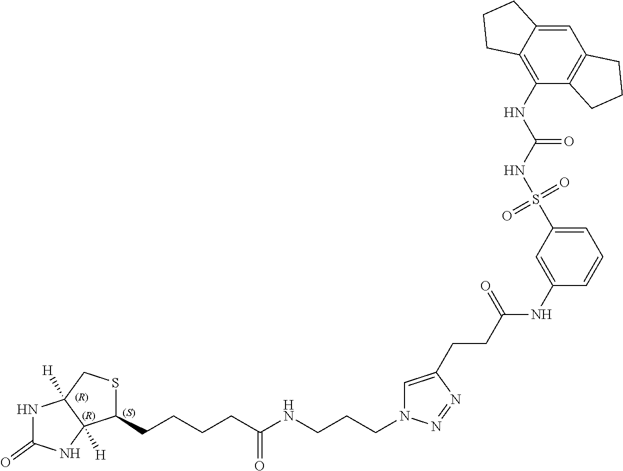 |

and pharmaceutically acceptable salts thereof.

### Pharmaceutical Compositions and Administration

### General

In some embodiments, a chemical entity (e.g., a compound that modulates (e.g., antagonizes) NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, γ, and γ-cyclodextrin, or chemically modified derivatives such as hydroxyalkylcyclodextrins, including 2- and 3-hydroxypropyl-p-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art, for example, see Remington; The Science and Practice of Pharmacy, 22^{nd} Edition (Pharmaceutical Press, London, U K. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intraeoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared, and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SEN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as tillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG'S, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drags from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marc-oat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories; Inc.), Purite (stabilized oxychloro complex; Allergan; Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (I) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g, natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently seleleted from thickeners, viscosity enhancing agents, bulking agents, mucoadhesive agents, penetration enhanceers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently seleleted from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently seleleted from thickeners, viscosity enhancing agents, bulking agents, mucoadhesive agents, buffers, preservatives, and fillers.

In certain embodiments, each of the one or more pharmaceutically acceptable excipients can be independently seleleted from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxy ethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxy ethyl cellulose, hydroxypropyl cellulose, hydroxy ethyl ethyl cellulose, carboxymethyl cellulose, hydroxypropylmethyl cellulose or cellulose esters or ethers or derivatives or salts thereof: clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g, lactate or glutamate or carboxymethyl chitin, glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as poly acrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methyl ethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric-nitrate, phenylmercuric acetate, phenylmercuric borate, methylparaben, propylparaben, chlorobutanol, benzyl alcohol, phenyl ethyl alcohol, chiorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, days, cellulose, alginine, gums or cross linked polymers, such as cross-linked PVT (Polyplasdone XL from GAF Chemical Corp). In certain embodiments, the disintegrate is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica, e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dxtrose, lactose and sorbitol), polyethylene glycol, waxes, natural and synthetic gums such as acacia, tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone):
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phospahate dehydrate, disodium phosphate dodecahydrate);
One or more (e.g., one or two, e.g., two) glidants and/or lubricants, such as magnesium stearate and/or talc,
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula AA, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the chemical entity is a compound of Formula AA, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (it) is provided in its own separate kit or pack.

In certain of these embodiments, component (1) includes the chemical entity (e.g., a compound of Formula AA, or a pharmaceutically acceptable salt and/or hydrate and/or cocrystal thereof; e.g., a compound of Formula AA) and one or more (e.g., all) of the following excipients:
(a) One or more (e.g., one) binders (e.g., a polyvinyl polymer, such as polyvinylpyrrolidone (povidone),
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula AA, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent, e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound of | 40 weight percent to about 80 |
| Formula AA | weight percent (e.g., from about |
| | 50 weight percent to about 70 |
| | weight percent, from about 55 |
| | weight percent to about 70 weight |
| | percent; from about 60 |
| | weight percent to about 65 weight |
| | percent; e.g., about 62.1 |
| | weight percent) |
| Crospovidone | 0.5 weight percent to about 5 weight |
| (Kollidon CL) | percent (e.g., from about |
| | 0.5 weight percent to about 3 weight |
| | percent, from about 1 |
| | weight percent to about 3 weight |
| | percent; about 1.93 weight |
| | percent |
| lactose monohydrate | about 10 weight percent to about 50 |
| (Pharmatose 200M) | weight percent e.g., from |
| | about 20 weight percent to about 40 |
| | weight percent, from |
| | about 25 weight percent to about 35 |
| | weight percent; e.g., |
| | about 31.03 weight percent |
| Povidone (Kollidon | about 0.5 weight percent to about 5 |
| K30) | weight percent (e.g., from |
| | about 1.5 weight percent to about |
| | 4.5 weight percent, from |
| | about 2 weight percent to about 3.5 |
| | weight percent; e.g.; about |
| | 2.76 weight percent |
| talc | 0.5 weight percent to about 5 weight |
| | percent (e.g., from about |
| | 0.5 weight percent to about 3 |
| | weight percent, from about 1 |
| | weight percent to about 3 weight |
| | percent; from about 1.5 |
| | weight percent to about 2.5 weight |
| | percent; from about 1.8 |
| | weight percent to about 2.2 weight |
| | percent; e.g., about 1.93 |
| | weight percent |
| Magnesium stearate | about 0.05 weight percent to about |
| | 1 weight percent (e.g., |
| | from about 0.05 weight percent |
| | to about 1 weight percent; |
| | from about 0.1 weight percent |
| | to about 1 weight percent; from |
| | about 0.1 weight percent to |
| | about 0.5 weight percent; e.g., |
| | about 0.27 weight percent |

In certain embodiments, component (I) includes the ingredients and amounts as shown in Table B.

TABLE B

| Ingredient | Weight Percent |
|---|---|
| A compound of Formula AA | About 62.1 weight percent) |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not washing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging,

In certain of the foregoing embodiments, component (it) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone),
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phospahate dihydrate, disodium phosphate dodecahydrate);

In certain of the foregoing embodiments, component (it) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phospahate dehydrate).

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent, e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″) -(c′″) is present.

In certain embodiments, component (ii) includes water (up to 100%) and the ingredients and amounts as shown in Table C.

TABLE C

| Ingredient | Weight Percent |
|---|---|
| methyl cellulose | 0.05 weight percent to about 5 weight |
| (Methocel A15C | percent (e.g., from about |
| premium) | 0.05 weight percent to about 3 weight |
| | percent, from about 0.1 |
| | weight percent to about 3 weight |
| | percent; e.g., about 1.4 weight |
| | percent |
| Povidone | 0.05 weight percent to about 5 |
| (Kollidon K30) | weight percent (e.g., from about |
| | 0.05 weight percent to about 3 |
| | weight percent, from about 0.1 |
| | weight percent to about 2 weight |
| | percent; e.g., about 1.0 weight |
| | percent |
| propyl 4- | about 0.005 weight percent to about |
| hydroxybenzoate | 0.1 weight percent e.g., from |
| | about 0.005 weight percent to about |
| | 0.05 weight percent, e.g., |
| | about 0.02 weight percent) |
| methyl 4- | about 0.05 weight percent to about 1 |
| hydroxybenzoate | weight percent (e.g., from |
| | about 0.05 weight percent to about |
| | 0.5 weight percent; e.g., about |
| | 0.20 weight percent) |
| disodium | about 0.05 weight percent to about |
| phosphate | 1 weight percent (e.g., from |
| dodecahydrate | about 0.05 weight percent to about |
| | 0.5 weight percent; e.g., about |
| | 0.15 weight percent) |
| sodium dihydrogen | about 0.005 weight percent to about |
| phospahate | 0.5 weight percent (e.g., from |
| dihydrate | about 0.005 weight percent to about |
| | 0.3 weight percent; e.g., about |
| | 0.15 weight percent) |

In certain embodiments, component (ii) includes water (up to 100%) and the ingredients and amounts as shown in Table D,

TABLE D

| Ingredient | Weight Percent |
|---|---|
| methyl cellulose (Methocel A15C | about 1.4 weight percent |
| premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in a device, for example, a plastic bottle, a breakable capsule, and a rectal cannula and single flow pack.

Dosages

The dosages may be varied depending on the requirement, of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg, from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg, from about 0.01 mg/Kg to about 1 mg/Kg; from about 0.01 mg/Kg to about 0.5 mg/Kg; from about 0.01 mg/Kg to about 0.1 mg/Kg; from about 0, 1 mg/Kg to about 200 mg/Kg; from about 0, 1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about. 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg, from about 5 mg to about 750 mg; from about 5 mg to about. 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg. from about 50 mg to about 750 mg, from about 50 mg to about 600 mg, from about 50 mg to about 500 mg, from about 50 mg to about 400 mg, from about. 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 ml to about 500 mL, from about 1 mL to about 250 mL, from about 1 mL to about 100 mL, from about 10 mL to about 1000 mL, from about 10 ml. to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid earner.

In certain embodiments, enema formulations include from about. 50 mg to about 250 mg (e.g., from about 100 mg to about 200; e.g., about 150 mg) of the chemical entity in from about 10 ml to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 150 mg of a compound of Formula AA in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 450 mg of a compound of Formula AA in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is a compound of Formula AA, or a pharmaceutically acceptable salt and/or hydrate and/or cocrystal thereof. For example, enema formulations can include about 2.5 mg/mL or about 7.5 mg/mL of a compound of Formula AA in liquid carrier.

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease, auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs, host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis). Without being bound by theory, it is believed that compounds of the present invention that are gut restricted or have lower absorption into systemic circulation would be beneficial where the condition, disease, or disorder is a bowel or intestinal disorder such as inflammatory bowel diseases (IBDs) comprising Crohn disease (CD) and ulcerative colitis (UC), autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from major adverse cardiovascular events such as cardiovascular death, non-fatal myocardial infarction and non-fatal stroke in patients with a prior hear attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity.

In some embodiments, the condition, disease or disorder is Type 2 Diabetes.

In some embodiments, the condition, disease or disorder is NASH.

In some embodiments, the condition, disease or disorder is Alzheimer's disease.

In some embodiments, the condition, disease or disorder is gout.

In some embodiments, the condition, disease or disorder is SLE.

In some embodiments, the condition, disease or disorder is rheumatoid arthritis.

In some embodiments, the condition, disease or disorder is IBD.

In some embodiments, the condition, disease or disorder is multiple sclerosis.

In some embodiments, the condition, disease or disorder is COPD.

In some embodiments, the condition, disease or disorder is asthma.

In some embodiments, the condition, disease or disorder is scleroderma.

In some embodiments, the condition, disease or disorder is pulmonary fibrosis.

In some embodiments, the condition, disease or disorder is age related macular degeneration (AMD).

In some embodiments, the condition, disease or disorder is cystic fibrosis.

In some embodiments, the condition, disease or disorder is Muckle Wells syndrome.

In some embodiments, the condition, disease or disorder is familial cold autoinflammatory syndrome (FCAS).

In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's ceil histiocytosis (LCH); multiple myeloma, promyeiocytic leukemia, acute myeloid leukemia (AML) chronic myeloid leukemia (CML); gastric cancer; and lung cancer metastasis.

In some embodiments, the condition, disease or disorder is selected from: myelodysplastic syndromes (AIDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyeiocytic leukemia; gastric cancer; and lung cancer metastasis.

In some embodiments, the indication is AIDS.

In some embodiments, the indication is non-small lung cancer in patients carrying mutation or overexpression of NLRP3.

In some embodiments, the indication is ALL in patients resistant to glucocorticoids treatment.

In some embodiments, the indication is LCH.

In some embodiments, the indication is multiple myeloma.

In some embodiments, the indication is promyelocytic leukemia.

In some embodiments, the indication is gastric cancer.

In some embodiments, the indication is lung cancer metastasis.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20.

In some embodiments, the methods described herein further include the step of identifying a subject (e.g., a patient) in need of treatment for an indication related to NLRP3 activity, such as an indication related to point mutation of NLRP3 signaling.

### Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibitreduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (INK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a ceil), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et ah, Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB,

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), INK, lipopolysaceharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that, results in TNFα mRNA transcription, TNFα mRNA stabilization, and TNFα mRNA translation (e.g., a component selected from the group of CD14, c-Jun, ERK1/2, IKK, IKB, IRAK, INK, LBP, MEK1/2, MEK3/6, MEK4/7, MK2, My 1)88, NF-κB, NIK, IRAK, lipopolysaceharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, INK, lipopolysaceharide binding protein (LBP), MEK1/2, MEK3/6, MEK4/7, MK2, MyD88, NF-κB, NIK, IRAK, lipopolysaceharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα; In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH3, a Diabody-CH3, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kλ-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)-IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-FIAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2. Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et ah, Autoimmunity Rev. 13(1):24-30, 2014; Bongartz et ah, JAMA 295(19):2275-2285, 2006; Butler et al., Eur. Cytokine Network 6(41:225-230, 1994; Cohen et al., Canadian J. Gastroenterol Hepatol 15(6):376-384, 2001; Elliott et al., Lancet 1994; 344: 1125-1127, 1994; Feldmann et al, Ann. Rev Immunol 19(1): 163-196, 2001, Rankin et al, Br. J. Rheumatol 2:334-342, 1995; Knight et al, Molecular Immunol 30(16): 1443-1453, 1993; Lorenz et al., J. Immunol. 156(4): 1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12): 1947-1955, 2013; Palladino et al., Nature Rev. Drug Discovery 2(9):736-746, 2003; Sandbom et al., Inflammatory Bowel Diseases 5(2): 119-133, 1999; Atzeni et al., Autoimmunity Reviews 12(7):703-708, 2013; Maini et al., Immunol Rev. 144(1): 195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (Golimumab™), adalimumab (Humira™), infliximab (Rernicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GSQ71 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadiia, BMO-2 or MYL-1401-A from Biocon/Mylan, CHS-1420 from Coherus, FKB327 from Kyowa Kirin, and BI 695501 from Boehringer Ingelheim;Solymbic®, SB5 from Samsung Bioepis, GP-2017 from Sandoz, ONS-3010 from Oncobiologics, M923 from Momenta, PF-06410293 from Pfizer, and etanercept biosimilars such as Ereizi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵M (e.g., less than 0.5×10⁻⁵ M, less than 1×10⁻⁶ M, less than 0.5×10⁻⁶ M, less than 1×10⁻⁷M, less than 0.5×10⁻⁷ M, less than 1×10⁻⁸ M, less than 0.5×10⁻⁸ M, less than 1×10⁻⁹M, less than 0.5×10⁻⁻⁹ M, less than 1×10⁻¹⁰ M, less than 0.5×10⁻¹⁰ M, less than 1×10⁻¹¹ M, less than 0.5×10⁻¹¹M, or less than 1×10⁻¹²M), e.g., as measured in phosphate buffered saline using surface plasmon resonance (SPR).

In some embodiments, any of the antibodies or antigen-binding fragments described herein has a K_{D} of about 1×10⁻¹² M to about 1×10⁻⁵M, about 0.5×10⁻⁵M, about 1×10⁻⁶ M, about 0.5×10⁻⁶M, about 1×10⁻⁷M, about 0.5×10⁻⁷M, about 1×10⁻⁸ M, about 0.5×10⁻⁸ M, about 1×10⁻⁹M, about 0.5×10⁻⁹M, about 1×10⁻¹° M, about 0.5×10⁻¹° M, about 1×10⁻¹¹ M, or about 0.5×10⁻¹¹M (inclusive); about 0.5×10⁻¹¹ M to about 1×10⁻⁵M, about 0.5×10⁻⁵M, about 1×10⁻⁶M, about 0.5×10⁻⁶M, about 1×10⁻¹¹ M, about 0.5×10⁻¹¹ M, about 1×10⁻⁸ M, about 0.5×10⁻⁸M, about 1×10⁻⁹M, about 0.5×10⁻⁹M, about 1×10⁻¹⁰ M, about 0.5×10⁻¹⁰ M, or about 1×10⁻¹¹M (inclusive); about 1×10⁻¹¹ M to about 1×10⁻⁵M, about 0.5×10⁻⁵ M, about 1×10⁻⁶ M, about 0.5×10⁻⁷ M, about 1×10⁻⁷M, about 0.5×10⁻⁷M, about 1×10⁻⁸M, about 0.5×10⁻⁸ M, about 1×10⁻⁹M, about 0.5×10⁻⁹ M, about 1×10⁻¹⁰M, or about 0.5×10⁻¹⁰ M (inclusive); about 0.5×10⁻¹° M to about 1×10⁻⁵M, about 0.5×10⁻⁵ M, about 1×10⁻⁶ M, about 0.5×10⁻⁷ M, about 1×10⁻⁷M, about 0.5×10⁻⁷M, about 1×10⁻⁸ M, about 0.5×10⁻⁸M, about 1×10⁻⁹M, about 0.5×10⁻⁹M, or about 1×10⁻¹⁰ M (inclusive); about 1×10⁻¹⁰ M to about 1×10⁻⁵M, about 0.5×10⁻⁵ M, about 1×10⁻⁶ M, about 0.5×10⁻⁶ M, about 1×10⁻⁷M, about 0.5×10⁻⁷M, about 1×10⁻⁸ M, about 0.5×10⁻⁸ M, about 1×10⁻⁹M, or about 0.5×10⁻⁹ NI (inclusive); about 0.5×10⁻⁹M to about 1×10⁻⁵M, about 0.5×10⁻⁵ M, about 1×10⁻⁶ M, about 0.5×10⁻⁶ M, about 1×10⁻⁷N about 0.5×10⁻⁷M, about 1×10⁻⁸M, about 0.5×10⁻⁸M, or about 1×10⁻⁹M (inclusive); about 1×10⁻⁹M to about 1×10⁻⁵M, about 0.5×10⁻⁵ M, about 1×10⁻⁶ M, about 0.5×10⁻⁶ M, about 1×10⁻⁷M, about 0.5×10⁻⁷M, about 1×10⁻⁸M, or about 0.5×10⁻⁸M (inclusive); about 0.5×10⁻⁸ M to about 1×10⁻⁵M, about 0.5×10⁻⁵M, about 1×10 M, about 0.5×10⁻⁶ M, about 1×10⁻⁷ M, about 0.5×10⁻⁷M, or about 1×10⁻⁸ M (inclusive); about 1×10⁻⁸ M to about 1×10⁻⁵M, about 0.5×10⁻⁵ M, about 1×10⁻⁶ M, about 0.5×10⁻⁶ M, about 1×10⁻⁸ M, or about 0.5×10⁻⁵ M (inclusive); about 0.5×10⁻⁷ M to about 1×10⁻⁵M, about 0.5×10⁻⁵M, about 1×10⁻⁶ M, about 0.5×10⁻⁶ M, or about 1×10⁻⁷ M (inclusive); about 1×10⁻⁷M to about 1×10⁻⁵M, about 0.5×10⁻⁵ M, about 1×10⁻⁶M, or about 0.5×10⁻⁶ M (inclusive); about 0.5×10⁻⁶ M to about 1×10⁻⁵ M, about 0.5×10⁻⁵ M, or about 1×10⁻⁷ M (inclusive); about 1×10⁻⁶ M to about 1×10⁻⁶ M or about 0.5×10⁻⁵ M (inclusive); or about 0.5×10⁻⁵M to about 1×10⁻⁵M (inclusive), e.g., as measured in phosphate buffered saline using surface plasmon resonance (SPR).

In some embodiments, any of the antibodies or antigen-binding fragments described herein has a K_{off} of about 1×10⁻⁶ s⁻¹ to about 1×10 s⁻¹, about 0.5×10⁻³ s⁻¹, about 1×10⁻⁴ s⁻¹, about 0.5×10⁻⁴ s⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹; about 1×10⁻⁴ s⁻¹, about 0.5×10⁻⁵ s⁻¹, or about 1×10⁻⁵ s⁻¹ (inclusive); about 1×10⁻⁵ s⁻¹ to about 1×10⁻³ s⁻¹, about 0.5×10⁻³ s⁻¹; about 1×10⁻⁴ s⁻¹, or about 0.5×10⁻⁴ s⁻¹ (inclusive); about 0.5×10's⁻¹ to about 1×10⁻³ s⁻¹, about 0.5×10⁻³ s⁻¹, or about 1×10⁻⁴ s⁻¹ (inclusive); about 1×10⁻⁴ to about 1×10⁻³ s⁻¹; or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10²M⁻¹ s⁻¹ to about 1×10⁶M⁻¹ s⁻¹, about 0.5×10⁶ M⁻¹ s⁻¹, about 1×10⁵M⁻¹ s⁻¹, about 0.5×10⁵ M⁻¹ s⁻¹, about 1×10⁴ M⁻¹ s⁻¹, about 0.5×10⁴M⁻¹ s⁻¹, about 1×10³ M⁻¹ s⁻¹, or about 0.5×10³ M⁻¹ s⁻¹ (inclusive); about 0.5×10¹ M⁻¹ s⁻¹ to about 1×10⁶M⁻¹ s⁻¹, about 0.5×10⁶ M⁻¹ s⁻¹; about 1×10⁻⁵M⁻¹ s⁻¹, about 0.5×10⁵ M⁻¹ s⁻¹, about 1×10⁴ M⁻¹ s⁻¹, about 0.5×10⁴ M⁻¹ s⁻¹, or about 1×10³ M⁻¹ s⁻¹ (inclusive); about 1×10³ M⁻¹ s⁻¹ to about 1×10⁶M⁻¹ s⁻¹, about 0.5×10⁶M⁻¹ s⁻¹, about 1×10⁵M⁻¹ s⁻¹, about 0.5×10⁵M⁻¹ s⁻¹, about 1×10⁴ M⁻¹ s⁻¹; or about 0.5×10⁴ M⁻¹ s⁻¹ (inclusive); about 0.5×10⁴ M⁻¹ s⁴ to about 1×10⁶M⁻¹ s⁻¹, about 0.5×10⁶M⁻¹ s⁻¹, about 1×10⁵M⁻¹ s⁻¹, about 0.5×10⁵ M⁻¹ s⁻¹; or about 1×10⁴M⁻¹ s⁻¹ (inclusive); about 1×10⁴ M⁻¹ s⁻¹ to about 1×10⁶ M⁻¹ s⁻¹, about 0.5×10⁶M⁻¹ s⁻¹, about 1×10⁵M⁻¹ s⁻⁴, or about 0.5×10⁵ M⁻¹ s⁻¹ (inclusive); about 0.5×10⁵M⁻¹ s⁻¹ to about 1×10⁶M⁻¹ s⁻¹, about 0.5×10⁶M⁻¹ s⁻¹, or about 1×10⁵ M⁻¹ s⁻¹ (inclusive); about 1×10⁵M⁻¹ s⁻¹ to about 1×10⁶ M⁻¹ s⁻¹; or about 0.5×10⁶M⁻¹ s⁻¹ (inclusive); or about 0.5×10⁶M⁻¹ s⁻¹ to about 1×10⁶M⁻¹ s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNTR2) that binds specifically to TNFα. In some embodiments, the anti-TNFα, agent includes or is a soluble TNRα receptor (e.g., Bjornberg et al., Lymphokine Cytokine Res. 13(3):203-211; 1994; Kozak et al., Am. J Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013; 1999; Mohler et al., J. Immunol. 151 (3): 1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, INK, LBP, MAPK, MEK1/2, MEKK 1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is complementary to all or part of a AP-1, ASK1, CD14, c-jun, ERK1/2, IκB, IKK, IRAK, INK, LBP, MAPK, MEK1/2, MEKK 1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., complementary to all or a part of any one of SEQ ID NOs: 1-37).

The nucleotides characterized by the Sequences ID NO: 1-37 are listed below and are being submitted in a separate and machine readable file.

### List, of Nucleotides with Sequence SEQ ID NO:1-37

Human TNFα CDS (SEQ ID NO: 1), Human TNFR1 CDS (SEQ ID NO: 2), Human TNFR2 CDS (SEQ ID NO: 3), Human TRADD CDS (SEQ ID NO: 4), Human TRAF2 CDS (SEQ ID NO: 5), Human AP-1 CDS (SEQ ID NO: 6), Human ASK1 CDS (SEQ ID NO: 7), Human CD14 CDS (SEQ ID NO: 8), Human ERK1 CDS (SEQ ID NO: 9), Human ERK2 CDS (SEQ ID NO: 10), Human IKK CDS (SEQ ID NO: 11), Human IKB CDS (SEQ ID NO: 12), Human IRAK CDS (SEQ ID NO: 13), Human INK CDS (SEQ ID NO: 14), Human LBP CDS (SEQ ID NO: 15), Human MEK1 CDS (SEQ ID NO: 16), Human MEK2 CDS (SEQ ID NO: 17), Human MEK3 CDS (SEQ ID NO: 18), Human MEK6 CDS (SEQ ID NO: 19), Human MEKK1 CDS (SEQ ID NO: 20), Human MEKK 3 CDS (SEQ ID NO: 21), Human MEKK4 CDS (SEQ ID NO: 22), Human MEKK 6 CDS (SEQ ID NO: 23), Human MEKK7 CDS (SEQ ID NO: 24), Human MK2 CDS (SEQ ID NO: 25), Human MyD88 CDS (SEQ ID NO: 26), Human NF-κB CDS (SEQ ID NO: 27), Human NIK CDS (SEQ ID NO: 28), Human p38 CDS (SEQ ID NO: 29), Human PKR CDS (SEQ ID NO: 30), Human Rac CDS (SEQ ID NO: 31), Human Raf CDS (SEQ ID NO: 32), Human K-Ras CDS (SEQ ID NO: 33), Human N-Ras CDS (SEQ ID NO: 34), Human RIP CDS (SEQ ID NO: 35), Human TRAF6 CDS (SEQ ID NO: 36), and Human TTP CDS (SEQ ID NO: 37).

An antisense nucleic acid molecule can be complementary to all or part of a non-coding region of the coding strand of a nucleotide sequence encoding an AP-1, ASK1, CD14, c-jun, ERK1/2, IκB, IKK, IRAK, INK, LBP, MAPK, MEK1/2, MEKK 1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding an AP-1, ASK1, CD14, c-jun, ERK1/2, IκB, IKK, IRAK, JNK, EBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, JNK, LBP, MAPK, MEK1/2, MEKK 1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methyl cytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, e-jun, ERK1/2, IκB, IKK, IRAK, INK, EBP, MARK, MEK1/2, MEKK1/4, MEKK4/7, MEKK 3/6, MK2, MyD88, NF-κB, NIK, p38, PKR, rac, ras, raf, RIP, INFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an a-anomeric nucleic acid molecule. An α-anomeric nucleic acid molecule forms specific double-stranded hybrids with complementary RNA in which, contrary to the usual, p-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, INK, LBP, MAPK, MEK1/2, MEKK 1/4, MEKK4/7, MEKK 3/6, MK2. MyD88, NF-κB, NIK, p38, PKR, rac, ras, raf, RIP, TNFα, TNFR1, TNFR2, TRADD, TRAF2, TRAF6, or TIP mRNA, e.g., specificity for any one of SEQ ID NQs: 1-37). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IKB, IKK, IRAK, INK, LBP, MAPK, MEK1/2, MEKK 1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein. For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, INK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or FTP polypeptide can be inhibited by targeting nucleotide sequences complementary to the regulatory region of the gene encoding the AP-1, ASK1, CD14, c-jun, ERK1/2, IκB, IKK, IRAK, INK, LBP, MAPK, MEK1/2, MEKK 1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1.991; and Helene, Ann. N.Y. Acad Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18); 12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, INK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vernurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, R05126766 (CH5126766), PLX7904, and MLN2480).

In some examples, the anti-TNFα agent TNFα inhibitor is a small molecule that inhibits the activity of one of MK2 (PF 3644022 and PHA 767491), JNK (e.g., AEG 3482, BI 78D3, CEP 1347, c-JUN peptide, IQ 1S, JIP-1 (153-163), SP600125, Si 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX Oil, TA 01, TA 02, TAK 715, VX 702, and VX 745), PKR (e.g., 2-aminopurine or CAS 608512-97-6), TTP (e.g., CAS 329907-28-0), MEK1/2 (e.g., Faeciorusso et al., Expert Review Gastroentrol. Hepatol 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), IκB (e.g., Suzuki et al., Expert. Opin. Invest Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 28 nucleotides, about 15 nucleotides to about 26 nucleotides, about 15 nucleotides to about 25 nucleotides, about 15 nucleotides to about 24 nucleotides, about 15 nucleotides to about 22 nucleotides, about 15 nucleotides to about 20 nucleotides, about 15 nucleotides to about 18 nucleotides, about 15 nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about. 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM, Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L, Paquette, ed., Encyclopedia of Reagents for Organic Synthesis, John Wiley and Sons (1995), and subsequent editions thereof.

### Preparative Examples

### Abbreviation of Chemicals

ACM=acetonitrile
AcOH=acetic acid
BTC=trichloromethyl chloroformate
DBU=1,8-diazabicycloundec-7-ene
DCM=dichloromethane
Dess-Martin=(1,1,1-triacetoxy)-1,1-dihydro-1,2-benziodoxol-3(1H)-one
DMEDA=N, N′-dimethylethylenediamine

### DMF=N,N-dimethylformamide

DMSO=dimethyl sulfoxide
Et=ethyl
EtOH=ethanol
LC-MS=liquid chromatography-mass spectrometry
LDA=lithium diisopropylamide
Me=methyl
MeOH=methanol
n-Bu=n-butyl

### NBS=N-bromosuccinimide

### NCS=N-chlorosuccinimide

### NIS=N-iodosuccinimide

NMR=nuclear magnetic resonance
Pd(dppf)Cl₂=dichloro[1,1′-bis(diphenylphosphino)ferrocene]palladium
Pd(PPh₃)₄=tetrakis(triphenylphosphine)Palladium(0)
PE=petroleum ether
Ph=phenyl
HPLC=high performance liquid chromatography
PTSA=p-toluenesulfonic acid
Py=pyridine
RT=room temperature
TBAF=tetrabutylammonium fluoride
TBDPSCl=tert-butyidiphenylsilyl chloride
t-Bu=text-butyl
TEA=tri ethyl amine
TEA=trifluoroacetic acid
THF=tetrahydrofuran
Ti(i-PrO)₄=tetraisopropyl titanate
TLC=thin layer chromatography Materials and Methods

The progress of reactions was often monitored by TLC or LC-MS. The identity of the products was often confirmed by LC-MS. The LC-MS was recorded using one of the following methods.

Method A: Shim-pack XR-ODS, CIS, 3×50 mm, 2.5 um column, 1.0 uL injection, 1.5 mL/min flow rate, 90-900 amu scan range, 190-400 am UV range, 5-100% (1.1 min), 100% (0.6 min) gradient with ACN (0.05% TFA) and water (0.05% TFA), 2 minute total run time.

Method B: Kinetex EVG, C18, 3×50 mm, 2.2 um column, 1.0 uL Injection, 1.5 mL/min flow rate, 90-900 amu scan range, 190-400 am UV range, 10-95% (1.1 min), 95% (0.6 min) gradient with ACN and water (0.5% NH₄HCO₃), 2 minute total run time.

Method C: Shim-pack XR-ODS, CIS, 3×50 mm, 2.5 um column, 1.0 uL injection, 1.5 mL/min flow rate, 90-900 amu scan range, 190-400 nm UV range, 5-100% (2.1 min), 100% (0.6 min) gradient with ACN (0.05% TFA) and water (0.05% TFA), 3 minute total run time.

Method D: Kinetex EVO, CIS, 3×50 mm, 2.2 um column, 1.0 uL injection, 1.5 mL/min flow rate, 90-900 amu scan range, 190-400 nm UV range, 10-95% (2.1 min), 95′% (0.6 min) gradient with ACN and water (0.5% NH₄HCO₃), 3 minute total run time.

The final targets were purified by Prep-HPLC. The Prep-HPLC was carried out using the following method.

Method E: Pre-HPLC: Column, XBridge Shield RP18 OBD (19×250 mm, 10 um); mobile phase, Water (10 mmol/L NH₄HCO₃) and ACN, UV detection 254/210 nm.

NMR was recorded on BROKER NMR 300.03 Mz, DUL-C-H, ULTRASHIELD™ 300, A VANCE II 300 B-ACS™ 120 or BROKER NMR 400.13 Mz, BBFO, ULTRASHIELD™ 400, A VANCE III 400, B-ACS™ 120.

### Preparative Examples

### A. Preparation of Intermediates

Scheme for the preparation of Sulfonamide Intermediates: Schemes below illustrate the preparation of sulfonamide intermediates.

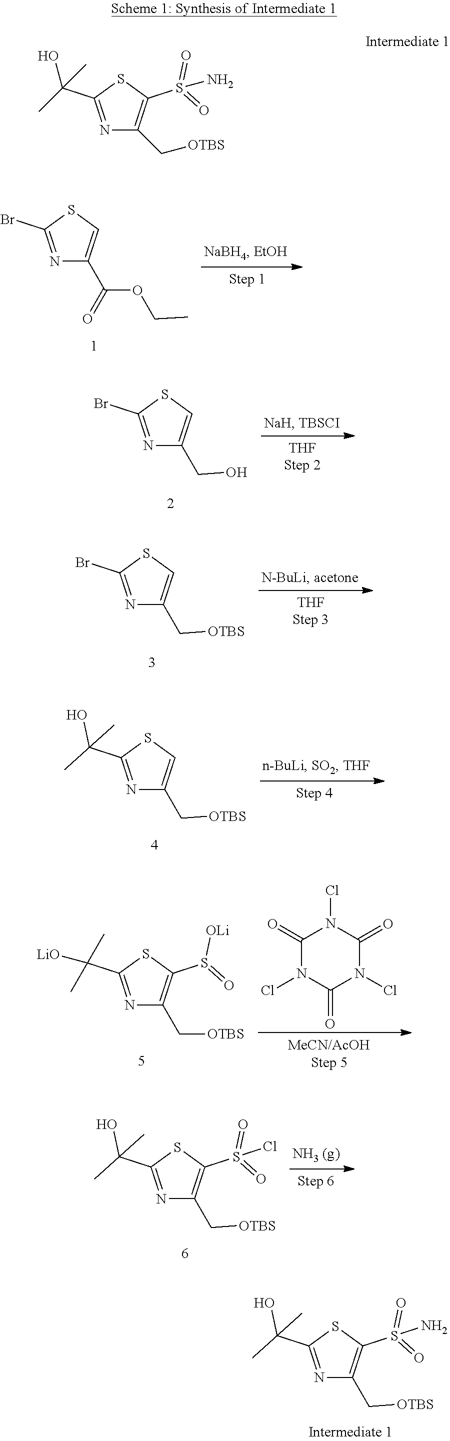

### 1. Synthesis of (2-bromo-1,3-thiazol-4-yl)methanol

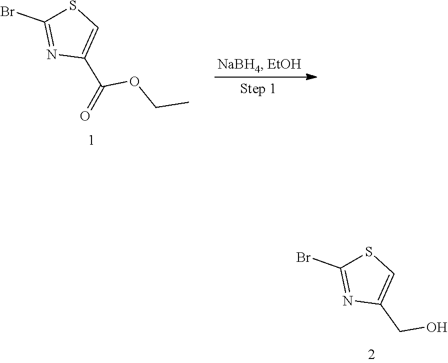

Into a 1 L round-bottom flask, was placed a solution of ethyl 2-bromo-1,3-thiazole-4-carboxylate (50 g, 211.79 mmol. 1 equiv) in EtOH (500 mL). NaBH₄ (16.0 g, 423.59 mmol, 2 equiv) was added to the solution in portions at 0° C. The resulting solution was stirred for 3 hr at room temperature. The reaction was then quenched by the addition of 1 L of ice-water. The resulting solution was extracted with 3×500 ml of ethyl acetate, and the combined organic layers were dried over NaSO₄ and concentrated under vacuum. This resulted in 35 g (85.1%) of (2-bromo-1,3-thiazol-4-yl)methanol as yellow oil.

LCMS of (2-bromo-1,3-thiazol-4-yl)methanol (Method A): 194.0, 196.0 [M+H]⁺, retention time 0.581 min. Method: Kinetex@ 2.6 um EVO C18, 50*3.0 mm, 0.3 uL injection, 1.5 mL/min flowrate, 90-900 arnu scan range, 254 ran UV detection. Mobile phase A: Water (5 mmoL/L NH₄HCO₃) and Mobile Phase B: MeCN. 10% MPB to 95.0% in 1.1 min, hold at 95% MPB for 0.53 min, 95% MPB to 10% in 0.06 min, then equilibration to 10% MPB for 0.11 min.

### 2. Synthesis of 2-bromo-4-[[(tert-butyldimethylsilyl)oxy]methyl]1,3thiazole

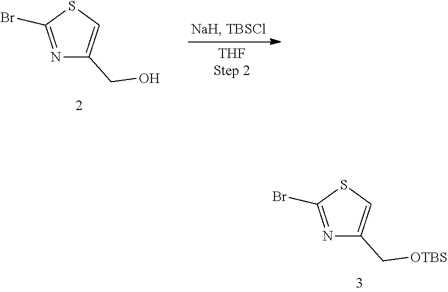 [SMILES: OCc1csc(Br)n1 ; CC(C)(C)[Si](C)(C)OCc1csc(Br)n1]

Into a 1-L round-bottom flask, was placed a solution of (2-bromo-1,3-thiazol-4-yl)methanol (35 g, 80.37 mmol, 1 equiv) in THF (400 mL). NaH (10.8 g, 70.86 mmol, 1.5 equiv, 60%) was added to the mixture in portions at 0° C. The mixture was stirred at 0° C. for another 1 h, when TBSCl (43.5 g, 88.59 mmol, 1.6 equiv) was added to the mixture in portions at 0° C. The resulting solution was stirred for 2 hr at room temperature. The reaction was then quenched by the addition of 300 mL of ice-water. The resulting solution was extracted with 3×300 ml of ethyl acetate; and the combined organic phase was dried over NaSO₄ and concentrated. The residue was applied onto a silica gel column with ethyl acetate/petroleum ether (1:30). This resulted in 30.0 g (53.9%) of 2-bromo-4-[[(tert-butyldimethylsilyl)oxy]methyl]-1,3-thiazole as yellow oil.

H-NMR of 2-bromo-4-[[(tert-butyldimethylsilyl)oxy]methyl]-1,3-thiazole: (CDCl₃, 300 MHz, ppm): δ 7.12 (t, 0.7=1.5 Hz, 1H), 4.81 (d, J=1.5 Hz, 2H), 0.93 (s, 9H), 0.10 (s, 6H).

### 3. Synthesis of 2(4[[(tert-butyldimethylsilyl)oxy]methyl]-1,3-thiazol-2-yl)propan-2-ol

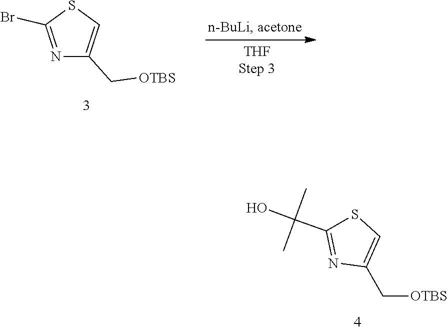 [SMILES: CC(C)(C)[Si](C)(C)OCc1csc(Br)n1 ; CC(C)(O)c1nc(CO[Si](C)(C)C(C)(C)C)cs1]

Into a 500-mL round-bottom flask purged and maintained with an inert atmosphere of nitrogen, was placed a solution of 2-bromo-4-[[(tert-butyldimethylsilyl)oxy]methyl]-1,3-thiazole (15.0 g, 48.65 mmol, 1 equiv) in THF (150 mL). n-BuLi (23.4 mL, 58.38 mmol, 2.5 M, 1.2 equiv) was added to the mixture in dropwise at −78° C. and the resulting mixture stirred for 30 min at −78° C. Then propan-2-one (3.4 g, 58.38 mmol, 1.2 equiv) was added to the mixture dropwise at −78° C., The mixture was stirred for another 1 h at room temperature. The reaction was then quenched by the addition of 200 mL of water. The resulting solution was extracted with 3×300 ml of ethyl acetate, the combined organic phase was dried over NaSO₄ and concentrated. The residue was applied onto a silica gel column with ethyl acetate/petroleum ether (1:10). This resulted in 12 g (85.7%) of 2-(4-[[(tert-butyldimethylsilyl)oxy]methyl]-1,3-thiazol-2-yl)propan-2-ol as yellow oil.

LC-MS of 2-(4-[[(tert-butyldimethylsilyl)oxy]methyl]-1,3-thiazol-2-yl)propan-2-ol (Method B): 288.2 [M+H]+, retention time 1.29 min. Method: Kinetex EVO C18, 50*3.0 mm, 0.3 uL injection, 1.2 mL/min flowrate, 90-900 amu scan range, 254 nm UV detection. Mobile phase A: Water (6.5 mmoL/L NH₄HCO₃), mobile phase B: MeCN. 10% MPB to 95.0% in 1.1 min, hold at 95% MPB for 0.5 min, 95% MPB to 10% in 0.01 min, then equilibration to 10% MPB for 0.21 min.

### 4. Synthesis of 4-[[(tert-butyldimethylsilyl)oxy]methyl]-2-(2-hydroxypropan-2-yl)-1,3-thiazole-5sulfonyl chloride

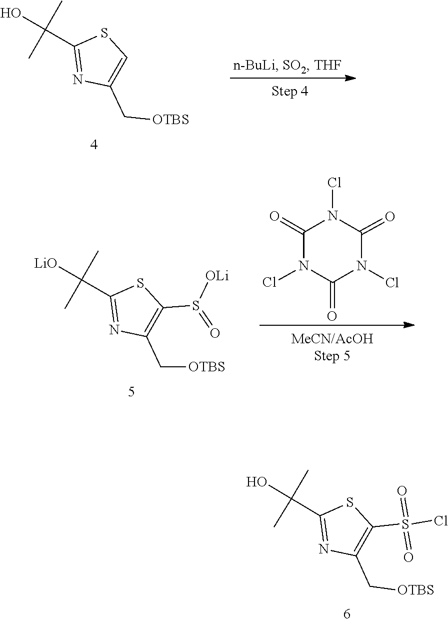

Into a 250-mL round-bottom flask purged and maintained with an inert atmosphere of nitrogen, was placed a solution of 2-(4-[[(tert-butyldimethylsilyl)oxy]methyl]-1,3-thiazol-2-yl)propan-2-ol (10 g, 32.43 mmol, 1 equiv) in THF (100 mL). n-BuLi (8.4 mL, 20.87 mmol, 2.5 M, 3 equiv) was added to the mixture at −78° C. and the mixture stirred for another 30 min at −78° C. Then SO₂ was bubbled for 30 min; and the reaction was stirred for another 2 h at room temperature. The resulting mixture was concentrated. Then the residue was dissolved in MeCN/AcOH(200 mL/10 mL). 1,3,5-trichloro-1,3,5-triazinane-2,4,6-trione (15.1 g, 64.86 mmol, 2 equiv) was added to the mixture in portions at 0° C., and the mixture stirred for another 30 min at 0° C. The resulting mixture was concentrated at 0° C. This resulted in 12.5 g (92.9%) of 4-[[(tert-butyldimethylsilyl)oxy]methyl]-2-(2-hydroxypropan-2-yl)-1,3-thiazole-5-sulfonyl chloride as a yellow solid. This crude material was used directly for the next step without further purifications.

LC-MS of 4-[[(tert-butyldimethylsilyl)oxy]methyl]-2-(2-hydroxypropan-2-yl)-1,3-thiazole-5-sulfonyl chloride (Method B): 386.1 [M+H]+, retention time 1.456 min. Method: Kinetex EVO 08, 50*3.0 mm, 0.3 uL injection, 1.2 mL/min flowrate, 90-900 amu scan range, 254 nm UV detection. Mobile phase A: Water (6.5 mmoL/L NH₄HCO₃), mobile phase B; MeCN. 10% MPB to 95.0% in 1.1 min, hold at 95% MPB for 0.5 min, 95% MPB to 10% in 0.01 min, then equilibration to 10% MeCN for 0.21 min.

### 5. Synthesis of 4-[[(tert-butyldimethylsilyl)oxy]methyl]-2-(2-hydroxypropan-2-yl)-1,3-thiazole-5sulfonamide

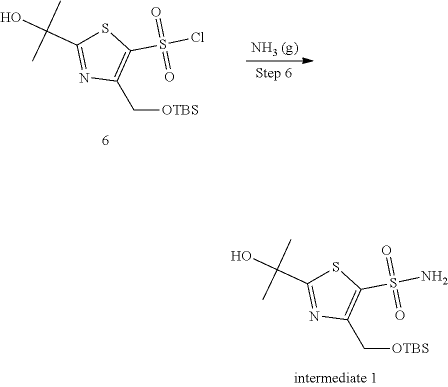 [SMILES: CC(C)(O)c1nc(CO[Si](C)(C)C(C)(C)C)c(S(=O)(=O)Cl)s1 ; CC(C)(O)c1nc(CO[Si](C)(C)C(C)(C)C)c(S(N)(=O)=O)s1]

Into a 250-mL round-bottom flask, was placed a solution of 4-[[(tert-butyldimethylsilyl)oxy]methyl]-2-(2-hydroxypropan-2-yl)-1,3-thiazole-5-sulfonyl chloride (12.5 g, 32.38 mmol, 1 equiv) in DCM (130 mL). NH₃ was bubbled for 10 min into the solution. The resulting solution was stirred for another 1 hr at room temperature. The resulting mixture was concentrated. The residue was applied onto a silica gel column with ethyl acetate/petroleum ether (1:5). This resulted in 5.8 g (49.1%) of 4-[[(tert-butyldimethylsilyl)oxy]methyl]-2-(2-hydroxypropan-2-yl)-1,3-thiazole-5-sulfonamide as yellow oil.

LC-MS of 4-[[(tert-butyldimethylsilyl)oxy]methyl]-2-(2-hydroxypropan-2-yl)-1,3-thiazole-5-sulfonamide (Method B): 367.1 [M+H]+, retention time 1.184 min. Method: Kinetex EVO C18, 50*3.0 mm, 0.3 uL injection, 1.2 mL/min flowrate, 90-900 amu scan range, 254 nm UV detection. Mobile phase A: Water (6.5 mmoL/L NH₄HCO₃), mobile phase B: MeCN. 10% MPB to 95.0% in 1.1 min, hold at 95% MPB for 0.5 min, 95% MPB to 10% in 0.01 min, then equilibration to 10% MPB for 0.21 min.

H-NMR-4-[[(tert-butyldimethylsilyl)oxy]methyl]-2-(2-hydroxypropan-2-yl)-1,3-thiazole-5-sulfonamide: (CD₃OD-d4, 400 MHz, ppm): δ 4.99 (s, 2H), 1.59 (s, 6H), 0.92 (s, 9H), 0.12 (s, 6H).

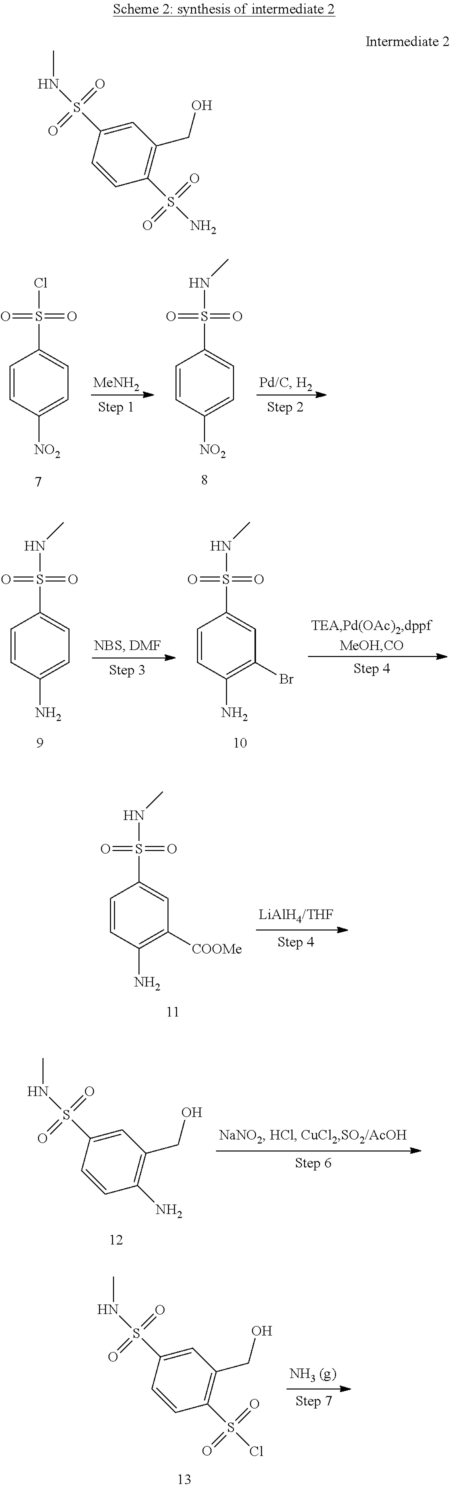

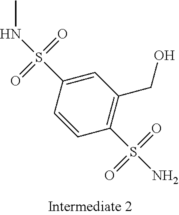 [SMILES: CNS(=O)(=O)c1ccc(S(N)(=O)=O)c(CO)c1]

### 1. Synthesis of N-methyl-4-nitrobenzene-1-sulfonamide

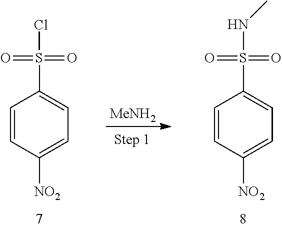

Into a 250 mL round-bottom flask was added methanamine(91 mL, 54.2 mmol, 2 equiv) at room temperature, after which 4-nitrobenzene-1-sulfonyl chloride (7.0 g, 31.7 mmol, 1.2 equiv) was added in portions at 0° C. Then the resulting mixture was stirred for 1 h at room temperature. The residue was purified by silica gel column chromatography, eluted with PE/EtOAc (2:1) to afford N-methyl-4-nitrobenzene-1-sulfonamide(5.8 g, 84.7%) as a light yellow solid.

### 2. Synthesis of 4-amino-N-methylbenzene-1-sulfonamide

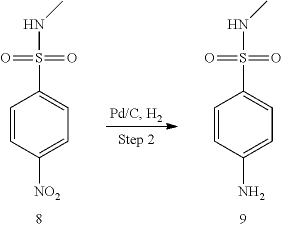

Into a 250 mL round-bottom flask was added N-methyl-4-nitrobenzene-1-sulfonamide(5.8 g, 26.8 mmol, 1 equiv) and isopropanol(50 mL) at room temperature. To the stirred solution was added Pd/C(580 mg, 5.5 mmol, 0.20 equiv) at room temperature under nitrogen. The resulting mixture was stirred overnight at room temperature under hydrogen atmosphere, after which it was filtered to remove the solid. The filtrate was concentrated under reduced pressure to afford 4-amino-N-methylbenzene-1-sulfonamide (4.9 g, 84.5%) as yellow solid.

### 3. Synthesis of 4-amino-3-bromo-N-methylbenzene-1-sulfonamide

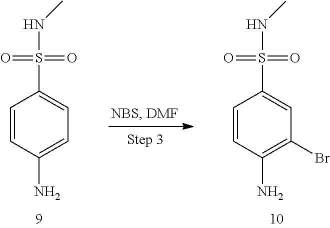

Into a 100 mL round-bottom flask were added 4-amino-N-methylbenzene-1-sulfonamide (5.8 g, 26.8 mmol, 1 equiv) and DMF(25 mL) at room temperature. To this stirred solution was added NBS (4.3 g, 24.1 mmol, 0.9 equiv) in portions at room temperature. The residue was purified by silica gel column chromatography, eluted with PE/EtOAc (2:1) to afford 4-amino-3-bromo-N-m ethylbenzene-1-sulfonamide (6 g, 84.4%) as a dark yellow solid.

### 4. Synthesis of methyl 2-amino-5-(methylsulfamoyl)benzoate

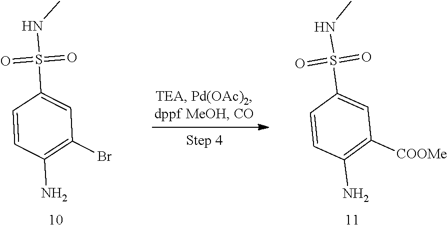 [SMILES: CNS(=O)(=O)c1ccc(N)c(Br)c1 ; CNS(=O)(=O)c1ccc(N)c(C(=O)OC)c1]

Into a 250 mL pressure tank reactor were added 4amino-3bromo-N-methylbenzene-1-sulfonamide (6.0 g, 22.6 mmol, 1 equiv) and TEA (2.2 g, 22.6 mmol, 1 equiv) at room temperature. To this stirred solution was added Pd(OAc)₂(1.0 g, 4.5 mmol, 0.2 equiv) and dppf (3.8 g, 6.8 mmol, 0.3 equiv) in one portion under nitrogen atmosphere. Then the resulting mixture was stirred at 110° C. under CO atmosphere (10 atm) overnight. The resulting mixture was concentrated under reduced pressure. The residue was purified by silica gel column chromatography, eluted with PE/EtOAc (2:1) to afford methyl 2-amino-5-(methylsulfamoyl)benzoate (4.7 g, 74.4%) as a light yellow solid,

### 5. Synthesis of 4-amino-3-(hydroxymethyl)-N-methylbenzene-1-sulfonamide

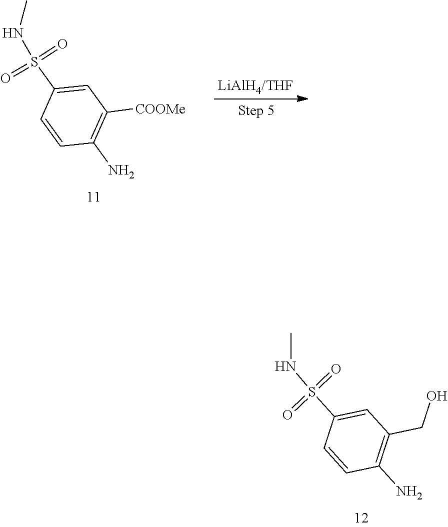 [SMILES: CNS(=O)(=O)c1ccc(N)c(C(=O)OC)c1 ; CNS(=O)(=O)c1ccc(N)c(CO)c1]

Into a 500 mL round-bottom flask was added methyl-2-amino-5-(methylsulfamoyl)benzoate (4.5 g, 18.4 mmol, 1 equiv) and THF(100 mL) at room temperature. To this stirred solution was added LiAlH₄ (1398.4 mg, 36.84 mmol, 2 equiv) in portions at 0° C. under nitrogen atmosphere. Then the resulting mixture was stirred for 4 h. The resulting mixture was concentrated under reduced pressure. The crude product was purified by reverse phase HPLC with the following conditions (column, C18 silica gel, mobile phase, acetonitrile in water, 0% to 15% gradient in 7 min) to afford 4-amino-3-(hydroxymethyl)-N-methylbenzene-1-sulfonamide 2.2 g (55.3%) as a light yellow solid.

### 6. Synthesis of 2-(hydroxymethyl)-4-(N-methylsulfamoyl)benzene-1-sulfonyl chloride

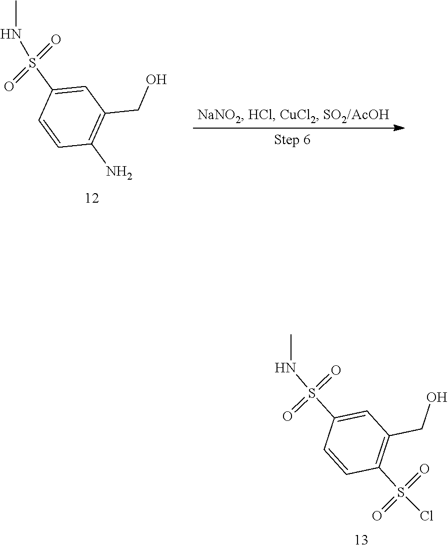 [SMILES: CNS(=O)(=O)c1ccc(N)c(CO)c1 ; CNS(=O)(=O)c1ccc(S(=O)(=O)Cl)c(CO)c1]

To a stirred solution of 4-amino-3-(hydroxymethyl)-N-methylbenzene-1-sulfonamide (1 g, 4.62 mmol, 1 equiv) in HCl (6M) (TO mL) was added NaNO₂ (382.8 mg, 5.55 mmol, 1.20 equiv) dropwise at −10° C. for 20 min. Then the resulting mixture was added to a solution of CuCl₂ in SO₂/AcOH (15 mL) (that had been stirred together for 15 min) in one portion at −10° C. over 30 min. The resulting mixture was diluted with water (50 mL) and extracted with CH₂Cl₂ (3×25 mL). The combined organic layers were washed with water (3×50 mL) and dried over anhydrous Na2SO4. After filtration, the filtrate was concentrated under reduced pressure. The crude product was used in the next step directly without further purification.

### 7. Synthesis of 3-(hydroxymethyl)-N1-methylbenzene-1,4-disulfonamide

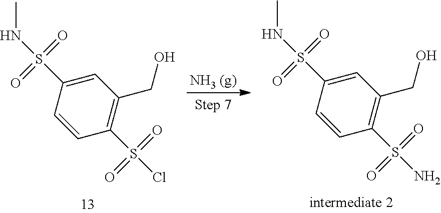 [SMILES: CNS(=O)(=O)c1ccc(S(=O)(=O)Cl)c(CO)c1 ; CNS(=O)(=O)c1ccc(S(N)(=O)=O)c(CO)c1]

To a stirred solution of NH₃ in THF(40 mL) was added 2-(hydroxymethyl)-4-(methylsulfamoyl)benzene-1-sulfonyl chloride (1 g, 3.34 mmol, 1 equiv) in THF(6 mL) dropwise at 0° C. The resulting mixture was stirred overnight at room temperature. The resulting residue was purified by Prep-TLC (EtOAc) to afford 3-(hydroxymethyl)-N1-methylbenzene-1,4-disulfonamide (400 mg, 42.7%) as a yellow solid.

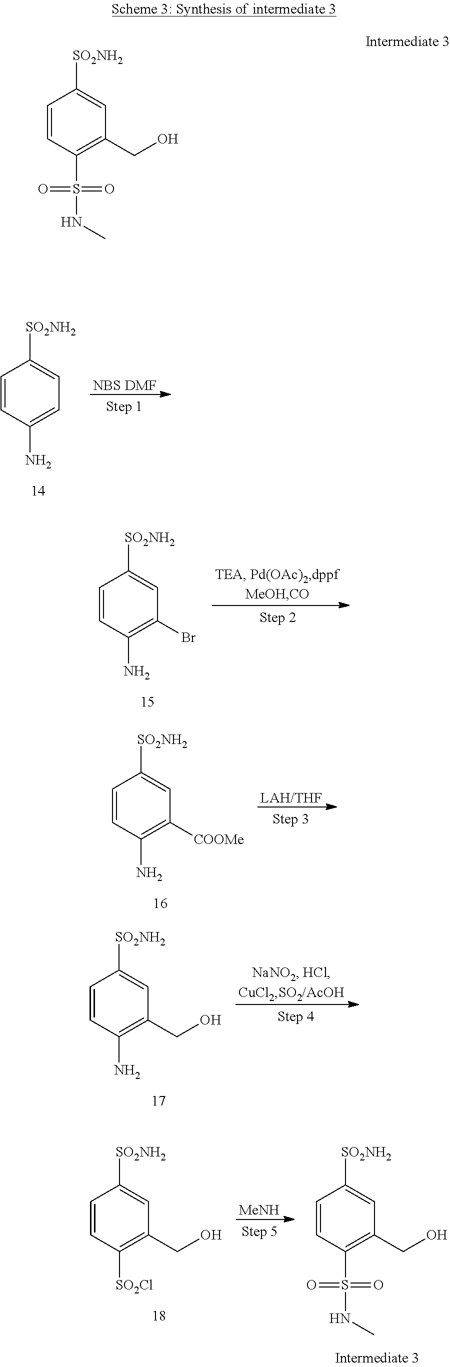

### 1. Synthesis of 4-amino-3-bromo-N-methylbenzene-1-sulfonamide

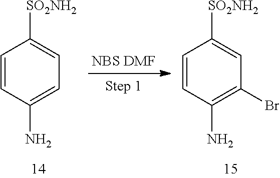

To a stirred solution of 4-amino-3-(hydroxymethyl)-N-methylbenzene-1-sulfonamide(5.8 g, 26.8 mmol, 1 equiv) in DMF(25 mL) was added NBS(4.3 g, 24.1 mmol, 0.9 equiv) in portions at room temperature. The resulting residue was purified by silica gel column chromatography, eluted with PE/EtOAc (2:1) to afford 4-amino-3-bromo-N-methylbenzene-1-sulfonamide(6 g, 84.4%) as a dark yellow solid.

### 2. Synthesis of methyl 2-amino-5-(methylsulfamoyl)benzoate

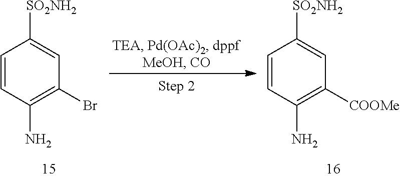 [SMILES: Nc1ccc(S(N)(=O)=O)cc1Br ; "TEA, Pd(OAc)2, dppfMeOH, CO" ; COC(=O)c1cc(S(N)(=O)=O)ccc1N]

Into a 250 mL pressure tank reactor were added 4-amino-3-bromo-N-methylbenzene-1-sulfonamide (6 g, 22.63 mmol, 1 equiv) in MeOH (150 mL). TEA(2.3 mg, 22.63 mmol, 1 equiv), Pd(OAc)₂(1016.2 mg, 4.53 mmol, 0.2 equiv) and dppf(3.8 g, 6.79 mmol, 0.3 equiv) were added to the mixture. Then the resulting mixture was stirred at 110° C. under CO atmosphere(10 atm) overnight. The resulting mixture v/as concentrated under reduced pressure. The residue was purified by silica gel column chromatography, eluted with PE/EtOAc (2:1) to afford methyl 2-amino-5-(methylsulfamoyl)benzoate(4.7 g, 74.4%) as a light yellow solid.

### 3. Synthesis of 4-amino-3-(hydroxymethyl)-N-methylbenzene-1-sulfonamide

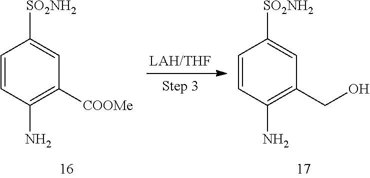 [SMILES: COC(=O)c1cc(S(N)(=O)=O)ccc1N ; "LAH/THF" ; Nc1ccc(S(N)(=O)=O)cc1CO]

Into a 500 mL round-bottom flask were added methyl 2-amino-5-(methylsulfamoyl)benzoate (4.5 g, 18.4 mmol, 1 equiv) and THF (100 mL) at room temperature. LiAlH₄ (1.4 g, 36.8 mmol, 2 equiv) was added to the solution in portions at 0 degrees C. under nitrogen atmosphere. Then the resulting mixture was stirred for 4 h, The resulting mixture was concentrated under reduced pressure. The crude product was purified by reverse phase HPLC with the following conditions (column, C18 silica gel; mobile phase, acetonitrile in water, 0% to 15% gradient in 7 min) to afford 4-amino-3-(hydroxymethyl)-N-methylbenzene-1-sulfonamide (1.8 g, 56.4%) as a light yellow solid.

### 4. Synthesis of 2-(hydroxymethyl)-4-(methylsulfamoyl)benzene-1-sulfonyl chloride

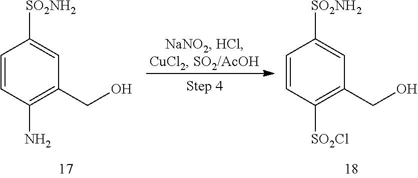 [SMILES: Nc1ccc(S(N)(=O)=O)cc1CO ; "NaNO2, HCl,CuCl2, SO2/AcOH" ; NS(=O)(=O)c1ccc(S(=O)(=O)Cl)c(CO)c1]

Into a 50 mL 3-necked round-bottom flask was added 4-amino-3-(hydroxymethyl)-N-methylbenzene-1-sulfonamide (1 g, 4.6 mmol, 1 equiv) in HCl (10 mL, aqueous, 6 M) at room temperature. NaNO₂ (382.8 mg, 5.6 mmol, 1.2 equiv) was added to the solution in portions at −10 degrees C. over 20 min. Then the resulting mixture was added to a solution of CuCl₂ (966.0 mg, 9.2 mmol, 2.0 equiv) in SO₂/AcOH (15 mL) (that had been stirred together for 15 min) in one portion at −10 degrees C. for 30 min. The resulting mixture was diluted with water (50 mL). The resulting mixture was extracted with CH₂Cl₂ (3×25 mL), The combined organic layers were washed with water (3×50 mL), dried over anhydrous Na₂SO₄. After filtration, the filtrate was concentrated under reduced pressure. The crude product was used in the next step directly without further purification.

### 5. Synthesis of 3-(hydroxymethyl)-N1-methylbenzene-1,4-disulfonamide

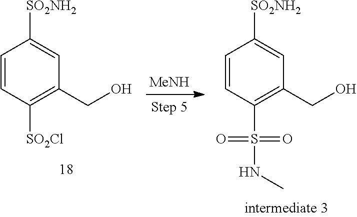 [SMILES: "MeNH" ; NS(=O)(=O)c1ccc(S(=O)(=O)Cl)c(CO)c1 ; CNS(=O)(=O)c1ccc(S(N)(=O)=O)cc1CO]

Into a 100 mL round-bottom flask was added NH₃ in THF(40 mL, 0.5M) at 0 degrees C. 2-(hydroxymethyl)-4-(methylsulfamoyl)benzene-1-sulfonyl chloride(l g, 3.3 mmol, 1 equiv) was added to the solution at 0 degrees C. The resulting mixture was stirred overnight at room temperature. The residue was purified by SiO₂-gel column, eluted with PE/EtOAc (2:1) to afford 3-(hydroxymethyl)-N1-methylbenzene-1,4-disulfonamide(400 mg, 42.8%) as a yellow solid.

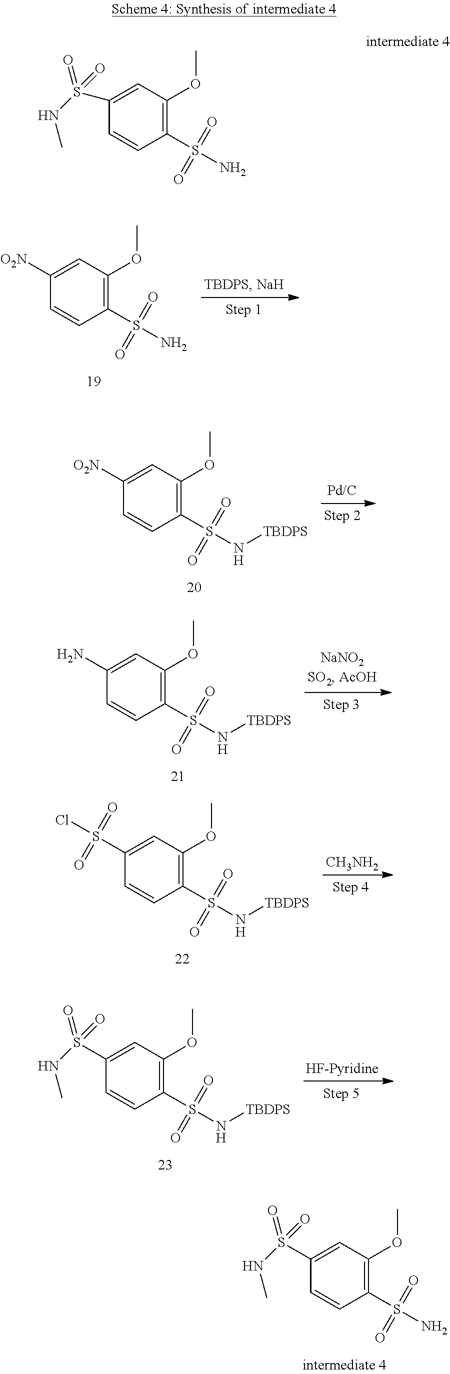

### 1. Synthesis of N-(tert-butyldiphenylsilyl)-2-methoxy-4-nitrobenzenesulfonamide

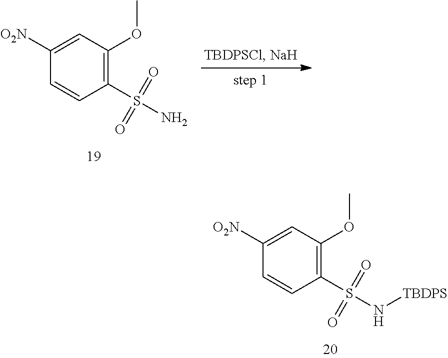

Into a 1 L round-bottom flask, was placed a solution of 2-methoxy-4-nitrobenzenesulfonamide (23.2 g, 100 mmol, 1 equiv) in THF (250 mL). NaH (8.0 g, 200.0 mmol, 2 equiv, 60%) was added to the solution in portions at 0° C. TBDPSCl (54.8 g, 200.0 mmol, 2 equiv) was added to the mixture at 0° C. The resulting solution was stirred overnight at room temperature. The reaction was then quenched by the addition of 1 L of ice-water. The resulting solution was extracted with 3×500 ml of ethyl acetate; the combined organic layers were dried over NaSO₄ and concentrated under vacuum. The residue was applied onto a silica gel column with ethyl acetate/petroleum ether (1:5) to give N-(tert-butyldiphenylsilyl)-2-methoxy-4-nitrobenzenesulfonamide (28 g, 59.6%) as yellow solid.

### 2. Synthesis of 4-amino-N-(tert-butyidiphenylsilyl)-2-methoxybenzenesulfonamide

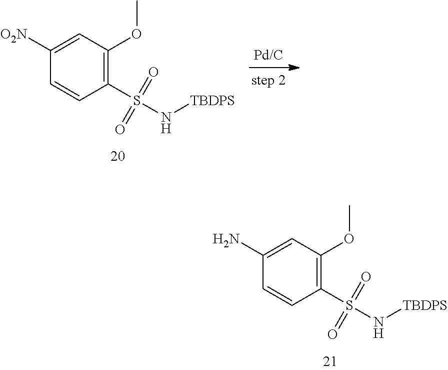

Into a 250 mL round-bottom flask were added N-(tert-butyldiphenylsilyl)-2-methoxy-4-nitrobenzenesulfonamide (12.6 g, 26.8 mmol, 1 equiv) and isopropanol(250 mL) at room temperature, Pd/C (580 mg, 5.5 mmol, 0.20 equiv) was added to the solution at room temperature under nitrogen atmosphere. The resulting mixture was stirred overnight at room temperature under hydrogen atmosphere, after which it was filtered to remove the solid. The filtrate was concentrated under reduced pressure to afford 4-amino-N-(tert-butyldiphenylsilyl)-2-methoxybenzenesulfonamide (11.8 g, 84.6%) as yellow solid.

### 3. Synthesis of 4-(N-(tert-butyldiphenylsilyl)sulfamoyl)-3-methoxybenzene-1-sulfonyl chloride

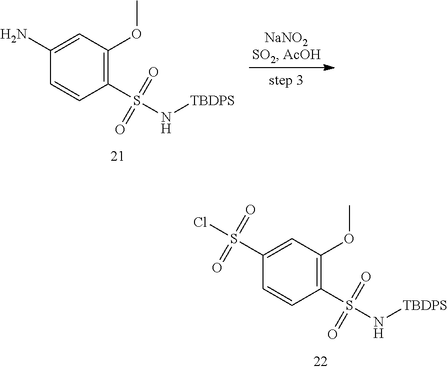

Into a 50 mL 3-necked round-bottom flask were added 4-amino-N-(tert-butyldiphenylsilyl)-2-methoxybenzenesulfonamide (2.0 g, 4.61 mmol, 1 equiv) in HCl (6 M, 20 mL) at room temperature. To a stirred solution was added NaNO₂ (382.8 mg, 5.55 mmol, 1.20 equiv) in portions at −10 degrees C. over 20 min. Then the resulting mixture was added to the solution of CuCl₂ in SO₂/AcOH(15 mL) (that had been stirred together for 15 min) in one portion at −10 degrees C. for 30 min. The resulting mixture was diluted with water (50 mL). The resulting mixture was extracted with CH₂Cl₂ (3×25 mL). The combined organic layers were washed with water (3×50 mL) and dried over anhydrous Na2SO4. After filtration, the filtrate was concentrated under reduced pressure. The crude product (2.8 g) was used in the next step directly without further purification.

### 4. Synthesis of N1-(tert-butyldiphenylsilyl)-2-methoxy-N4-methylbenzene-1,4-disulfonamide

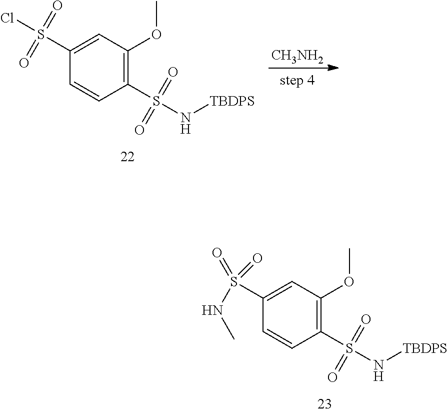

Into a 100 mL round-bottom flask were added methanamine in THF (40 mL, 0.5M) at 0 degrees C. To a stirred solution of NH₃ in THF (40 mL) was added crude 2-(hydroxymethyl)-4-(methylsulfamoyl)benzene-1-sulfonyl chloride (2.8 g) in THF(10 mL) dropwise at 0 degrees C., The resulting mixture was stirred overnight at room temperature. The residue was applied onto a silica gel column with ethyl acetate/petroleum ether (9:1) to give N1-(tert-butyldiphenylsilyl)-2-methoxy-N4-methylbenzene-1,4-disulfonamide (1.2 g, 52.0%) as yellow solid,

### 5. Synthesis of 3-methoxy-N1-methylbenzene-1,4-disulfonamide

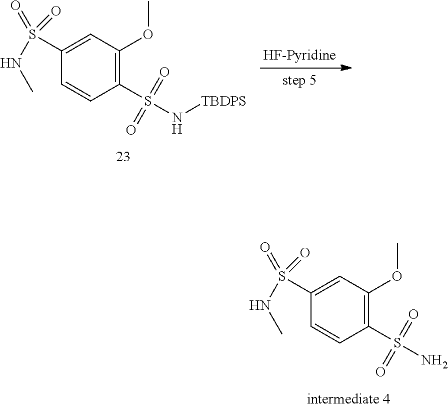

Into a 50-mL round-bottom flask, was placed a solution of N1-(tert-butyldiphenylsilyl)-2-methoxy-N4-methylbenzene-1,4-disulfonamide (218 mg, 0.42 mmol, 1 equiv) in THF (5 mL), and HF-Pyridine (417.9 mg, 4.22 mmol, 10 equiv). The resulting solution was stirred for 1 hr at room temperature. The resulting mixture was concentrated. The residue was applied onto a silica gel column with MeOH/DCM (1:10) to give 3-methoxy-N1-methylbenzene-1,4-disulfonamide (85.8 mg, 73.0%) as yellow solid.

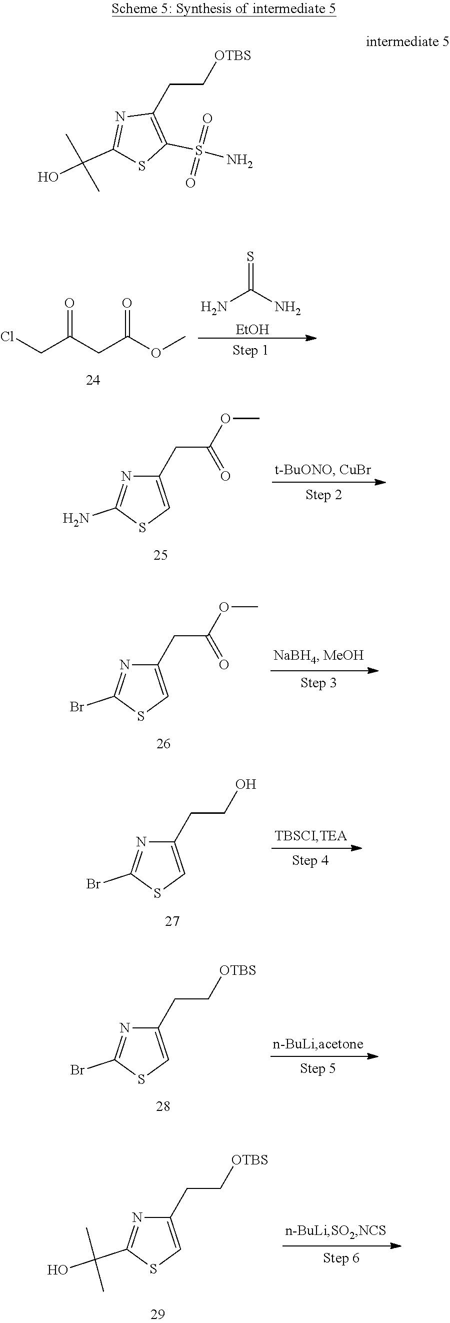

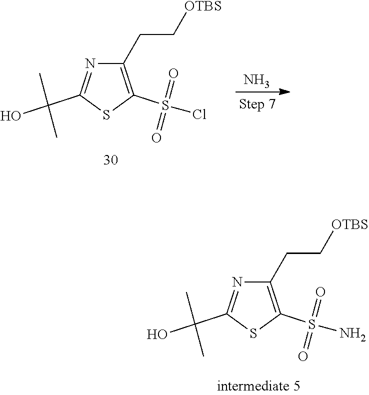

### 1. Synthesis of methyl 2-(2-aminothiazol-4-yl)acetate

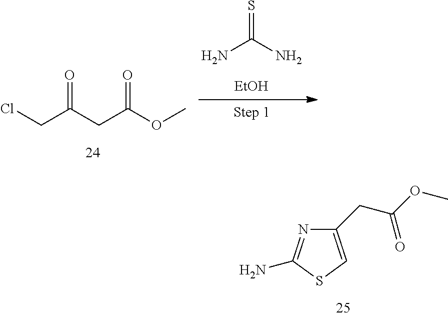

Into a 1 L round-bottom flask, was placed a solution of methyl 4-chloro-3-oxobutanoate (15.0 g, 100 mmol, 1 equiv) in EtOH (350 mL). Thiourea (7.6 g, 100 mmol, 1.0 equiv) was added to the solution. The resulting solution was refluxed overnight, after which it was cooled to room temperature. The resulting mixture was filtered to collect the solid, which was washed with Et₂O (200 mL*2) and dried over oven at 50° C. overnight to give methyl 2-(2-aminothiazol-4-yl)acetate (15.4 g, 89.5%) as yellow solid.

### 2. Synthesis of methyl 2-(2-bromothiazol-4-yl)acetate

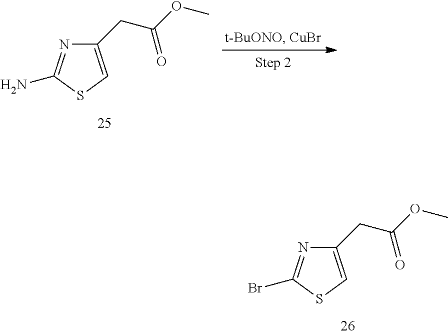

Into a 500 mL round-bottom flask, was placed a solution of 2-(2-aminothiazol-4-yl)acetate (15.4 g, 89.5 mmol, 1 equiv) in MeCN(250 mL). CuBr was added to the solution, and then t-BuONO was added to the solution dropwise in 0° C. The resulting solution was stirred for 30 min at room temperature and then stirred for 2 h at 70° C. The resulting mixture was concentrated in vacuo, purified with silica gel column, and eluted with EtOAc/PE (1:10) to give methyl 2-(2-bromothiazol-4-yl)acetate (12.3 g, 58.2%) as white solid.

### 3. Synthesis of 2-(2bromothiazol-4-yl)ethanol

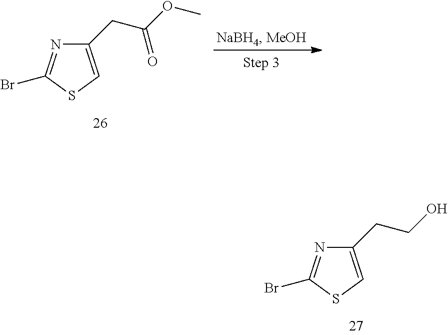

Into a 1 L round-bottom flask, was placed a solution of methyl 2-(2-bromothiazol-4-yl)acetate (12.3 g, 51.9 mmol, 1 equiv) in EtOH (200 mL). NaBH4 (3.9 g, 103.8 mmol, 2 equiv) was added to the solution in portions at 0° C. The resulting solution was stirred for 3 hr at room temperature. The reaction was then quenched by the addition of 1 L of ice-water. The resulting solution was extracted with 3×500 ml of ethyl acetate, and the combined organic layers were dried over NaSO₄ and concentrated under vacuum. This resulted in 8.9 g (82.1%) of 2-(2-bromothiazol-4-yl)ethanol as yellow oil.

### 4. Synthesis of 2-bromo-4-(2-(tert-butyldimethylsilyloxy)ethyl)thiazole

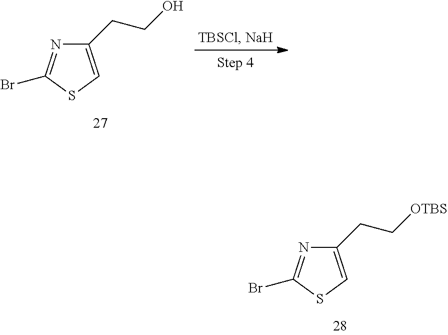 [SMILES: OCCc1csc(Br)n1 ; CC(C)(C)[Si](C)(C)OCCc1csc(Br)n1]

Into a 500 mL round-bottom flask, was placed a solution of 2-(2-bromothiazol-4-yl)ethanol (8.9 g, 42.6 mmol, 1 equiv) in THF (400 mL). NaH (2.56 g, 63.9 mmol, 1.5 equiv, 60%) was added to the mixture in portions at 0° C. The mixture was stirred at 0° C. for another 1 h, when TBSCl (10.2 g, 68.2 mmol, 1.6 equiv) was added to the mixture in portions at 0° C. The resulting solution was stirred for 2 hr at room temperature. The reaction was then quenched by the addition of 300 mL of ice-water. The resulting solution was extracted with 3×300 mi of ethyl acetate; the combined organic phase was dried over NaSO4 and concentrated. The residue was applied onto a silica gel column with ethyl acetate/petroleum ether (1:30). This resulted in 7.6 g (55.1%) of 2-bromo-4-(2-(tert-butyldimethylsilyloxy)ethyl)thiazole as a yellow oil,

### 5. Synthesis of 2-(4-(2-(tert-butyldimethylsilyloxy)ethyl)thiazol-2-yl)propan-2-ol

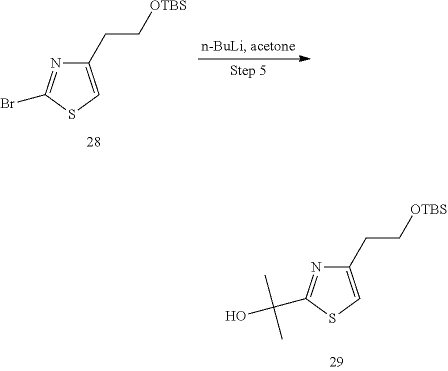 [SMILES: CC(C)(C)[Si](C)(C)OCCc1csc(Br)n1 ; CC(C)(O)c1nc(CCO[Si](C)(C)C(C)(C)C)cs1]

Into a 500-mL round-bottom flask purged and maintained with an inert atmosphere of nitrogen, was placed a solution of 2-bromo-4-(2-(tert-butyldimethylsilyloxy)ethyl)thiazole (7.6 g, 23.4 mmol, 1 equiv) in THF (50 mL). n-BuLi (11.2 mL, 28.1 mmol, 2.5 M, 1.2 equiv) was added to the mixture in dropwise at −78° C.; and the mixture was stirred for 30 min at −78° C. Then acetone (1.6 g, 28.1 mmol, 1.2 equiv) was added to the mixture dropwise at −78° C. After an additional hour of stirring, the reaction was quenched by the addition of 200 mL of water. The resulting solution was extracted with 3×300 ml of ethyl acetate: the combined organic phase was dried over NaSO4 and concentrated. The residue was applied onto a silica gel column with ethyl acetate/petroleum ether (1:10). This resulted in 6.1 g (86.2%) of 2-(4-(2-(tert-butyldimethylsilyloxy)ethyl)thiazol-2-yl)propan-2-ol as yellow oil.

### 6. Synthesis of 4-(2-(tert-butyldimethylsilyloxy)ethyl)-2-(2-hydroxypropan-2-yl)thiazole-5-sulfonyl chloride

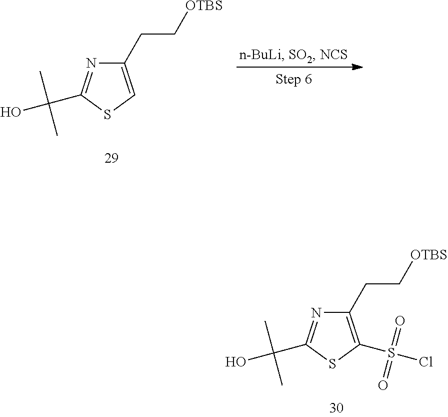 [SMILES: CC(C)(O)c1nc(CCO[Si](C)(C)C(C)(C)C)cs1 ; CC(C)(O)c1nc(CCO[Si](C)(C)C(C)(C)C)c(S(=O)(=O)Cl)s1]

Into a 250-mL round-bottom flask purged and maintained with an inert atmosphere of nitrogen, was placed a solution of 2-(4-(2-(tert-butyldimethylsilyloxy)ethyl)thiazol-2-yl)propan-2-ol (6.1 g, 20.2 mmol, 1 equiv) in THF (100 mL). n-BuLi (24.2 mL, 60.6 mmol, 2.5 M, 3 equiv) was added to the mixture at −78° C. and the resulting mixture stirred for another 30 min at −78° C. Then into the reaction mixture, SO₂ was bubbled for 30 min. This was followed by another 2 h of stirring at room temperature. The resulting mixture was concentrated. Then the resulting residue was dissolved in DCM(200 mL). NCS (5.39 g, 40.4 mmol, 2 equiv) was added to the mixture in portions at 0° C., and the mixture stirred for another 30 min at 0° C. The resulting mixture was concentrated at 0° C. This resulted in 4-(2-(tert-butyldimethylsilyloxy)ethyl)-2-(2-hydroxypropan-2-yl)thiazole-5-sulfonyl chloride (12.5 g) as a yellow solid, which was used directly for the next step without further purifications.

### 7. Synthesis of 4-(2-(tert-butyldimethylsilyloxy)ethyl)-2-(2-hydroxypropan-2-yl)thiazole-5-sulfonamide

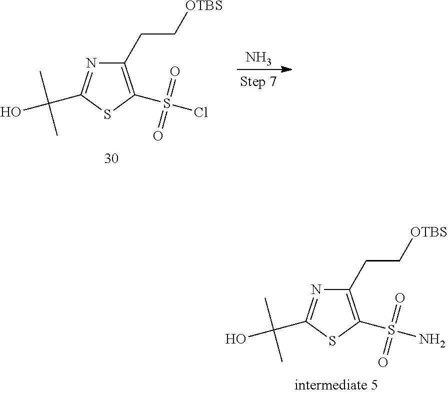

Into a 250-mL round-bottom flask, was placed a solution of 4-(2-(tert-butyldimethylsilyloxy)ethyl)-2-(2-hydroxypropan-2-yl)thiazole-5-sulfonyl chloride (12.5 g, 32.38 mmol, 1 equiv) in DCM (130 mL). NH3 was bubbled into the mixture for 10 min. The resulting solution was stirred for another 1 hr at room temperature, after which it was concentrated. The residue was applied onto a silica gel column with ethyl acetate/petroleum ether (1:5). This resulted in 4.8 g (62.5% for 2 steps) of 4-(2-(tert-butyldimethylsilyloxy)ethyl)-2-(2-hydroxypropan-2-yl)thiazole-5-sulfonamide as a yellow oil.

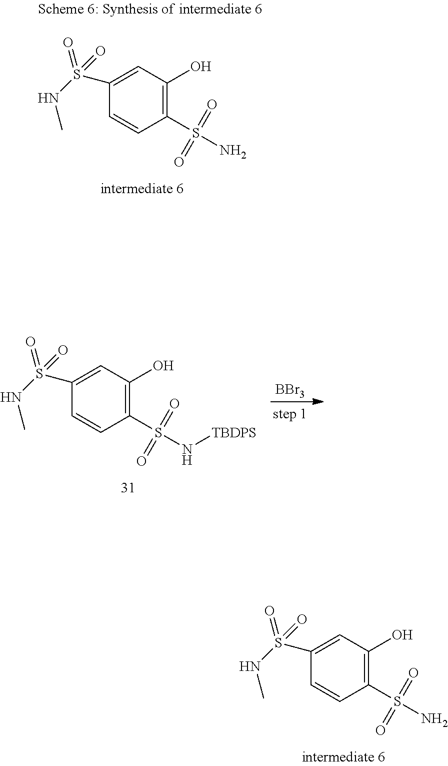

### 1. Synthesis of 3-(hydroxymethyl)-N1-methylbenzene-1,4-disulfonamide

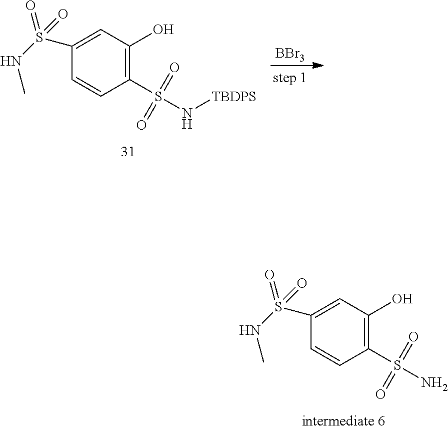

Into a 100 mL round-bottom flask were added N1-(tert-butyldiphenylsilyl)-2-methoxy-N4-methylbenzene-1,4-disulfonamide (518 mg, 1.0 mmol, 1 equiv) in DCM (20 mL). BBr3 (1 M in DCM) was added to the solution in dropwise at 0° C. The resulting mixture was stirred overnight at room temperature. The reaction mixture was purified by SiO₂-gel column, eluted with PE/EtOAc (1:1) to afford 3-hydroxy-N1-methylbenzene-1,4-disulfonamide (160 mg, 60.1%) as a yellow solid.

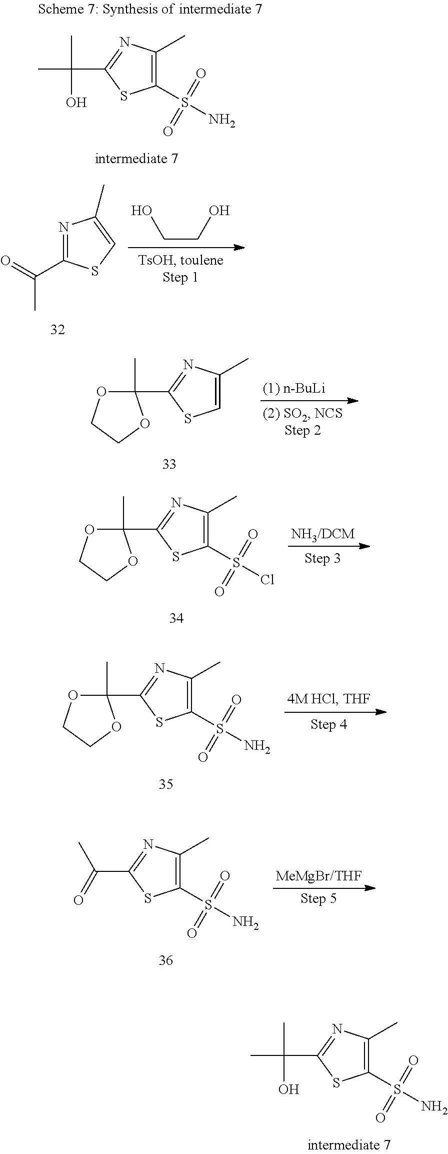

### 1. Synthesis of 4-methyl-2-(2-methyl-1,3-dioxolan-2-yl)-1,3-thiazole

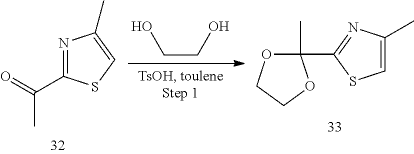

Into a 500-mL round-bottom flask purged and maintained with an inert, atmosphere of nitrogen, was placed 1-(4-methyl-1,3-thiazol-2-yl)ethan-1-one (11.7 g, 82.9 mmol, 1 equiv), toluene (200 mL), TsOH (1.4 g, 8.3 mmol, 0.1 equiv), and ethane-1,2-diol (25.5 g, 410.8 mmol, 5.0 equiv). The resulting solution was stirred for 16 hr at 110 degrees C. in an oil bath. The resulting mixture was concentrated. The resulting solution was diluted with 100 ml, of H₂O and extracted with 3×150 mL of ethyl acetate, dried over anhydrous sodium sulfate, filtered, and concentrated in vacuo. The resulting residue was applied onto a silica gel column with ethyl acetate/petroleum ether (1:10). This resulted in 14.2 g (92.5%) of 4-methyl-2-(2-methyl-1,3-dioxolan-2-yl)-1,3-thiazole as yellow oil.

### 2. Synthesis of 4-methyl-2-(2-methyl-1,3-dioxolan-2-yl)-1,3-thiazole-5-sulfonamide

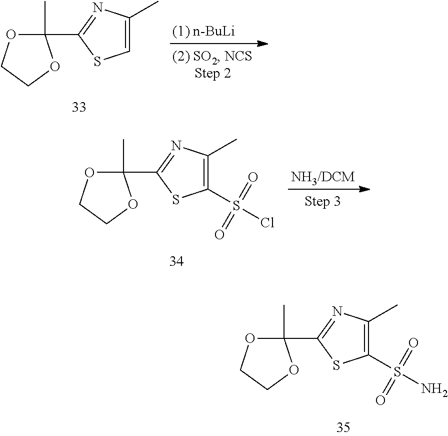 [SMILES: Cc1csc(C2(C)OCCO2)n1 ; Cc1nc(C2(C)OCCO2)sc1S(=O)(=O)Cl ; Cc1nc(C2(C)OCCO2)sc1S(N)(=O)=O]

Into a 250-mL 3-necked round-bottom flask purged and maintained with an inert atmosphere of nitrogen, was placed 4-methyl-2-(2-methyl-1,3-dioxolan-2-yl)-1,3-thiazole (14.2 g, 76.7 mmol, 1 equiv), THF (250 mL), n-BuLi (36.6 mL, 92 mmol, 1.2 equiv, 2.5M). The resulting solution was stirred for 30 min at −78 degrees C. with a liquid nitrogen bath, after which SO₂ (17.3 g, 270.0 mmol, 20 equiv) was at −50 degrees C. using a a liquid nitrogen bath. The resulting mixture was concentrated. The residue was dissolved in DCM, and NCS (12.2 g, 92 mmol, 1.2 equiv) was added at room temperature. The resulting solution was allowed to react, with stirring, for an additional 30 min at room temperature, after which NH₃/DCM (150 mL) was added at room temperature. The resulting solution was allowed to react for an additional 48 hr while the temperature was maintained at 45 degrees C. using an oil bath. The resulting mixture was concentrated. The residue was applied onto a silica gel column with ethyl acetate/petroleum ether (1:2). This resulted in 16.7 g (82.4%) of 4-methyl-2-(2methyl-1,3-dioxolan-2-yl)-1,3-thiazole-5-sulfonamide as a yellow solid.

### 3. Synthesis of 2-acetyl-4-methyl-1,3-thiazole-5-sulfonamide

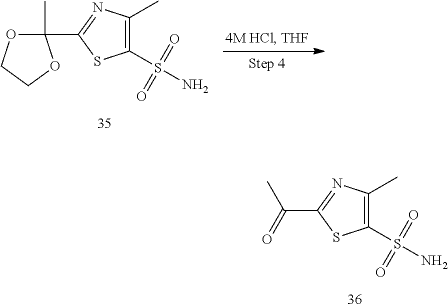 [SMILES: Cc1nc(C2(C)OCCO2)sc1S(N)(=O)=O ; CC(=O)c1nc(C)c(S(N)(=O)=O)s1]

Into a 500-mL round-bottom flask, was placed 4-methyl-2-(2-methyl-1,3-dioxolan-2-yl)-1,3-thiazole-5-sulfonamide (16.7 g, 63.2 mmol, 1 equiv) in THF (150 mL). HCl (100 mL, 3.3 mmol, 52 equiv, 4 M. in dioxane) was added to the solution. The resulting solution was stirred for 16 hr at 60 degrees C. in an oil bath. The resulting mixture was concentrated. The resulting solution was diluted with 100 mL of H₂O. The resulting solution was extracted with 3×150 ml of ethyl acetate dried over anhydrous sodium sulfate. The resulting mixture was concentrated. The residue was applied onto a silica gel column with ethyl acetate/petroleum ether (1:3). This resulted in 9.8 g (69.7%) of 2-acetyl-4methyl-1,3-thiazole-5-sulfonamide as a yellow solid.

### 4. Synthesis of 2-(2-hydroxypropan-2-yl)-4-methyl-1,3-thiazole-5-sulfonamide

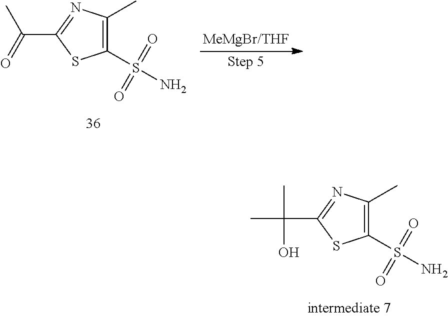

Into a 500-mL 3-necked round-bottom flask purged and maintained with an inert atmosphere of nitrogen, was placed 2-acetyl-4methyl-1,3-thiazole-5-sulfonamide (9.75 g, 44.3 mmol, 1 equiv), THF (400 mL), and MeMgBr (88 mL, 264.0 mmol, 6.0 equiv, 3M). The resulting solution was stirred for 16 h at room temperature. The reaction was then quenched by the addition of 100 mL of NH₄Cl. The pH value of the solution was adjusted to 4 with HCl (1 M in water). The resulting solution was extracted with 3×100 ml of ethyl acetate dried over anhydrous sodium sulfate and concentrated. The resulting residue was applied onto a silica gel column with ethyl acetate/petroleum ether (1:1). This resulted in 4 g (38.24%) of 2-(2-hydroxypropan-2-yl)-4-methyl-1,3-thiazole-5-sulfonamide as a yellow solid.

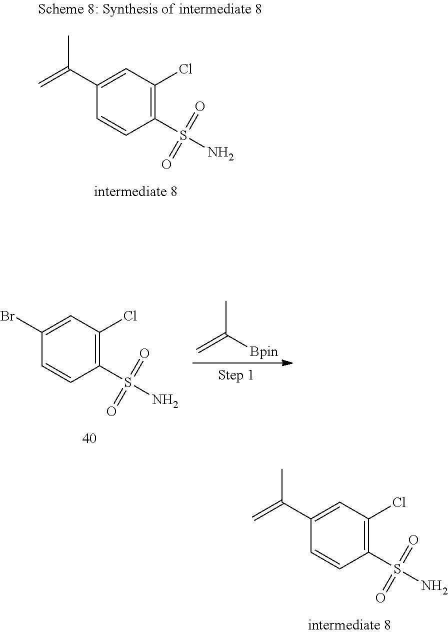

### 1. Synthesis of 2-chloro-4-(prop-1-en-2-yl)benzenesulfomide

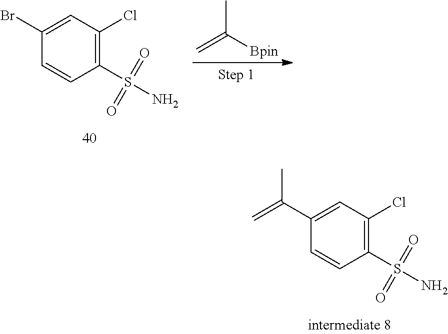

Into a 50-mL round-bottom flask, was added a solution of 4-bromo-2-chlorobenzenesulfonamide (1.0 g, 3.7 mmol, 1.0 equiv) in dioxane (20 mL)/water (2 mL). Pd(dppf)Cl2 (540.9 mg, 0.74 mmol, 0.2 equiv) and Cs2CO3 (2.4 g, 7.4 mmol, 2.0 equiv) were added to the solution. The resulting mixture was stirred for 6 h at 90 degrees C., after which it was concentrated and purified with SiO₂-gel column. This resulted in 720 mg (84.2%) of 2-chloro-4-(prop-1-en-2-yl)benzenesulfonamide as a yellow solid.

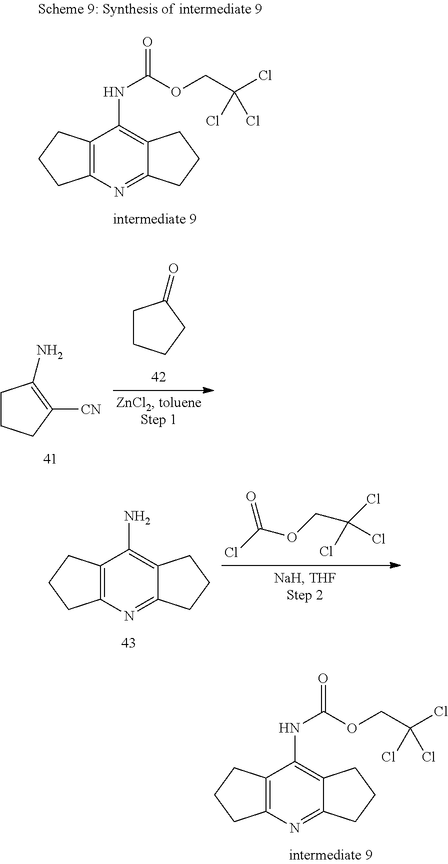

### 1. Synthesis of 1,2,3,5,6,7-hexahydrodicyclopenta[b,e]pyridin-8-amine

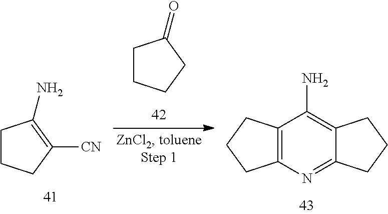 [SMILES: N#CC1=C(N)CCC1 ; O=C1CCCC1 ; Nc1c2c(nc3c1CCC3)CCC2]

Into a 2000-mL round-bottom flask purged and maintained with an inert atmosphere of nitrogen, was placed 2-aminocyclopent-1-enecarbonitrile (20.0 g, 185 mmol, 1 equiv) in toluene (1.0 L). To the solution were added cyclopentanone (15.5 g, 185 mmol, 1 equiv) and zinc(II) chloride (50.3 g, 370 mmol, 2 equiv). The resulting solution was stirred for 24 hr at 120° C. in an oil bath, after which the reaction was cooled to room temperature and filtered to collected the solid. The solid obtained was washed with water (500 mL×3) and Et₂O (500 mL), and dried in oven to give 1,2,3,5,6,7-hexahydrodicyclopenta[b,e]pyridin-8-amine (12 g, yield=37.2%) as a brown solid

LCMS of 1,2,3,5,6,7-hexahydrodicyclopenta[b,e]pyridin-8-amine (Method G): 175.2 [M+H]+, retention time 0.833 min. Method: Agilent Poroshell HPH-C18, 50*3.0 mm, 0.2 uL injection, 1.0 mL/min flowrate, 90-900 amu scan range, 254 nm UV detection. Mobile phase A: Water (5 mmoL/L NH4HCO3) and Mobile Phase B: MeCN. 5% MPB to 95.0% in 1.1 min, hold at 95% MPB for 0.5 min, 95% MPB to 10% in 0.1 min, then equilibration to 10% MPB for 0.1 min.

H-NMR (300 MHz, DMSO-d6) δ 5.41 (s, 2H), 2.65 (t, J=7.6 Hz, 4H), 2.57 (t, J=7.3 Hz, 4H), 1.94 (p, J=7.5 Hz, 4H).

### 2. Synthesis of 2,2,2-trichloromethyl (1,2,3,5,6,7-hexahydrodicyclopenta[b,e]pyridin-8-yl)carbamate

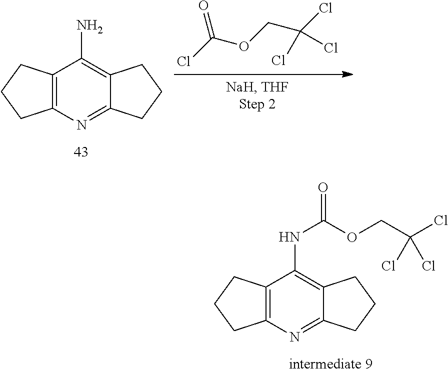

Into a 250-mL round-bottom flask, was placed a solution of 1,2,3,5,6,7-hexahydrodicyclopenta[b,e]pyridin-8-amine (12 g, 68.9 mmol, 1 equiv) in THF (500 mL). NaH(3.1 g, 77.8 mmol, 2 equiv, 60%) was added to the solution in portions at 0° C. The reaction was stirred at 40° C. for 48 h, after which it was quenched by the addition of 300 mL of ice-water. The resulting mixture was extracted with 3×100 ml of ethyl acetate; the combined organic phase was washed with water (3×300 mL) dried over Na₂SO₄, and concentrated. The residue was applied onto a silica gel column with ethyl acetate/petroleum ether (1:1). This resulted in 4.8 g (20.0%) of 2,2,2-trichloroethyl (1,2,3,5,6,7-hexahydrodicyclopenta[b,e]pyridin-8-yl)carbamate as brown solid and 6 g (50.0%) of 1,2,3,5,6,7-hexahydrodicyclopenta[b,e]pyridin-8-amine recycled.

LCMS of 2,2,2-trichloroethyl (1,2,3,5,6,7-hexahydrodicyclopenta[b,e]pyridin-8-yl)carbamate (Method H): 349.2, 351.2 [M+H]+, retention time 1.158 min. Method: Kinetex EVO C18, 50*3.0 mm, 0.3 uL injection, 1.2 mL/min flowrate, 90-900 amu scan range, 254 nm UV detection. Mobile phase A: Water (6.5 mmoL/L NH4HCO3, pH=10) and Mobile Phase B: MeCN. 5% MPB to 95.0% in 1.1 min, hold at 95% MPB for 0.5 min, 95% MPB to 10% in 0.1 min, then equilibration to 10% MPB for 0.21 min.

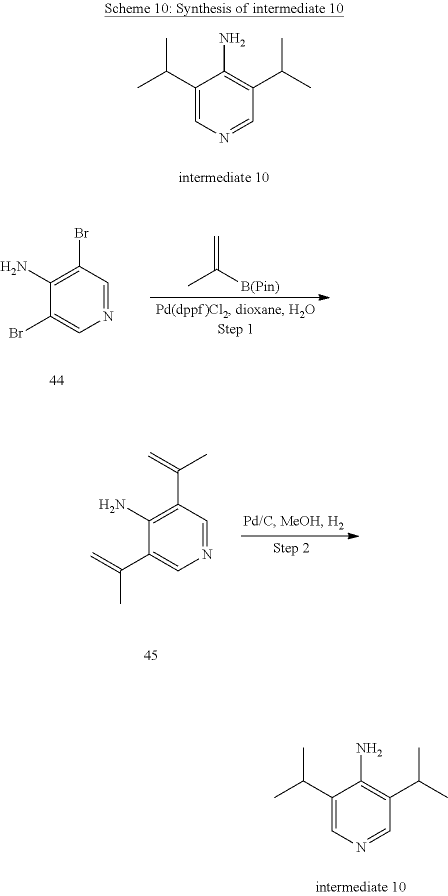

### 1. Synthesis of 3,5-bis(prop-1-en-2-yl)pyridin-4-amine

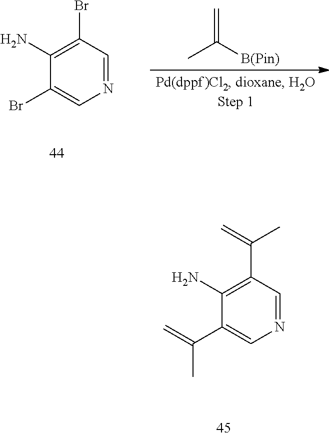

Into a 500-mL round-bottom flask, was placed 3,5-dibromopyridin-4-amine (5 g, 19.9 mmol, 1.0 equiv) in dioxane (150 mL) and water(15 mL). 4,4,5,5-tetramethyl-2-(prop-1-en-2-yl)-1,3,2-dioxaborolane (10.1 g, 60.0 mmol, 3.0 equiv), Cs₂CO₃ (19.6 g, 60.0 mmol, 3.0 equiv) and Pd(dppf)Cl₂ (1.5 g, 2.00 mmol, 0.03 equiv) were added to the solution. The resulting solution was stirred for 15 h at 90° C. in an oil bath. The resulting mixture was concentrated under vacuum. The residue was applied onto a silica gel column with ethyl acetate/petroleum ether (1:3). This resulted in 3.0 g (87.0%) of 3,5-bis(prop-1-en-2-yl)pyridin-4-amine as light yellow oil.

### 2. Synthesis of 3,5-bis(propan-2-yl)pyridin-amine

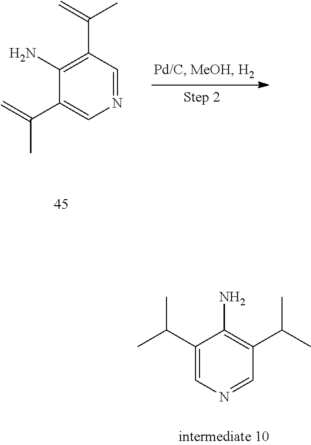

Into a 250-mL round-bottom flask, was placed 3,5-bis(prop-1-en-2-yl)pyridin-4-amine (3.0 g, 17.2 mmol, 1.0 equiv) in methanol (50 mL), Pd/C (300 mg, 5%) was added to the solution in one portion under N₂ atom sphere. The resulting solution was stirred for 1 overnight at room temperature under H₂ atmosphere. The solids were filtered out. The filterate was concentrated under vacuum. This resulted in 2.8 g (91%) of 3,5-bis(propan-2-yl)pyridin-4-amine as a light yellow solid.

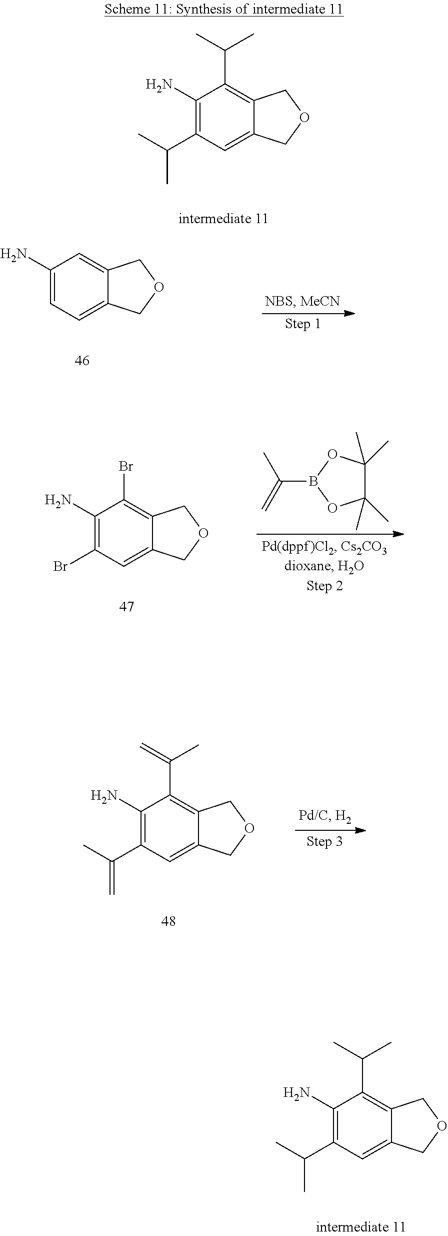 [SMILES: CC(C)c1cc2c(c(C(C)C)c1N)COC2 ; Nc1ccc2c(c1)COC2 ; Nc1c(Br)cc2c(c1Br)COC2 ; C=C(C)B1OC(C)(C)C(C)(C)O1 ; C=C(C)c1cc2c(c(C(=C)C)c1N)COC2 ; CC(C)c1cc2c(c(C(C)C)c1N)COC2]

### 1. Synthesis of 4,6-dibromo-1,3-dihydroisobenzofuran-5-amine

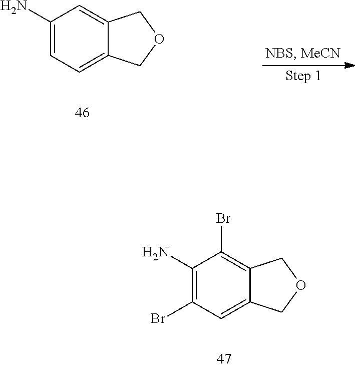 [SMILES: Nc1ccc2c(c1)COC2 ; Nc1c(Br)cc2c(c1Br)COC2]

Into a 250-mL round-bottom flask, was placed a solution of 1,3dihydroisobenzofuran-5-amine (13.5 g, 100 mmol, 1 equiv) in MeCN (200 mL). NBS (44.5 g, 250 mmol, 2.5 equiv) was added to the solution in portions. The resulting solution was stirred for another 5 h at room temperature. The resulting mixture was concentrated. The residue was applied onto a silica gel column with ethyl acetate/petroleum ether (1:5). This resulted in 26.3 g (91.0%) of 4,6-dibromo-1,3-dihydroisobenzofuran-5-amine as brown solid.

### 2. Synthesis of 4,6-di(prop-1-en-2-yl)-1,3-dihydroisobenzofuran-5-amine

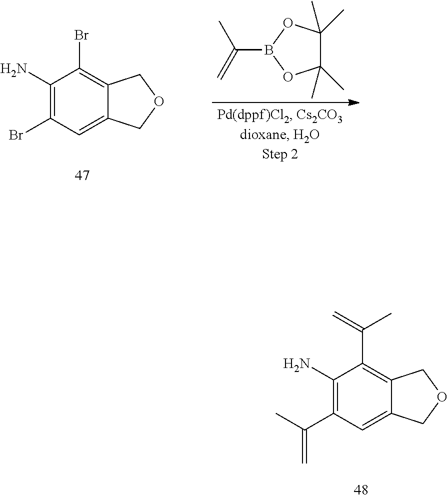 [SMILES: Nc1c(Br)cc2c(c1Br)COC2 ; C=C(C)B1OC(C)(C)C(C)(C)O1 ; C=C(C)c1cc2c(c(C(=C)C)c1N)COC2]

Into a 500-mL round-bottom flask, was placed 4,6-dibromo-1,3-dihydroisobenzofuran-5-amine (9.96 g, 34.0 mmol, 1.0 equiv) in dioxane (200 mL)/water (20 mL), Pd(dppf)Cl2 (5.0 g, 6.8 mmol, 0.2 equiv), 4,4,5,5-tetramethyl-2-(prop-1-en-2-yl)-1,3,2-dioxaborolane, and Cs₂CO₃ (22.2 g, 68.0 mmol, 2.0 equiv) were added to the solution. The resulting solution was stirred for 16 h at 90 degrees C. The resulting mixture was concentrated and purified with SiO₂-gel column. This resulted in 5.9 g (80.0%) of 4,6-di(prop-1-en-2-yl)-1,3-dihydroisobenzofuran-5-amine as a white solid.

### 3. Synthesis of 4,6-diisopropyl-1,3-dihydroisobenzofuran-5-amine

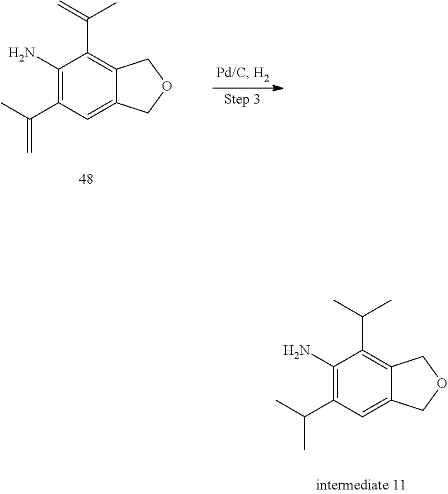 [SMILES: C=C(C)c1cc2c(c(C(=C)C)c1N)COC2 ; CC(C)c1cc2c(c(C(C)C)c1N)COC2]

Into a 500 mL round-bottom flask were added 4,6-di(prop-1-en-2-yl)-1,3-dihydroisobenzofuran-5-amine (5.9 g, 27.5 mmol, 1 equiv) and isopropanol(250 mL) at room temperature. Pd/C(580 mg, 5.5 mmol, 0.20 equiv) was added to the solution at room temperature under nitrogen atmosphere. The resulting mixture was stirred overnight at room temperature under hydrogen atmosphere, and then filtered to remove any solid. The filtrate was concentrated under reduced pressure to afford 4,6-diisopropyl-1,3-dihydroisobenzofuran-5-amine (5.4 g, 90.0%) as yellow solid.

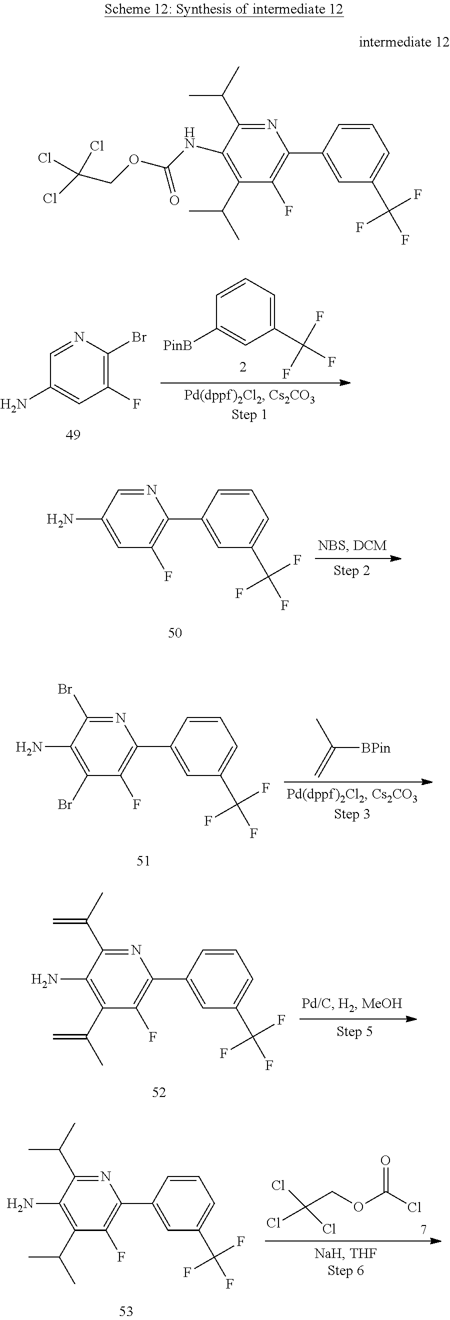

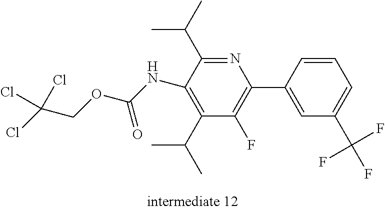 [SMILES: CC(C)c1nc(-c2cccc(C(F)(F)F)c2)c(F)c(C(C)C)c1NC(=O)OCC(Cl)(Cl)Cl]

### 1. Synthesis of 5-fluoro-6-(3-(trifluoromethyl)phenyl)pyridin-3-amine

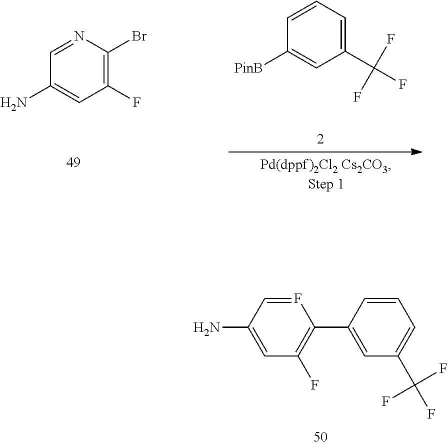

To a stirred solution of 6-bromo-5-fluoropyridin-3-amine (3 g, 15.7 mmol, 1 equiv) in dioxane (200 mL):H₂O (20 mL) were added Pd(dppf)₂Cl₂ (1.2 g, 0.1 equiv) and Cs₂CO₃ (10.2 g, 31.4 mmol, 2 equiv) at room temperature under nitrogen atmosphere. Then 4,4,5,5-tetramethyl-2-[3-(trifluoromethyl)phenyl]-1,3,2-dioxaborolane (17.1 mg, 62.8 mmol, 4 equiv) was added to the above mixture. After the addition was complete and the resulting mixture was stirred at 80 degrees C. in an oil bath overnight. The reaction was then to room temperature, and concentrated in vacuo. The resulting residue was purified by silica gel column chromatography, eluted with PE/EtOAc (12:1) to afford 5-fluoro-6-[3-(trifluoromethyl)phenyl]pyridin-3-amine (4.1 g, 94.7%) as a yellow oil.

### 2. Synthesis of 2,4-dibromo-5-fluoro-6-(3-(trifluoromethyl)phenyl)pyridin-3-amine

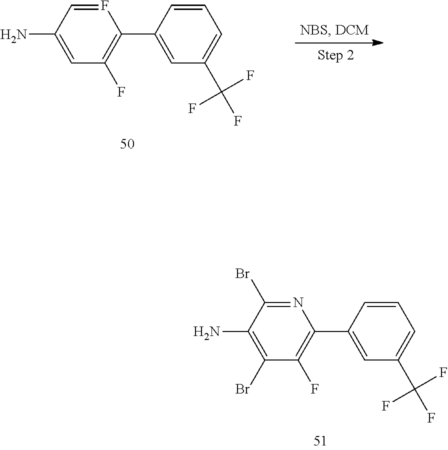

Into a 250 mL round-bottom flask was added a solution of 5-fluoro-6-[3-(trifluoromethyl)phenyl]pyridin-3-amine (4.1 g, 16 mmol, 1 equiv) in THF. HCl (13.5 mL, aqueous, 2M) was added to the solution. To this mixture was added Br₂ (2.5 mL, 48 mmol, 3.0 equiv) in dropwise. After the addition was complete and the resulting mixture was stirred for 4 h. The reaction was quenched with sat. Na₂S₂O₃ (aq.) at 0 degrees C. The resulting mixture was extracted with EtOAc(3×90 mL). The combined organic layers were dried over anhydrous Na₂SO₄. After filtration, the filtrate was concentrated under reduced pressure. The residue was purified by silica gel column chromatography, eluted with PE/EtOAc (20:1) to afford 2,4-dibromo-5-fluoro-6-[3-(trifluoromethyl)phenyl]pyridin-3-amine (2.3 g, 62.5%) as a yellow solid.

### 3. Synthesis of 5-fluoro-2,4-bis(prop-1-en-2-yl)-6-[3-(trifluoromethyl)phenyl]pyridin-3-amine

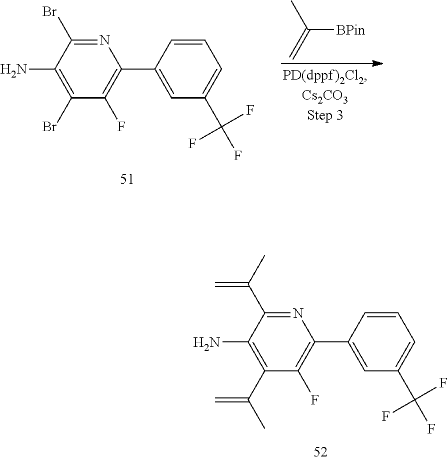

Into a 500 mL round-bottom flask were added 2,4-dibromo-5-fluoro-6-[3-(trifluoromethyl)phenyl]pyridin-3-amine(4.0 g, 9.7 mmol, 1 equiv) and Cs₂CO₃ (6.3 g, 19.3 mmol, 2 equiv) in dioxane(200 mL) and 1 LO(20 mL) at room temperature. Pd(dppf)₂C₁₋₂ (2.1 g, 0.3 equiv) and 4,4,5,5-tetramethyl-2-(prop-1-en-2-yl)-1,3,2-dioxaborolane (6.5 g, 38.7 mmol, 4 equiv) were added to the mixture in one portion at room temperature under nitrogen atmosphere. After the addition was complete and the resulting mixture was stirred at 100 degrees C. overnight. The residue was purified by silica gel column chromatography, eluted with PE/EtOAc (6:1) to afford 5-fluoro-2,4-bis(prop-1-en-2-yl)-6-[3-(trifluoromethyl)phenyl]pyridin-3-amine(2.9 g, 89.2%) as a yellow oil.

### 4. Synthesis of 5fluoro-2,4-diisopropyl-6(3(trifluoromethyl)phenyl)pyridin-3-amine

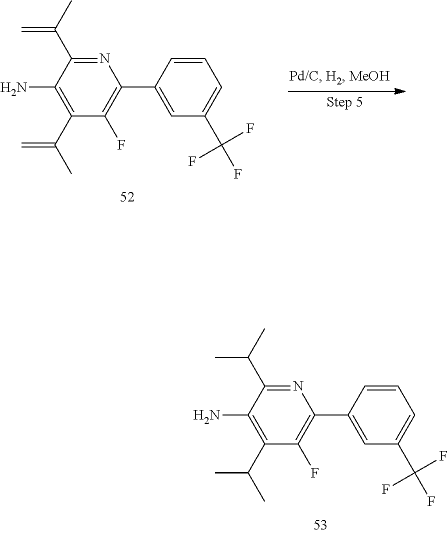 [SMILES: C=C(C)c1nc(-c2cccc(C(F)(F)F)c2)c(F)c(C(=C)C)c1N ; CC(C)c1nc(-c2cccc(C(F)(F)F)c2)c(F)c(C(C)C)c1N]

Into a 250 mL round-bottom flask were added 5-fluoro-2,4-bis(prop-1-en-2-yl)-6-[3-(trifluoromethyl)phenyl]pyridin-3-amine(2.9 g, 8.6 mmol, 1 equiv) and MeOH(150 mL) at room temperature. Pd/C(290 mg, 2.73 mmol, 0.32 equiv) was added to the solution in one portion at room temperature under nitrogen atmosphere. After the addition was complete and the resulting mixture was stirred for 3 days at room temperature under hydrogen atmosphere. The resulting mixture was filtered; the filter cake was washed with MeOH (20 mL). The filtrate was concentrated under reduced pressure and afford 3-bromo-5-fluoro-2,4-bis(propan-2-yl)-6-[3-(trifluoromethyl)phenyl]pyridine(2.1 g, 95.0%) as a yellow oil.

### 5. Synthesis of 2,2,2-trichloroethyl 5-fluoro-2,4-diisopropyl-6-(3-(trifluoromethyl)phenyl)pyridin-3-ylcarbamate

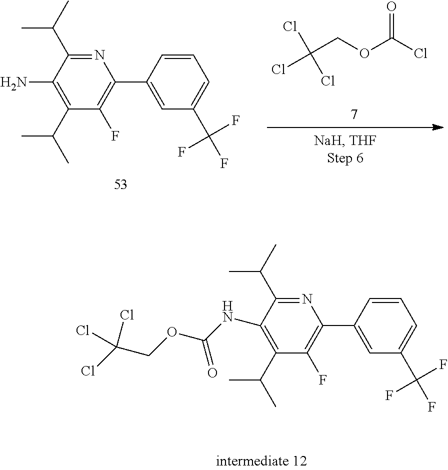 [SMILES: CC(C)c1nc(-c2cccc(C(F)(F)F)c2)c(F)c(C(C)C)c1N ; O=C(Cl)OCC(Cl)(Cl)Cl ; CC(C)c1nc(-c2cccc(C(F)(F)F)c2)c(F)c(C(C)C)c1NC(=O)OCC(Cl)(Cl)Cl]

Into a 50 mL round-bottom flask were added 5-fluoro-2,4-bis(propan-2-yl)-6-[3-(trifluoromethyl)phenyl]pyridin-3-amine (200 mg, 0.6 mmol, 1 equiv) at room temperature. To a stirred solution of PE/EtOAc in THF(10 mL) was added NaH (42.3 mg, 1.7 mmol, 3 equiv) in one portion at 0 degrees C. under nitrogen atmosphere. Then 2,2,2-trichloroethyl carbonochloridate (373.5 mg, 1.8 mmol, 3 equiv) was added to the above resulting mixture. After the addition was complete and the resulting mixture was stirred for 2 h. The reaction was quenched with ice-water (10 mL) at 0 degrees C. Extracted with EtOAc (10 mL*3), the combined organic phase was dried over Na₂SO₄, concentrated under reduced pressure. The residue was purified by SiO₂-gel column, eluted with PE/EtOAc (2:1) to afford 2,2,2-trichloroethyl N-[5-fluoro-2,4-bis(propan-2-yl)-6-[3-(trifluoromethyl)phenyl]pyri din-3-yl]carbamate (280 mg, 90.1%) as a white solid.

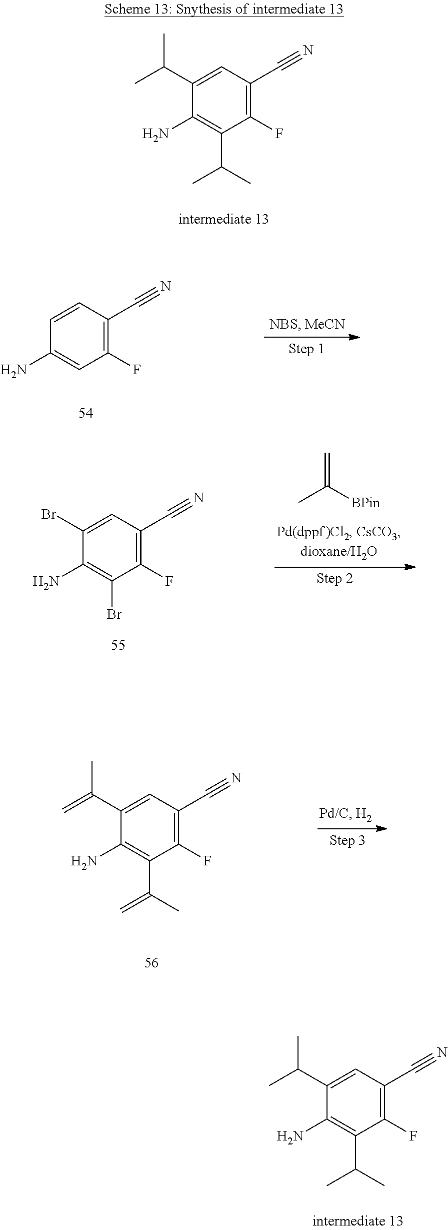

### 1. Synthesis of 4-amino-3,5dibromo-2fluorobenzonitrile

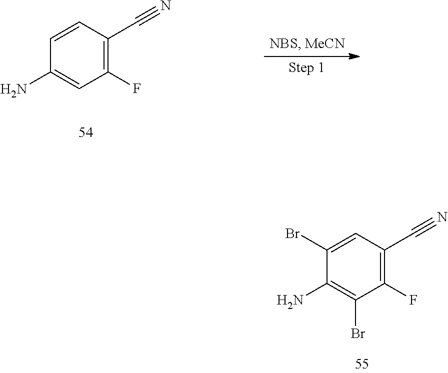 [SMILES: N#Cc1ccc(N)cc1F ; N#Cc1cc(Br)c(N)c(Br)c1F]

Into a 250-mL round-bottom flask, was placed a solution of 4-amino-2-fluorobenzonitrile (13.6 g, 100 mmol, 1 equiv) in MeCN (200 mL). NBS (44.5 g, 250 mmol, 2.5 equiv) was added to the solution in portions. The resulting solution was stirred for another 5 hr at room temperature. The resulting mixture was concentrated. The residue was applied onto a silica gel column with ethyl acetate/petroleum ether (1:5). This resulted in 26.4 g (91.0%) of 4-amino-3,5-dibromo-2-fluorobenzonitrile as brown solid

### 2. Synthesis of 4amino-2-fluoro-3,5-di(prop-1-en-2-yl)benzonitrile

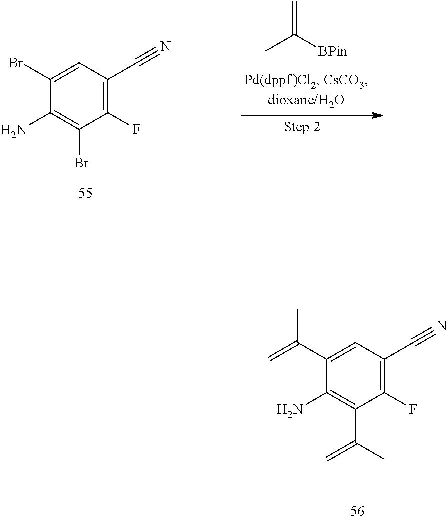

Into a 500-mL round-bottom flask, was placed 4-amino-3,5-dibromo-2-fluorobenzonitrile (10.0 g, 34.0 mmol, 1.0 equiv) in dioxane (200 mL), water (20 mL). Pd(dppf)Cl₂ (5.0 g, 6.8 mmol, 0.2 equiv) and Cs₂CO₃ (22.2 g, 68.0 mmol, 2.0 equiv) were added to the solution. The resulting solution was stirred for 16 hr at 90 degrees C., The resulting mixture was concentrated and purified with SiO₂-gel column. This resulted in 5.9 g (81.0%) of 4-amino-2-fluoro-3,5-di(prop-1-en-2-yl)benzonitrile as a white solid.

### 3. Synthesis of 4-amino-2-fluoro-3,5-diisopropylbenzonitrile

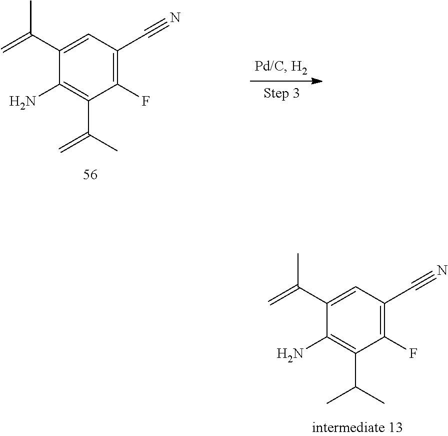 [SMILES: C=C(C)c1cc(C#N)c(F)c(C(=C)C)c1N ; C=C(C)c1cc(C#N)c(F)c(C(C)C)c1N]

Into a 500 mL round-bottom flask were added 4-amino-2-fluoro-3,5-di(prop-1-en-2-yl)benzonitrile (5.9 g, 27.5 mmol, 1 equiv) and isopropanol (250 mL) at room temperature. Pd/C (580 mg, 5.5 mmol, 0.20 equiv) was added to the solution at room temperature under nitrogen atmosphere. The resulting mixture was stirred overnight at room temperature under hydrogen atmosphere, and then filtered off the solid. The filtrate was concentrated under reduced pressure to affored 4-amino-2-fluoro-3,5-diisopropylbenzonitrile (5.1 g, 84.0%) as yellow solid.

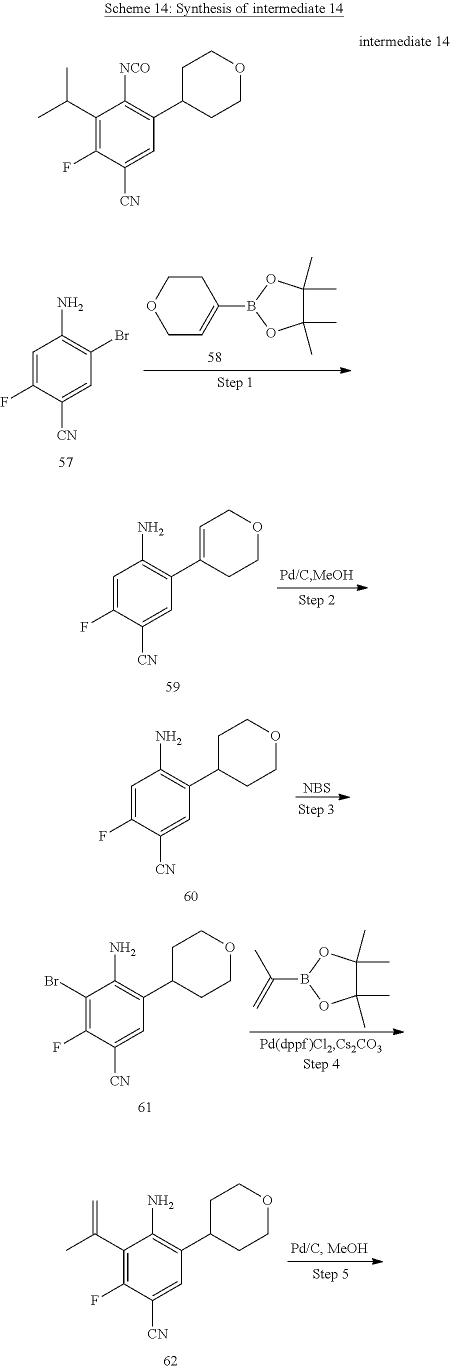

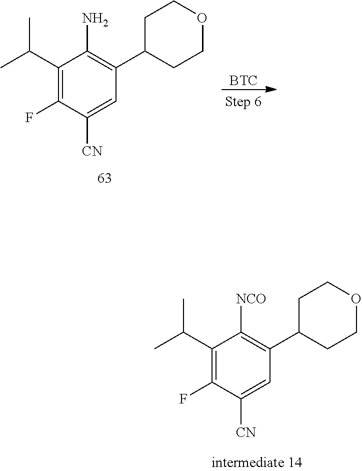

### 1. Synthesis of 4-amino-5-(3,6-dihydro-2H-pyran-4-yl)-2-fluorobenzonitrile

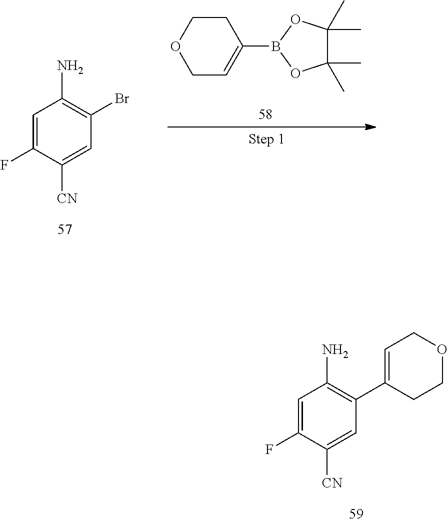 [SMILES: N#Cc1cc(Br)c(N)cc1F ; CC1(C)OB(C2=CCOCC2)OC1(C)C ; N#Cc1cc(C2=CCOCC2)c(N)cc1F]

Into a 100-mL round-bottom flask purged and maintained with an inert atmosphere of nitrogen, was placed 4-amino-5-bromo-2-fluorobenzonitrile (3 g, 14.0 mmol, 1 equiv), 2-(3,6-dihydro-2H-pyran-4-yl)-4,4,5,5-tetramethyl-1,3,2-dioxaborolane (3.5 g, 16.7 mmol, 1.2 equiv), and dioxane (30 mL): H2O (3 mL), Pd(dppf)Cl2 (1.0 g, 1.4 mmol, 0.1 equiv) and Cs₂CO₃ (13.6 g, 41.9 mmol, 3 equiv) were added to the mixture under N₂ atmosphere. The resulting solution was stirred for 12 h at 90 degrees C. The resulting mixture was concentrated. The residue was applied onto a silica gel column with ethyl acetate/petroleum ether (1:5). This resulted in 2.5 g (82.1%) of 4-amino-5-(3,6-dihydro-2H-pyran-4-yl)-2-fluorobenzonitrile as a yellow solid.

### 2. Synthesis of 4-amino-2-fluoro-5-(tetrahydro-2H-pyran-4-yl)benzonitrile

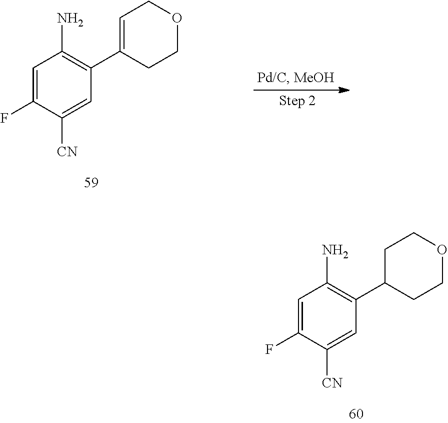 [SMILES: N#Cc1cc(C2=CCOCC2)c(N)cc1F ; N#Cc1cc(C2CCOCC2)c(N)cc1F]

Into a 100-mL round-bottom flask, was placed a solution of 4-amino-5-(3,6-dihydro-2H-pyran-4-yl)-2-fluorobenzonitrile (2.5 g, 11.5 mmol, 1 equiv) in MeOH (20 mL). Pd/C (0.3 g, 2.4 mmol, 0.21 equiv) was added to the solution under N₂ atmosphere. The resulting solution was stirred for 12 h at room temperature under H₂ atmosphere. The solids were filtered out. The filtrate v/as concentrated. This resulted in 1 g (39.6%) of 4-amino-2-fluoro-5-(oxan-4-yl)benzonitrile as a yellow solid,

### 3. Synthesis of 4-amino-3-bromo-2-fluoro-5-(tetrahydro-2H-pyran-4-yl)benzonitrile

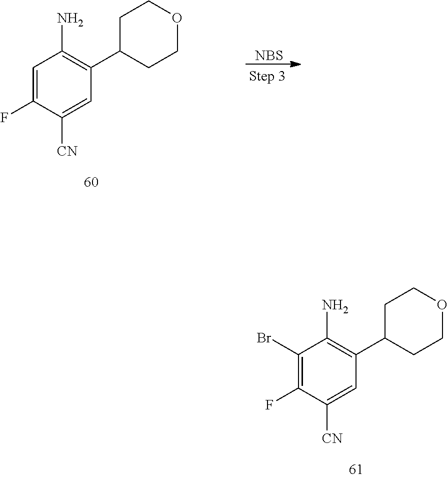 [SMILES: N#Cc1cc(C2CCOCC2)c(N)cc1F ; "NBS" ; N#Cc1cc(C2CCOCC2)c(N)c(Br)c1F]

Into a 50-mL round-bottom flask, was placed a solution of 4-amino-2-fluoro-5-(oxan-4-yl)benzonitrile (1 g, 4.5 mmol, 1 equiv) in MeCN (10 mL) and NBS (1.0 g, 5.5 mmol, 1.2 equiv). The resulting solution was stirred for 30 min at room temperature. The reaction was then quenched by the addition of 10 ml, of Na₂SO₃/H₂O. The resulting solution was extracted with of EtOAc (50 mL*3), the combined organic phase was dried over Na₂SO₄ and concentrated under reduced pressure. This resulted in 1.2 g (88.4%) of 4amino-3-bromo-2-fluoro-5-(oxan-4-yl)benzonitrile as a yellow solid.

### 4. Synthesis of 4-amino-2-fluoro-3-(prop-1-en-2-yl)-5-(tetrahydro-2H-pyran-4-yl)benzonitrile

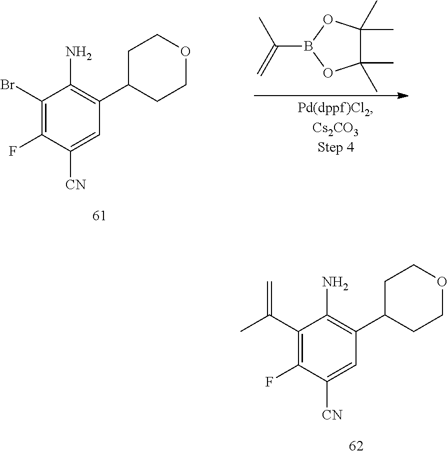 [SMILES: N#Cc1cc(C2CCOCC2)c(N)c(Br)c1F ; C=C(C)B1OC(C)(C)C(C)(C)O1 ; C=C(C)c1c(N)c(C2CCOCC2)cc(C#N)c1F]

Into a 50-mL round-bottom flask purged and maintained with an inert atmosphere of nitrogen, was added a solution of 4-amino-3-bromo-2-fluoro-5-(oxan-4-yl)benzonitrile (1.2 g, 4.0 mmol, 1 equiv) and 4,4,5,5-tetramethyl-2-(prop-1-en-2-yl)-1,3,2-dioxaborolane (0.8 g, 4.8 mmol, 1.2 equiv) in dioxane (10 mL): H₂O(l mL). Pd(dppf)Cl2 (0.3 g, 0.40 mmol, 0.1 equiv) and Cs₂CO3 (3.9 g, 12.0 mmol, 3 equiv) were added to the mixture. The resulting solution was stirred for 12 h at 90 degrees C., The resulting mixture was concentrated. The residue was applied onto a silica gel column with ethyl acetate/petroleum ether (1:5). This resulted in 500 mg (47.9%) of 4-amino-2-fluoro-5-(oxan-4-yl)-3-(prop-1-en-2-yl)benzonitrile as a yellow solid.

### 5. Synthesis of 4-amino-2-fluoro-3-isopropyl-5-(tetrahydro-2H-pyran-4-yl)benzonitrile NH₂ NH₂

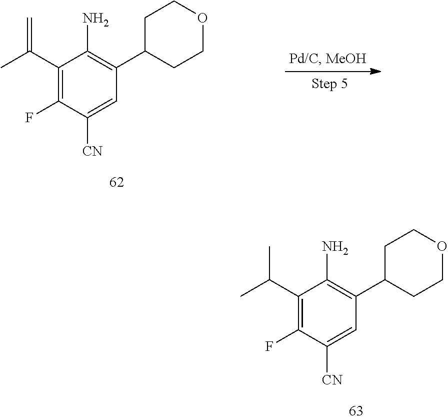 [SMILES: C=C(C)c1c(N)c(C2CCOCC2)cc(C#N)c1F ; CC(C)c1c(N)c(C2CCOCC2)cc(C#N)c1F]

Into a 25-mL round-bottom flask, was placed 4-amino-2-fluoro-5-(oxan-4-yl)-3-(prop-1-en-2-yl)benzonitrile (500 mg, 1.9 mmol, 1 equiv) in MeOH (20 mL). Pd/C (49.1 mg, 0.46 mmol, 0.24 equiv) was added to the solution in one portion under N2 atmosphere. The resulting solution was stirred for 24 h at room temperature under H2 atmosphere. The solids were filtered out. The resulting mixture was concentrated. The residue was applied onto a silica gel column with ethyl acetate/petroleum ether (1:5). This resulted in 400 mg (79.4%) of 4-amino-2-fluoro-5-(oxan-4-yl)-3-(propan-2-yl)benzonitrile as a light yellow solid.

### 6. Synthesis of 2-fluoro-4-isocyanato-3-isopropyl-5-(tetrahydro-2H-pyran-4-yl)benzonitrile

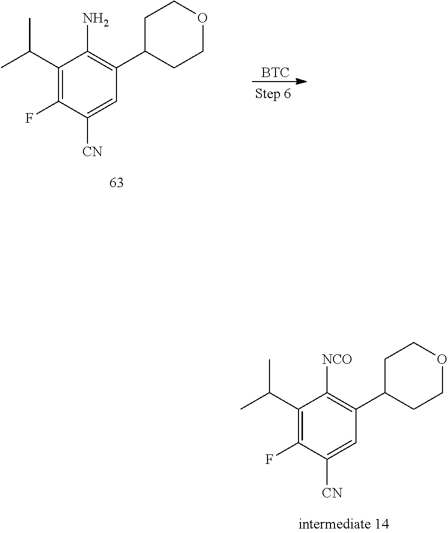

Into a 50-mL round-bottom flask, was placed a solution of 4-amino-2-fluoro-5-(oxan-4-yl)-3-(propan-2-yl)benzonitrile (100 mg, 0.38 mmol, 1 equiv) in THF (5 mL), ditrichloromethyl carbonate (56.6 mg, 0.19 mmol, 0.5 equiv), and TEA (77.1 mg, 0.76 mmol, 2 equiv). The resulting solution was stirred for 2 h at 60 degrees C. The resulting mixture was concentrated. This resulted in 105 mg (95.5%) of 2-fluoro-4-isocyanato-5-(oxan-4-yl)-3-(propan-2-yl)benzonitrile as a yellow solid.

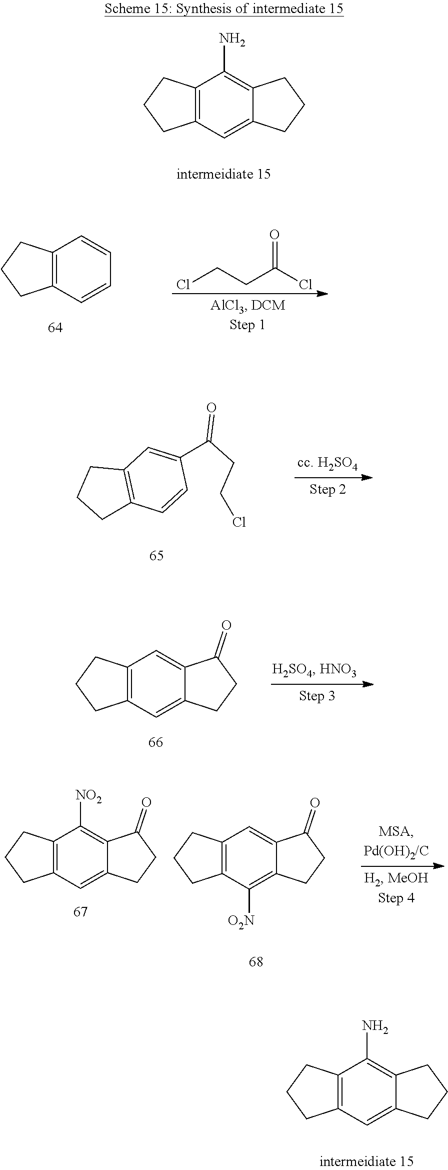

### 1. Synthesis of 3-Chloro-1-(2,3-dihydro-1H-inden-5-yl)propan-1-one

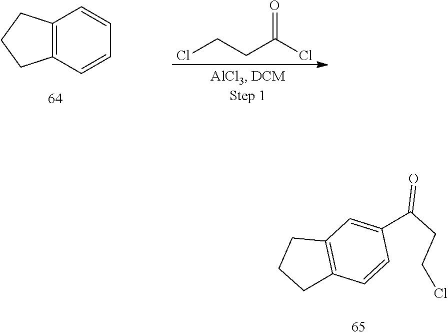 [SMILES: c1ccc2c(c1)CCC2 ; O=C(Cl)CCCl ; O=C(CCCl)c1ccc2c(c1)CCC2]

Into a 3000-mL round-bottom flask was placed a solution of AlCl₃ (111.0 g, 834.0 mmol) in DCM (1200 mL). This was followed by the addition of a solution of 2,3-dihydro-1H-indene (90.0 g, 762.0 mmol) and 3-chloropropanoyl chloride (96.3 g, 759.0 mmol) in DCM (300 mL) dropwise with stirring at −10° C. in 30 min. The resulting solution was stirred for 16 h at RT. Then the reaction mixture was added dropwise to cold HCl (3 N, 1200 mL) over 45 min at −10° C. The resulting solution was extracted with 3×600 mL of DCM and the organic layers were combined, dried over anhydrous Na₂SO₄, then concentrated under vacuum. This resulted in 160.5 g (crude) of the title compound as a yellow solid. The crude product was used in the next step. MS-ESI: 209, 211 (M+1).

### 2. Synthesis of 1,2,3,5,6,7-Hexahydro-s-indacen-1-one

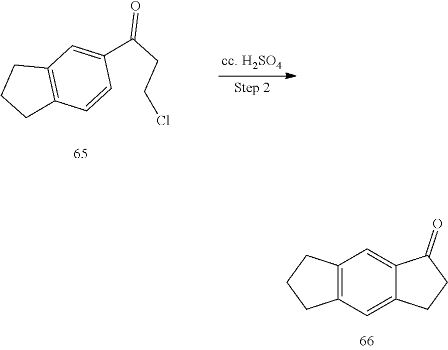 [SMILES: O=C(CCCl)c1ccc2c(c1)CCC2 ; O=C1CCc2cc3c(cc21)CCC3]

Into a 1000-mL round-bottom flask was placed a solution of 3-chloro-1-(2,3-dihydro-1H-inden-5-yl)propan-1-one (160.5 g, 759.0 mmol) in cone. H₂SO₄ (900 mL). The resulting solution was stirred for 16 h at 55° C. and was then quenched by adding the reaction mixture carefully to 4500 mL of water/ice. The solids were collected by filtration and dried over infrared lamp for 24 h. This resulted in 112.2 g (85%) of the title compound as a yellow solid,

### 3. Synthesis of 4-nitro-2,3,6,7-tetrahydros-indacen-1(5H)-one (68) and § nitro-2,3,6,7-tetrahydros-indacen-1(5H)-one (67)

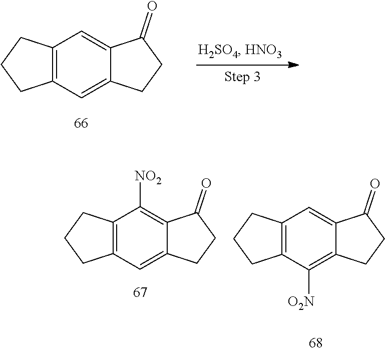

Into a 1000-mL round-bottom flask was placed a solution of 1,2,3,5,6,7-hexahydro-s-indacen-1-one (80.0 g, 464.5 mmol) in H2SO₄ (500 mL). Then HNO₃ (58.5 g, 929.0 mmol) was added dropwise over 1 h at 0° C. The resulting solution was stirred for 1 h at 0° C. The reaction mixture was slowly added to a mixture of water/ice (1000 mL) and DCM (500 mL) with ice bath cooling. The organic layer was collected, dried over Na₂SO₄ and concentrated under vacuum. This resulted in 90.0 g (90%) of the mixture of 4-nitro-1,2,3,5,6,7-hexahydro-s-indacen-1-one and 8-nitro-2,3,6,7-tetrahydros-indacen-1(5H)-one as a yellow solid.

### 4. Synthesis of 1,2,3,5,6,7-hexahydros-indacen-4-amine

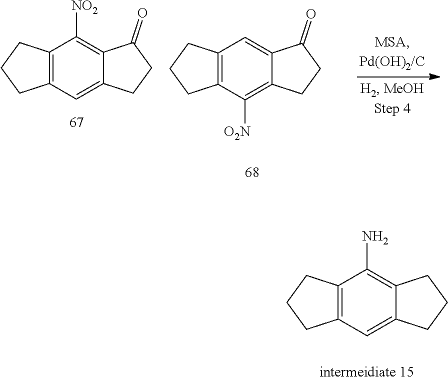

Into a 1000-mL round-bottom flask was placed a solution of the mixture of 4-nitro-1,2,3,5,6,7-hexahydro-s-indacen-1-one and 8-nitro-2,3,6,7-tetrahydros-indacen-1(5H)-one (21.7 g, 100.0 mmol) in MeOH (300 mL). To the solution was added MSA (11.5 g, 120.0 mmol). Then Pd(OH)₂/C (20% wt, 5.5 g) was added. The flask was evacuated and filled three times with hydrogen. The resulting mixture was stirred for 16 h at 25° C. under hydrogen (50 psi). The solids were filtered out and washed with methanol. The methanol filtrate and wash was diluted with water (500 mL) and the pH was adjusted to 10.6 with 2N NaOH. The resulting slurry was filtered and the crude solids were recrystallized from methanol/water (9:1) with heating. This resulted in 13.7 g (79%) of the title compound as an off-white solid. MS-ESI: 174 (M+1).

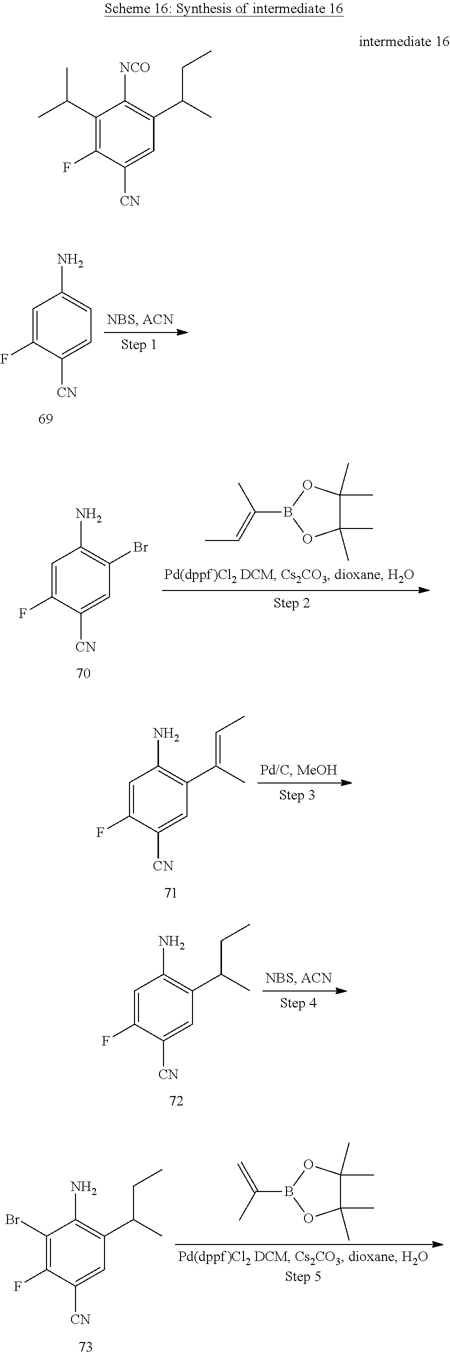

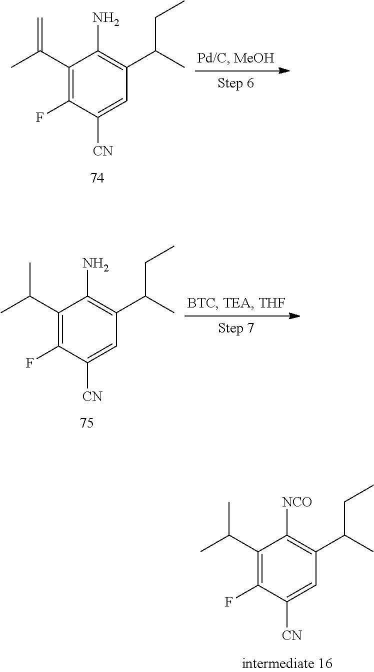

### 1. Synthesis of 4-amino-5-bromo-2-fluorobenzonitrile

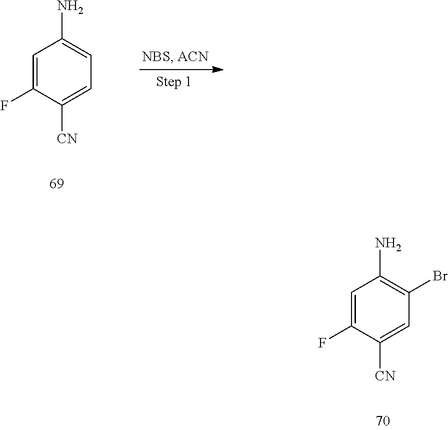 [SMILES: N#Cc1ccc(N)cc1F ; N#Cc1cc(Br)c(N)cc1F]

A solution of 4-amino-2-fluorobenzonitrile(2 g, 14.7 mmol, 1 equiv) and NBS (2.6 g, 14.7 mmol, 1 equiv) in MeCN (100 mL) was stirred for 4 h at 65° C. under nitrogen atmosphere. The resulting mixture was concentrated under vacuum. The residue was purified by silica gel column chromatography, eluted with PE/EtOAc (13:1) to afford 4-amino-5-bromo-2-fluorobenzonitrile(3.1 g, 97.2%) as a yellow solid.

### 2. Synthesis of (E)-4-amino-5-(but-2-en-2-yl)-2-fluorobenzonitrile

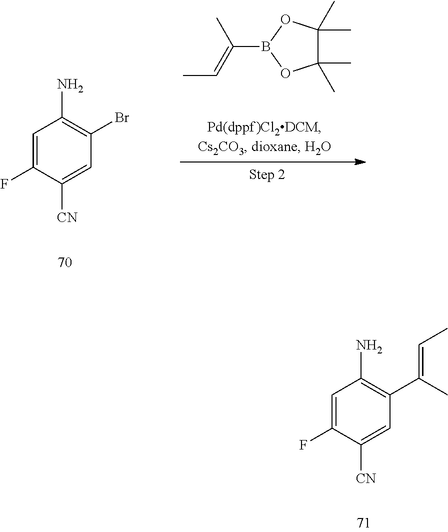

To a stirred solution/mixture of 4-amino-5-bromo-2-fluorobenzonitrile(1.0 g, 4.65 mmol, 1.0 equiv), Cs₂CO₃(454.6 mg, 1.4 mmol, 3.0 equiv) and 2-[(2Z)-but-2-en-2-yl]-4,4,5,5-tetramethyl-1,3,2-dioxaborolane (1.0 g, 5.6 mmol, 1.2 equiv) in 1,4-dioxane and H₂O (0.6 mL) were added Pd(dppf)Cl₂ DCM (76.0 mg, 0.1 mmol, 0.2 equiv) under nitrogen atmosphere. The resulting mixture was stirred overnight at 90° C. under nitrogen atmosphere. The residue was purified by silica gel column chromatography, eluted with PE/EtOAc (50:1) to afford 4-amino-5-[(2E)-but-2-en-2-yl]-2-fluorobenzonitrile (600 mg, 67.8%) as a yellow solid.

LC-MS-4-amino-5-[(2E)-but-2-en-2-yl]-2-fluorobenzonitrile: (ES, m/z): [M+H]⁺=191.1

### 3. Synthesis of 4-amino-5-sec-butyl-2-fluorobenzonitrile

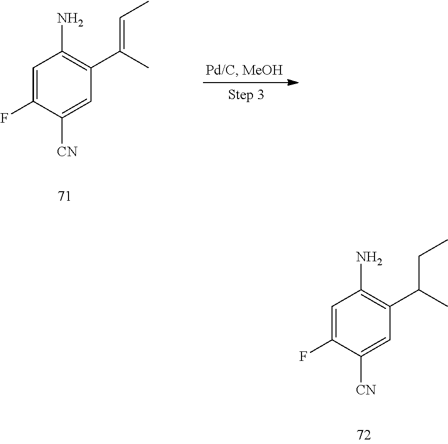

A solution/mixture of 4-amino-5-[(2E)-but-2-en-2-yl]-2-fluorobenzonitrile (1.2 g, 6.3 mmol, 1 equiv) in MeOH (20 mL). Pd/C was added to the solution in one portion under N₂ atmosphere. The mixture was stirred overnight at room temperature under hydrogen atmosphere. The resulting mixture was filtered; the filter cake was washed with MeOH (3×20 mL). The filtrate was concentrated under reduced pressure. This resulted in 4-amino-5-(butan-2-yl)-2-fluorobenzonitrile(l g, 82.5%) as a yellow solid.

LC-MS-4-amino-5-(butan-2-yl)-2-fluorobenzonitrile: (ES, m/z): [M+H]⁺=193.1

### 4. Synthesis of 4-amino-3-bromo-5-(butan-2-yl)-2-fluorobenzonitrile

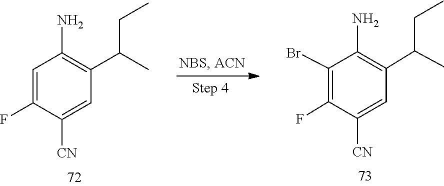

A solution/mixture of 4-amino-5-(butan-2-yl)-2-fluorobenzonitrile(l g, 5.20 mmol, 1 equiv) and NBS(1.4 g, 7.8 mmol, 1.5 equiv) in ACN (100 mL) was stirred for 3 h at 65° C. under nitrogen atmosphere. The resulting mixture was concentrated under vacuum. The resulting residue was purified by silica gel column chromatography and eluted with PE/EtOAc (100:1) to afford 4-amino-3-bromo-5-(butan-2-yl)-2-fluorobenzonitrile(1.2 g, 85.1%) as a yellow solid.

### 5. Synthesis of 4-amino-5-sec-butyl-2-fluoro-3-(prop-1-en-2-yl)benzonitrile

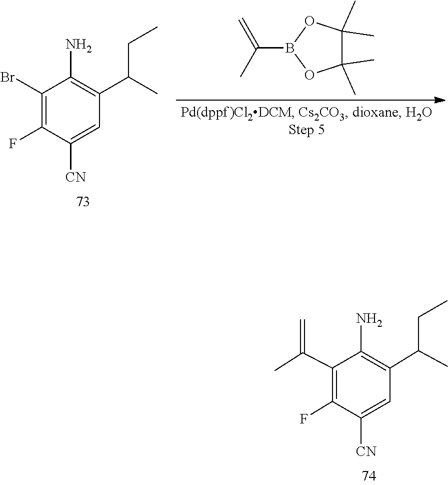

To a stirred solution/mixture of 4-amino-3-bromo-5-(butan-2-yl)-2-fluorobenzonitrile (600 mg, 2.2 mol, 1 equiv), 4,4,5,5-tetramethyl-2-(prop-1-en-2-yl)-1,3,2-dioxaborolane (557.8 mg, 3.3 mol, 1.5 equiv), Cs₂CO₃ (2.2 g, 6.6 mmol, 3.0 equiv) and H2O (0.6 mL) in 1,4-dioxane was added Pd(dppf)Cl₂ DCM (361.6 mg, 0.44 mmol, 0.2 equiv) at room temperature under nitrogen atmosphere. The resulting mixture was stirred for overnight at 90° C. under nitrogen atmosphere. The residue was purified by silica gel column chromatography, eluted with PE/EtOAc (5:1) to afford 4-amino-5-(butan-2-yl)-2-fluoro-3-(prop-1-en-2-yl)benzonitrile(350 mg, 68.08%) as a yellow solid.

LC-MS-4-amino-5-(butan-2-yl)-2-fluoro-3-(prop-1-en-2-yl)benzonitrile: (ES, m/z) [M+H]⁺=233.2

### 6. Synthesis of 4-amino-5-sec-butyl-2-fluoro-3-isopropylbenzonitrile

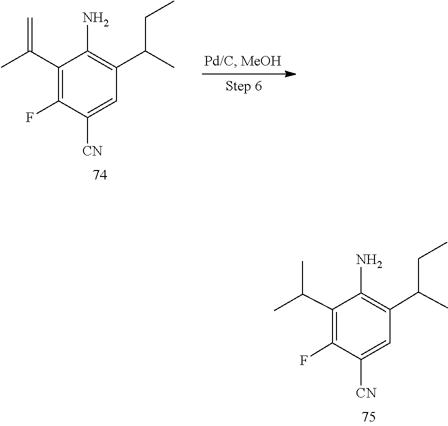 [SMILES: C=C(C)c1c(N)c(C(C)CC)cc(C#N)c1F ; CCC(C)c1cc(C#N)c(F)c(C(C)C)c1N]

A solution/mixture of 4-amino-5-(butan-2-yl)-2-fluoro-3-(prop-1-en-2-yl)benzonitrile (700 mg, 3.01 mol, 1 equiv) in MeOH (20 mL). Pd/C was added to the solution in one portion under N₂ atmosphere. The mixture was stirred for overnight at room temperature under hydrogen atmosphere. The resulting mixture was filtered; the filter cake was washed with MeOH (3×30 mL). The filtrate was concentrated under reduced pressure. This resulted in 4-amino-5-(butan-2 yl)-2-fluoro-3-(propan-2-yl)benzonitrile (700 mg, crude) as a yellow solid.

LC-MS: (ES, m/z): [M+H]⁺=235.2

### 7. Synthesis of 5-(sec-butyl)-2-fluoro-4-isocyanato-3-isopropylbenzonitrile

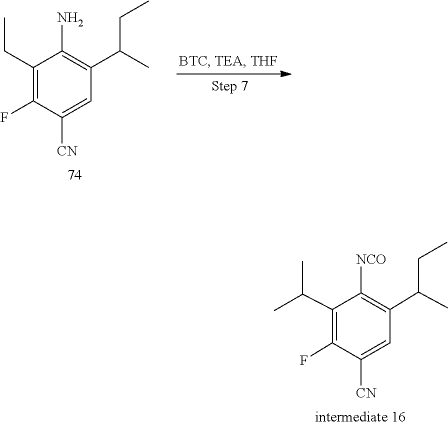

To a stirred solution/mixture of 4-amino-5-(butan-2-yl)-2fluoro-3-(propan-2-yl)benzonitrile (300 mg, 1.28 mol, 1.0 equiv) and TEA (259.1 mg, 2.6 mmol, 2.0 equiv) in THF (15 mL) was added BTC(190.1 mg, 0.6 mmol, 0.5 equiv) at room temperature under nitrogen atmosphere. The resulting mixture was stirred for 3 h at 65° C. under nitrogen atmosphere. The resulting mixture was concentrated under vacuum to provide 5-(sec-butyl)-2fluoro-4-isocyanato-3-isopropylbenzonitrile.

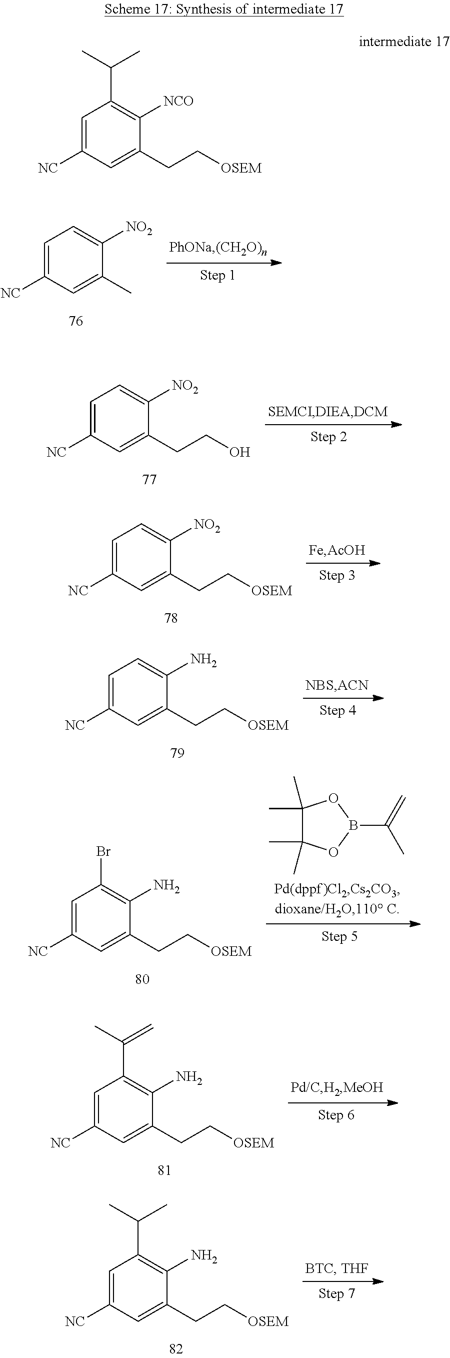

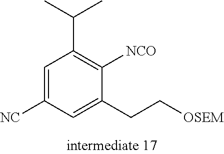

### 1. Synthesis of 3-(2-hydroxyethyl)-4-nitrobenzonitrile

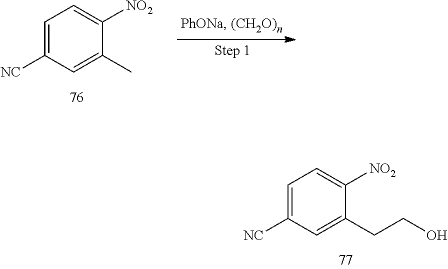

Into a 250-mL round-bottom flask, was placed a solution of 3-methyl-4-nitrobenzonitrile (10 g, 61.7 mmol, 1.0 equiv) in DMSO (100 mL), formaldehyde (5.6 g, 185.0 mmol, 3.0 equiv), and (sodiooxy)benzene (0.7 g, 6.2 mmol, 0.1 equiv). The resulting solution was stirred for 2 hr at 90 degrees C., after which it was diluted with 500 mL of water, extracted with 3×300 ml of ethyl acetate, and concentrated. This resulted in 3 g (25.3%) of 3-(2-hydroxyethyl)-4-nitrobenzonitrile as yellow oil.

### 2. Synthesis of 4-nitro-3-(2-((2-(trimethylsilyl)ethoxy)methoxy)ethyl)benzonitrile

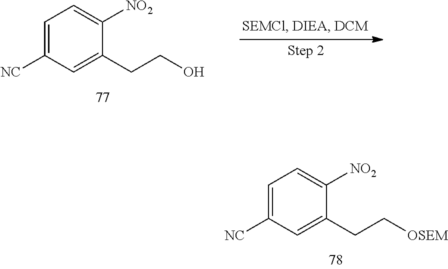

Into a 100-mL round-bottom flask, was placed a solution of 3-(2-hydroxyethyl)-4-nitrobenzonitrile (3.0 g, 15.6 mmol, 1 equiv) in THF (30 mL), SEM-Cl (5.2 g, 31.2 mmol, 2 equiv), and DIE A (8.1 g, 62.4 mmol, 4 equiv). The resulting solution was stirred for 2 h at room temperature, after which it was diluted with 50 mL of water, extracted with 3×100 ml of ethyl acetate, and concentrated. This resulted in 4 g (79.5%) of 4-nitro-3-(2-[[2-(trimethylsilyl)ethoxy]methoxy]ethyl)benzonitrile as yellow oil.

### 3. Synthesis of 4-amino-3-(2-((2-(trimethylsilyl)ethoxy)methoxy)ethyl)benzonitrile

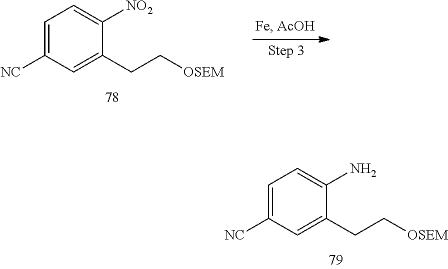

Into a 250-mL round-bottom flask, was placed 4-nitro-3-(2-[[2-(trimethylsilyl)ethoxy]methoxy]ethyl)benzonitrile (4 g, 12.4 mmol, 1 equiv), Fe (3.5 g, 62.0 mmol, 5 equiv), CH₃COOH (40 mL), and H2O (40 mL). The resulting solution was stirred for 2 h at 50 degrees C. The resulting solution was extracted with 3×100 ml of ethyl acetate and concentrated. This resulted in 3.5 g (96.5%) of 4-amino-3-(2-[[2-(trimethylsilyl)ethoxy]methoxy]ethyl)benzonitrile as yellow oil.

### 4. Synthesis of 4-amino-3-bromo-5-(2-((2-(trimethylsilyl)ethoxy)methoxy)eethyl) benzonitrile

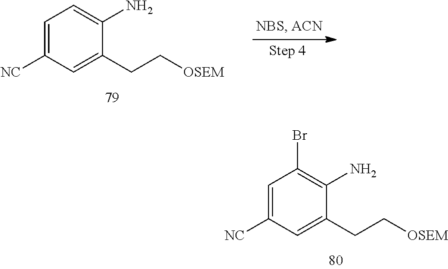

Into a 50-mL round-bottom flask, was placed a solution of 4-amino-3-(2-[[2-(trimethylsilyl)ethoxy]methoxy]ethyl)benzonitrile (500 mg, 1.7 mmol, 1 equiv) in ACN (10 mL), followed by NBS (608.6 mg, 3.4 mmol, 2 equiv). The resulting solution was stirred for 15 min at room temperature. The reaction was then quenched by the addition of 10 mL of Na₂SO₃. The resulting solution was extracted with 3×30 ml of ethyl acetate; and the combined organic phase was concentrated. This resulted in 600 mg (94.5%) of 4-amino-3-bromo-5-(2-[[2-(trimethylsilyl)ethoxy]methoxy]ethyl)benzonitrile as yellow oil.

### 5. Synthesis of 4-amino-3-(prop-1-en-2-yl)-5-(2-((2-(trimethylsilyl)ethoxy)methoxy)ethyl) benzonitrile

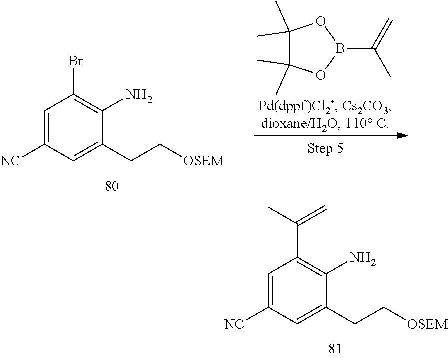

Into a 50-mL round-bottom flask purged and maintained with an inert atmosphere of nitrogen, was placed 4-amino-3-bromo-5-(2-[[2-(trimethylsilyl)ethoxy]methoxy]ethyl)benzonitrile (600 mg, 1.62 mmol, 1 equiv), 4,4,5,5-tetramethyl-2-(prop-1-en-2-yl)-1,3,2-dioxaborolane (325.8 mg, 1.94 mmol, 1.2 equiv), Pd(dppf)Cl2 (59.1 mg, 0.08 mmol, 0.05 equiv), Cs₂CO₃ (1.6 g, 4.85 mmol, 3 equiv), dioxane (6 mL), and H₂O (0.6 mL). The resulting solution was stirred for 12 h at 90 degrees C., after which it was diluted with 10 mL of water and extracted with 3×30 ml of ethyl acetate. The combined organic phase was dried over anhydrous sodium sulfate and concentrated. The resulting residue was applied onto a silica gel column with ethyl acetate/petroleum ether (1:5). This resulted in 300 mg (55.8%) of 4-amino-3-(prop-1-en-2-yl)-5-(2-[[2-(trimethylsilyl)ethoxy]methoxy]ethyl)benzonitrile as light yellow oil.

### 6. Synthesis of 4-amino-3-isopropyl-5-(2-((2-(trimethylsilyl)ethoxy)methoxy)ethyl) benzonitrile

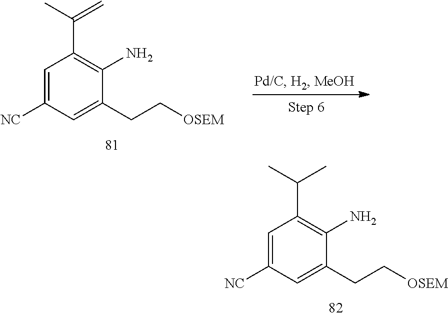

Into a 50-mL round-bottom flask, was placed a solution of 4-amino-3-(prop-1-en-2-yl)-5-(2-[[2-(trimethylsilyl)ethoxy]methoxy]ethyl)benzonitrile (300 mg, 0.9 mmol, 1 equiv) in MeOH (5 mL) and Pd/C (30 mg, 0.3 mmol, 0.3 equiv). The resulting mixture was stirred for 12 hr at 45 degrees C. under a hydrogen atmosphere. The solids were filtered out. The filtrate was concentrated. This resulted in 240 mg (79.5%) of 4-amino-3-(propan-2-yl)-5-(2-[[2-(trimethylsilyl)ethoxy]methoxy]ethyl)benzonitrile as light yellow oil.

### 7. Synthesis of 4-isocyanato-3-(propan-2-yl)-5-(2-[[2-(trimethylsilyl)ethoxy]methoxy]ethyl) benzonitrile

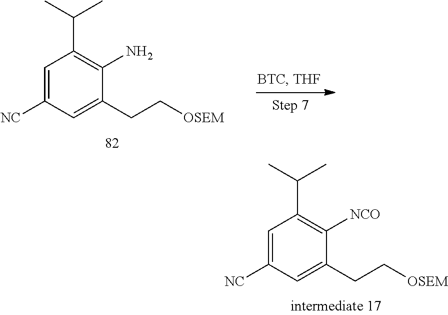

Into a 50-mL round-bottom flask, was placed a solution of 4-amino-3-(propan-2-yl)-5-(2-[[2-(trimethylsilyl)ethoxy]methoxy]ethyl)benzonitrile (240 mg, 0.7 mmol, 1 equiv) in THF (10 mL), ditrichloromethyl carbonate (106.4 mg, 0.36 mmol, 0.5 equiv), and TEA (145.2 mg, 1.4 mmol, 2 equiv). The resulting solution was stirred for 2 h at 60 degrees C. The resulting mixture was concentrated. This resulted in 250 mg (96.7%) of 4-isocyanato-3-(propan-2-yl)-5-(2-[[2-(trimethylsilyl)ethoxy]methoxy]ethyl)benzonitrile as a light yellow solid.

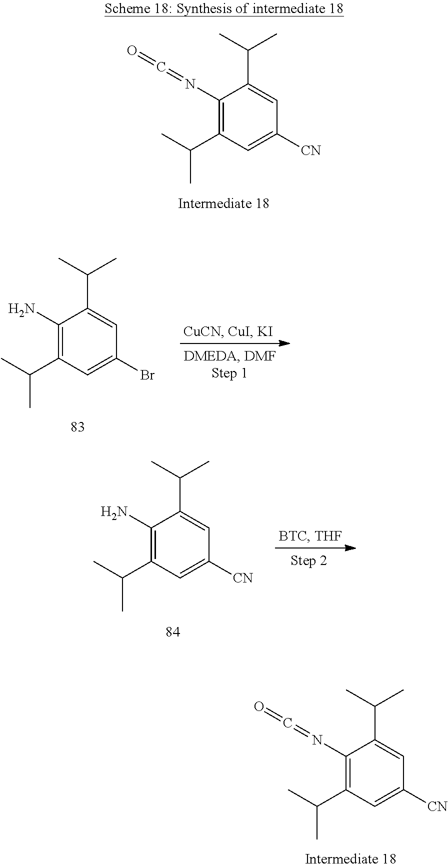 [SMILES: CC(C)c1cc(C#N)cc(C(C)C)c1N=C=O ; CC(C)c1cc(Br)cc(C(C)C)c1N ; CC(C)c1cc(C#N)cc(C(C)C)c1N ; CC(C)c1cc(C#N)cc(C(C)C)c1N=C=O]

### 1. Synthesis of 4-Amino-3,5-diisopropylbenzonitrile

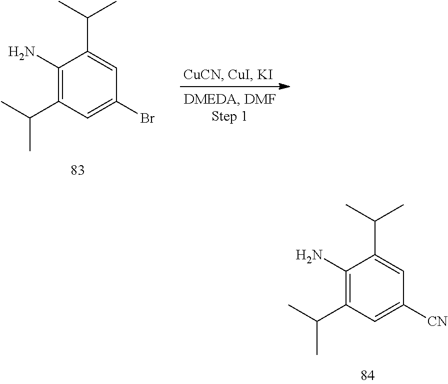

Into a 100-mL round-bottom flask purged with and maintained under nitrogen, was placed 4-bromo-2,6-diisopropylbenzenamine (commercially available, 5.1 g, 19.9 mmol), DMF (30 mL), CuCN (2.16 g, 23.9 mmol), Cut (380 mg, 2.00 mmol), KI (664 mg, 3.98 mmol), and DMEDA (2.0 mL). The resulting solution was stirred for 24 h at 100° C. and was then diluted with 30 mL of water. The solution was extracted with 3×30 ml of ethyl acetate and the organic layers combined and concentrated under vacuum. The resulting residue was applied onto a silica gel column and eluted with ethyl acetate/petroleum ether (1:30 to 1:20). This resulted in 1.2 g (30%) of the title compound as a yellow solid, MS-ESI: 203.1 (M+1).

### 2. Synthesis of 4-isocyanato-3,5-diisopropylbenzonitrile

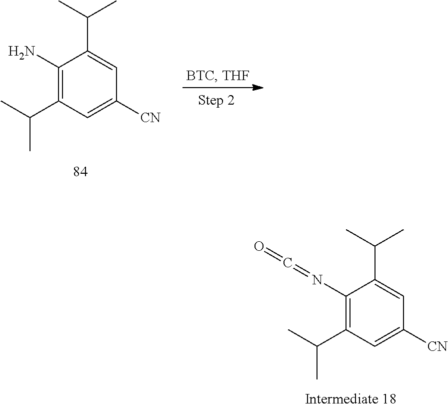 [SMILES: CC(C)c1cc(C#N)cc(C(C)C)c1N ; CC(C)c1cc(C#N)cc(C(C)C)c1N=C=O]

To a stirred solution/mixture of 4-amino-5-(butan-2-yl)-2-fluoro-3-(propan-2-yl)benzonitrile(300 mg, 1.28 mol, 1.0 equiv) and TEA (259.1 mg, 2.6 mmol, 2.0 equiv) in THF (15 mL) was added BTC (190.1 mg, 0.6 mmol, 0.5 equiv) at room temperature under nitrogen atmosphere. The resulting mixture was stirred for 3 h at 65° C. under nitrogen atmosphere. The resulting mixture was concentrated under vacuum. This give 4-isocyanato-3,5-diisopropyl benzonitrile (260 mg, 89%) as a brown solid.

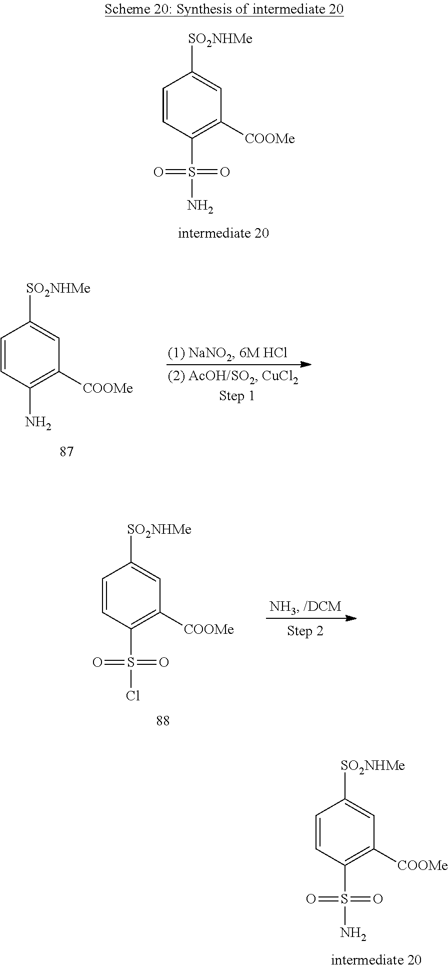

### 1. Synthesis of methyl 2-(chlorosulfonyl)₅-(methylsulfamoyl)benzoate

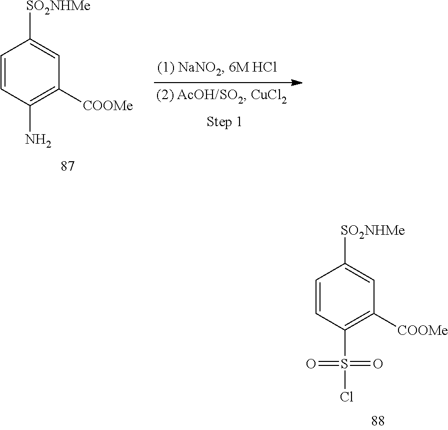

Into a 250-mL round-bottom flask, was placed methyl 2-amino-5-(methylsulfamoyl)benzoate (2 g), HCl(20 mL, aqueous, 6 M), NaNO₂ (1.2 g), SO₂/CH₃COOH (20 mL), and CuCl₂ (550 mg). The resulting solution was stirred for 2 hours at 0 degrees C. The resulting residue was applied onto a silica gel column with ethyl acetate/petroleum ether (1/1). This resulted in 600 mg of methyl 2-(chlorosulfonyl)-5-(methylsulfamoyl)benzoate as a solid.

### 2. Synthesis of methyl 5-(methylsulfamoyl)-2-sulfamoylbenzoate

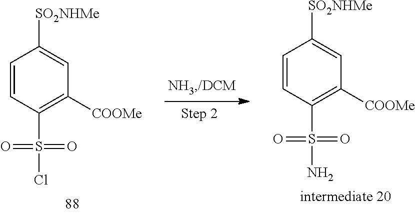

Into a 250-mL round-bottom flask, was placed methyl 2-(chlorosulfonyl)-5-(methylsulfamoyl)benzoate (300 mg) and NH₃/THF (20 mL). The resulting solution was stirred for 4 hours at room temperature. The residue was applied onto a silica gel column with ethyl acetate/petroleum ether (1/1). This resulted in 300 mg of methyl 5-(methylsulfamoyl)-2-sulfamoylbenzoate as a white solid.

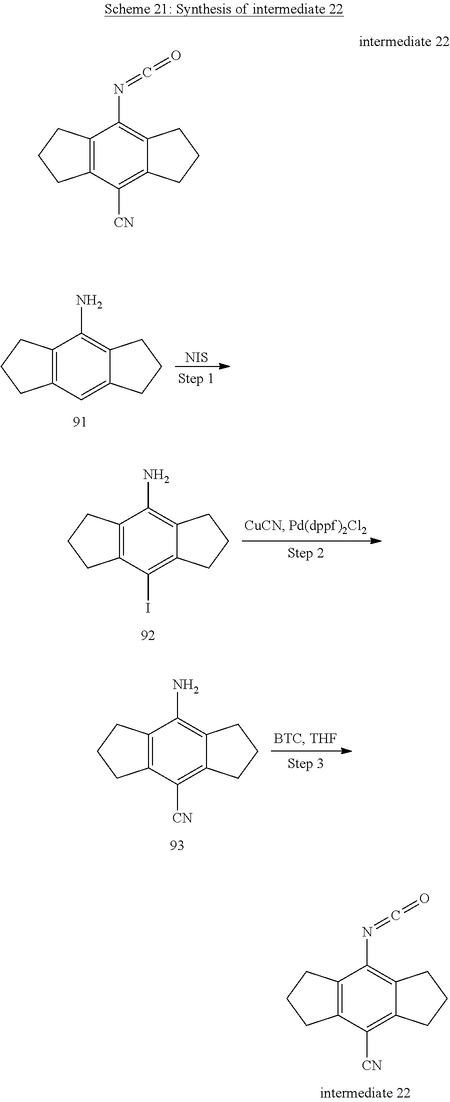

### 1. Synthesis of methyl 5-sulfamoylisoxazole-3-carboxylate

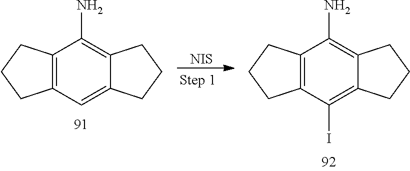

Into a 50-mL round-bottom flask, was placed 1,2,3,5,6,7-hexahydro-s-indacen-4-amine (2.076 g, 11.98 mmol, 1.00 equiv), N,N-dimethylformamide (10 mL), and NIS (2.97 g, 13.20 mmol, 1.10 equiv). The resulting solution was stirred for 3 h at room temperature. The resulting solution was diluted with 100 mL of ethyl acetate. The resulting mixture was washed with 3×10 mL of brine. The combined organic phase was concentrated under vacuum. The residue was applied onto a silica gel column with ethyl acetate/petroleum ether (1:20). This resulted in 2.837 g (79%) of 8-iodo-1,2,3,5,6,7-hexahydro-s-indacen-4-amine as orange solid.

### 2. Synthesis of 8-amino-1,2,3,5,6,7-hexahydros-indacene-4-carbonitrile

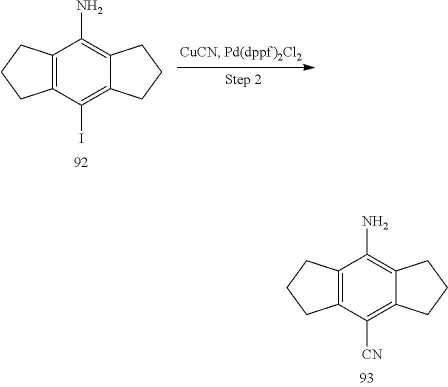 [SMILES: Nc1c2c(c(I)c3c1CCC3)CCC2 ; N#Cc1c2c(c(N)c3c1CCC3)CCC2]

Into a 50-mL round-bottom flask purged and maintained with an inert atmosphere of nitrogen, was placed 8-iodo-1,2,3,5,6,7-hexahydro-s-indacen-4-amine (438 mg, 1.46 mmol, 1.00 equiv), CuCN (195 mg, 1.50 equiv), t-BuOK (16 mg, 0.14 mmol, 0.10 equiv), Pd(dppf)Cl2 (255 mg, 0.35 mmol, 0.20 equiv), Pd(PPh₃)₄ (169 mg, 0.15 mmol, 0.10 equiv), and N,N dimethylformamide (15 mL). The resulting solution was stirred for 12 min at 120° C. using an oil bath. The resulting solution was extracted with ethyl acetate and the organic layers combined. The resulting mixture was washed with H₂O. The mixture was dried over anhydrous sodium sulfate and concentrated under vacuum. The crude product was purified by Prep-HPLC with the following conditions (2 #-AnalyseHPLC-SHIMADZU(HPLC-10)): Column, XBridge C18 OBD Prep Column, 100&Aring, 10 &micro;m, 19 mm X 250 mm; mobile phase, Water(10MMOL/L NH4HCO3) and ACN (20% ACN up to 60% in 8 min); Detector, UV 254 nm. This resulted in 145 mg (50%) of 8-amino-1,2,3,5,6,7-hexahydro-s-indacene-4-carbonitrile as a yellow solid.

### 3. Synthesis of 8-amino-1,2,3,5,6,7-hexahydros-indacene-4-carbonitrile

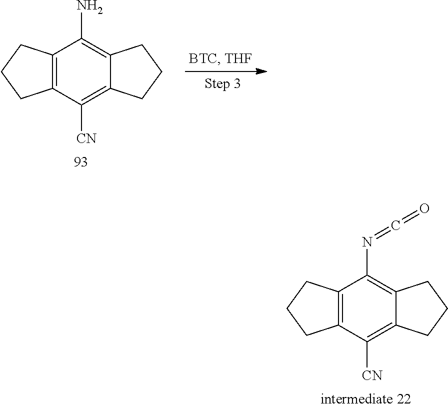 [SMILES: N#Cc1c2c(c(N)c3c1CCC3)CCC2 ; N#Cc1c2c(c(N=C=O)c3c1CCC3)CCC2]

To a stirred solution/mixture of 8-amino-1,2,3,5,6,7-hexahydro-s-indacene-4-carbonitrile (253 mg, 1.28 mol, 1.0 equiv) and TEA (259.1 mg, 2.6 mmol, 2.0 equiv) in THF(15 mL) was added BTC(190.1 mg, 0.6 mmol, 0.5 equiv) at room temperature under nitrogen atmosphere. The resulting mixture was stirred for 3 h at 65° C. under nitrogen atmosphere. The resulting mixture was concentrated under vacuum to give 8-amino-1,2,3,5,6,7-hexahydros-indacene-4-carbonitrile (280 mg, 98%) as brown solid

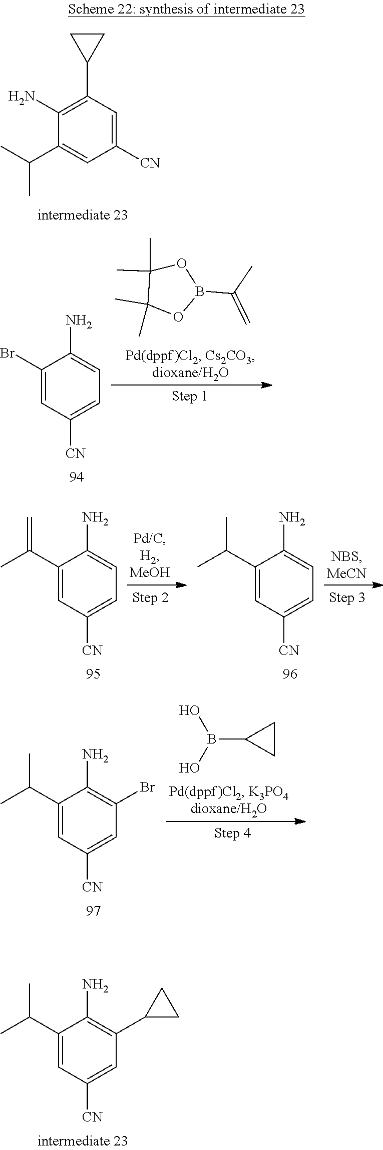

### 1. 4-amino-3-(prop-1-en-2-yl)benzonitrile

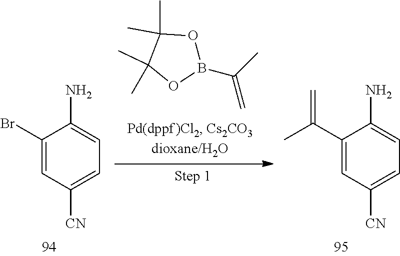

Into a 1000-mL round-bottom flask purged with and maintained under nitrogen was placed 4-amino-3-bromobenzonitrile (19.7 g, 100 mmol, 1 equiv), dioxane (300 mL), water (30 mL), Cs₂CO₃ (65.2 g. 200 mmol, 2 equiv), 4,4,5,5-tetramethyl-2-(prop-1-en-2-yl)-1,3,2-dioxaborolane (25.2 g, 150 mmol, 1.5 equiv), and Pd(dppf)Cl₂ (3.7 g, 5 mmol, 0.05 equiv). The resulting solution was stirred overnight at 110° C. and then concentrated under vacuum. The residue was applied onto a silica gel column and eluted with ethyl acetate/petroleum ether (1:40 to 1:20). This resulted in 17 g (70%) of the title compound as light yellow solid. MS-ESI: 159 (M+1).

### 2. 4-amino-3-isopropylbenzonitrile

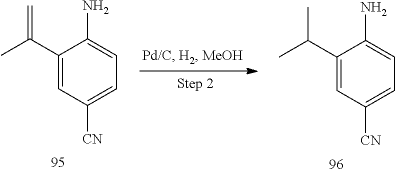

Into a 500-mL round-bottom flask was placed 4-amino-3-(prop-1-en-2-yl)benzonitrile (17 g, 106 mmol) and MeOH (300 mL), Then Pd/C (10% wt, 1.7 g) was added. The flask was evacuated and flushed three times with hydrogen. The resulting solution was stirred for overnight at RT under an atmosphere of hydrogen. The solids were filtered out. The resulting mixture was concentrated under vacuum. This resulted in 16 g (94%) of the title compound as yellow solid. MS-ESI: 161 (M+1).

### 3. 4amino-3-bromo-5-isopropylbenzonitrile

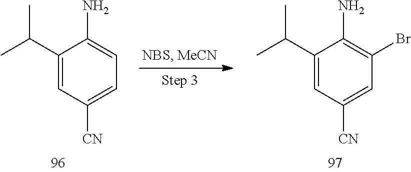

Into a 500-mL round-bottom flask purged with and maintained under nitrogen was placed 4-amino-3-isopropylbenzonitrile (16 g, 100 mmol, 1 equiv) in MeCN (200 mL). To this mixture was added NBS (26.7 g, 150 mmol, 1.5 equiv) in portions at room temperature. The resulting solution was stirred overnight at room temperature and then was concentrated under vacuum, washed with sat. NaHCO₂ (100 mL), the solid was collected by filtration. The crude solid was applied onto a silica gel column and eluted with ethyl acetate/petroleum ether (1:40 to 1:20). This resulted in 20 g (85%) of the title compound as light yellow oil. MS-ESI: 239 (M+1).

### 4. Synthesis of 4-amino-3-cyclopropyl-5-isopropylbenzonitrile

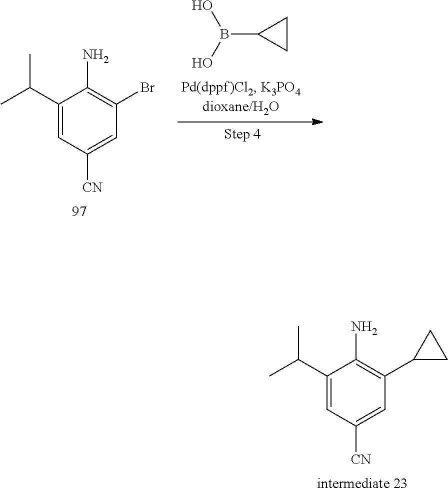

Into a 500-mL round-bottom flask purged with and maintained under nitrogen was placed 4-amino-3-bromo-5-isopropylbenzonitrile (2.4 g, 10 mmol, 1 equiv), 1,4-dioxane (200 mL), K₃PO₄ (3.18 g, 15 mmol, 1.5 equiv), cyclopropylboronic acid (1.3 g, 15 mmol, 1.5 equiv), and Pd(dppf)C₁₋₂ (0.73 g, 1 mmol, 0.1 equiv). The resulting solution was stirred overnight at 90° C. and then was concentrated under vacuum. The residue was applied onto a silica gel column and eluted with ethyl acetate/petroleum ether (1:40 to 1:20). This resulted in 1 g (50%) of the title compound as light yellow oil. MS-ESI: 201 (M+1).

### B. Preparation of Exemplary Compounds

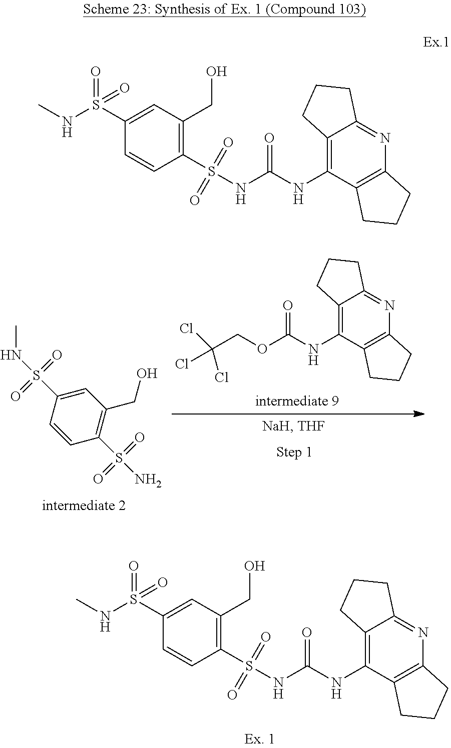

### 1. 3-[2-azatrieyclo[7.3.0.0\hat{ }[3,7]]dodeca-1,3(7),8-trien-8-yl]-1-[2-(hydroxymethyl)-4-(methylsulfamoyl)benzenesulfonyl]urea

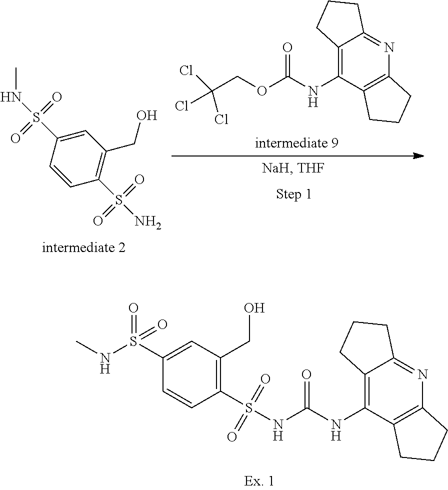 [SMILES: CNS(=O)(=O)c1ccc(S(N)(=O)=O)c(CO)c1 ; O=C(Nc1c2c(nc3c1CCC3)CCC2)OCC(Cl)(Cl)Cl ; CNS(=O)(=O)c1ccc(S(=O)(=O)NC(=O)Nc2c3c(nc4c2CCC4)CCC3)c(CO)c1]

Into a 50 mL round-bottom flask were added 3-(hydroxymethyl)-N1-methylbenzene-1,4-disulfonamide (100 mg, 0.36 mmol, 1 equiv) and THF (10 mL) at room temperature. To a stirred solution of 3-(hydroxymethyl)-N1-methylbenzene-1,4-disulfonamide (100 mg, 0.36 mmol, 1 equiv) in THF (10 mL) was added NaH (17.1 mg, 0.71 mmol, 2.00 equiv) in one portion at 0 degrees C. under nitrogen atmosphere. The resulting mixture was stirred overnight at 0 degrees C. under nitrogen atmosphere. Then 2,2,2-trichloroethyl N-[2-azatrieyclo[7.3.0.0\hat{ }[3,7]]dodeca-1,3(7),8-trien-8-yl]carbamate(124.7 mg, 0.36 mmol, 1.00 equiv) in THF (5 mL) was added to the abovementioned mixture. The resulting mixture was stirred for 2 h at room temperature. The reaction was quenched with water (1 mL) at 0 degrees C. The resulting mixture was concentrated under reduced pressure. The crude product (mg) was purified by Prep-HPLC with the following conditions (Column: XBridge Prep C18 OBD Column 19 150 mm 5 um, Mobile Phase A:Water(10 MMOL/L NH4HCO3+0.1% NH3.H2O), Mobile Phase B: ACN; Flow rate: 25 mL/min, Gradient: 10% B to 24% B in 8 min; 254/210 nm; Rt: 6.13 min) to afford 3-[2-azatricyclo[7.3.0.0\hat{ }[3,7]]dodeca-1,3(7),8-trien-8-yl]-1-[2-(hydroxymethyl)-4-(methylsulfamoyl)benzenesulfonyl]urea (24.1 mg, 13.82%) as a white solid.

LC-MS3-[2-azatricyclo[7.3.0.0\hat{ }[3,7]]dodeca-1,3(7),8trien-8-yl]-1-[2-(hydroxymethyl)-4-(methylsulfamoyl)benzenesulfonyl]urea: (ES, m/z): 480

H-NMR-3-[2-azatricyclo[7.3.0.0\hat{ }[3,7]]dodeca-1,3(7),8-trien-8-yl]-1-[2-(hydroxymethyl)-4-(methylsulfamoyl)benzenesulfonyl]urea: (300 MHz, DMSO-d) δ 8.410 (m, 1H), 8.018 (s, 1H), 7,955-7,928 (d, J=8.1 Hz, 1H), 7.631-7.609 (d, J=6.6 Hz, lit), 7.479-7.463 (d, J=4.8 Hz, 1H), 4.987 (s, 2H), 2.817-2.767 (t, J=6.9 Hz, 4H), 2.651-2.601 (t, J=7.5 Hz, 4H), 2.428-2.398 (m, 3H), 1.942-1.892 (t, J=8.4 Hz, 4H).

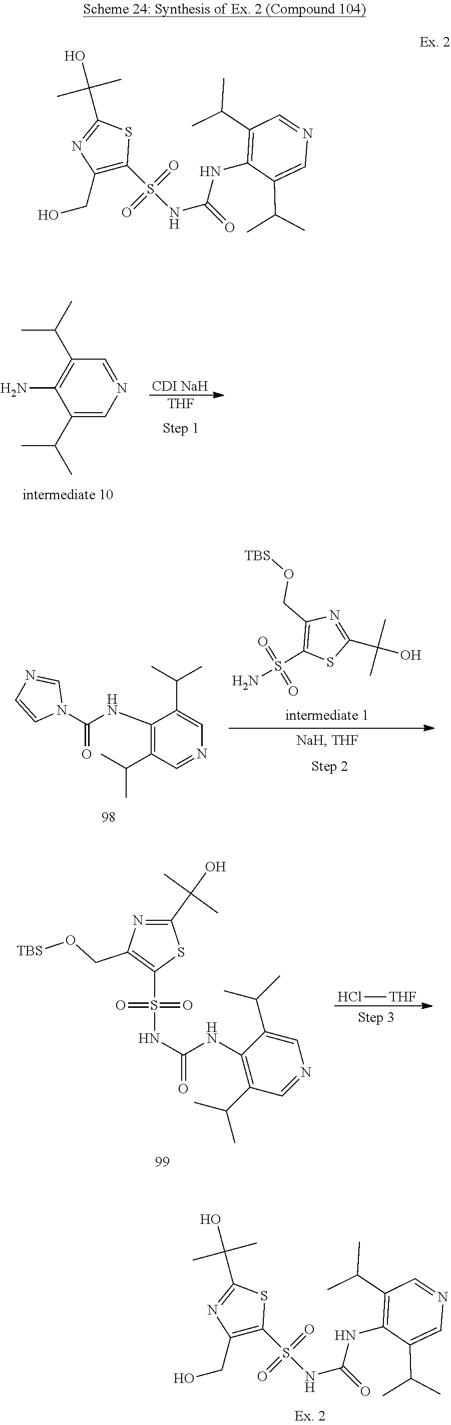

### 1. Synthesis of N-(3,5-diisopropylpyridin-4-yl)-1H-imidazole-1-carboxamide

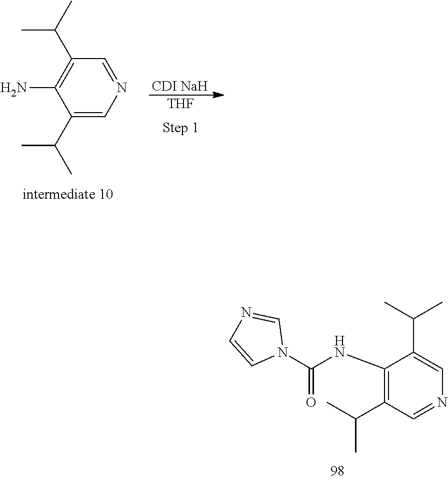

Into a 25-mL round-bottom flask, was placed 3,5-bis(propan-2-yl)pyridin-4-amine (200 mg), N,N-dimethylformamide (2 mL), NaH (89 mg), and CDI (161.87 mg). The resulting solution was stirred for 16 hr at room temperature. The resulting solution was used directly in the next step.

### 2. Synthesis of 1-[3,5-bis(propan-2-yl)pyridin-4-yl]-3-[(4-[[(tert-butyldimethylsilyl)oxy]methyl]-2-(2-hydroxypropan-2-yl)-1,3-thiazol-5-yl)sulfonyl]urea

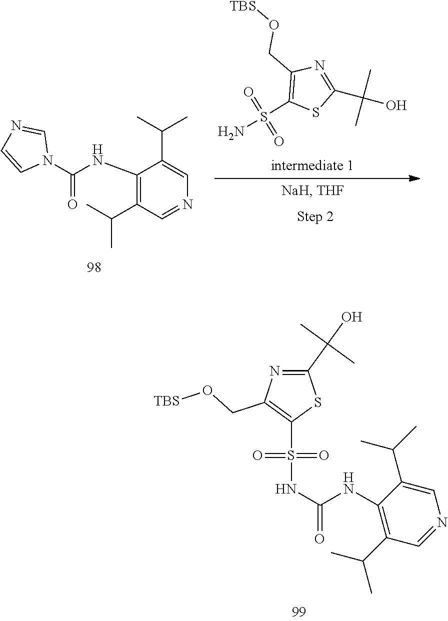

Into a 50-mL round-bottom flask, was placed 4-[[(tert-butyldimethylsilyl)oxy]methyl]-2-(2-hydroxypropan-2-yl)-1,3-thiazole-5-sulfonamide (224.26 mg), THF (5 mL), and NaH (58.82 mg). This was followed by the addition of a solution of N-[3,5-bis(propan-2-yl)pyridin-4-yl]-1H-imidazole-1-carboxamide (200 mg) in DMF (2 mL) dropwise with stirring. The resulting solution was stirred for 3 hr at room temperature, after which it was concentrated in vacuo. The residue was applied onto a silica gel column with ACN:H₂O (1:2). This resulted in 55 mg of 1-[3,5-bis(propan-2-yl)pyridin-4-yl]-3-[(4-[[(tert-butyldimethylsilyl)oxy]methyl]-2-(2-hydroxypropan-2-yl)-1,3-thiazol-5-yl)sulfonyl]urea as a white solid.

### 3. Synthesis of 1-[3,5-bis(propan-2-yl)pyridin-4-yl]-3-[(4-[[(tert-butyldimethylsilyl)oxy]methyl]-2-(2-hydroxypropan-2-yl)-1,3-thiazol-5-yl)sulfonyl]urea

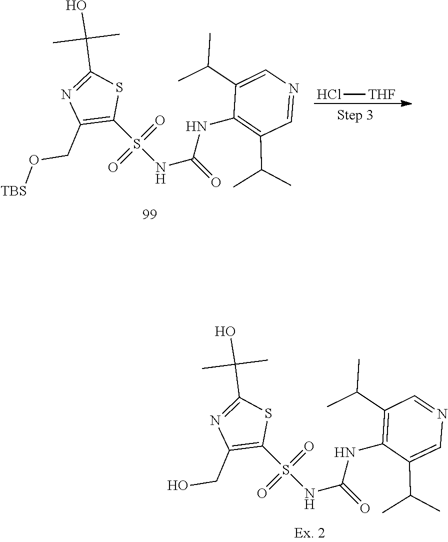

Into a 50-mL round-bottom flask, was placed 1-[3,5-bis(propan-2-yl)pyridin-4-yl]-3-[(4-[[(tert-butyldimethylsilyl)oxy]methyl]-2-(2-hydroxypropan-2-yl)-1,3-thiazol-5-yl)sulfonyl]urea (40 mg, 0.07 mmol) in THF(3 mL) with dioxane-HCl (0.5 mL). The resulting solution was stirred for 30 min at room temperature. The resulting mixture was concentrated under vacuum. The residue was treated with 5 mL of DMF and the solids were filtered out. The crude product was purified by Prep-HPLC with the following conditions (2 #SHIMADZU (HPLC-01)): Column, XBridge Shield RP18 OBD Column, 19*250 mm, 10 um; mobile phase, Water(10MMOL/L NH4HCO3) and ACN (30% PhaseB up to 50% in 10 min); Detector, UV 220/254 nm. This resulted in 1-[3,5-bis(propan-2-yl)pyridin-4-yl]-3-[(4-[[(tert-butyldimethylsilyl)oxy]methyl]-2-(2-hydroxypropan-2-yl)-1,3-thiazol-5-yl)sulfonyl]urea (5 mg, 14%) as a white solid.

LC-MS-1-[3,5-bis(propan-2-yl)pyridin-4-yl]-3-[(4-[[(tert-butyldimethylsilyl)oxy]methyl]-2-(2-hydroxypropan-2-yl)-1,3-thiazol-5-yl)sulfonyl]urea: (ES, m/z): 509.1

H-NMR-1-[3,5-bis(propan-2-yl)pyridin-4-yl]-3-[(4-[[(tert-butyldimethylsilyl)oxy]methyl]-2-(2-hydroxypropan-2-yl)-1,3-thiazol-5-yl)sulfonyl]urea: (300 MHz, DMSO-d₆) δ 8.28 (s, 1H), 4.64 (s, 2H), 3.12-3.01 (m, 2H), 1.47 (s, 6H), 1.09 (d, J=6.9 Hz, 12H).

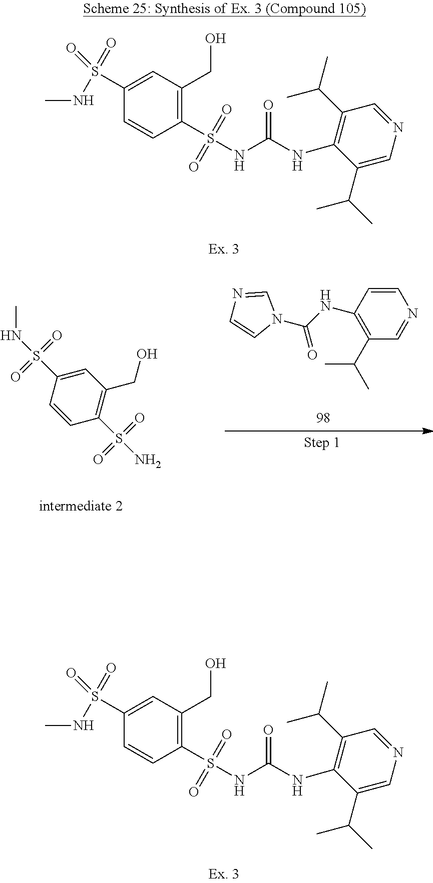 [SMILES: CNS(=O)(=O)c1ccc(S(=O)(=O)NC(=O)Nc2c(C(C)C)cncc2C(C)C)c(CO)c1 ; CNS(=O)(=O)c1ccc(S(N)(=O)=O)c(CO)c1 ; CC(C)c1cnccc1NC(=O)n1ccnc1 ; CNS(=O)(=O)c1ccc(S(=O)(=O)NC(=O)Nc2c(C(C)C)cncc2C(C)C)c(CO)c1]

### 1. Synthesis of 3-[3,5-bis(propan-2-yl)pyridin-4-yl]-1-[2-(hydroxymethyl)-4-(methylsulfamoyl)benzenesulfonyl]urea

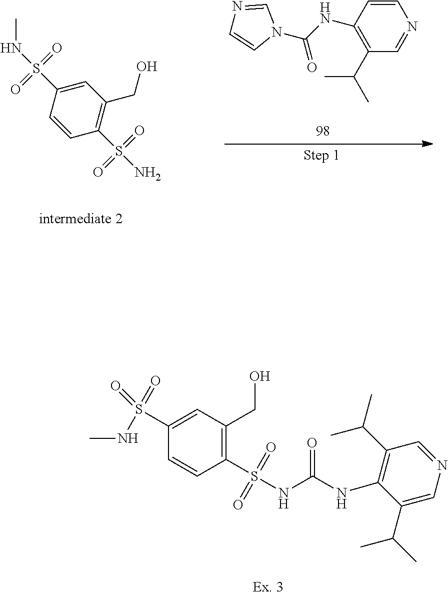 [SMILES: CNS(=O)(=O)c1ccc(S(N)(=O)=O)c(CO)c1 ; CC(C)c1cnccc1NC(=O)n1ccnc1 ; CNS(=O)(=O)c1ccc(S(=O)(=O)NC(=O)Nc2c(C(C)C)cncc2C(C)C)c(CO)c1]

To a stirred solution of 3-(hydroxymethyl)-N1-methylbenzene-1,4-disulfonamide(100 mg, 0.36 mmol, 1 equiv) in THF (10 mL) was added NaH (17.1 mg, 0.71 mmol, 2 equiv) in one portion at 0 degrees C. under nitrogen atmosphere. The resulting mixture was stirred for 30 min at 0 degrees C. under nitrogen atmosphere. Then N-[3,5-bis(propan-2-yl)pyridin-4-yl]-1H-imidazole-1-carboxamide (97.2 mg, 0.36 mmol, 1 equiv) (which can be prepared according to Scheme 25, step 1) in THF (5 mL) was added to the above resulting mixture. The resulting mixture was stirred for additional 4 h at room temperature. The resulting mixture was concentrated under reduced pressure. The crude product (mg) was purified by Prep-HPLC with the following conditions (Column: XBridge Prep C18 OBD Column 19×150 mm 5 um; Mobile Phase A: Water(10 MMOL/L NH4HCO3+0.1% NH3.H2O), Mobile Phase B: ACN; Flow rate: 25 mL/min; Gradient: 10% B to 26% B in 10.5 min; 254/210 ran; Rt: 7.58 min) to afford 3-[3,5-bis(propan-2-yl)pyridin-4-yl]-1-[2-(hydroxymethyl)-4-(methylsulfamoyl)benzenesulfonyl]urea(17.0 mg, 9.12%) as a white solid.

LC-MS-3-[3,5-bis(propan-2-yl)pyridin-4-yl]-1-[2-(hydroxymethyl)-4-(methylsulfamoyl)benzenesulfonyl]urea: (ES, m/z): 484.1

H-NMR-3-[3,5-bis(propan-2-yl)pyridin-4-yl]-1-[2-(hydroxymethyl)-4-(methylsulfamoyl)benzenesulfonyl]urea: (300 MHz, DMSO-d) δ 8.20 (s, 1H), 8.04 (s, 1H), 7.97-7.94 (d, J=8.7 Hz, 1H), 7.64-7.61 (d, J=7.5 Hz, 1H), 3.84 (s, 2H), 3.11 (s, 2H), 1.59 (s, 6H), 1.15 (d, J 6.8 Hz, 1211).

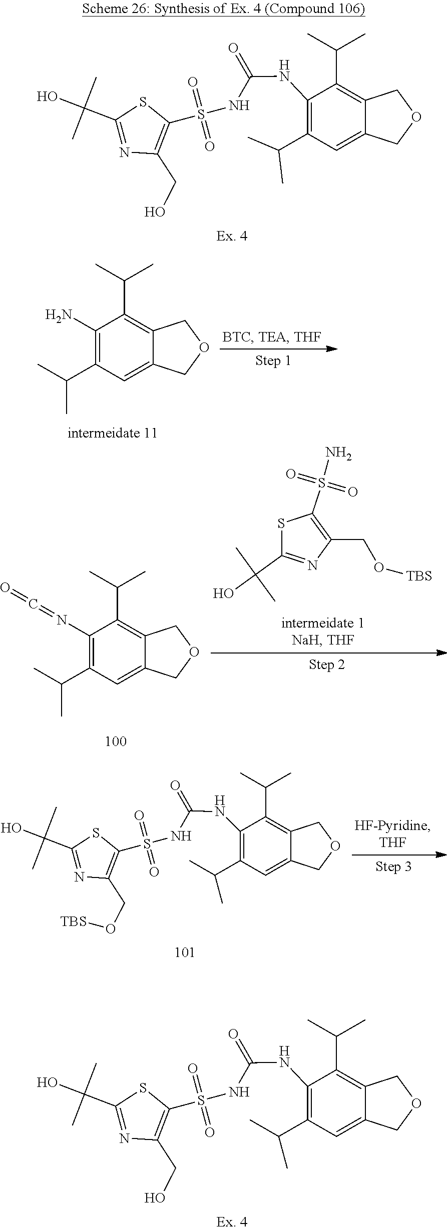

### 1. Synthesis of 5-isocyanato-4,6-diisopropyl-1,3-dihydroisobenzofuran

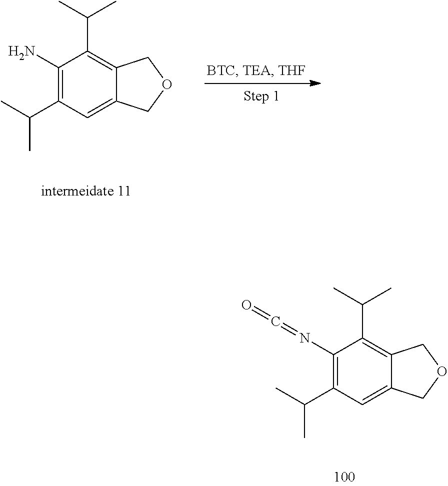 [SMILES: CC(C)c1cc2c(c(C(C)C)c1N)COC2 ; CC(C)c1cc2c(c(C(C)C)c1N=C=O)COC2]

Into a 50-mL round-bottom flask, was placed 4,6-bis(propan-2-yl)-1,3-dihydro-2-benzofuran-5-amine (100 mg, 1 equiv), THF (10 mL), TEA (0.2 mL), and BTC (44.6 mg, 0.33 equiv). The resulting solution was stirred for 1 hr at 70 degrees C. The resulting mixture was concentrated under vacuum to afford a crude residue of 5-isocyanato-4,6-diisopropyl-1,3-dihydroisobenzofuran, which was directly used for the next step without further purifications.

### 2. Synthesis of 3-[4,6-bis(propan-2-yl)-1,3-dihydro-2-benzofuran-5-yl]-1-[(4-[[(tert-butyldimethylsilyl)oxy]methyl]-2-(2-hydroxypropan-2-yl)-1,3-thiazol-5-yl)sulfonyl]urea

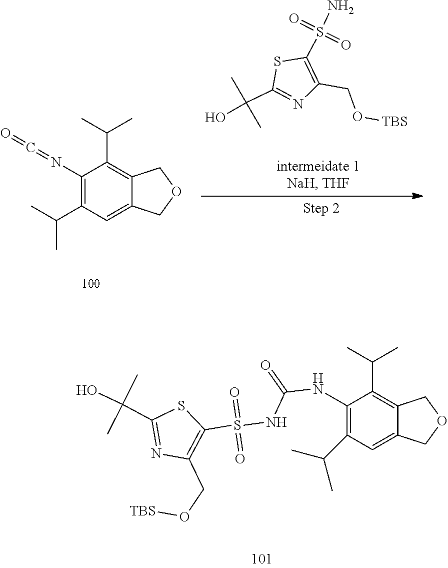

Into a 50-mL round-bottom flask, was placed 5-isocyanato-4,6-bis(propan-2-yl)-1,3-dihydro-2-benzofuran (100 mg, 1 equiv), THF (15 mL), NaH (27 mg, 3 equiv), and 4-[[(tert-butyldimethylsilyl)oxy]methyl]-2-(2-hydroxypropan-2-yl)-1,3-thiazole-5-sulfonamide (125 mg, 1.2 equiv). The resulting solution was stirred for 2 h at room temperature. The reaction was then quenched by the addition of 10 mL of water. The resulting solution was extracted with 3×15 mL of ethyl acetate and the organic layers combined and dried over anhydrous sodium sulfate. The solids were filtered out. The filtrate was concentrated under vacuum, and the crude product was purified by Prep-HPLC with the following conditions (2 #SHIMADZU (HPLC-01)): Column, XBridge Prep 08 OBD Column, 150 mm 5 um; mobile phase, Water(10MMOL/L NH4HCO3+0.1% NH3.H2O) and ACN (10% PhaseB up to 90% in 6 min); Detector, UV 220/254 nm. This resulted in 50 mg of 3-[4,6-bis(propan-2-yl)-1,3-dihydro-2-benzofuran-5-yl]-1-[(4-[[(tert-butyldimethylsilyl)oxy]methyl]-2-(2-hydroxypropan-2-yl)-1,3-thiazol-5-yl)sulfonyl]urea as a white solid.

LC-MS-3-[4,6-bis(propan-2-yl)-1,3-dihydro-2-benzofuran-5-yl]-1-[(4-[[(tert-butyldimethylsilyl)oxy]methyl]-2-(2-hydroxypropan-2-yl)-1,3-thiazol-5-yl)sulfonyl]urea: (ES, m/z): 453.18

H-NMR-3-[4,6-bis(propan-2-yl)-1,3-dihydro-2-benzofuran-5-yl]-1-[(4-[[(tert-butyldimethylsilyl)oxy]methyl]-2-(2-hydroxypropan-2-yl)-1,3-thiazol-5-yl)sulfonyl]urea: (300 MHz, Methanol-d₄) δ 7.07 (s, 1H), 5.14 (s, 2H), 5.07 (s, 2H), 4.96 (s, 2H), 3.03 (m, 2H), 1.59 (s, 6H), 1.12 (d, J=6.9 Hz, 12H), 0.92 (s, 9H), 0.11 (s, 6H),

### 3. Synthesis of P3-[4,6-bis(propan-2-yl)-1,3-dihydro-2-benzofuran-5-yl]-1-[[4-(hydroxymethyl)-2-(2-hydroxypropan-2-yl)-1,3-thiazol-5-yl]sulfonyl]urea

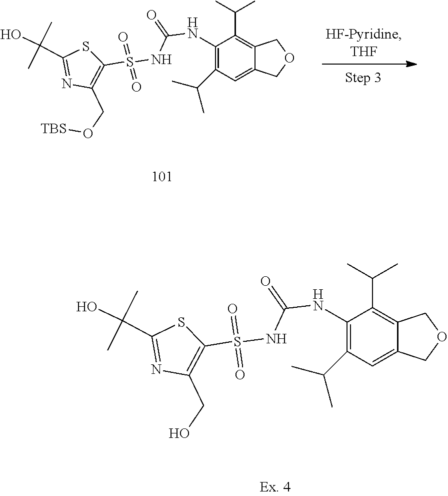

Into a 50-mL round-bottom flask, was placed 3-[4,6-bis(propan-2-yl)-1,3-dihydro-2-benzofuran-5-yl]-1-[(4-[[(tert-butyldimethylsilyl)oxy]methyl]-2-(2-hydroxypropan-2-yl)-1,3-thiazol-5-yl)sulfonyl]urea (40 mg), THF (10 mL), and HF Pyridine (0.5 mL). The resulting solution was stirred for 10 h at room temperature, after which it was concentrated under vacuum. The resulting residue was treated with 5 mL of ACN, and the solids were filtered out. The filtrate was concentrated and applied onto a silica gel column with ACN:H2O (1:2). This resulted in 15 mg of 3-[4,6-bis(propan-2-yl)-1,3-dihydro-2-benzofuran-5-yl]-1-[[4-(hydroxymethyl)-2-(2-hydroxypropan-2-yl)-1,3-thiazol-5-yl]sulfonyl]urea as a white solid.

LC-MS-3-[4,6-bis(propan-2-yl)-1,3-dihydro-2-benzofuran-5-yl]-1-[[4-(hydroxymethyl)-2-(2-hydroxypropan-2-yl)-1,3-thiazol-5-yl]sulfonyl]urea: (ES, m/z): 497.2

H-NMR-3-[4,6-bis(propan-2-yl)-1,3-dihydro-2-benzofuran-5-yl]-1-[[4-(hydroxymethyl)-2-(2-hydroxypropan-2-yl)-1,3-thiazol-5-yl]sulfonyl]urea: (400 MHz, Methanol-d₄) δ 7.10 (s, 2H), 5.15 (s, 2H), 4.95 (s, 2H), 4.82 (s, 2H), 3.18 (t, J=6.5 Hz, 3H), 3.05 (t, J=6.3 Hz, 3H),1.62 (s, 6H), 1.15 (d, J=6.9 Hz. 12H).

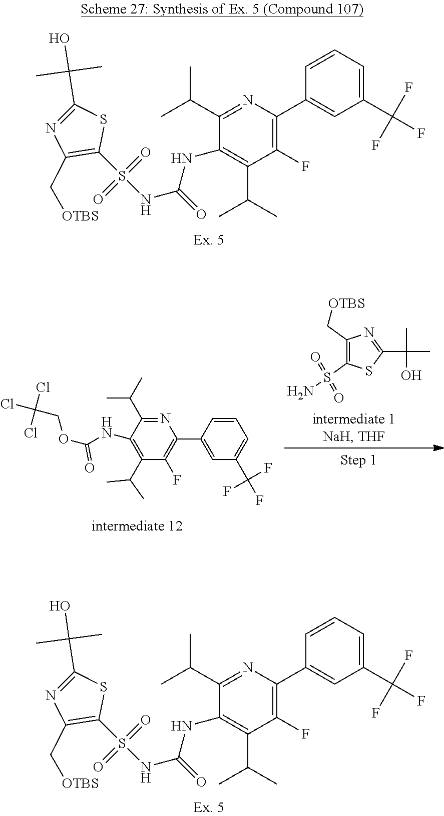 [SMILES: CC(C)c1nc(-c2cccc(C(F)(F)F)c2)c(F)c(C(C)C)c1NC(=O)NS(=O)(=O)c1sc(C(C)(C)O)nc1CO[Si](C)(C)C(C)(C)C ; CC(C)c1nc(-c2cccc(C(F)(F)F)c2)c(F)c(C(C)C)c1NC(=O)OCC(Cl)(Cl)Cl ; CC(C)(O)c1nc(CO[Si](C)(C)C(C)(C)C)c(S(N)(=O)=O)s1 ; CC(C)c1nc(-c2cccc(C(F)(F)F)c2)c(F)c(C(C)C)c1NC(=O)NS(=O)(=O)c1sc(C(C)(C)O)nc1CO[Si](C)(C)C(C)(C)C]

### 1. Synthesis of 3-[(4-[[(tert-butyldimethylsilyl)oxy]methyl]-2-(2-hydroxypropan-2-yl)-1,3-thiazol-5-yl)sulfonyl]-1-[5-fluoro-2,4-bis(propan-2-yl)-6-[3-(trifluoromethyl)phenyl]pyridin-3-yl]urea

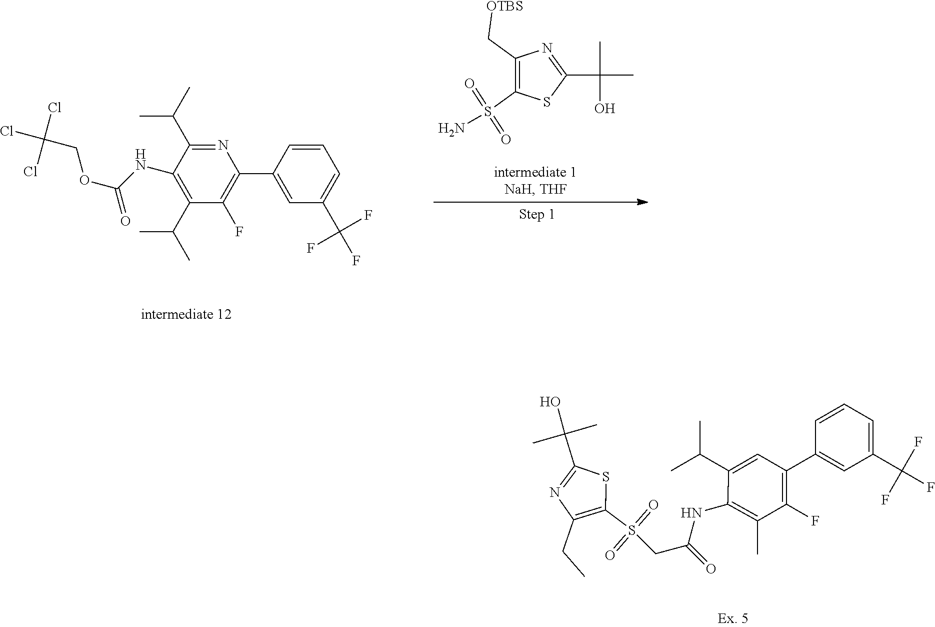 [SMILES: CC(C)c1nc(-c2cccc(C(F)(F)F)c2)c(F)c(C(C)C)c1NC(=O)OCC(Cl)(Cl)Cl ; CC(C)(O)c1nc(CO[Si](C)(C)C(C)(C)C)c(S(N)(=O)=O)s1 ; CCc1nc(C(C)(C)O)sc1S(=O)(=O)CC(=O)Nc1c(C(C)C)cc(-c2cccc(C(F)(F)F)c2)c(F)c1C]

To a stirred solution of 4-[[(tert-butyldimethylsilyl)oxy]methyl]-2-(2-hydroxypropan-2-yl)-1,3-thiazole-5-sulfonamide(85.3 mg, 0.23 mmol, 1.2 equiv) in THF(5 mL) was added NaH(14.0 mg, 0.58 mmol, 3 equiv) in portions at 0 degrees C. under a nitrogen atmosphere for 30 min. Then 2,2,2-trichloroethyl N-[5-fluoro-2,4-bis(propan-2-yl)-6-[3-(trifluoromethyl)phenyl]pyridin-3-yl]carbamate (100 mg, 0.19 mmol, 1 equiv) in THF (5 mL) was added to the above resulting mixture. After the addition was complete, the resulting mixture was stirred for 4 h. The reaction was quenched with water (0.5 mL) at 0 degrees C. The resulting mixture was concentrated under reduced pressure. The crude product (100 mg) was purified by Prep-HPLC with the following conditions (Column: XBridge Shield RP18 OBD Column 19*250 mm, 10 um; Mobile Phase A:Water(10 MMOL/L NH4HCO3+0.1% NH3.H2O), Mobile Phase B: ACN; Flow rate: 25 mL/min; Gradient: 60% B to 70% B in 8 min, 254/210 nm; Rt: 6.07 min) to afford 3-[(4-[[(tert-butyldimethylsilyl)oxy]methyl]-2-(2-hydroxypropan-2-yl)-1,3-thiazol-5-yl)sulfonyl]-1-[5-fluoro-2,4-bis(propan-2-yl)-6-[3-(trifluoromethyl)phenyl]pyridin-3-yl]urea (21 mg, 14.19%) as awhile solid.

LC-MS-3-[(4-[[(tert-butyldimethylsilyl)oxy]methyl]-2-(2-hydroxypropan-2-yl)-1,3-thiazol-5-yl)sulfonyl]-1-[5-fluoro-2,4-bis(propan-2-yl)-6-[3-(trifluoromethyl)phenyl]pyridin-3-yl]urea: (ES, m/z): 732.0

H-NMR-3-[(4-[[(tert-butyldimethylsilyl)oxy]methyl]-2-(2-hydroxypropan-2-yl)-1,3-thiazol-5-yl)sulfonyl]-1-[5-fluoro-2,4-bis(propan-2-yl)-6-[3-(trifluoromethyl)phenyl]pyridin-3-yl]urea: (300 MHz, DMSO-d) δ 8.17 (s, 2H), 7.82-7.73 (m, 2H), 4.94 (s, 2H), 3.22-3.07 (m, 2H), 1.48 (s, 6H), 1.19-1.18 (d, J=4.2 Hz, 12H), 1.02-1.00 (d, J=5.7 Hz, 12H), 0.87 (s, 9H).

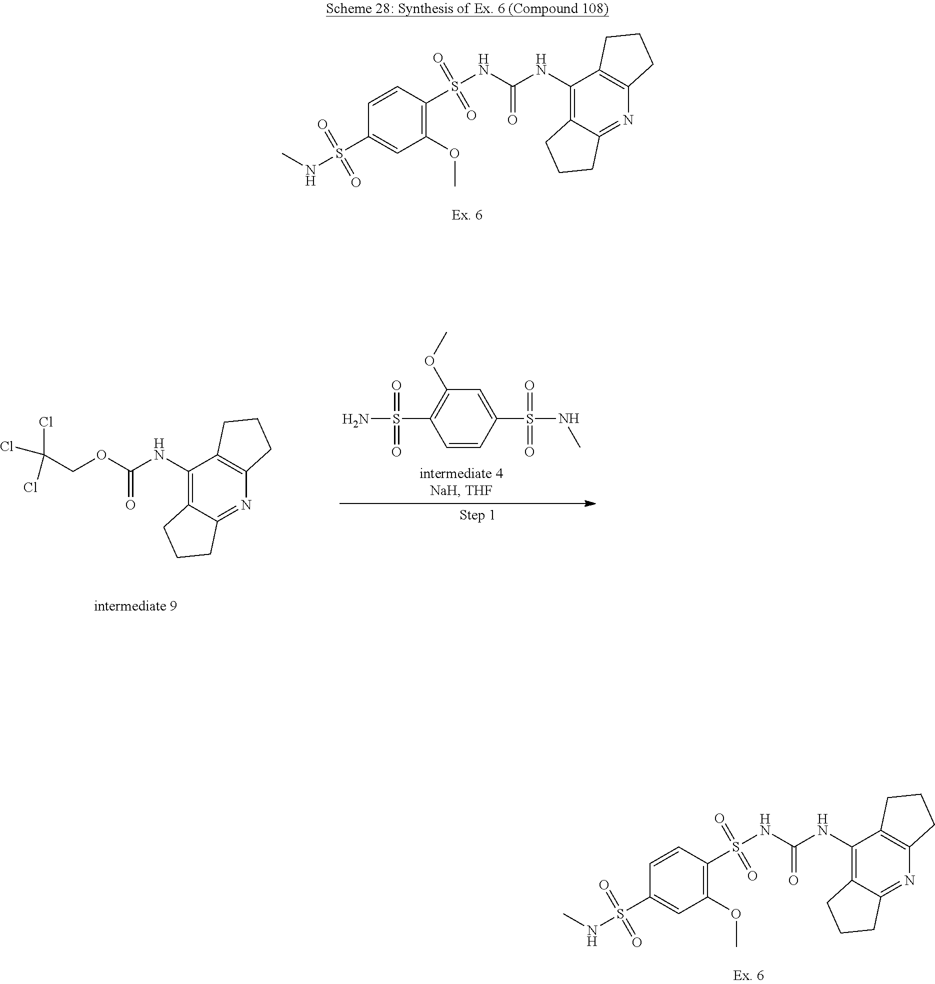 [SMILES: CNS(=O)(=O)c1ccc(S(=O)(=O)NC(=O)Nc2c3c(nc4c2CCC4)CCC3)c(OC)c1 ; O=C(Nc1c2c(nc3c1CCC3)CCC2)OCC(Cl)(Cl)Cl ; CNS(=O)(=O)c1ccc(S(N)(=O)=O)c(OC)c1 ; CNS(=O)(=O)c1ccc(S(=O)(=O)NC(=O)Nc2c3c(nc4c2CCC4)CCC3)c(OC)c1]

### 1. Synthesis of 3-[2azatricydo[7.3.0.0\hat{ }[3,7]]dodeca-1,3(7),8-trien-8-yl]1-[2-methoxy-4-(methylsulfamoyl)benezenesulfonyl]urea

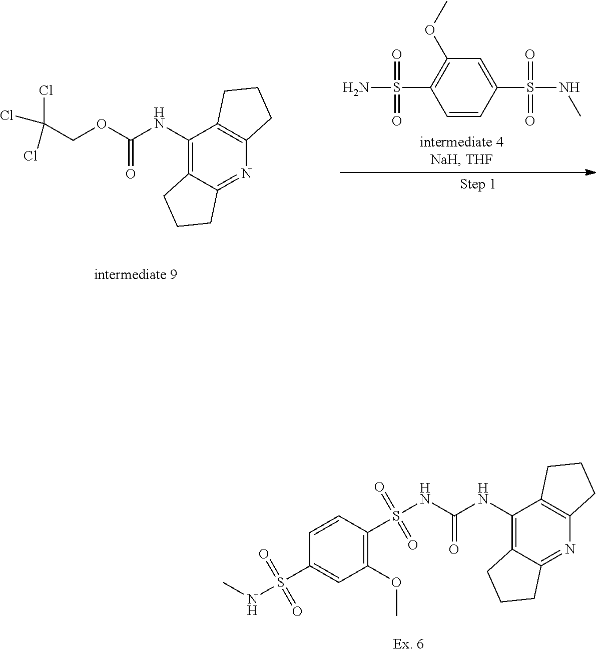 [SMILES: O=C(Nc1c2c(nc3c1CCC3)CCC2)OCC(Cl)(Cl)Cl ; CNS(=O)(=O)c1ccc(S(N)(=O)=O)c(OC)c1 ; CNS(=O)(=O)c1ccc(S(=O)(=O)NC(=O)Nc2c3c(nc4c2CCC4)CCC3)c(OC)c1]

Into a 50-mL round-bottom flask, was placed 3-methoxy-N1-methylbenzene-1,4 disulfonamide (90 mg, 0.32 mmol, 1 equiv), THF (10 mL, 123.43 mmol, 384.43 equiv), NaH (23.1 mg, 0.96 mmol, 3 equiv), and phenyl N-[2-azatricyclo[7.3.0.0\hat{ }[3,7]]dodeca-1,3(7),8-trien-8-yl]carbamate (122.9 mg, 0.42 mmol, 1.3 equiv). The resulting solution was stirred for 2 hr at 40 degrees C. using an oil bath. The reaction was then quenched by the addition of 10 mL of water. The resulting mixture was concentrated. The resulting residue was applied onto a silica gel column with dichloromethane/methanol (10/1). The crude product was purified by Prep-HPLC with the following conditions (2 #SHIMAZU (HPLC-01)): Column, XBridge Prep C18 OBD Column, 150 mm 5 um; mobile phase, Water(10MMOL/L, NH4HCO3+0.1% NH3.H2O) and ACN (9% PhaseB up to 23% in 6 min); Detector, UV. This resulted in 30 mg (19.44%) of 3-[2-azatricyclo[7.3.0.0\hat{ }[3,7]]dodeca-1,3(7),8-trien-8-yl]-1-[2-methoxy-4-(methylsulfamoyl)benzenesulfonyl]urea as a white solid.

LC-MS-3-[2-azatricyclo[7.3.0.0\hat{ }[3,7]]dodeca-1,3(7),8-trien-8-yl]-1-[2-methoxy-4-(methylsulfamoyl)benzenesulfonyl]urea: (ES, m/z): 480.11

H-NMR-3-[2-azatricyclo[7.3.0.0\hat{ }[3,7]]dodeca-1,3(7), 8-trien-8-yl]-1-[2-methoxy-4-(methylsulfamoyl)benzenesulfonyl]urea: (400 MHz, Methanol-d⁴) δ 8.15-8.03 (m, 1H), 7.52-7.49 (m, 2H), 4.07 (s, 3H), 3.02-3.00 (t, J=8.8 Hz, 4H), 2.91-2.88 (t, J=10.8 Hz, 4H), 2.59 (s, 3H), 2.57-2.18 (m, 4H).

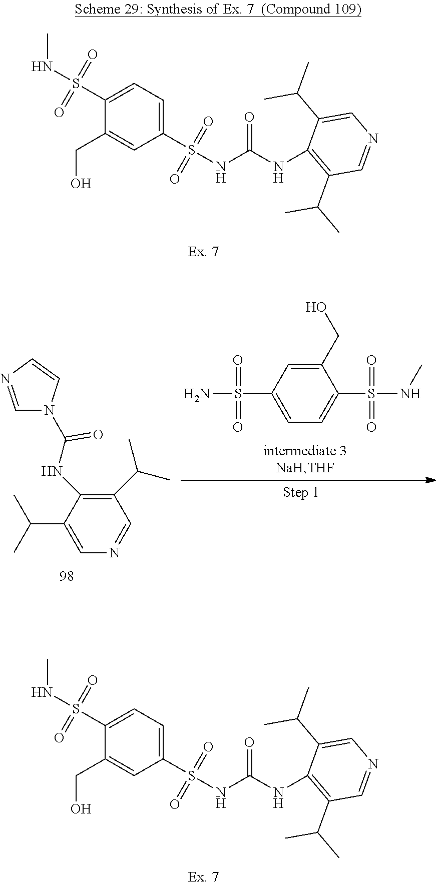 [SMILES: CNS(=O)(=O)c1ccc(S(=O)(=O)NC(=O)Nc2c(C(C)C)cncc2C(C)C)cc1CO ; CC(C)c1cncc(C(C)C)c1NC(=O)n1ccnc1 ; CNS(=O)(=O)c1ccc(S(N)(=O)=O)cc1CO ; CNS(=O)(=O)c1ccc(S(=O)(=O)NC(=O)Nc2c(C(C)C)cncc2C(C)C)cc1CO]

### 1. Synthesis of 1-[3,5-bis(propan-2-yl)pyridin-4-yl]-3-[2-(hydroxymethyl)-4 (methylsulfamoyl)benzenesulfonyl]urea

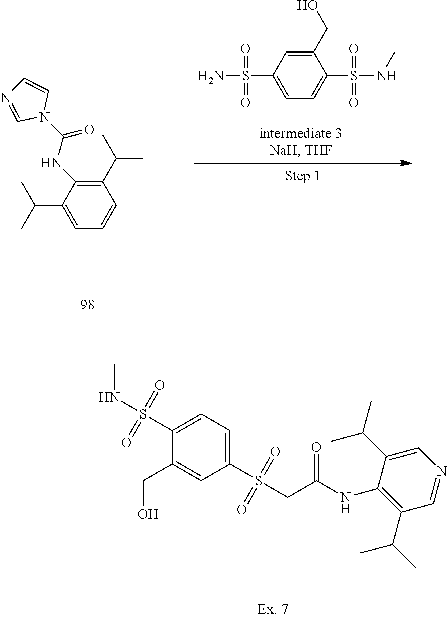 [SMILES: CC(C)c1cccc(C(C)C)c1NC(=O)n1ccnc1 ; CNS(=O)(=O)c1ccc(S(N)(=O)=O)cc1CO ; CNS(=O)(=O)c1ccc(S(=O)(=O)CC(=O)Nc2c(C(C)C)cncc2C(C)C)cc1CO]

Into a 25-mL round-bottom flask, was placed N-[3,5-bis(propan-2-yl)pyridin-4-yl]-1H-imidazole-1-carboxamide (100 mg, 0.38 mmol, 1 equiv), THF (15 ml), NaH (29.4 mg, 0.73mmoL 2 equiv, 60%), and 3-(hydroxymethyl)-N1-methylbenzene-1,4-disulfonamide (102.9 mg, 0.38 mmol, 1 equiv). The resulting solution was stirred for 6 hr at 25 degrees C. The reaction was quenched with ice-water and extracted with 3×100 mL of ethyl acetate. The organic layers were combined, dried over NaSO4, and concentrated under vacuum. The crude product (5 mL) was purified by Flash-Prep-HPLC with the following conditions (IntelFlash-1): Column, silica gel; mobile phase, ACN/H2O=10/90 increasing to ACN/H2O==90/10 within 1 hr; Detector, UV254. 200 mL of product-containing fractions were obtained. This resulted in 50 mg (28.10%) of 1-[3,5-bis(propan-2-yl)pyridin-4-yl]-3-[2-(hydroxymethyl)-4-(methylsulfamoyl)benzenesulfonyl]urea as a white solid.

LC-MS: (ES, m/z) 485.2

H-NMR (300 MHz, DMSO): 8.27 (d, J=6.3 Hz, 3H), 7.84 (m, 3H), 7.12 (d, J=3.6, 1H), 5.63 (m, 1H), 4.86 (d, J=4.8; Hz, 2H), 2.89 (m, 2H), 2.46 (m, 3H), 1.20 (m, 1H), 1.01 (m, 12H)

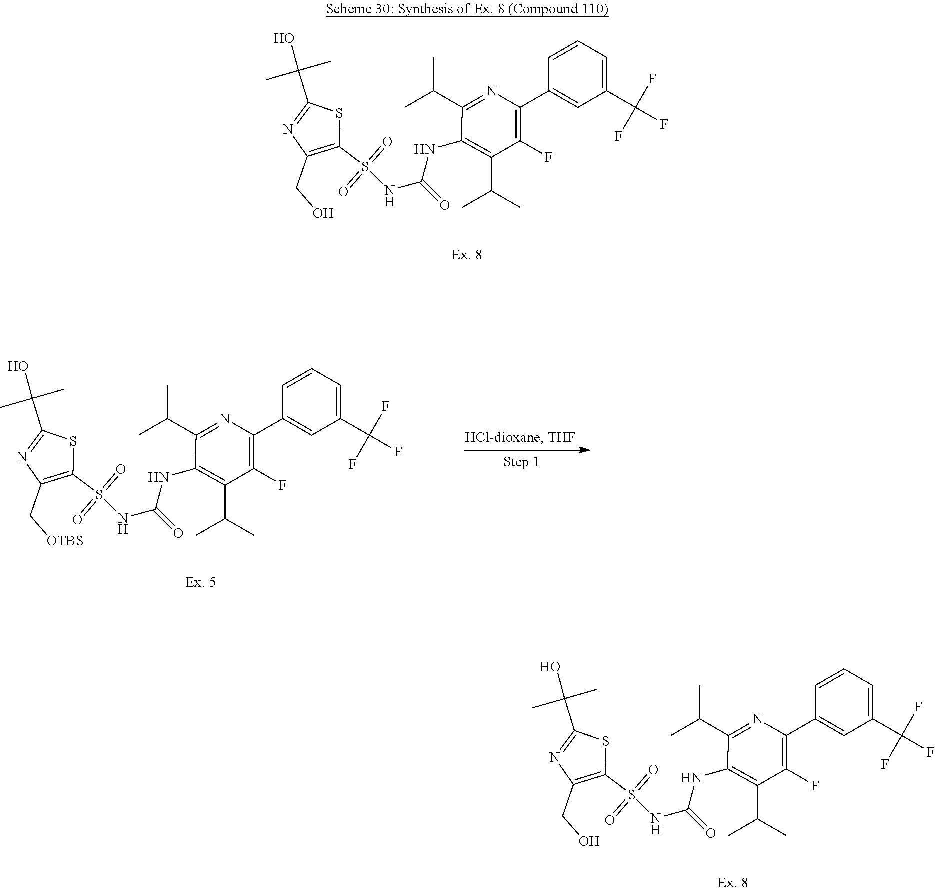 [SMILES: CC(C)c1nc(-c2cccc(C(F)(F)F)c2)c(F)c(C(C)C)c1NC(=O)NS(=O)(=O)c1sc(C(C)(C)O)nc1CO ; CC(C)c1nc(-c2cccc(C(F)(F)F)c2)c(F)c(C(C)C)c1NC(=O)NS(=O)(=O)c1sc(C(C)(C)O)nc1CO[Si](C)(C)C(C)(C)C ; CC(C)c1nc(-c2cccc(C(F)(F)F)c2)c(F)c(C(C)C)c1NC(=O)NS(=O)(=O)c1sc(C(C)(C)O)nc1CO]

### 1. Synthesis of 1-[5-fluoro-2,4-bis(propan-2-yl)-6-[3-(trifluoromethyl)phenyl]pyridin-3-yl]-3-[[4-(hydroxymethyl)-2-(2-hydroxypropan-2-yl)-1,3-thiazol-5-yl]sulfonyl]urea

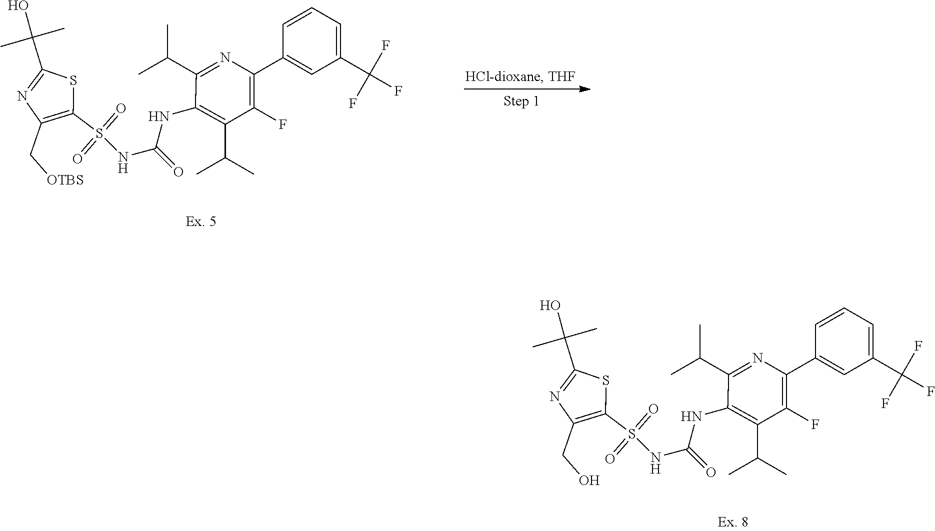 [SMILES: CC(C)c1nc(-c2cccc(C(F)(F)F)c2)c(F)c(C(C)C)c1NC(=O)NS(=O)(=O)c1sc(C(C)(C)O)nc1CO[Si](C)(C)C(C)(C)C ; CC(C)c1nc(-c2cccc(C(F)(F)F)c2)c(F)c(C(C)C)c1NC(=O)NS(=O)(=O)c1sc(C(C)(C)O)nc1CO]

Into a 50 mL round-bottom flask were added 3-[(4-[[(tert-butyldimethylsilyl)oxy]methyl]-2-(2-hydroxypropan-2-yl)-1,3-thiazol-5-yl)sulfonyl]-1-[5-fluoro-2,4-bis(propan-2-yl)-6-[3-(trifluoromethyl)phenyl]pyridin-3-yl]urea (15 mg, 0.02 mmol, 1 equiv) and THF(4 mL) at room temperature. To a stirred solution of 3-[(4-[[(tert-butyldimethylsilyl)oxy]methyl]-2-(2-hydroxypropan-2-yl)-1,3-thiazol-5-yl)sulfonyl]-1-[5-fluoro-2,4-bis(propan-2-yl)-6-[3-(trifluoromethyl)phenyl]pyridin-3-yl]urea (15 mg, 0.02 mmol, 1 equiv) in THF(4 mL) was added HCl (2M) in dioxane (2 mL, 65.82 mmol, 3216.28 equiv) dropwise at room temperature for 30 min. The resulting mixture was concentrated under reduced pressure. The crude product (15 mg) was purified by Prep-HPLC with the following conditions (Column: XBridge Shield RP18 OBD Column, 5 um, 19*150 mm; Mobile Phase A: Water (10MMOL/L NH4HCO3), Mobile Phase B: ACN; Flow rate: 25 mL/min; Gradient: 35% B to 55% B in 7 min; 254/210 nm; Rt: 6.45 min) to afford 1-[5-fluoro-2,4-bis(propan-2-yl)-6-[3-(trifluoromethyl)phenyl]pyridin-3-yl]-3-[[4-(hydroxymethyl)-2-(2-hydroxypropan-2-yl)-1,3-thiazol-5-yl]sulfonyl]urea(6.8 mg, 51.56%) as a white solid,

LC-MS-1-[5-fluoro-2,4-bis(propan-2-yl)-6-[3-(trifluoromethyl)phenyl]pyridin-3-yl]-3-[[4-(hydroxymethyl)-2-(2-hydroxypropan-2-yl)-1,3-thiazol-5-yl]sulfonyl]urea: (ES, m/z): 618

H-NMR-1-[5-fluoro-2,4-bis(propan-2-yl)-6-[3-(trifluoromethyl)phenyl]pyridin-3-yl]-3-[[4-(hydroxymethyl)-2-(2-hydroxypropan-2-yl)-1,3-thiazol-5-yl]sulfonyl]urea: (300 MHz, DMSO-d) δ 8.13 (s, 2H), 7.76-7.72 (m, 2H), 4.60 (d, 2H), 3.29-3.02 (m, 2H), 1.45 (s, 6H), 1.16-1.08 (m, 12H).

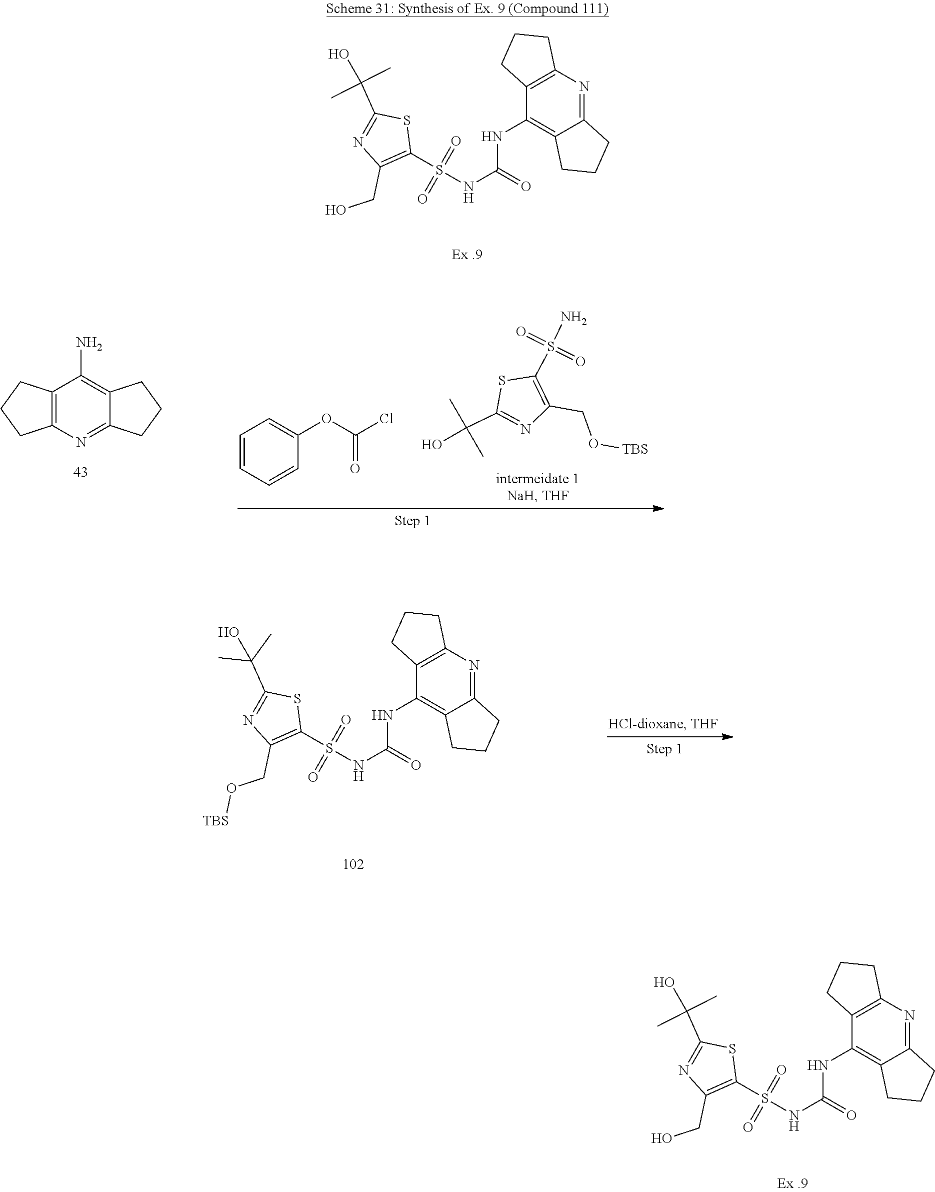

### 1. Synthesis of 3-[2-azatricyclo[7.3.0.0\hat{ }[3,7]]dodeca-1,3(7),8-trien-8-yl]1-[(4-[[(tert-butyldimethylsilyl)oxy]methyl]-2-(2-hydroxypropan-2-yl)-1,3-thiazol-5-yl)sulfonyl]urea

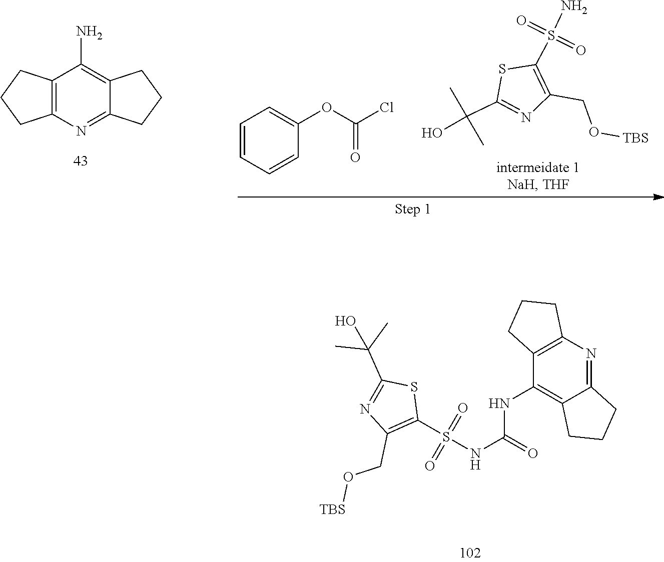

Into a 50-mL round-bottom flask, was placed 2-azatricyclo[7.3.0.0\hat{ }[3,7]]dodeca-1,3(7),8-trien-8-amine (26.1 mg, 0.15 mmol, 1.1 equiv), THF (10 mL, 0.14 mmol, 1.02 equiv), NaH (6.5 mg, 0.27 mmol, 2 equiv), and phenyl carbonochloridate (23.5 mg, 0.15 mmol, 1.1 equiv). The resulting solution was stirred for 1 overnight at room temperature. Then to the resulting solution was added 4-[[(tert-butyldimethylsilyl)oxy]methyl]-2-(2-hydroxypropan-2-yl)-1,3-thiazole-5-sulfonamide (50 mg, 0.14 mmol, 1 equiv). The resulting mixture was stirred for 4 hr at room temperature. The reaction was then quenched by the addition of 5 mL of water. The resulting solution was extracted with 3×15 of ethyl acetate. The organic layers were combined, dried over anhydrous sodium sulfate, and concentrated. The resulting residue was applied onto a silica gel column with ethyl acetate/hexane (1/1). The crude product was purified by Prep-HPLC with the following conditions (2 #SHIMADZU (HPLC-01)): Column, XBridge Shield RP18 OBD Column, 19*250 mm, 10 um; mobile phase, Water/10MMOL/L NH4HCO3) and ACN (30% PhaseB up to 46% in 8 min); Detector, UV. This resulted in 1.3 mg (1.68%) of 3-[2-azatricyclo[7.3.0.0\hat{ }[3,7]]dodeca-1,3(7),8-trien-8-yl]-1-[(4-[[(tert-butyldimethylsilyl)oxy]methyl]-2-(2-hydroxypropan-2-yl)-1,3-thiazol-5-yl)sulfonyl]urea as a white solid.

LCMS-3[2 azatricyclo[7.3.0.0\hat{ }[3,7]]dodeca-1,3(7),84Tien-8-yl]1-[(4-[[(tert-butyldimethylsilyl)oxy]methyl]-2-(2-hydroxypropan-2-yl)-1,3-thiazol-5-yl)sulfonyl]urea: (ES, m/z): 566.21

H-NMR-3-[2-azatricyclo[7.3.0.0\hat{ }[3,7]]dodeca-1,3(7),8-trien-8-yl]-1-[(4-[[(tert-butyldimethylsilyl)oxy]methyl]-2-(2-hydroxypropan-2-yl)-1,3-thiazol-5-yl)sulfonyl]urea: (400 MHz, Methanol-d₄) δ 5.06 (s, 2H), 2.97-2.93 (t, J=7.8 Hz, 4H), 2.84-2.82 (t, J=6.8 Hz, 4H), 2.14-2.07 (m, 4H), 1.58 (s, 6H), 0.90 (s, 9H), 0.10 (s, 6H).

### 2. Synthesis of 3-[2-azatricyclo[7.3.0.0\hat{ }[3,7]]dodeca-1,3(7),8-trien-8-yl]-1-[[4 (hydroxymethyl)-2-(2-hydroxypropan-2-yl)-1,3-thiazol-5-yl]sulfonyl]urea

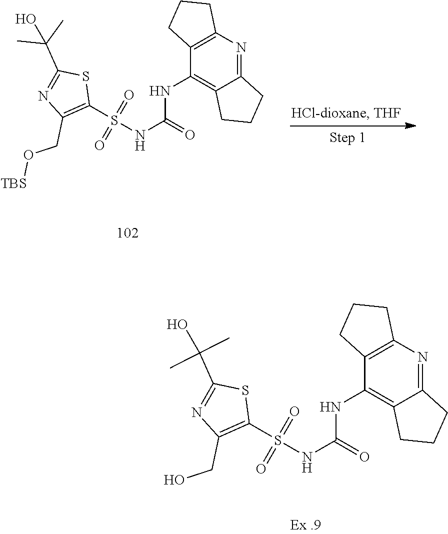

Into a 50-mL round-bottom flask, was placed 3-[2-azatrieyclo[7.3.0.0\hat{ }[3,7]]dodeca-1,3(7),8-trien-8-yl]-1-[(4-[[(tert-butyldimethylsilyl)oxy]methyl]-2-(2-hydroxypropan-2-yl)-1,3-thiazol-5-yl)sulfonyl]urea (10 mg, 0.02 mmol, 1 equiv), THF (5 mL, 61.71 mmol, 3495.07 equiv), and HCl(gas) in 1,4-dioxane(2 mL, 65.82 mmol, 4 M). The solution was stirred for 30 min at room temperature and was then quenched by the addition of 5 mL of water. The resulting mixture was extracted with 3×ml of ethyl acetate and the organic layers combined, dried over anhydrous sodium sulfate, and concentrated to provide a residue which was applied onto a silica gel column with chloroform/methanol (10/1). The crude product was purified by Prep-HPLC with the following conditions (2 #SHIMADZU (HPLC-01)): Column, XBridge Shield RP18 OBD Column, 19*250 mm, 10 um; mobile phase, Water(10MMOL/L NH4HCO3) and ACN (30% PhaseB up to 46% in 8 min); Detector, UV. This resulted in 6.2 mg (10.77%) of 3-[2-azatricyclo[7.3.0.0\hat{ }[3,7]]dodeca-1,3(7),8-trien-8-yl]-1-[[4-(hydroxymethyl)-2-(2-hydroxypropan-2-yl)-1,3-thiazol-5-yl]sulfonyl]urea as a white solid.

LC-MS-3-[2-azatricyclo[7.3.0.0\hat{ }[3,7]]dodeca-1,3(7),8-trien-8-yl]-1-[[4-(hydroxymethyl)-2-(2-hydroxypropan-2-yl)-1,3-thiazol-5-yl]sulfonyl]urea: (ES, m/z): 452.12

H-NMR-3-[2-azatricyclo[7.3.0.0\hat{ }[3,7]]dodeca-1,3(7),8-trien-8-yl]-1-[[4-(hydroxymethyl)-2-(2-hydroxypropan-2-yl)-1,3-thiazol-5-yl]sulfonyl]urea; (400 MHz, Dimethylsulfoxide-d₆) δ 8.05 (s, 1H), 6.16 (s, 2H), 4.66 (s, 2H). 2.78-2.76 (t, J=7.1 Hz, 4H), 2.72-2.71 (t, J=6 Hz, 4H), 1.99-1.86 (m, 4H), 1.46 (s, 6H).

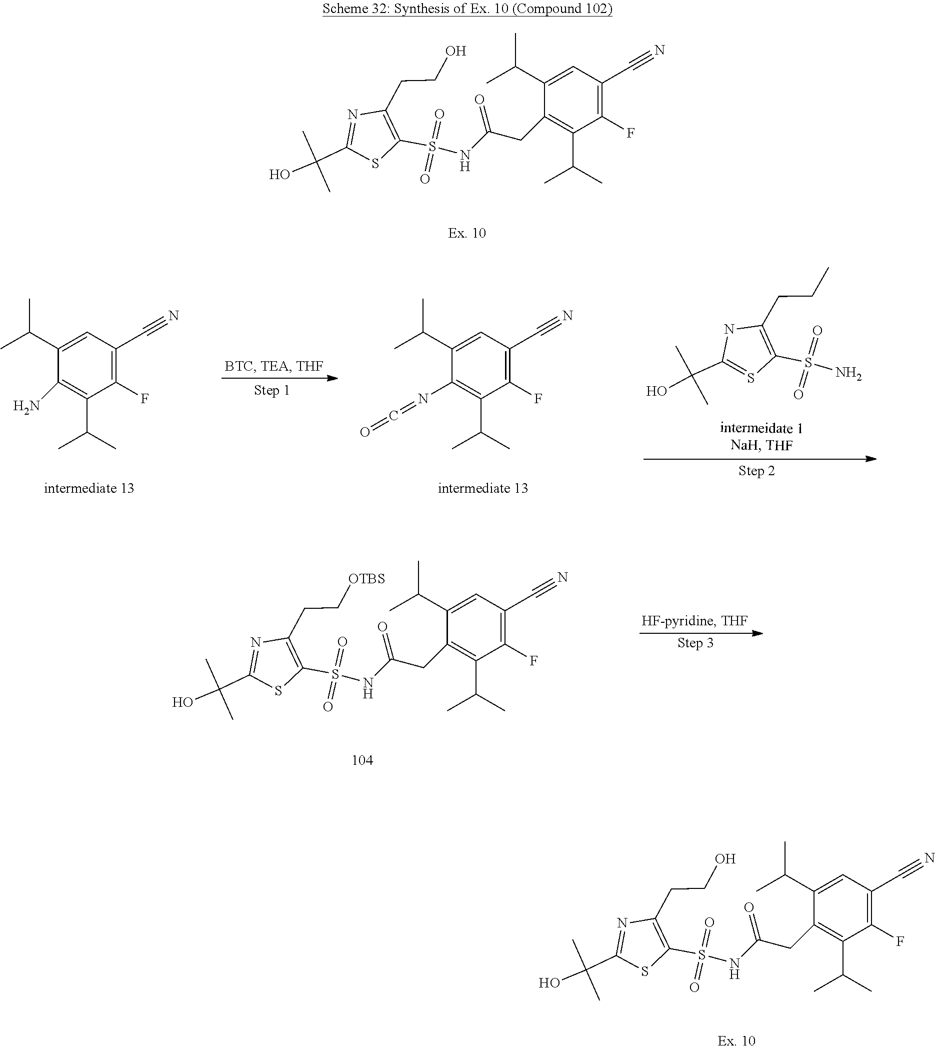

### 1. Synthesis of 2-fluoro-4-isocyanato-3,5-diisopropylbenzonitrile

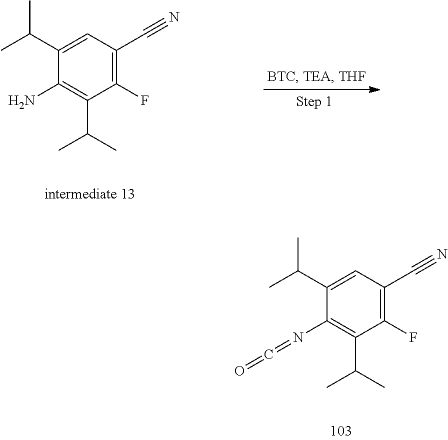 [SMILES: CC(C)c1cc(C#N)c(F)c(C(C)C)c1N ; CC(C)c1cc(C#N)c(F)c(C(C)C)c1N=C=O]

Into a 50-mL round-bottom flask purged and maintained with an inert atmosphere of nitrogen, was placed 4-amino-2-fluoro-3,5-bis(propan-2-yl)benzonitrile (100 mg), THF (44.4 mg), and TEA (0.1 mL). The resulting solution was stirred for 40 min at 70 degrees C. The resulting mixture was concentrated under vacuum. This resulted in 100 mg of 2-fluoro-4-isocyanato-3,5-bis(propan-2-yl)benzonitrile as a yellow solid.

### 2. Synthesis of 3-[(4-[2-[(tert-butyldimethylsilyl)oxy]ethyl]-2-(2-hydroxypropan-2-yl)-1,3-thiazol-5-yl)sulfonyl]1-[4-cyano-3-fluoro-2,6-bis(propan-2-yl)phenyl]urea

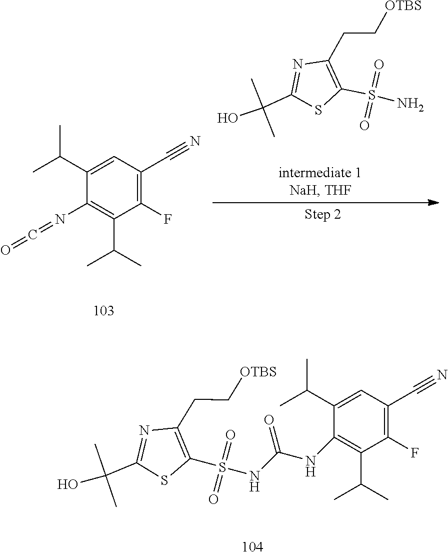 [SMILES: CC(C)c1cc(C#N)c(F)c(C(C)C)c1N=C=O ; CC(C)(O)c1nc(CCO[Si](C)(C)C(C)(C)C)c(S(N)(=O)=O)s1 ; CC(C)c1cc(C#N)c(F)c(C(C)C)c1NC(=O)NS(=O)(=O)c1sc(C(C)(C)O)nc1CCO[Si](C)(C)C(C)(C)C]

Into a 50-mL round-bottom flask, was placed 2-fluoro-4-isocyanato-3,5-bis(propan-2-yl)benzonitrile (100 mg), and THF (5 mL). This was followed by the addition of NaH (48.78 mg, 3 equiv) in portions at 0° C. To this was added 4-[2-[(tert-butyldimethylsilyl)oxy]ethyl]-2-(2-hydroxypropan-2-yl)-1,3-thiazole-5-sulfonamide (154 mg). The resulting solution was stirred for 1 hr at room temperature, quenched with ice-water, and extracted with EtOAc. The combined organic phase was dried over Na₂SO₄ and concentrated under vacuo. The residue was applied onto a silica gel column with ethyl acetate/petroleum ether (1:3). This resulted in 100 mg of 3-[(4-[2-[(tert-butyldimethylsilyl)oxy]ethyl]-2-(2-hydroxypropan-2-yl)-1,3-thiazol-5-yl)sulfonyl]-1-[4-cyano-3-fluoro-2,6-bis(propan-2-yl)phenyl]urea as a yellow solid,

### 3. Synthesis of 1-[4-cyano-3-fluoro-2,6-bis(propan-2-yl)phenyl]-3-[[4-(2-hydroxyethyl)-2-(2-hydroxypropan-2-yl)-1,3-thiazol-5-yl]sulfonyl]urea

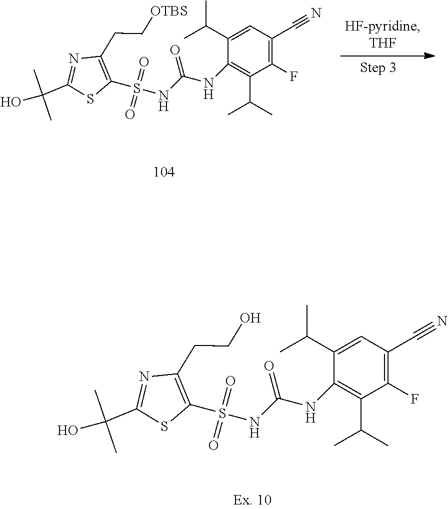 [SMILES: CC(C)c1cc(C#N)c(F)c(C(C)C)c1NC(=O)NS(=O)(=O)c1sc(C(C)(C)O)nc1CCO[Si](C)(C)C(C)(C)C ; CC(C)c1cc(C#N)c(F)c(C(C)C)c1NC(=O)NS(=O)(=O)c1sc(C(C)(C)O)nc1CCO]

Into a 50-mL round-bottom flask, was placed 3-[(4-[2-[(tert-butyldimethylsilyl)oxy]ethyl]-2-(2-hydroxypropan-2-yl)-1,3-thiazol-5-yl)sulfonyl]-1-[4-cyano-3-fluoro-2,6-bis(propan-2-yl)phenyl]urea (100 mg), THF (15 mL), and HF-Pyridine (0.25 mL). The resulting solution was stirred for 10 h at room temperature and was subsequently concentrated under vacuum. The residue was dissolved in 10 mL of MeOH, The solids were filtered out. The crude product was purified by Prep-HPLC with the following conditions (Prep-HPLC-018): Column, XBridge Prep C18 QBD Column, 5 um, 19*150 mm; mobile phase. Water (10 mmol/L NH4HCO3) and ACN (9% Phase B up to 45% in 7 min). This resulted in 20.6 mg (30%) of 1-[4-cyano-3-fluoro-2,6-bis(propan-2-yl)phenyl]-3-[[4-(2-hydroxyethyl)-2-(2-hydroxypropan-2-yl)-1,3-thiazol-5-yl]sulfonyl]urea as a white solid.

LC-MS-1-[4-cyano-3-fluoro-2,6-bis(propan-2-yl)phenyl]-3-[[4-(2-hydroxyethyl)-2-(2-hydroxypropan-2-yl)-1,3-thiazol-5-yl]sulfonyl]urea: (ES, m/z): 512.2

H-NMR-1-[4-cyano-3-fluoro-2,6-bis(propan-2-yl)phenyl]-3-[[4-(2-hydroxyethyl)-2-(2-hydroxypropan-2-yl)-1,3-thiazol-5-yl]sulfonyl]urea: (300 MHz, Methanol-d₄) δ 7.45 (d, J=6.8 Hz, 1H), 3.90 (s, 4H), 1.58 (s, 6H), 1.26 (d, J=6.9 Hz, 6H), 1.16 (d, J=6.9 Hz, 6H).

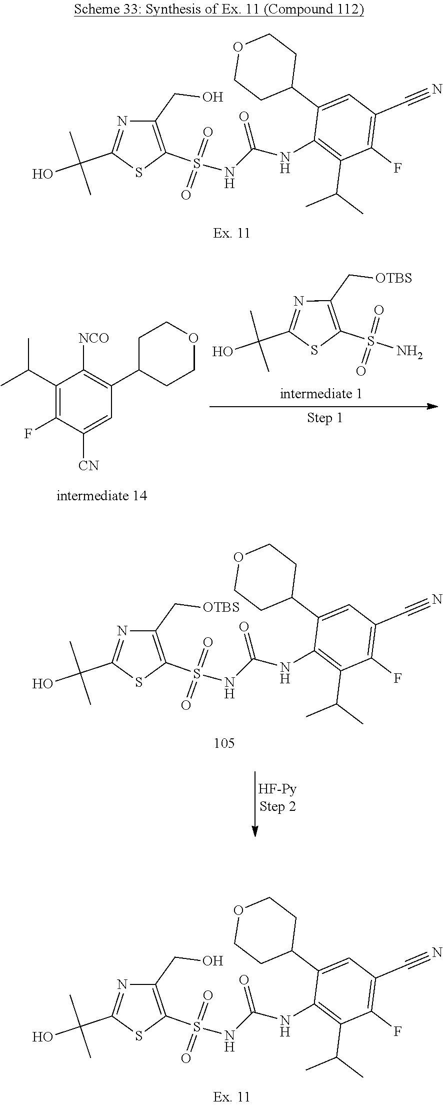

### 1. Synthesis of 3-(4-[[(tert-butyldimethylsilyl)oxy]methyl]-2-(2-hydroxypropan-2-yl)-1,3-thiazole-5-sulfonyl)-1-[4-cyano-3-fluoro-6-(oxan-4-yl)-2-(propan-2-yl)phenyl]urea

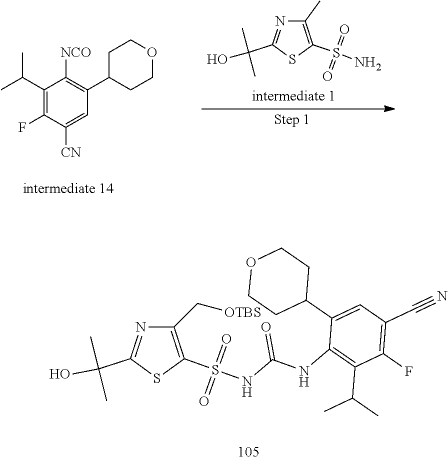

Into a 50-mL round-bottom flask purged and maintained with an inert atmosphere of nitrogen, was placed a solution of 2-fluoro-4-isocyanato-5-(oxan-4-yl)-3-(propan-2-yl)benzonitrile (105 mg, 0.36 mmol, 1 equiv) in ACN (5 mL), 4-[[(tert-butyldimethylsilyl)oxy]methyl]-2-(2-hydroxypropan-2-yl)-1,3-thiazole-5-sulfonamide (133.5 mg, 0.36 mmol, 1 equiv), and K₂CO₃ (100.7 mg, 0.73 mmol, 2 equiv). The resulting solution was stirred for 2 hr at room temperature. The solids were filtered out. The resulting mixture was concentrated. This resulted in 150 mg (62.89%) of 3-(4-[[(tert-butyldimethylsilyl)oxy]methyl]-2-(2-hydroxypropan-2-yl)-1,3-thiazole-5-sulfonyl)-1-[4-cyano-3-fluoro-6-(oxan-4-yl)-2-(propan-2-yl)phenyl]urea as yellow oil.

### 2. Synthesis of 1-[4-cyano-3-fluoro-6-(oxan-4-yl)-2-(propan-2-yl)phenyl]-3-[4-(hydroxymethyl)-2-(2-(hydroxypropan-2-yl)-1,3-thiazole-5-sulfonyl]urea

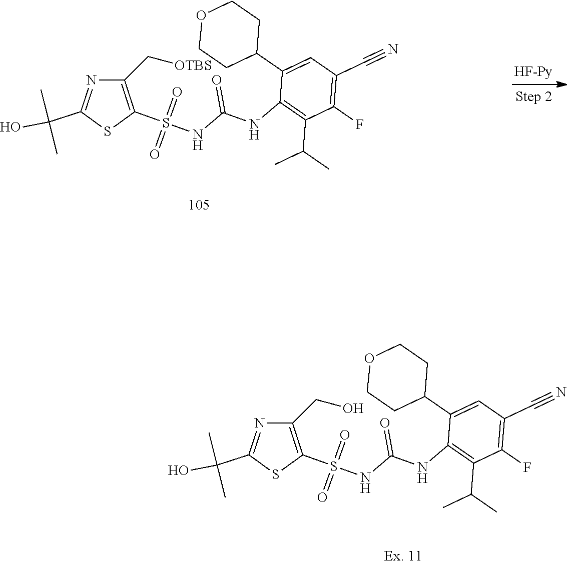 [SMILES: CC(C)c1c(F)c(C#N)cc(C2CCOCC2)c1NC(=O)NS(=O)(=O)c1sc(C(C)(C)O)nc1CO[Si](C)(C)C(C)(C)C ; CC(C)c1c(F)c(C#N)cc(C2CCOCC2)c1NC(=O)NS(=O)(=O)c1sc(C(C)(C)O)nc1CO]

Into a 50-mL round-bottom flask, was placed a solution of 3-(4-[[(tert-butyldimethylsilyl)oxy]methyl]-2-(2-hydroxypropan-2-yl)-1,3-thiazole-5-sulfonyl)-1-[4-cyano-3-fluoro-6-(oxan-4-yl)-2-(propan-2-yl)phenyl]urea (150 mg, 0.23 mmol, 1 equiv) in THF (5 mL) and HF-Pyridine (227.0 mg, 2.29 mmol, 10 equiv). The resulting solution was stirred for 1 hr at room temperature. The resulting mixture was concentrated. The crude product (100 mg) was purified by Prep-HPLC with the following conditions (Prep-HPLC-018): Column, XBridge Prep OBD CIS Column, 19*250 mm, 5 um; mobile phase, Water(10 MMOL/L NH4HCO3) and ACN (12% PhaseB up to 38% in 7 min), Detector, UV. This resulted in 35.4 mg (28.59%) of 1-[4-cyano-3-fluoro-6-(oxan-4-yl)-2-(propan-2-yl)phenyl]-3-[4-(hydroxymethyl)-2-(2-hydroxypropan-2-yl)-1,3-thiazole-5-sulfonyl]urea as awhile solid.

LC-MS-1-[4-cyano-3-fluoro-6-(oxan-4-yl)-2-(propan-2-yl)phenyl]-3-[4-(hydroxymethyl)-2-(2-hydroxypropan-2-yl)-1,3-thiazole-5-sulfonyl]urea (ES, m/z) [M+Z]⁺=541.1

H-NMR-1-[4-cyano-3-fluoro-6-(oxan-4-yl)-2-(propan-2-yl)phenyl]-3-[4-(hydroxymethyl)-2-(2-hydroxypropan-2-yl)-1,3-thiazole-5-sulfonyl]urea: ¹HNMR (400 MHz, Methanol-d₄) δ 7.48 (d, J=6.8 Hz, 1H), 3.98 (d, J=11.3 Hz, 2H), 3.47 (s, 2H), 3.32 (s, 1H), 3.04 (s, 1H), 1.66 (s, 4H), 1.61 (s, 6H), 1.26 (d, J=7.0 Hz, 6H),

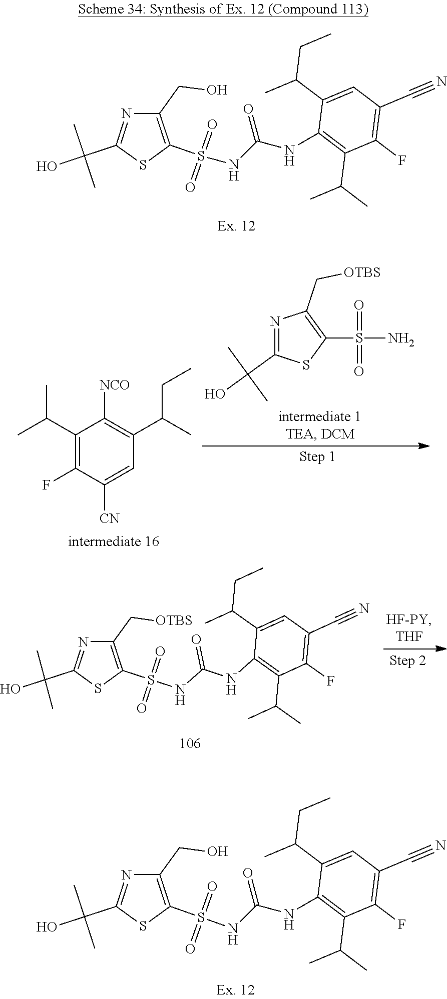

### 1. Synthesis of 1-[6-(butan-2-yl)-4-cyano-3-fluoro-2-(propan-2-yl)phenyl]-3-(4-[[(tert-butyldimethylsilyl)oxy]methyl]-2-(2-hydroxypropan-2-yl)-1,3-thiazole-5-sulfonyl)urea

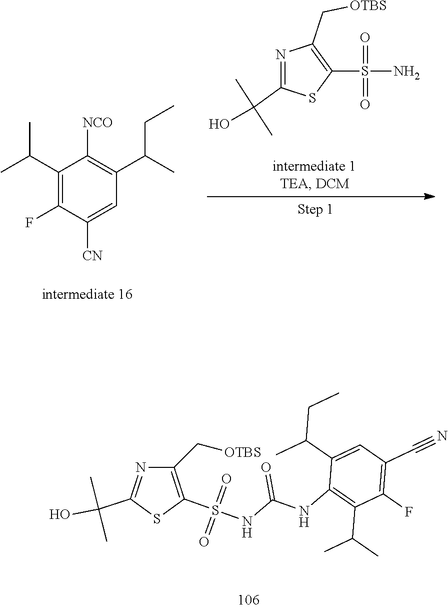

A solution/mixture of 4-[[(tert-butyldimethylsilyl)oxy]methyl]-2-(2-hydroxypropan-2-yl)-1,3-thiazole-5-sulfonamide (443.6 mg, 1.21 mol, 0.900 equiv), TEA (17.1 mg, 0.17 mmol, 2 equiv) and 5-(butan-2-yl)-2-fluoro-4-isocyanato-3-(propan-2-yl)benzonitrile(350 mg, 1.34 mol, 1 equiv) in DCM (10 mL) was stirred for 3 h at room temperature. The resulting mixture was diluted with water (50 mL). The resulting mixture was extracted with CH₂Cl₂ (2×50 mL). The combined organic layers were concentrated under vacuum. This resulted in 1-[6-(butan-2-yl)-4-cyano-3-fluoro-2-(propan-2-yl)phenyl]-3-(4-[[(tert-butyldimethylsilyl)oxy]methyl]-2-(2-hydroxypropan-2-yl)-1,3-thiazole-5-sulfonyl)urea(700 mg, crude) as a yellow solid.

LC-MS-1-[6-(butan-2-yl)-4-cyano-3-fluoro-2-(propan-2-yl)phenyl]-3-(4-[[(tert-butyldimethylsilyl)oxy]methyl]-2-(2-hydroxypropan-2-yl)-1,3-thiazole-5-sulfonyl)urea: (ES, m/z): [M+H]⁺=627.2

### 2. Synthesis of 1-[6-(butan-2-yl)-4-cyano-3-fluoro-2-(propan-2-yl)phenyl]-3-[4-(hydroxymethyl)-2-(2-hydroxypropan-2-yl)-1,3-thiazole-5-sulfonyl]urea

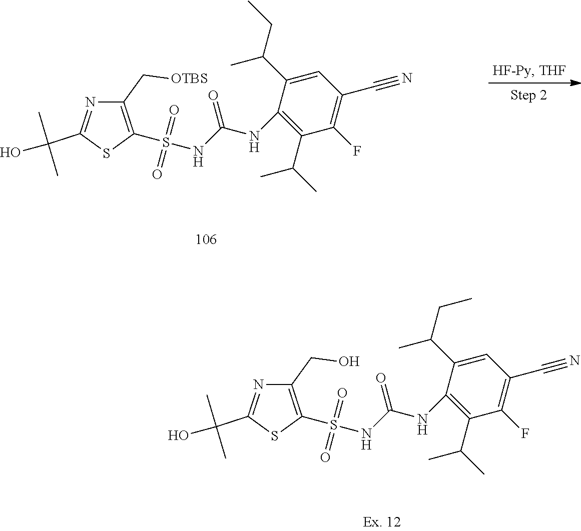 [SMILES: CCC(C)c1cc(C#N)c(F)c(C(C)C)c1NC(=O)NS(=O)(=O)c1sc(C(C)(C)O)nc1CO[Si](C)(C)C(C)(C)C ; CCC(C)c1cc(C#N)c(F)c(C(C)C)c1NC(=O)NS(=O)(=O)c1sc(C(C)(C)O)nc1CO]

Into a 100-mL round-bottom flask, was placed 1-[6-(butan-2-yl)-4-cyano-3-fluoro-2-(propan-2-yl)phenyl]-3-(4-[[(tert-butyldimethylsilyl)oxy]methyl]-2-(2-hydroxypropan-2-yl)-1,3-thiazole-5-sulfonyl)urea (650 mg, 1.04 mmol, 1 equiv, 80%), THF (8 mL), and TFA/Py (518.6 mg, 5.19 mmol, 5.002 equiv). The resulting solution was stirred for 1 h at room temperature and was then concentrated. The crude product was purified by Prep-HPLC with the following conditions (Prep-HPLC-018): Column, XBridgePrep OBD C18 Column, 19*250 mm, 5 um; mobile phase, Water(10 mmol/L NH₄HCO₃) and ACN (14% PhaseB up to 56% in 7 min); Detector, UV 254/210 nm. This resulted in 180 mg (42.3%) of 1-[6-(butan-2-yl)-4-cyano-3-fluoro-2-(propan-2-yl)phenyl]-3-[4-(hydroxymethyl)-2-(2-hydroxypropan-2-yl)-1,3-thiazole-5-sulfonyl]urea as a white solid.

LC-MS-1-[6-(butan-2-yl)-4-cyano-3-fluoro-2-(propan-2-yl)phenyl]-3-[4-(hydroxymethyl)-2-(2-hydroxypropan-2-yl)-1,3-thiazole-5-sulfonyl]urea: (ES, m/z): [M+H]⁺=513.2

H-NMR-1-[6-(butan-2-yl)-4-cyano-3-fluoro-2-(propan-2-yl)phenyl]-3-[4-(hydroxymethyl)-2-(2-hydroxypropan-2-yl)-1,3-thiazole-5-sulfonyl]urea; ¹H NMR (300 MHz, CD₃OD-d₄) S 7.41 (d, J=6.9 Hz, 1H), 4.91 (s, 2H), 3.24 (s, 1H), 2.95-2.86 (m, 1H), 1.60 (s, 6H), 1.52 (t, J=6.8 Hz, 2H), 1.27-1.25 (m, 6H), 1.12 (d, J=6.9 Hz, 3H), 0.79 (s, 3H).

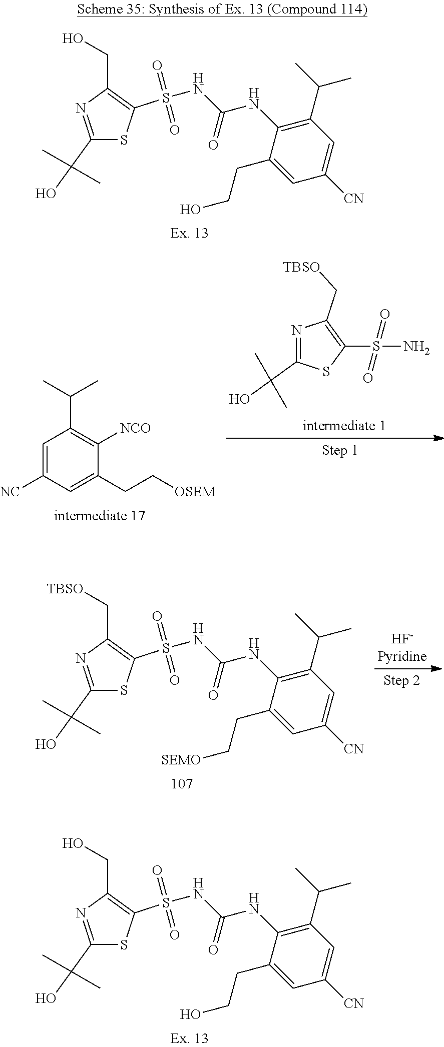

### 1. Synthesis of 3-(4-[[(tert-butyldimethylsilyl)oxy]methyl]-2-(2-hydroxypropan-2-yl)-1,3-thiazole-5-sulfonyl)-1-[4-cyano-2-(propan-2-yl)-6-(2-[[2-(trimethylsilyl)ethoxy]methoxy]ethyl)phenyl]urea

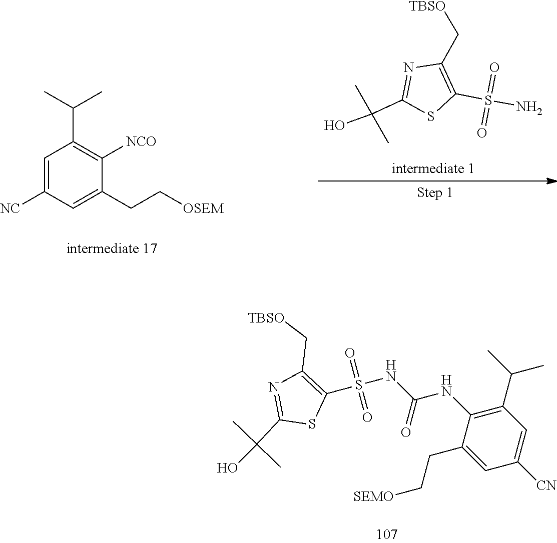

Into a 50-mL round-bottom flask, was placed a solution of 4-[[(tert butyldimethylsilyl)oxy]methyl]-2-(2-hydroxypropan-2-yl)-1,3-thiazole-5-sulfonamide (152.5 mg, 0.42 mmol, 1 equiv) in acetone (10 mL), K₂CO₃ (115.0 mg, 0.83 mmol, 2 equiv), and 4-isocyanato-3-(propan-2-yl)-5-(2-[[2-(trimethylsilyl)ethoxy]methoxy]ethyl)benzonitrile (150 mg, 0.42 mmol, 1 equiv). The resulting solution was stirred for 2 hr at room temperature. The resulting mixture was concentrated. The resulting residue was applied onto a silica gel column with ethyl acetate/hexane (1:20). This resulted in 130 mg (42.97%) of 3-(4-[[(tert-butyldimethylsilyl)oxy]methyl]-2-(2-hydroxypropan-2-yl)-1,3-thiazole-5-sulfonyl)-1-[4-cyano-2-(propan-2-yl)-6-(2-[[2-(trimethylsilyl)ethoxy]methoxy]ethyl)phenyl]urea as a yellow solid.

### 2. Synthesis of 1-[4-cyano-2-(2-hydroxyethyl)-6-(propan-2-yl)phenyl]-3-[4-(hydroxymethyl)-2-(2-hydroxypropan-2-yl)-1,3-thiazole-5-sulfonyl]urea

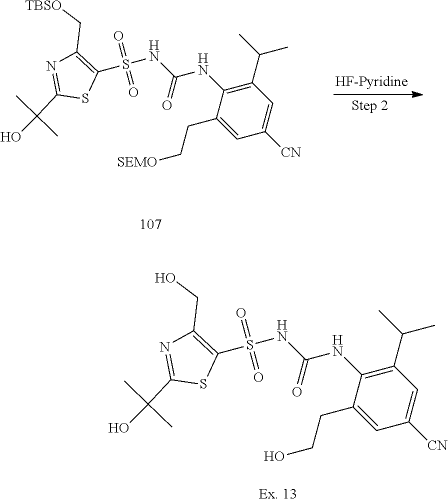

Into a 25-mL round-bottom flask, was placed 3-(4-[[(tert-butyidimethylsilyl)oxy]methyl]-2-(2-hydroxypropan-2-yl)-1,3-thiazole-5-sulfonyl)-1-[4-cyano-2-(propan-2-yl)-6-(2-[[2-(trimethylsilyl)ethoxy]methoxy]ethyl)phenyl]urea (110 mg, 0.15 mmol, 1 equiv), TFA (34.5 mg, 0.30 mmol, 2 equiv), and DCM (5 mL). The resulting solution was stirred for 2 hr at room temperature. The resulting mixture was concentrated. The crude product (150 mg) was purified by Prep-HPLC with the following conditions (2 #SHIMADZU (HPLC-01)): Column, XBridge Prep C18 OBD Column, 5 um, 19*150 mm; mobile phase, Water(10MMOL/L NH4HCO3) and ACN (8% PhaseB up to 50% in 7 min); Detector, UV. This resulted in 14.8 mg (20.3%) of 1-[4-cyano-2-(2-hydroxyethyl)-6-(propan-2-yl)phenyl]-3-[4-(hydroxymethyl)-2-(2-hydroxypropan-2-yl)-1,3-thiazole-5-sulfonyl]urea as a white solid,

LC-MS-1-[4-cyano-2-(2-hydroxyethyl)-6-(propan-2-yl)phenyl]-3-[4-(hydroxymethyl)-2-(2-hydroxypropan-2-yl)-1,3-thiazole-5-sulfonyl]urea (ES, m/z): [M+Z]⁺=483.1

H-NMR-1-[4-cyano-2-(2-hydroxyethyl)-6-(propan-2-yl)phenyl]-3-[4-(hydroxymethyl)-2-(2-hydroxypropan-2-yl)-1,3-thiazole-5-sulfonyl]urea: (400 MHz, Methanol-d₄) δ 7.50 (d, J=14.1 Hz, 2H), 4.86 (d, J=3.6 Hz, 2H), 3.73 (s, 2H), 3.16 (s, 1H). 2.83 (s, 2H), 1.61 (s, 6H), 1.15 (d, J=6.8 Hz, 6H),

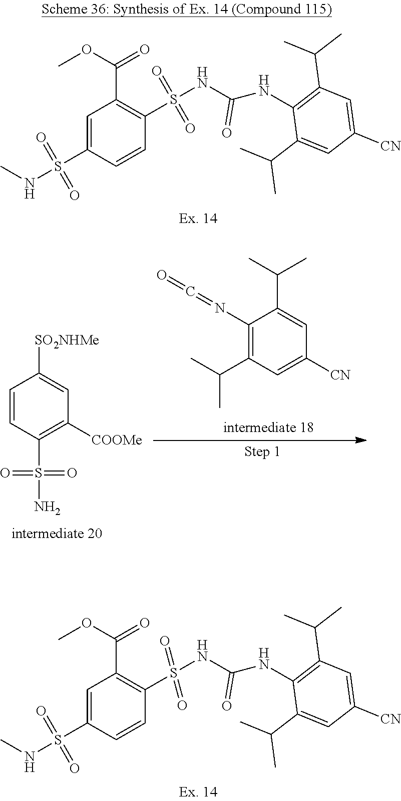

### 1. Synthesis of methyl 2-[([[4-cyano-2,6-bis(propan-2-yl)phenyl]carbamoyl]amino)sulfonyl]-5-(methylsulfamoyl)benzoate

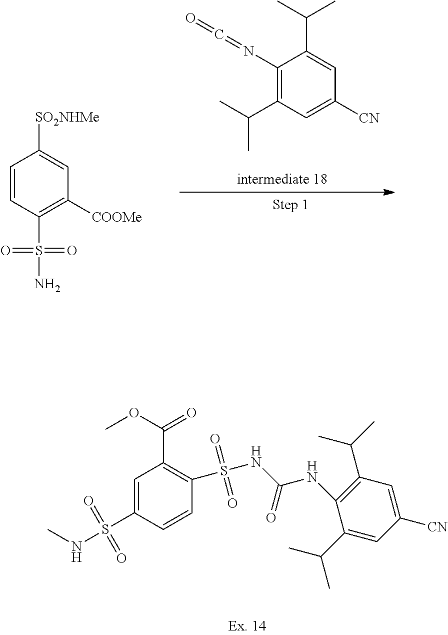

Into a 50-mL round-bottom flask, was placed methyl 5-(methylsulfamoyl)-2-sulfamoylbenzoate (50 mg), DCM (10 mL), TEA (30 mg), and 4-isocyanato-3,5-bis(propan-2-yl)benzonitrile (60 mg). The resulting solution was stirred for 2 hours. The crude product was purified by Prep-HPLC. This resulted in methyl 2-[([[4-cyano-2,6-bis(propan-2-yl)phenyl]carbamoyl]amino)sulfonyl]-5-(methylsulfamoyl)benzoate (5.1 mg, 6.2%) as awhile solid.

LC-MS-2-[([[4-cyano-2,6-bis(propan-2-yl)phenyl]carbamoyl]amino)sulfonyl]-5-(methylsulfamoyl)benzoate: (ES, m/z): [M+H]⁺=537

H-NMR-2-[([[4-cyano-2,6-bis(propan-2-yl)phenyl]carbamoyl]amino)sulfonyl]-5-(methylsulfamoyl)benzoate: (300 MHz, MeOH-d4): δ 8.35-8.32 (s, 1H), 8.16-7.95 (m, 2H), 7.57-7.45 (m, 2H). 4.00-3.91 (m, 3H), 3.00 (s, 2H), 2.56-2.50 (s, 3H), 1.33-0.90 (m, 1211).

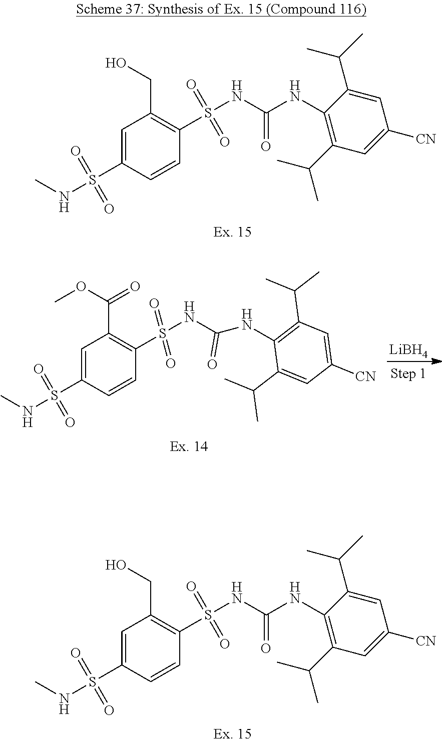

### 1. Synthesis of 1-[4-cyano-2,6-bis(propan-2-yl)phenyl]-3-[2-(hydroxymethyl)-4-(methylsulfamoyl)benzenesulfonyl]urea

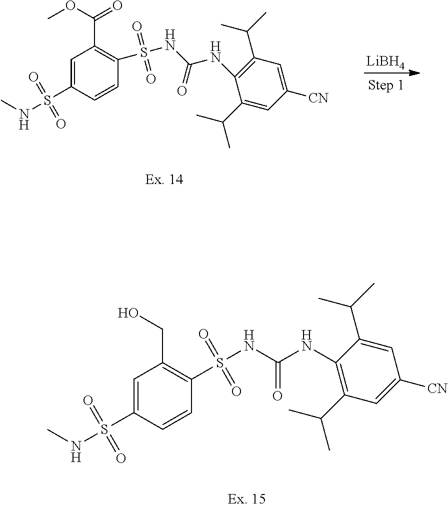 [SMILES: CNS(=O)(=O)c1ccc(S(=O)(=O)NC(=O)Nc2c(C(C)C)cc(C#N)cc2C(C)C)c(C(=O)OC)c1 ; "LiBH4" ; CNS(=O)(=O)c1ccc(S(=O)(=O)NC(=O)Nc2c(C(C)C)cc(C#N)cc2C(C)C)c(CO)c1]

Into a 100-mL round-bottom flask, was placed methyl 2-[([[4-cyano-2,6-bis(propan-2-yl)phenyl]carbamoyl]amino)sulfonyl]-5-(methylsulfamoyl)benzoate (100 mg), THF (20 mL), and LiBH4 (20 mg). The resulting solution was stirred for 3 hours. The crude product was purified by Prep-HPLC. This resulted in 51.2 mg of 1-[4-cyano-2,6-bis(propan-2-yl)phenyl]-3-[2-(hydroxymethyl)-4-(methylsulfamoyl)benzenesulfonyl]urea as a white solid, (m/z: 508.1) Scheme 38: Synthesis of Ex, 16 (Compound 117)

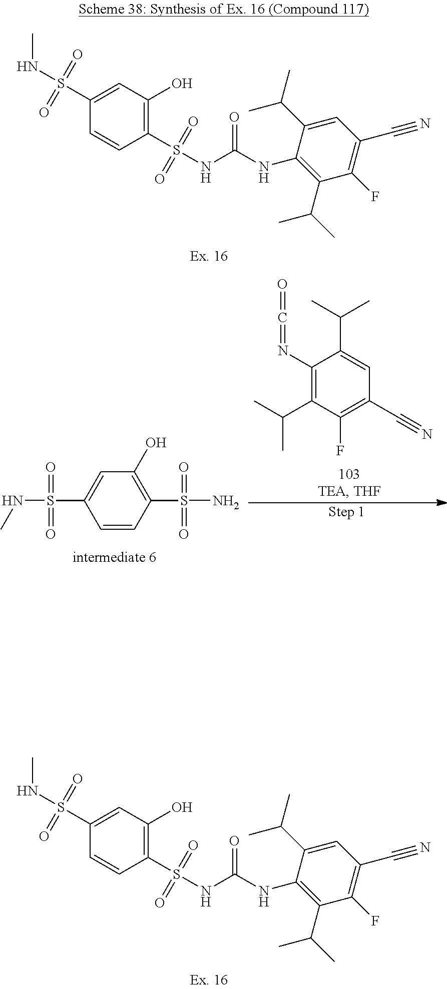 [SMILES: CNS(=O)(=O)c1ccc(S(=O)(=O)NC(=O)Nc2c(C(C)C)cc(C#N)c(F)c2C(C)C)c(O)c1 ; CNS(=O)(=O)c1ccc(S(N)(=O)=O)c(O)c1 ; CC(C)c1cc(C#N)c(F)c(C(C)C)c1N=C=O ; CNS(=O)(=O)c1ccc(S(=O)(=O)NC(=O)Nc2c(C(C)C)cc(C#N)c(F)c2C(C)C)c(O)c1]

### 1. Synthesis of 1-[4-cyano-3-fluoro-2,6-bis(propan-2-yl)phenyl]-3-[[2-methoxy-4(methylsulfamoyl)benezene]sulfonyl]urea

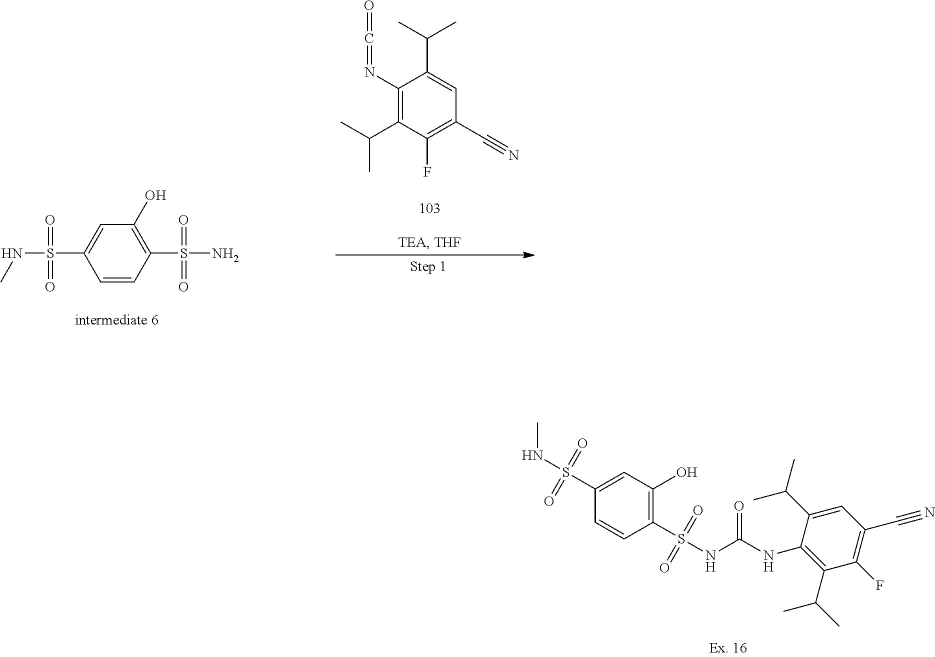 [SMILES: CC(C)c1cc(C#N)c(F)c(C(C)C)c1N=C=O ; CNS(=O)(=O)c1ccc(S(N)(=O)=O)c(O)c1 ; CNS(=O)(=O)c1ccc(S(=O)(=O)NC(=O)Nc2c(C(C)C)cc(C#N)c(F)c2C(C)C)c(O)c1]

Into a 25-mL round-bottom flask, was placed 3-hydroxy-1-N-methylbenzene-1,4-disulfonamide (100 mg, 0.38 mmol, 1 equiv), DCM, TEA, and 2-fluoro-4isocyanato-3,5-bis(propan-2-yl)benzonitrile (92.5 mg, 0.38 mmol, 1 equiv). The resulting solution was stirred overnight at 25 degrees C. The crude product was purified by Prep-HPLC with the following conditions, Column: XBridge Prep OBD C18 Column 19*250 mm, 5 um; Mobile Phase A:Water(10MMOL/L NH4HCO3), Mobile Phase B: ACN; Flow rate: 25 mL/min; Gradient: 13% B to 59% B in 7 min; 254/210 nm, Rt: 6.75 min. This resulted in 100 mg (50.57%) of 1-[4-cyano-3-fluoro-2,6-bis(propan-2-yl)phenyl]-3-[[2-methoxy-4(methylsulfamoyl)benzene]sulfonyl]urea as a white solid.

LC-MS-1-[4-cyano-3-fluoro-2,6-bis(propan-2-yl)phenyl]-3-[[2-methoxy-4(methylsulfamoyl)benzene]sulfonyl]urea (ES, m/z) (M−H)⁻=511.1

H-NMR-1-[4-cyano-3-fluoro-2,6-bis(propan-2-yl)phenyl]-3-[[2-methoxy-4(methylsulfamoyl)benzene]sulfonyl]urea (300 MHz; CD30D): 7.91 (s, 1H), 7.44 (d, J=6.6 Hz, 1H), 7.27 (s, 2H), 3.32 (m, 2H), 2.58 (s, 3H), 1.12 (m, 12H)

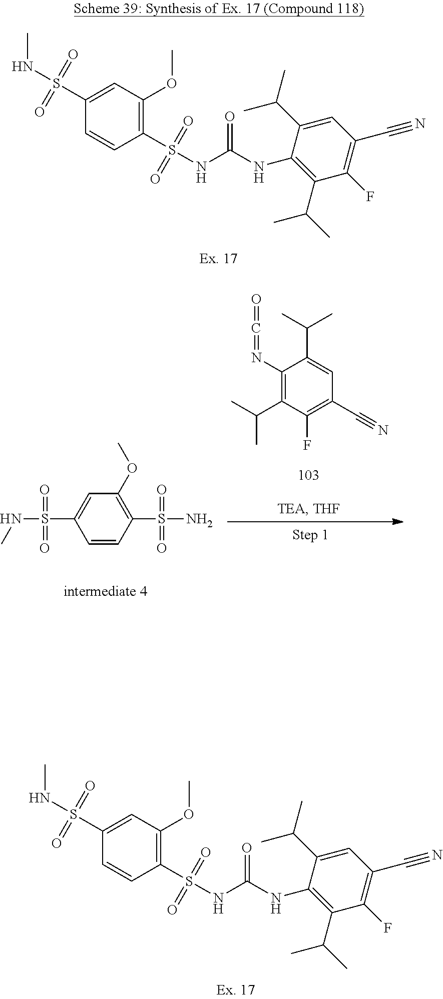 [SMILES: CNS(=O)(=O)c1ccc(S(=O)(=O)NC(=O)Nc2c(C(C)C)cc(C#N)c(F)c2C(C)C)c(OC)c1 ; CNS(=O)(=O)c1ccc(S(N)(=O)=O)c(OC)c1 ; CC(C)c1cc(C#N)c(F)c(C(C)C)c1N=C=O ; CNS(=O)(=O)c1ccc(S(=O)(=O)NC(=O)Nc2c(C(C)C)cc(C#N)c(F)c2C(C)C)c(OC)c1]

### 1. Synthesis of 1-[4-cyano-3-fluoro-2,6-bis(propan-2-yl)phenyl]-3-[2-(hydroxymethyl)-4-(methylsulfamoyl)benzenesulfonyl]urea

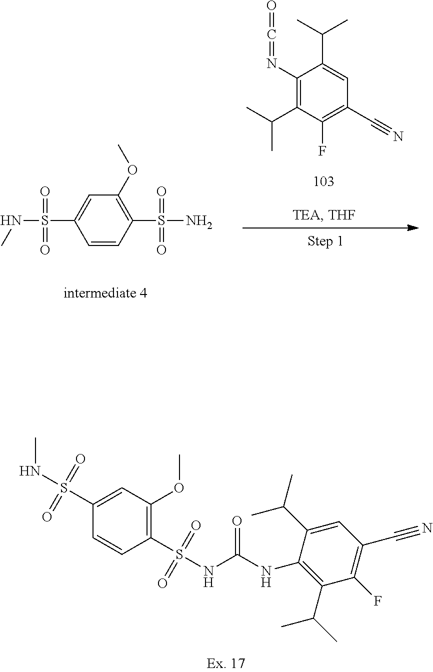 [SMILES: CNS(=O)(=O)c1ccc(S(N)(=O)=O)c(OC)c1 ; CC(C)c1cc(C#N)c(F)c(C(C)C)c1N=C=O ; CNS(=O)(=O)c1ccc(S(=O)(=O)NC(=O)Nc2c(C(C)C)cc(C#N)c(F)c2C(C)C)c(OC)c1]

Into a 50-mL round-bottom flask, was placed methyl 2-[([[4-cyano-3-fluoro-2,6-bis(propan-2-yl)phenyl]carbamoyl]amino)sulfonyl]-5-(methylsulfamoyl)benzoate (50 mg) and DCM (10 mL). The resulting solution was stirred for 2 hours. The crude product was purified by Prep-HPLC. This resulted in 13.8 mg of i-[4-eyano-3fluoro-2,6-bis(propan-2-yl)phenyl]-3-[2-(hydroxymethyl)-4-(methylsulfamoyl)benzenesulfonyl]urea as a white solid.

LCMS-PH-1-[4-cyano-3-fluoro-2,6-bis(propan-2-yl)phenyl]-3-[2-(hydroxymethyl)-4-(methylsulfamoyl)benzenesulfonyl]urea: (ES, m/z): [M+H]⁺=527.1

H-NMR-1-[4-cyano-3-fluoro-2,6-bis(propan-2-yl)phenyl]-3-[2-(hydroxymethyl)-4-(methylsulfamoyl)benzenesulfonyl]urea: (DMSO, ppm): ¹H NMR (300 MHz, DMSO) δ 8.09-7.96 (m, 3H), 7.67-7.54 (m, 3H), 7.22-6.88 (m, 1H), 5.50 (s, 2H), 5.00 (s, 2H), 3.00-2.96 (s, 2H), 2.41-2.37 (m, 3H), 1.20-0.96 (m, 12H).

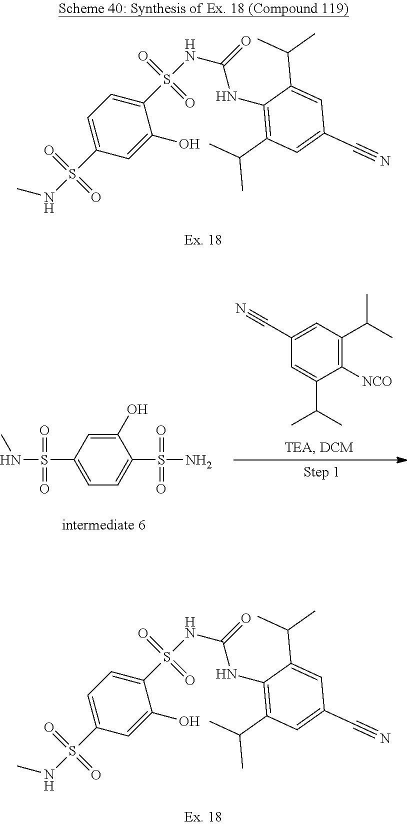

### 1. Synthesis of 1-[4-cyano-2,6-bis(propan-2-yl)phenyl]-3-[[2-hydroxy-4-(methylsulfamoyl)benzene]sulfonyl]urea

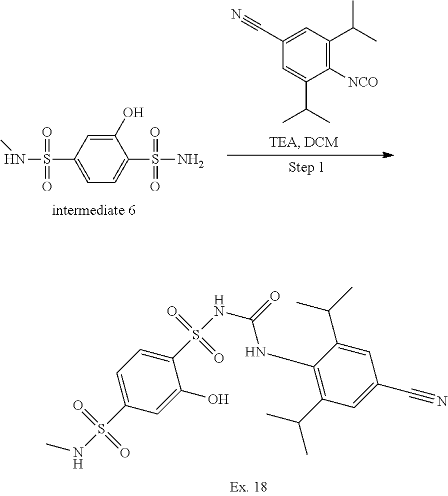

Into a 25-mL round-bottom flask, was placed 3-hydroxy-1-N-methylbenzene-1,4-disulfonamide (50 mg, 0.188 mmol, 1 equiv), DCM (30 mL), TEA (38.00 mg, 0.376 mmol, 2 equiv), and 2-fluoro-4-isocyanato-3,5-bis(propan-2-yl)benzonitrile (46.2 mg, 0.19 mmol, 1 equiv). The resulting solution was stirred for 2 hr at 25 degrees C. The resulting mixture was concentrated under vacuum. The crude product (3 mL) was purified by Flash-Prep-HPLC with the following conditions (IntelFlash-1): Column, CIS silica gel; mobile phase, MeCN/H2O=10/90 increasing to MeCN/H2O=90/10 within 1 hr; Detector, UV254. 500 mL product-containing fractions were obtained. This resulted in 50 mg (51.95%) of 1-[4-cyano-2,6-bis(propan-2-yl)phenyl]-3-[[2-hydroxy-4-(methylsulfamoyl)benzene]sulfonyl]urea as a white solid,

LCMS-1-[4-cyano-2,6-bis(propan-2-yl)phenyl]-3-[[2-hydroxy-4-(methylsulfamoyl)benzene]sulfonyl]urea (ES;m/s)(E-H)⁻=493.05

H-NMR-1-[4-cyano-3-fluoro-2,6-bis(propan-2-yl)phenyl]-3-[[2-hydroxy-4-(methylsulfamoyl)benzene]sulfonyl]urea (300 Hz. Methanol-d₄): δ 7.92 (s, 1H) 7.44 (m, 2H) 6.80 (d, J=1.65 Hz, 2H), 3.14 (m, 1H), 2.59 (s, 3H), 1.13 (m, 12H)

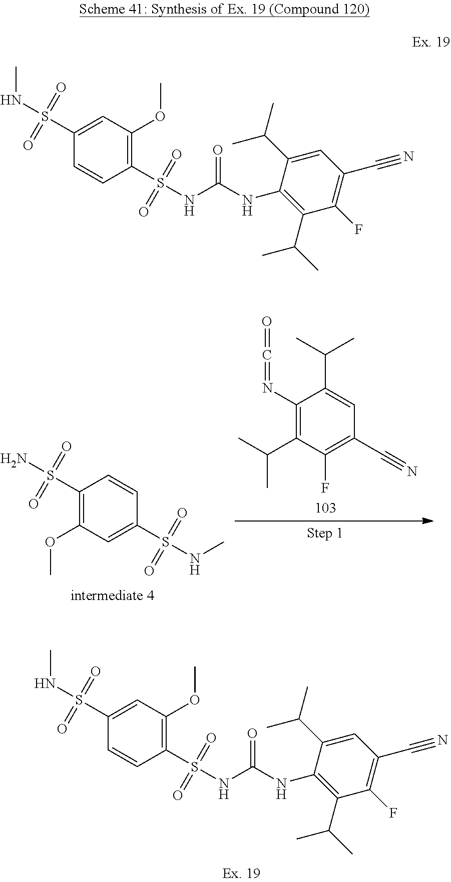 [SMILES: CNS(=O)(=O)c1ccc(S(=O)(=O)NC(=O)Nc2c(C(C)C)cc(C#N)c(F)c2C(C)C)c(OC)c1 ; CNS(=O)(=O)c1ccc(S(N)(=O)=O)c(OC)c1 ; CC(C)c1cc(C#N)c(F)c(C(C)C)c1N=C=O ; CNS(=O)(=O)c1ccc(S(=O)(=O)NC(=O)Nc2c(C(C)C)cc(C#N)c(F)c2C(C)C)c(OC)c1]

### 1. Synthesis of 1-[4cyano-3fluoro-2,6-bis(propan-2-yl)phenyl]-3-[2-methoxy-4-(methylsulfamoyl)benzenesulfonyl]urea

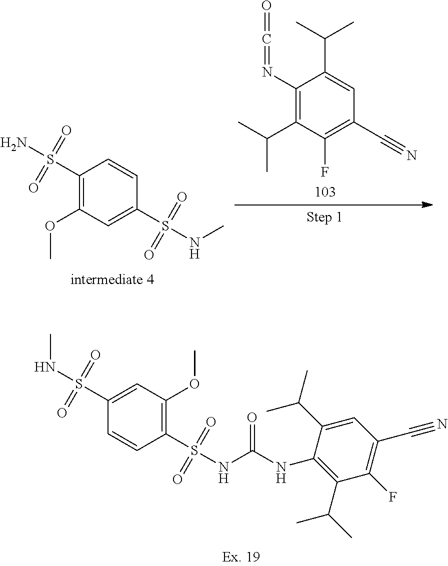 [SMILES: CNS(=O)(=O)c1ccc(S(N)(=O)=O)c(OC)c1 ; CC(C)c1cc(C#N)c(F)c(C(C)C)c1N=C=O ; CNS(=O)(=O)c1ccc(S(=O)(=O)NC(=O)Nc2c(C(C)C)cc(C#N)c(F)c2C(C)C)c(OC)c1]

Into a 50-mL round-bottom flask, was placed 2-fluoro-4-isocyanato-3,5-bis(propan-2-yl)benzonitrile (64 mg, 0.260 mmol, 1 equiv), DCM (20 mL), TEA (52.59 mg, 0.520 mmol, 2 equiv), and 3-methoxy-N1-methylbenzene-1,4-disulfonamide (87.41 mg, 0.312 mmol, 1.2 equiv). This solution was stirred for 1 hr at 25 degrees C. The resulting mixture was concentrated under vacuum. The crude product (2 mL) was purified by Flash-Prep-HPLC with the following conditions (IntelFlash-1): Column, silica gel; mobile phase, MeCN/H2O=10/90 increasing to MeCN/H2O=90/10 within 1 hr, Detector, 150 mL product-containing fractions were obtained. This resulted in 76 mg (55.5%) of 1-[4-cyano-3-fluoro-2,6-bis(propan-2-yl)phenyl]-3-[2-methoxy-4-(methylsulfamoyl)benzenesulfonyl]urea as a white solid.

MS: 1-[4-cyano-3-fluoro-2,6-bis(propan-2-yl)phenyl]-3-[2-methoxy-4-(methylsulfamoyl)benzenesulfonyl]urea (ES, m/z) (M−H)⁻=525.1

H-NMR: 1-[4-cyano-3-fluoro-2,6-bis(propan-2-yl)phenyl]-3-[2-methoxy-4-(methylsulfamoyl)benzenesulfonyl]urea: (300 MHz; CDCl₃), 8.14 (t, 1H), 7.52 (m, 3H), 4.09 (d, J=6.3 Hz, 3H), 3.28 (m, 2H), 2.58 (d, J=3.9 Hz, 3H), 1.29 (m, 12H)

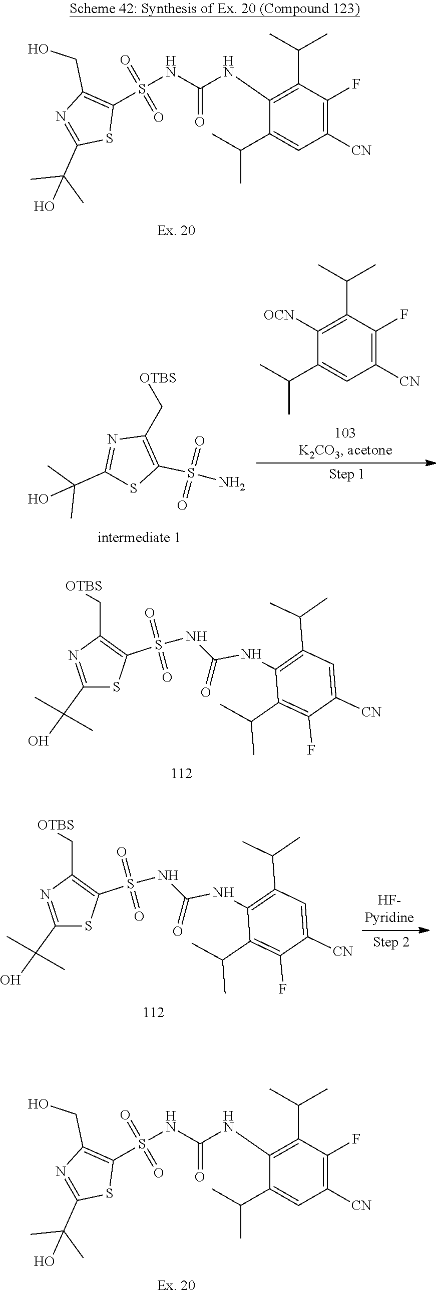

### 1. Synthesis of 4-((tert-butyldimethylsilyloxy)methyl)-N-(4-cyano-3-fluoro-2,6-diisopropylphenylcarbamoyl)-2-(2-hydroxypropan-2-yl)thiazole-5-sulfonamide

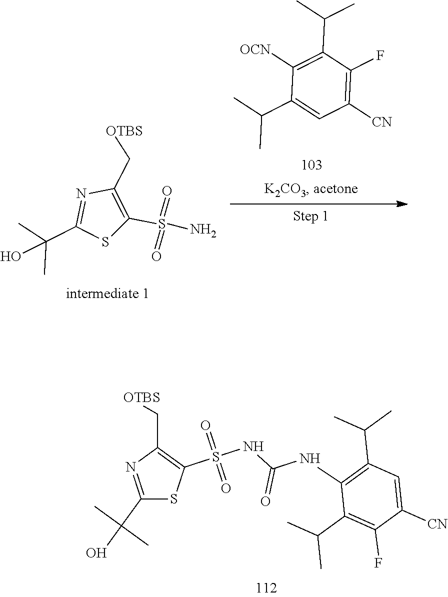

Into a 50-mL round-bottom flask, was placed 4-[[(tert-butyldimethylsilyl)oxy]methyl]-2-(2-hydroxypropan-2-yl)-1,3-thiazole-5-sulfonamide (60 mg, 0.16 mmol, 1 equiv), Acetone (10 mL). K₂CO₃(55 mg, 0.4 mmol, 2 equiv), 2-fluoro-4-isocyanato-3,5-bis(propan-2-yl)benzonitrile (48.4 mg, 0.2 mmol, 1.2 equiv). The resulting solution was stirred for 1 hr at room temperature. The solids were filtered out. The filtrate was concentrated to afford the crude product.

### 2. Synthesis of 1-[4-cyano-3-fluoro-2,6-bis(propan-2-yl)phenyl]-3-[4-(hydroxymethyl)-2-(2-hydroxypropan-2-yl)-1,3-thiazole-5-sulfonyl]urea

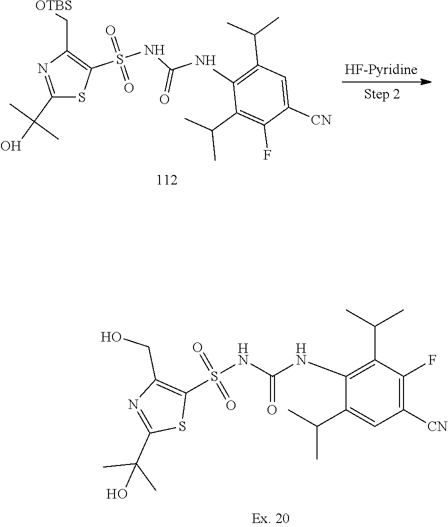 [SMILES: CC(C)c1cc(C#N)c(F)c(C(C)C)c1NC(=O)NS(=O)(=O)c1sc(C(C)(C)O)nc1CO[Si](C)(C)C(C)(C)C ; CC(C)c1cc(C#N)c(F)c(C(C)C)c1NC(=O)NS(=O)(=O)c1sc(C(C)(C)O)nc1CO]

Into a 50-mL round-bottom flask, was placed 3-(4-[[(tert-butyldimethylsilyl)oxy]methyl]-2-(2-hydroxypropan-2-yl)-1,3-thiazole-5-sulfonyl)-1-[4-cyano-3-fluoro-2,6-bis(propan-2-yl)phenyl]urea (50 mg, 0.08 mmol, 1 equiv), THF (5 mL), and HF-Pyridine (0.2 mL). The resulting solution was stirred for 1 h at room temperature. The resulting mixture was concentrated. The crude product was purified by Prep-HPLC with the following conditions (2 #SHIMADZU (HPLC-01)): Column, XBridge Shield RP18 OBD Column, 19*250 mm, 10 um; mobile phase, Water(10 MMOL/L NH₄HCO₃) and ACN (20% PhaseB up to 40% in 10 min); Detector, UV 220 nm. This resulted in 43 mg of 1-[4-cyano-3-fluoro-2,6-bis(propan-2-yl)phenyl]-3-[4-(hydroxymethyl)-2-(2-hydroxypropan-2-yl)-1,3-thiazole-5-sulfonyl]urea as a solid.

LC-MS-1-[4-cyano-3-fluoro-2,6-bis(propan-2-yl)phenyl]-3-[4-(hydroxymethyl)-2-(2-hydroxypropan-2-yl)-1,3-thiazole-5-sulfonyl]urea: (ES, m/z): [M+Z]⁺=499.2

H-NMR-1-[4-cyano-3-fluoro-2,6-bis(propan-2-yl)phenyl]-3-[4-(hydroxymethyl)-2-(2-hydroxypropan-2-yl)-1,3-thiazole-5-sulfonyl]urea: (DMSO, ppm): ¹H NMR (400 MHz, DMSO-d₆) δ 8.01 (s, 1H), 7.53 (d, J=6.9 Hz, 1H), 7.08 (s, 4H), 5.94 (s, 1H), 5.19 (s, 1H), 4.61. (d, J=5.8 Hz, 2H). 3.13 (s, 2H). 1.47 (s, 6H). 1.12-1.03 (m, 12H).

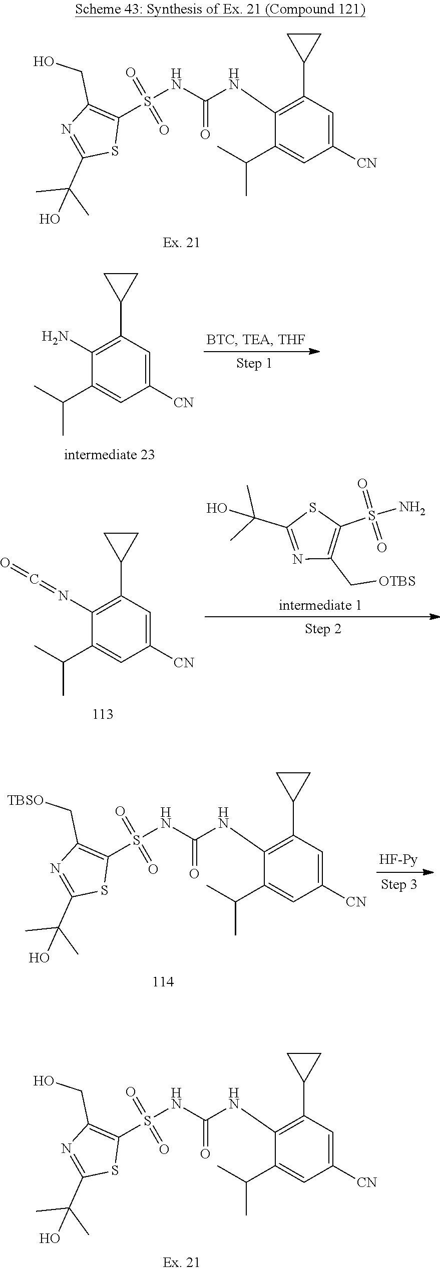 [SMILES: CC(C)c1cc(C#N)cc(C2CC2)c1NC(=O)NS(=O)(=O)c1sc(C(C)(C)O)nc1CO ; CC(C)c1cc(C#N)cc(C2CC2)c1N ; CC(C)c1cc(C#N)cc(C2CC2)c1N=C=O ; CC(C)(O)c1nc(CO[Si](C)(C)C(C)(C)C)c(S(N)(=O)=O)s1 ; CC(C)c1cc(C#N)cc(C2CC2)c1NC(=O)NS(=O)(=O)c1sc(C(C)(C)O)nc1CO[Si](C)(C)C(C)(C)C ; CC(C)c1cc(C#N)cc(C2CC2)c1NC(=O)NS(=O)(=O)c1sc(C(C)(C)O)nc1CO]

### 1. Synthesis of 3-cyclopropyl-4-isocyanato-5-(propan-2-yl)benzonitrile

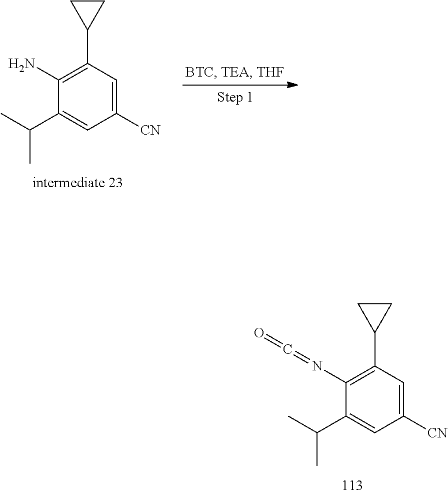 [SMILES: CC(C)c1cc(C#N)cc(C2CC2)c1N ; CC(C)c1cc(C#N)cc(C2CC2)c1N=C=O]

Into a 50-mL round-bottom flask purged and maintained with an inert atmosphere of nitrogen, was placed a solution of 4-amino-3cyclopropyl-5-(propan-2-yl)benzonitrile (100 mg, 0.50 mmol, 1 equiv) in THF (5 mL), ditrichloromethyl carbonate (74.1 mg, 0.25 mmol, 0.5 equiv), and TEA (1.01.0 mg, 1.00 mmol, 2 equiv). The resulting solution was stirred for 1 hr at 60 degrees C. The resulting mixture was concentrated. This resulted in 110 mg (97.4%) of 3-cyclopropyl-4-isocyanato-5-(propan-2-yl)benzonitrile as a yellow solid,

### 2. Synthesis of 3-(4-[[(tert-butyldimethylsilyl)oxy]methyl]-2-(2-hydroxypropan-2-yl)-1,3-thiazole-5-sulfonyl)-1-[4-cyano-2-cyclopropyl-6-(propan-2-yl)phenyl]urea

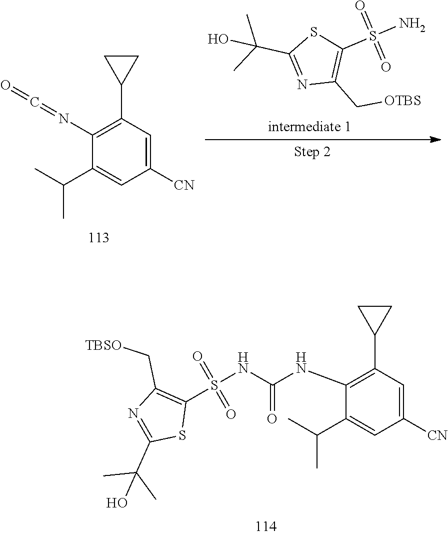 [SMILES: CC(C)c1cc(C#N)cc(C2CC2)c1N=C=O ; CC(C)(O)c1nc(CO[Si](C)(C)C(C)(C)C)c(S(N)(=O)=O)s1 ; CC(C)c1cc(C#N)cc(C2CC2)c1NC(=O)NS(=O)(=O)c1sc(C(C)(C)O)nc1CO[Si](C)(C)C(C)(C)C]

Into a 50-mL round-bottom flask, was placed a solution of 3-cyclopropyl-4-isocyanato-5-(propan-2-yl)benzonitrile (110 mg, 0.49 mmol, 1 equiv) in THF (5 mL), 4-[[(tert-butyldimethylsilyl)oxy]methyl]-2-(2-hydroxypropan-2-yl)-1,3-thiazole-5-sulfonamide (178.2 mg, 0.49 mmol, 1 equiv), and DBU (148.0 mg, 0.97 mmol, 2 equiv). The resulting solution was stirred for 2 hr at room temperature. The resulting mixture was concentrated. This resulted in 250 mg (86.7%) of 3-(4-[[(tert-butyldimethylsilyl)oxy]methyl]-2-(2-hydroxypropan-2-yl)-1,3-thiazole-5-sulfonyl)-1-[4-cyano-2-cyclopropyl-6-(propan-2-yl)phenyl]urea as yellow oil.

### 3. Synthesis of 1-[4-cyano-2-cyclopropyl-6-(propan-2-yl)phenyl]-3-[4-(hydroxymethyl)-2-(2-hydroxypropan-2-yl)-1,3-thiazole-5-sulfonyl]urea

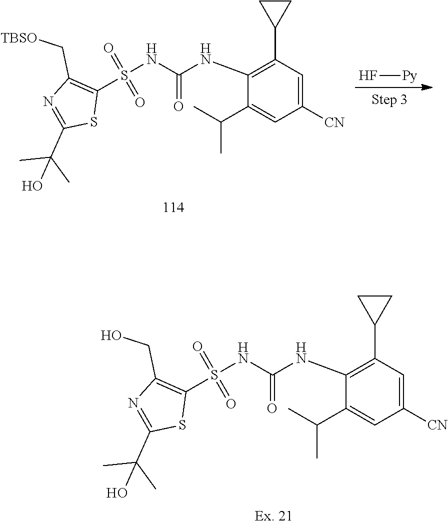

Into a 50-mL round-bottom flask, was placed a solution of 3-(4-[[(tert-butyldimethylsilyl)oxy]methyl]-2-(2-hydroxypropan-2-yl)-1,3-thiazole-5-sulfonyl)-1-[4-cyano-2-cyclopropyl-6-(propan-2-yl)phenyl]urea (250 mg, 0.42 mmol, 1 equiv) in THF (5 mL), and HF-Pyridine (417.9 mg, 4.22 mmol, 10 equiv). The resulting solution was stirred for 1 hr at room temperature. The resulting mixture was concentrated. The crude product (100 mg) was purified by Prep-HPLC with the following conditions (Prep-HPLC-018): Column, XBridge Shield RP18 OBD Column, 19*250 mm, 10 um; mobile phase, Water(10MMOL/L NH4HCO3) and ACN (14% PhaseB up to 36% in 7 min); Detector, UV. This resulted in 25.9 mg (12.8%) of 1-[4-cyano-2-cyclopropyl-6-(propan-2-yl)phenyl]-3-[4-(hydroxymethyl)-2-(2-hydroxypropan-2-yl)-1,3-thiazole-5-sulfonyl]urea as a white solid.

LC-MS-1-[4-cyano-2-cyclopropyl-6-(propan-2-yl)phenyl]-3-[4-(hydroxymethyl)-2-(2-hydroxypropan-2-yl)-1,3-thiazole-5-sulfonyl]urea (ES, m/z): [M+Z]⁺=479.1

H-NMR-1-[4-cyano-2-cyclopropyl-6-(propan-2-yl)phenyl]-3-[4-(hydroxymethyl)-2-(2-hydroxypropan-2-yl)-1,3-thiazole-5-sulfonyl]urea: (400 MHz, Methanol-d₄) δ 7.44 (d, J=1.8 Hz, 1H), 7.12 (d, J=1.8 Hz, 1H), 4.91 (s, 2H), 3.26 (d, J=6.7 Hz, 1H), 1.99 (s, 1H), 1.79 (s, 0H), 1.69 (s, 0H), 1.60 (s. 6H), 1.17 (d, J=6.9 Hz, 6H), 0.90 (d, J=8.5 Hz, 2H), 0.60 (d, J=5.6 Hz, 2H).

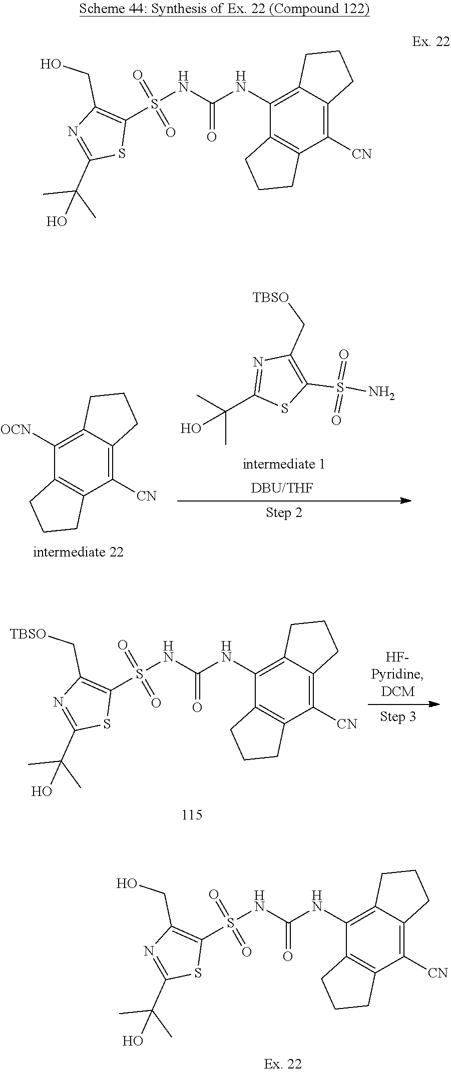

### 1. Synthesis of 4-((tert-butyldimethylsilyloxy)methyl)-N-(8-cyano-1,2,3,5,6,7-hexahydros-indacen-4-ylcarbamoyl)-2-(2-hydroxypropan-2-yl)thiazole-5-sulfonamide

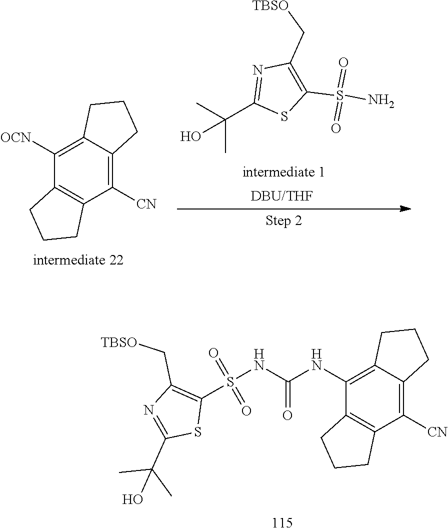

Into a 50-mL round-bottom flask, was placed 4-[[(tert-butyldimethylsilyl)oxy]methyl]-2-(2-hydroxypropan-2-yl)-1,3-thiazole-5-sulfonamide (75 mg, 0.20 mmol, 1 equiv), DBU (62.3 mg, 0.41 mmol, 2 equiv), THF (10 mL), and 8-isocyanato-1,2,3,5,6,7-hexahydro-s-indacene-4-carbonitrile (68.9 mg, 0.31 mmol, 1.501 equiv). The resulting solution was stirred for 1 hr at room temperature. The resulting mixture was concentrated. The crude was used for the next step directly.

### 2. Synthesis of 1-(8-cyano-1,2,3,5,6,7-hexahydro-s-indacen-4-yl)-3-[4-(hydroxymethyl)-2-(2-hydroxypropan-2-yl)-1,3-thiazole-5-sulfonyl]urea

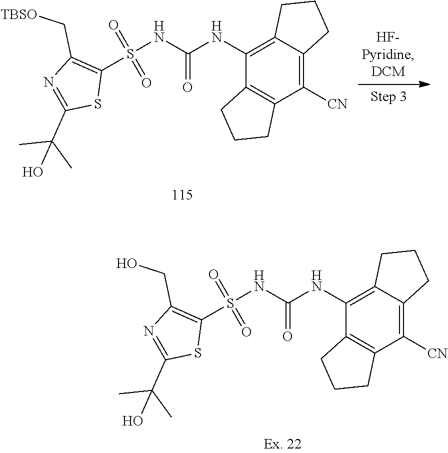 [SMILES: CC(C)(O)c1nc(CO[Si](C)(C)C(C)(C)C)c(S(=O)(=O)NC(=O)Nc2c3c(c(C#N)c4c2CCC4)CCC3)s1 ; CC(C)(O)c1nc(CO)c(S(=O)(=O)NC(=O)Nc2c3c(c(C#N)c4c2CCC4)CCC3)s1]

Into a 25-mL round-bottom flask, was placed 3-(4-[[(tert-butyldimethylsilyl)oxy]methyl]-2-(2-hydroxypropan-2-yl)-1,3-thiazole-5-sulfonyl)-1-(8-cyano-1,2,3,5,6,7-hexahydro-s-indacen-4-yl)urea (50 mg, 0.17 mmol, 1 equiv, crude), DCM (5 mL), and HF.Pyridine (0.3 mL). The resulting solution was stirred for 2 hr at room temperature and was then concentrated. The crude product v/as purified by Prep-HPLC with the following conditions (Prep-HPLC-008): Column, XBridge Shield RP18 OBD Column, 19*250 mm, 10 um; mobile phase, Water(10MMOL/L NH₄HCO₃) and ACN (10% PhaseB up to 40% in 7 min), Detector, UV 220 nm. This resulted in 20 mg of 1-(8-cyano-1,2,3,5,6,7-hexahydro-s-indacen-4-yl)-3-[4-(hydroxymethyl)-2-(2-hydroxypropan-2-yl)-1,3-thiazole-5-sulfonyl]urea as a white solid.

LC-MS-1-(8-cyano-1,2,3,5,6,7-hexahydro-s-indacen-4-yl)-3-[4-(hydroxymethyl)-2-(2-hydroxypropan-2-yl)-1,3-thiazole-5-sulfonyl]urea: (ES, m/z): [M+H]⁺=477.1

H-NMR-1-(8-cyano-1,2,3,5,6,7-hexahydro-5-indacen-4-yl)-3-[4-(hydroxymethyl)-2-(2-hydroxypropan-2-yl)-1,3-thiazole-5-sulfonyl]urea: (300 MHz, Methanol-d₄) δ 4.90 (d, J=6.3 Hz, 2H), 2.99 (q, J=7.5 Hz, 4H), 2.85-2.70 (m, 4H), 2.13 (dd J=8.0, 7.4 Hz, 4H), 1.61 (d, J=2.4 Hz, 6H)

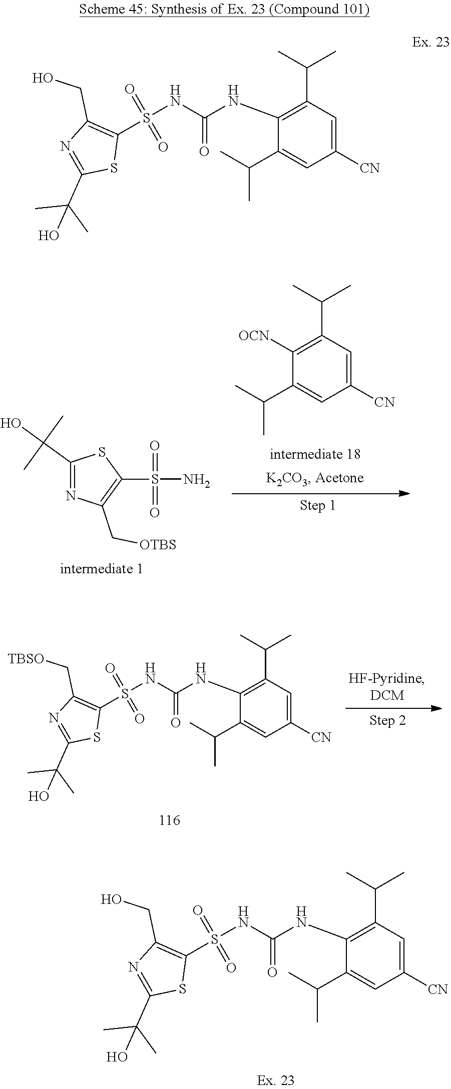

### 1. Synthesis of 4-((tert-butyldimethylsilyloxy)methyl)-N-(4-cyano-2,6-diisopropylphenylcarbamoyl)-2-(2-hydroxypropan-2-yl)thiazole-5-sulfonamide

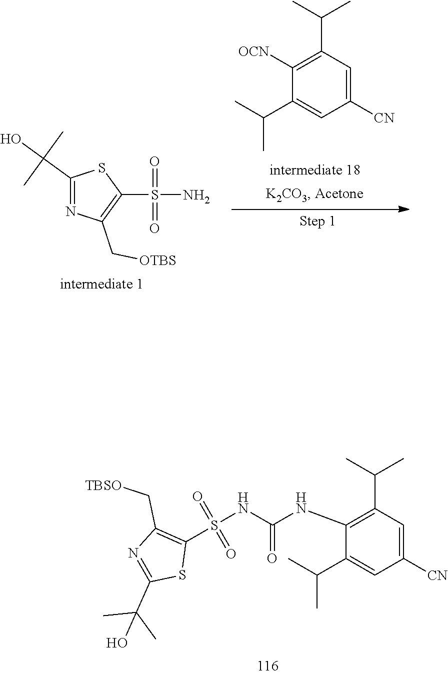

Into a 250-ml, round-bottom flask, was placed 4-[[(tert-butyldimethylsilyl)oxy]methyl]-2-(2-hydroxypropan-2-yl)-1,3-thiazole-5-sulfonamide (7.8 g, 34.45 mmol, 1 equiv) in acetone (100 mL). K₂CO₃ (9.5 g, 68.91 mmol, 2 equiv) was added in one portion at room temperature, and the resulting mixture stirred for 30 min, after which 4-isocyanato-3,5-bis(propan-2-yl)benzonitrile (11.3 g, 31.00 mmol, 0.9 equiv) was added. The resulting solution was stirred for 2 h at room temperature. The solids were filtered out. The filtrate was concentrated to give 4-((tert-butyldimethylsilyloxy)methyl)-N-(4-cyano-2,6-diisopropylphenylcarbamoyl)-2-(2-hydroxypropan-2-yl)thiazole-5-sulfonamide, which was used directly for the next step. LC-MS of 4-((tert-butyldimethylsilyloxy)methyl)-N-(4-cyano-2,6-diisopropylphenylcarbamoyl)-2-(2-hydroxypropan-2-yl)thiazole-5-sulfonamide (Method B): 595.3 [M+H]+, retention time 1.040 min. Method: Kinetex EVO C18, 50*3.0 mm, 0.3 uL injection, 1.2 mL/min flowrate, 90-900 amu scan range, 254 nm UV detection. Mobile phase A: Water (6.5 mmoL/L NH₄HCO₃), mobile phase B; MeCN. 10% MPB to 95.0% in 1.1 min, hold at 95% MPB for 0.5 min, 95% MPB to 10% in 0.01 min, then equilibration to 10% MPB for 0.21 min.

### 2. Synthesis of 1-[4-cyano-2,6-bis(propan-2-yl)phenyl]-3-[4-(hydroxymethyl)-2-(2-hydroxypropan-2-yl)-1,3-thiazole-5-sulfonyl]urea

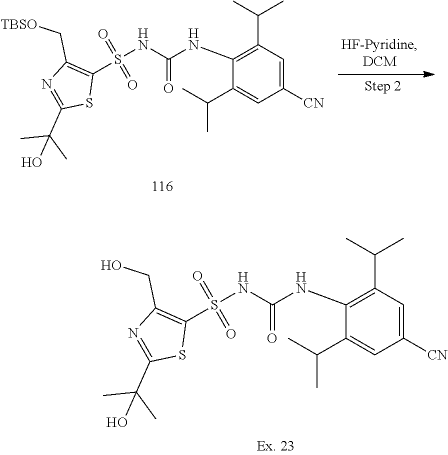 [SMILES: CC(C)c1cc(C#N)cc(C(C)C)c1NC(=O)NS(=O)(=O)c1sc(C(C)(C)O)nc1CO[Si](C)(C)C(C)(C)C ; CC(C)c1cc(C#N)cc(C(C)C)c1NC(=O)NS(=O)(=O)c1sc(C(C)(C)O)nc1CO]

Into a 250-mL round-bottom flask, was placed a solution of 3-(4-[[(tert-butyldimethylsilyl)oxy]methyl]-2-(2-hydroxypropan-2-yl)-1,3-thiazole-5-sulfonyl)-1-[4-cyano-2,6-bis(propan-2-yl)phenyl]urea (4.5 g, 7.56 mmol, 1 equiv) in DCM (40 mL). To the solution wad added HF Pyridine (1 mL, 11.10 mmol, 1.47 equiv). The resulting solution was stirred for 1 h at room temperature. The resulting mixture was concentrated. The crude product was purified by Flash-Prep-HPLC with the following conditions (IntelFlash-1): Column, Cl8; mobile phase, Water (10 mmoL/L NH₄HCO₃) and MeCN (0% MeCN up to 55.0% in 20 min); Detector, UV 210 nm. This resulted in 1.9 g (10.25%) of 1-[4-cyano-2,6-bis(propan-2-yl)phenyl]-3-[4-(hydroxymethyl)-2-(2-hydroxypropan-2-yl)-1,3-thiazole-5-sulfonyl]urea as a white solid. LC-MS (Method D): 481.2 [M+H]+, retention time 2.063 min. Method: Agilent Poroshell HPH-C18, 50*3.0 mm, 0.1 uL injection, 1.0 mL/min flowrate, 90-900 amu scan range, 210 nm UV detection. Mobile phase A: Water (5 mmoL/L NH₄HCO₃), mobile phase B: MeCN. 10% MPB to 40.0% in 2.9 min, 40% MPB to 95% MPB in 0.4 min, hold at 95% MPB for 0.4 min, 95% MPB to 10% in 0.1 min, then equilibration to 0.10% MPB for 0.2 min.

H-NMR: (CD₃OD-d₄, 400MHZ, ppm): δ 7.42 (s, 2H). 4.89 (s, 2H), 3.08-3.07 (m, 2H), 1.58 (s, 6H), 1.14 (d, J=6.6 Hz, 12H).

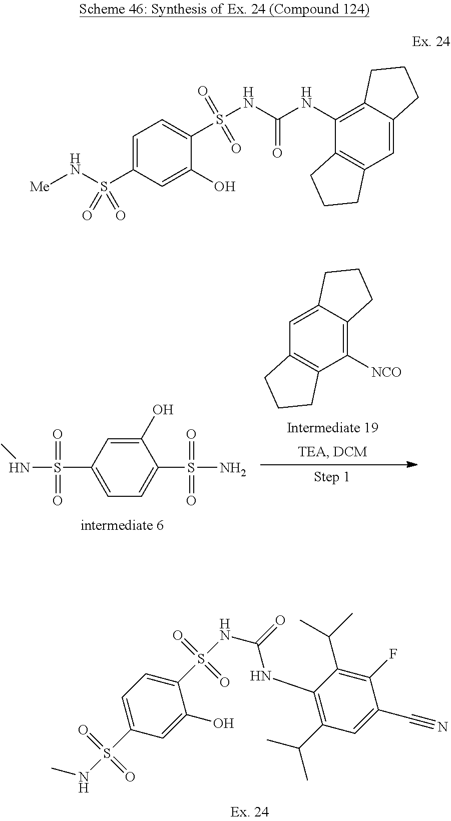

Into a 25-mL round-bottom flask, was placed 3-hydroxy-1-N-methylbenzene-1,4-disulfonamide (50 mg, 0.188 mmol, 1 equiv), DCM (30 mL), TEA (38.00 mg, 0.376 mmol, 2 equiv), and 4-isocyanato-1,2,3,5,6,7-hexahydro-x-indacene (46.2 mg, 0.19 mmol, 1 equiv). The resulting solution was stirred for 2 hr at 25 degrees C. The resulting mixture was concentrated under vacuum. The crude product (3 mL) was purified by Flash-Prep-HPLC with the following conditions (IntelFlash-1): Column, C₁₋₈ silica gel; mobile phase, MeCN/H2O=10/90 increasing to MeCN/H2O=90/10 within 1 hr; Detector, UV254. 500 mL product-containing fractions were obtained and isolated as a white solid.

LCMS-1 (ES; m/z)(M+H)⁻=466.01

### Assay 1

The following protocol s are suitable for testing the activity of the compounds disclosed herein.

### Procedure 1: IL-1β Production in PMA-Differentiated THP-1 Cells Stimulated with Gramicidin

THP-1 cells were purchased from the American Type Culture Collection and sub-cultured according to instructions from the supplier. Cells were cultured in complete RPMI 1640 (containing 10% heat inactivated FBS, penicillin (100 units/ml) and streptomycin (100 μg/ml)), and maintained in log phase prior to experimental setup. Prior to the experiment, compounds were dissolved in dimethyl sulfoxide (DMSO) to generate a 30 mM stock. The compound stock was first pre-diluted in DMSO to 3, 0.34, 0.042 and 0.0083 mM intermediate concentrations and subsequently spotted using Echo550 liquid handler into an empty 384-well assay plate to achieve desired final concentration (e.g. 100, 33, 11, 3,7, 1.2, 0.41, 0.14, 0.046, 0.015, 0.0051, 0.0017 μM). DMSO was backfilled in the plate to achieve a final DMSO assay concentration of 0.37%. The plate was then sealed and stored at room temperature until required. THP-1 cells were treated with PMA (Phorbol 12-myristate 13-acetate) (20 ng/ml) for 16-18 hours. On the day of the experiment the media was removed and adherent cells were detached with trypsin for 5 minutes. Cells were then harvested, washed with complete RPMI 1640, spun down, and resuspended in RPMI 1640 (containing 2% heat inactivated FBS, penicillin (100 units/ml) and streptomycin (100 μg/ml). The cells were plated in the 384-well assay plate containing the spotted compounds at a density of 50,000 cells/well (final assay volume 50 μl). Ceils were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). The plates were incubated for 18 h at 4° C. and read using the preset HTRF program (donor emission at 620 nm, acceptor emission at 668 nm) of the SpectraMax i3x spectrophotometer (Molecular Devices, software SoftMax 6). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

### Procedure 2: IL-1β Production in PMA-Differentiated THP-1 Cells Stimulated with Gramicidin

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 μg/ml)), and maintained in log phase prior to experimental setup. Prior to the experiment THP-1 were treated with PMA (Phorbol 12-myristate 13-acetate) (20 ng/mi) for 16-18 hours. Compounds were dissolved in dimethyl sulfoxide (DMSO) to generate a 30 mM stock. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 μg/ml), The cells were plated in a 384-well plate at a density of 50,000 cells/well (final assay volume 50 μl). Compounds were first dissolved in assay medium to obtain a 5× top concentration of 500 μM. 10 step dilutions (1:3) were then undertaken in assay medium containing 1.67% DMSO. 5× compound solutions were added to the culture medium to achieve desired final concentration (e.g. 100, 33, 11, 3.7, 1.2, 0.41, 0.14, 0.046, 0.015, 0.0051, 0.0017 μM). Final DMSO concentration was at 0.37%. Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

Table 14 shows the biological activity of compounds in hTHP-1 assay containing 2% fetal bovine serum: <0.008 μM=“++++++”; ≥0.008 and <0.04 μM=“+++++”; ≥0.04 and <0.2 μM=“++++”; ≥0.2 and <1 μM=“+++”; ≥1 and <5 μM=≥5 and <30 μM=“+”.

TABLE 14
Average IC₅₀ of compounds in hTHP-1 assay

| | | hTHP-1 IC₅₀ |
|---|---|---|
| Compound | Ex. # | μM |
| 101 | 23 | +++ |
| 102 | 10 | ++++ |
| 103 | 1 | +++ |
| 104 | 2 | ++ |
| 105 | 3 | ++ |
| 106 | 4 | +++ |
| 107 | 5 | ++ |
| 108 | 6 | ++ |
| 109 | 7 | ++ |
| 110 | 8 | +++ |
| 111 | 9 | ++ |
| 112 | 11 | ++ |
| 113 | 12 | +++ |
| 114 | 13 | + |
| 115 | 14 | + |
| 116 | 15 | +++ |
| 117 | 16 | ++++ |
| 118 | 17 | +++ |
| 119 | 18 | ++++ |
| 120 | 19 | ++++ |
| 121 | 21 | ++ |
| 122 | 22 | ++ |
| 123 | 20 | +++ |
| 124 | 24 | + |
| 201 | | +++ |
| 202 | | +++ |
| 203 | | ++ |
| 204 | | >15 μM |
| 301 | | +++ |
| 302 | | >30.0000 |
| 303 | | >30.0000 |
| 304 | | + |
| 324 | | >30.0000 |

### Assay 2: Colon Pharmacokinetics in Mice

The test compound was formulated in 0.5% methyl-cellulose in water and dosed via oral gavage at 30 mg/kg to Male C57BL/6 Mouse. At various time points (typically 15 min, 30 min, 1, 2, 4, 6 and 8 h) post dosing, blood samples were removed via cardiac 25 puncture and intact colons were excised from the rats. Blood samples were centrifuged at 1500 x g for 15 min to collect plasma. At the terminal time point each individual animal is anaesthetized, abdominal cavity is opened and from 2 cm below the caecum a 4 cm sample of the colon is dissected, cut open on the longitudinal axis and the solid contents removed by flushing with 2 mL of physiological fluid. The colon was further washed by putting it in 5 mL of physiological saline and shaken for 1 minute. The colon was pet dry weighed and transferred in 2 mL tubes. Colon will be weighted and homogenized with water by tissue weight (g) to water volume (mL) at ratio 1:3 before analysis. The actual concentration is the detected value multiplied by the dilution factor. A colon to plasma ratio was determined as the ratio of the colon cone, to the plasma cone, in pg hr/g, at 8 h time point.

| | | Mouse TD |
|---|---|---|
| Compound # | Ex. # | Colon/Plasma @ 8 |
| 102 | 10 | 802 |
| 124 | 24 | 26 |
| 113 | 12 | 4,561 |
| 116 | 15 | 18,645 |
| 118 | 17 | 5,950 |
| 123 | 20 | 12,731 |
| 122 | 22 | 3,529 |
| 101 | 23 | 3,003 |

### Assay 3: Determination of Absorption in Cannulated Rats

Oral bioavailability (F %), fraction absorbed (Fa %) and fraction escaping hepatic 25 clearance (Fh %) were determined in Sprague Dawley rats from the following two studies: (I) Pharmacokinetics in rats following an IV dose of test compound (i.e., the analog being tested): Following IV dosing, plasma samples were typically collected from 0-24 hr. Drug levels were determined using an LC-MS-MS method. The resulting drug levels were used to compute the IV pharmacokinetic parameters: AUC IV and Dose IV. Rats that have been cannulated in their portal vein (PV) and also in their jugular vein (TV) were dosed orally with test compound. Following oral dosing, plasma samples were typically collected from 0-6 hr from both the portal vein and the jugular vein. Drug levels were determined using an LC-MS-MS method. The resulting drug levels were used to compute the following pharmacokinetic parameters: AUC PO PV, AUC PO JV5 and Dose PO. Using data derived from the above studies, the oral bioavailability F %, and the quantities Fa % and Fh % were calculated from the following formulas:

5F%=(AUC PO JV/AUC IV)*(Dose IV/Dose PO)*100

Fa%=(AUC PO PV/AUC IV)*(Dose IV/Dose PO)*100

Fh%=AUC PO JV/AUC PO PV

where:
AUC PO JV=Area under the curve following oral dose and plasma collected from the jugular vein;
AUC PO PV=Area under the curve following oral dose and plasma collected from the portal vein;
AUC IV=Area under the curve following an intravenous dose,
Dose IV=Intravenous Dose in mg/kg; and
15 Dose PO=Oral Dose in mg/kg

The compounds were tested in this assay and exhibited oral bioavailability (F %) less than about 25%. In particular, Ex. 23 exhibited F % values less than about 5%. In addition, Ex. 23 exhibited absorption at the portal vein (Fa %) less than about 25%

We claim:

1. A compound of Formula AA 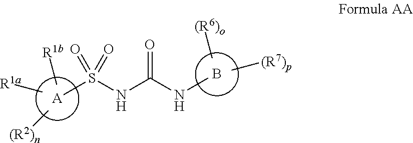
wherein
n=0 or 1;
o=1 or 2;
p=0, 1, 2, or 3;
wherein
A is a 5- to 10-membered heteroaryl or a C₆-C₁₀ aryl;
B is a 5- to 10-membered heteroaryl or a C₆-C₁₀ aryl;
wherein
R^{1a} is a C₁-C₆ alkyl or —SO₂NR¹¹R¹²;
wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²;
at least one R⁶ is ortho to the bond connecting the B ring to the NH(Co) group of Formula AA;
R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ and, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOC₁-C₆ alkyl, NH—(C=NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3 to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₅-C₁₀ cycloalkyl and 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from Q, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, (C=NR¹³)NR¹¹R¹², S(O)₂C₁-C₆ alkyl, S(O₂)NR¹¹R¹²COR¹³, CO₂R¹³ and CONR¹¹R¹²; wherein the C₁-C₆ alkyl is optionally substituted with one or more hydroxy, halo, C₁-C₆ alkoxy, C₆-C₁₀ and, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl; and
each of R¹¹ and R¹² at each occurrence is independently selected from hydrogen and C₁-C₆ alkyl optionally substituted with hydroxy;
with the proviso that the compound of Formula AA is not a compound selected from the group consisting of: 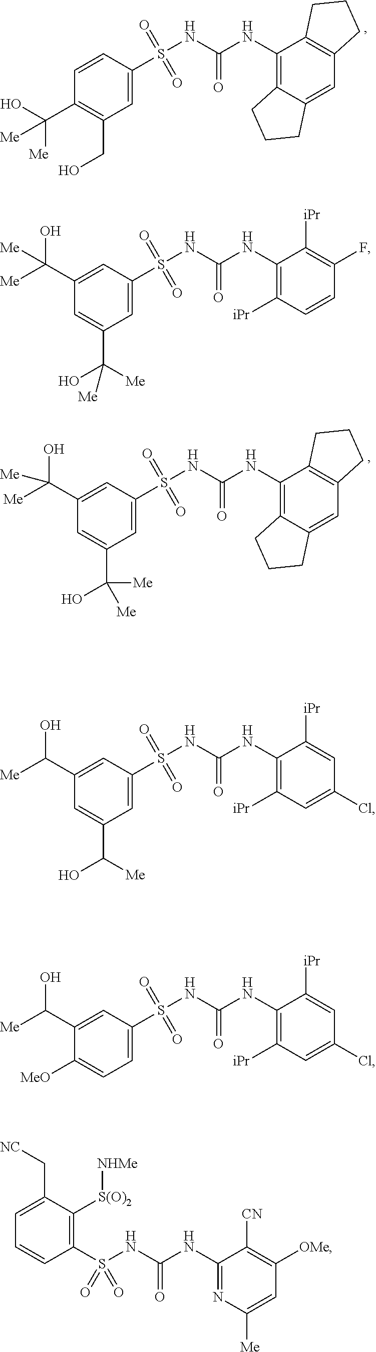 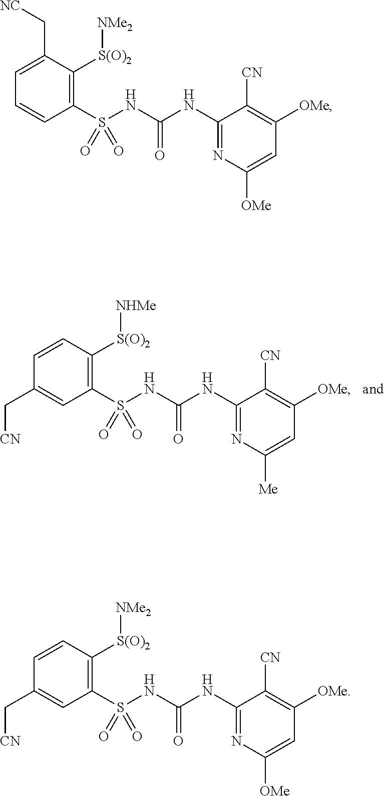
or a pharmaceutically acceptable salt thereof.

2. A compound of Formula AA 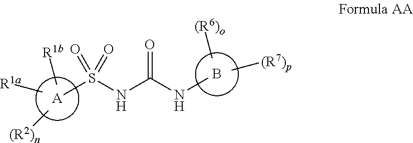
wherein the compound of Formula AA is selected from 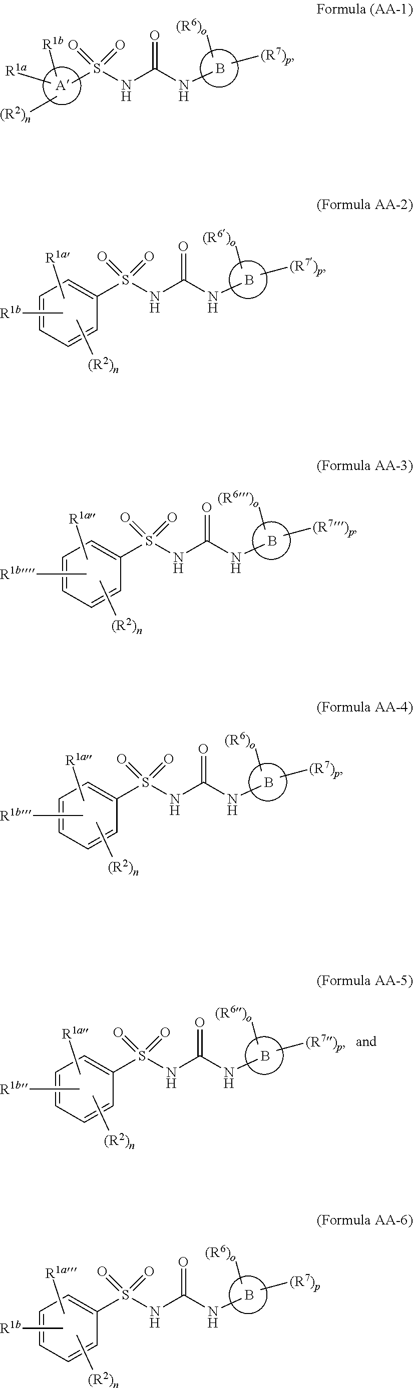
wherein
n=0 or 1;
o=1 or 2;
p=0, 1, 2, or 3;
wherein
A′ is a 5- to 10-membered heteroaryl;
B is a 5- to 10-membered heteroaryl or a C₆-C₁₀ aryl;
wherein
R^{1a} is a C₁-C₆ alkyl or —SO₂NR¹¹R¹²;
wherein the C₁-C₆ alkyl is substituted with one or more hydroxy or —OSi(R¹³)₃;
R^{1a′} is —SO₂NR¹¹R¹²;
R^{1a″} is a C₁-C₆ alkyl;
wherein the C₁-C₆ alkyl is substituted with one or more hydroxy;
R^{1a′″} is a C₁-C₆ alkyl;
wherein the C₁-C₆ alkyl is substituted with one or more-OSi(R¹³)₃;
R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²;
R^{1b′} is —OR¹¹;
R^{1b″} is a —SO₂NR¹¹R¹², —SO₂R¹³, —CONR¹¹R¹², —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², and —NR¹¹COR¹²;
R^{1b′″} is a C₁-C₆ alkyl substituted with one or more hydroxy;
at least one R⁵ is ortho to the bond connecting the B ring to the NH(Co) group of Formula AA-1 and Formula AA-4;
at least one R^{6′} is ortho to the bond connecting the B ring to the NH(CO) group of Formula AA-2;
at least one R^{6″} is ortho to the bond connecting the B ring to the NH(CO) group of Formula AA-5;
at least one R^{6′″} is ortho to the bond connecting the B ring to the NH(CO) group of Formula AA-3;
R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3 to 7-membered heterocycloalkyl), NHCOC₁-C₆ alkynyl, NHCOOC₁-C₆ alkyl, NH—(C=NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R² C₃-C₇ cycloalkyl or of the R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3- to 7-membered heterocycloalkyl are optionally substituted with one or more substituents independently selected from halo, C₁-C₆ alkyl, and OC₁-C₆ alkyl;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R⁶ and R⁷ are each optionally substituted with one or more substituents independently-selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₀ alkynyl, C₆-C₁₀ aryloxy, and S(O₂)C₁-C₆ alkyl, and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₁-C₆ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6′} or R^{7′} is substituted with is optionally substituted with one or more hydroxyl, halo, C₆-C₁₀ aryl or NR⁸R⁹, or wherein R^{6′} or R^{7′} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
or at least one pair of R⁶ and R on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R^{6″} and R^{7″} are each independently selected from C₁-C₆ alkyl, C₁-C₆ haloalkyl, C₁-C₆ alkoxy, C₁-C₆ haloalkoxy, F, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6″} and R^{7″} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6″} or R^{7″} is substituted with is optionally substituted with one or more hydroxyl, halo, C₆-C₁₀ aryl or NR⁸R⁹, or wherein R^{6″} or R^{7″} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl,
or at least one pair of R^{6″} and R^{7″} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₀ alkenyl,
wherein R^{6′″} and R^{7′″} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6′″} or R^{7′″} is substituted with is optionally substituted with one or more hydroxyl, halo, C₆-C₁₀ aryl or NR⁸R⁹, or wherein R^{6′″} or R^{7′″} is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5 to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R^{6′″} and R^{7′″} on adjacent atoms, taken together with the atoms connecting them, independently form at least one C₄, C₆, C₇, or C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, (C=NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ and, 5- to 10-membered heteroaryl, C₃-C₇ cycloalkyl or 3- to 7-membered heterocycloalkyl; or
R⁸ and R⁹ taken together with the nitrogen they are attached to form a 3- to 7-membered ring optionally containing one or more heteroatoms in addition to the nitrogen they are attached to, R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl optionally substituted with hydroxy;
or a pharmaceutically acceptable salt thereof.

3. The compound of claim 3, wherein the compound of Formula AA is

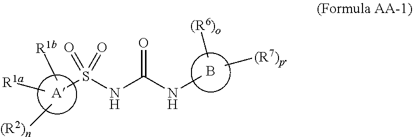

4. The compound of claim 3, wherein the compound of Formula AA is

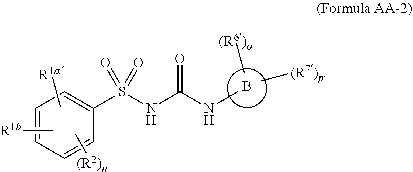

5. The compound of claim 3, wherein the compound of Formula AA is

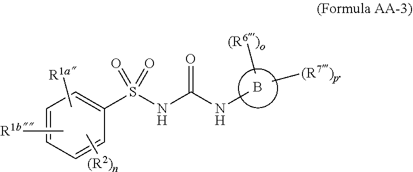

6. The compound of claim 3, wherein the compound of Formula AA is

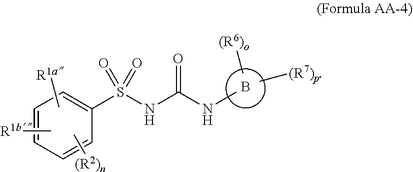

7. The compound of claim 3, wherein the compound of Formula AA is

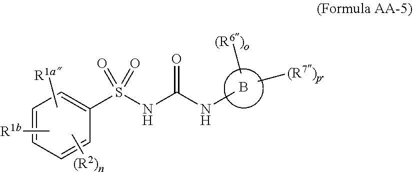

8. The compound of claim 3, wherein the compound of Formula AA is

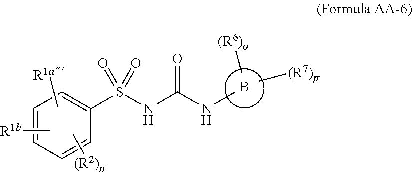

9. The compound of claim 1, wherein A is a 5- to 6-membered heteroaryl containing 1 sulfur ring member.

10. The compound of any one of claims 1-3, wherein A is thiazolyl.

11. The compound of any one of claims 1-3, wherein A is oxazolyl.

12. The compound of any one of claims 1-11, wherein n=0.

13. The compound of any one of claims 1, 9, and optionally 10, wherein the substituted ring A is 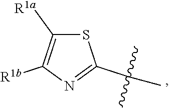
or wherein the substituted ring A is 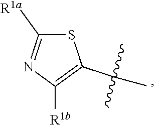
or wherein the substituted ring A is 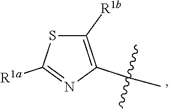
or wherein the substituted ring A is 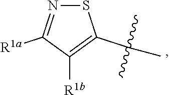
wherein the substituted ring A is 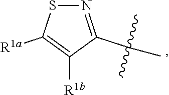
or wherein the substituted ring A is

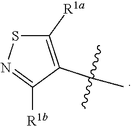

14. The compound of any one of claims 1-11, wherein n=1.

15. The compound of any one of claims 1, and 12, wherein the substituted ring A is

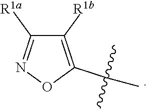

16. The compound of any one of claims 1, and 14, wherein the substituted ring A is 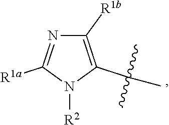
or wherein the substituted ring A is 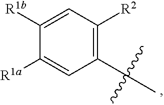
or wherein the substituted ring A is 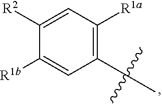
or wherein the substituted ring A is

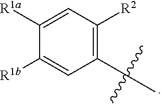

17. The compound of any one of claims 1, and 12, wherein the substituted ring A is 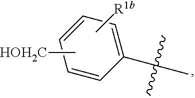
or wherein the substituted ring A is

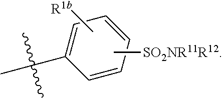

18. The compound of any one of claims 1-3 and 13, wherein the substituted ring A is 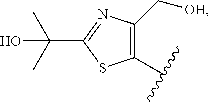 [SMILES: *c1sc(C(C)(C)O)nc1CO]
or wherein the substituted ring A is

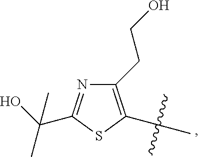 [SMILES: *c1sc(C(C)(C)O)nc1CCO]

19. The compound of any one of claims 1-2, and 4, wherein the substituted ring A is 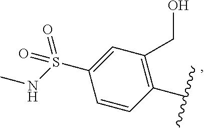 [SMILES: *c1ccc(S(=O)(=O)NC)cc1CO]
or wherein the substituted ring A is

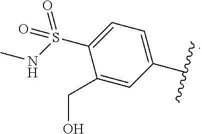 [SMILES: *c1ccc(S(=O)(=O)NC)c(CO)c1]

20. The compound of any one of claims 1-3 and 9-16, wherein R^{1a} is C₁-C₆ alkyl substituted with one or more hydroxy; or
wherein R^{1a} is C₁-C₆ alkyl substituted with one or more —OSi(R¹³)₃; or
wherein R^{1a} is —SO₂NR¹¹R¹².

21. The compound of any one of claims 1-3 and 9-17, wherein R^{1b} is independently selected from the group consisting of C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², and —NR¹¹COR¹²; or
wherein R^{1b} is independently selected from the group consisting of —SO₂NR¹¹R¹², —SO₂R¹³, —CONR¹¹R¹², —COR¹³, —CO₂R¹³, —NR¹³CONR¹¹R¹²; and —CR¹¹R¹²CN; or
wherein R^{1b} is —SO₂NHMe, SO₂NHCH₂CH₂OH, SO₂Me, CONHMe, or OMe; or
wherein R^{1b} is —SO₂NHMe or OMe.

22. The compound of any one of claims 1-10, and 16, wherein R² is independently selected from the group consisting of hydroxymethyl, C₂ alkyl substituted with hydroxy, C₃ alkyl substituted with hydroxy, C₄ alkyl substituted with hydroxy, C₅ alkyl substituted with hydroxy, and C₆ alkyl substituted with hydroxy; or
wherein R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, 5-hydroxy-1-pentyl, and 6-hydroxy-1-hexyl; or
wherein R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, and 6-hydroxy-1-hexyl; or
wherein R² is selected from the group consisting of C₁-C₆ alkyl optionally substituted with one or more hydroxy, halo, oxo, or C₁-C₆ alkoxy, C₃-C₇ cycloalkyl optionally substituted with one or more hydroxy, halo, oxo, C₁-C₆ alkoxy, or C₁-C₆ alkyl wherein the C₁-C₆ alkoxy or C₁-C₆ alkyl is further optionally substituted with one to three hydroxy, halo, or oxo; 3- to 7-membered heterocycloalkyl optionally substituted with one or more hydroxy, halo, oxo, or C₁-C₆ alkyl, wherein the C₁-C₆ alkoxy or C₁-C₆ alkyl is further optionally substituted with one to three hydroxy, halo, or oxo, C₁-C₆ haloalkyl;
C₁-C₆ alkoxy; C₁-C₆ haloalkoxy; halo; CN; CO—C₁-C₆ alkyl; CO—C₆-C₁₀ aryl; CO(5- to 10-membered heteroaryl); CO₂C₁-C₆ alkyl, CO₂C₃-C₈ cycloalkyl; OCOC₁-C₆ alkyl;
OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl;
N(C₁-C₆ alkyl)₂; CONR⁸R⁹; SF₅; S(O₂)NR¹¹R¹²; S(O)C₁-C₆ alkyl; and S(O₂)C₁-C₆ alkyl; or
wherein R² is selected from the group consisting of fluoro; chloro; cyano; methyl;
methoxy; ethoxy; isopropyl; 1-hydroxy-2-methylpropan-2-yl; 2-hydroxy-2-propyl;
hydroxymethyl; 1-hydroxyethyl; 2-hydroxyethyl; l-hydroxy-2-propyl; 1-hydroxy-1-cyclopropyl; COCH₃; COPh; 2-methoxy-2-propyl; phenyl; S(O₂)CH₃; and S(O₂)NR¹¹R¹².

23. The compound of any one of claims 1-3, 6, 8, and 9-22, wherein B is phenyl substituted with 1 or 2 R⁶ and optionally substituted with 1, 2, or 3 R⁷.

24. The compound of claim 23, wherein o=2 and p=0.

25. The compound of any one of claims 23 and 24, wherein the optionally substituted ring B is

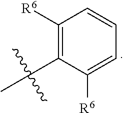

26. The compound of claim 25, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4-to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ and, NHCO(5- to 10-membered heteroaryl), NHCO(4- to 6-membered heterocycloalkyl), and NHCOC₂-C₆ alkynyl; or wherein each R⁶ is independently selected from the group consisting of: C₁-C₆ alkyl, C₃-C₇ cycloalkyl, C₁-C₆ haloalkyl, C₁-C₆ alkoxy, and C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

27. The compound of claim 23, wherein o=1 and p=₁; or
wherein o=2 and p=1.

28. The compound of claim 27, wherein the optionally substituted ring B is

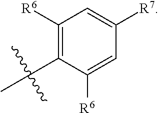

29. The compound of claim 28, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

30. The compound of claim 23, wherein o=2 and p=2.

31. The compound of any one of claims 1-3, wherein the optionally substituted ring B is

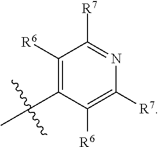

32. The compound of any one of claims 30 and 31, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4-to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R is independently selected from C₁-C₆ alkyl, C₁-C₆ haloalkyl, C₁-C₆ alkoxy, C₁-C₆ haloalkoxy, halo, CN, COC₁-C₆ alkyl, C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ and, and CONR⁸R⁹.

33. The compound of claim 30, wherein the optionally substituted ring B is

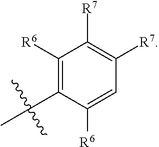

34. The compound of claim 33, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₃-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ and, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or at least one 5-to-7membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, =NR¹⁰, COOC₁-C₆ alkyl, C₆-C₁₀ and, and CONR⁸R⁹.

35. The compound of claim 23, wherein o=2 and p=3

36. The compound of claim 35, wherein the optionally substituted ring B is

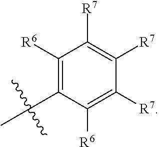

37. The compound of claim 36, wherein the optionally substituted ring B is

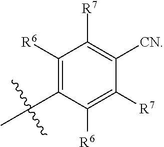

38. The compound of any one of claims 1-3, wherein the optionally substituted ring B is

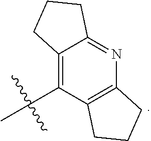 [SMILES: *c1c2c(nc3c1CCC3)CCC2]

39. The compound of claim 36, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4 to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

40. The compound of any one of claims 1-3, 6, 8-25, 30-31, and 36-37, wherein two pairs of R⁶ and R⁷ on adjacent atoms, taken together with the atoms connecting them, independently form at least one C₄-C₈ carbocyclic ring or at least one 5-to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, —NR¹⁰, COOC₁-C₆ alkyl, C₆-C₁₀ aryl, and CONR⁸R⁹.

41. The compound of any one of claims 1-3, 6, 8-25, 27-28, 30, 31, 33, and 36-37, wherein each R⁶ is independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy; or
wherein each R⁷ is independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

42. The compound of any one of the preceding claims, wherein R³ is hydrogen.

43. A compound selected from the group consisting of the compound s below:

| Compound | Structure |
|---|---|
| 101 | 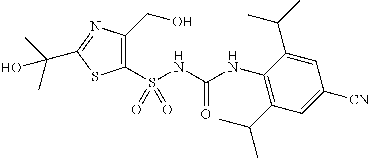 [SMILES: CC(C)c1cc(C#N)cc(C(C)C)c1NC(=O)NS(=O)(=O)c1sc(C(C)(C)O)nc1CO] |
| 102 | 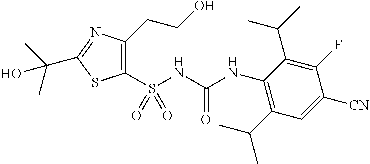 [SMILES: CC(C)c1cc(C#N)c(F)c(C(C)C)c1NC(=O)NS(=O)(=O)c1sc(C(C)(C)O)nc1CCO] |
| 103 | 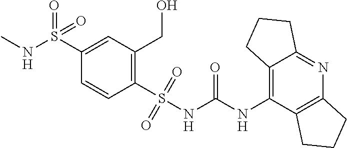 [SMILES: CNS(=O)(=O)c1ccc(S(=O)(=O)NC(=O)Nc2c3c(nc4c2CCC4)CCC3)c(CO)c1] |
| 104 | 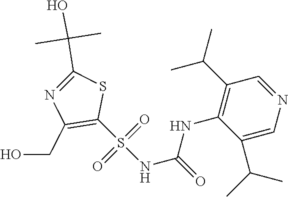 [SMILES: CC(C)c1cncc(C(C)C)c1NC(=O)NS(=O)(=O)c1sc(C(C)(C)O)nc1CO] |
| 105 | 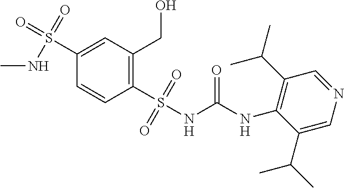 [SMILES: CNS(=O)(=O)c1ccc(S(=O)(=O)NC(=O)Nc2c(C(C)C)cncc2C(C)C)c(CO)c1] |
| 106 | 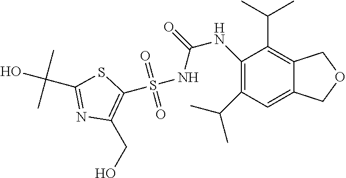 [SMILES: CC(C)c1cc2c(c(C(C)C)c1NC(=O)NS(=O)(=O)c1sc(C(C)(C)O)nc1CO)COC2] |
| 107 | 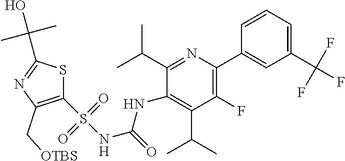 [SMILES: CC(C)c1nc(-c2cccc(C(F)(F)F)c2)c(F)c(C(C)C)c1NC(=O)NS(=O)(=O)c1sc(C(C)(C)O)nc1CO[Si](C)(C)C(C)(C)C] |
| 108 | 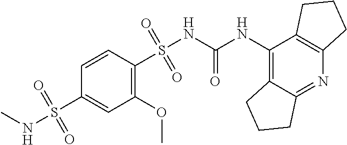 [SMILES: CNS(=O)(=O)c1ccc(S(=O)(=O)NC(=O)Nc2c3c(nc4c2CCC4)CCC3)c(OC)c1] |
| 109 | 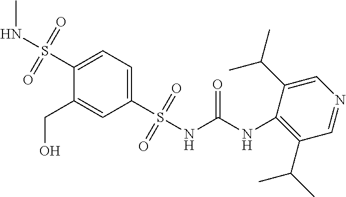 [SMILES: CNS(=O)(=O)c1ccc(S(=O)(=O)NC(=O)Nc2c(C(C)C)cncc2C(C)C)cc1CO] |
| 110 | 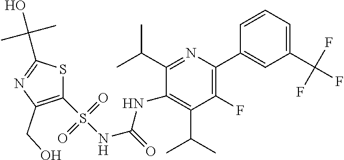 [SMILES: CC(C)c1nc(-c2cccc(C(F)(F)F)c2)c(F)c(C(C)C)c1NC(=O)NS(=O)(=O)c1sc(C(C)(C)O)nc1CO] |
| 111 | 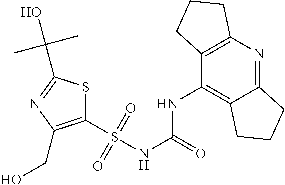 [SMILES: CC(C)(O)c1nc(CO)c(S(=O)(=O)NC(=O)Nc2c3c(nc4c2CCC4)CCC3)s1] |
| 112 | 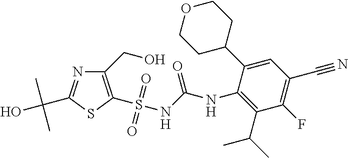 [SMILES: CC(C)c1c(F)c(C#N)cc(C2CCOCC2)c1NC(=O)NS(=O)(=O)c1sc(C(C)(C)O)nc1CO] |
| 113 | 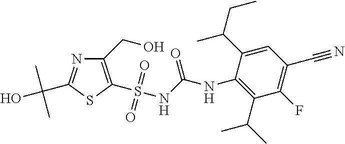 [SMILES: CCC(C)c1cc(C#N)c(F)c(C(C)C)c1NC(=O)NS(=O)(=O)c1sc(C(C)(C)O)nc1CO] |
| 114 | 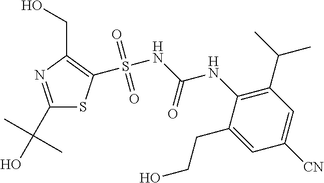 [SMILES: CC(C)c1cc(C#N)cc(CCO)c1NC(=O)NS(=O)(=O)c1sc(C(C)(C)O)nc1CO] |
| 115 | 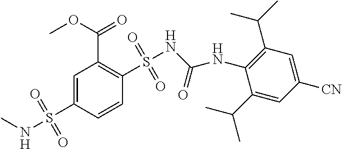 [SMILES: CNS(=O)(=O)c1ccc(S(=O)(=O)NC(=O)Nc2c(C(C)C)cc(C#N)cc2C(C)C)c(C(=O)OC)c1] |
| 116 | 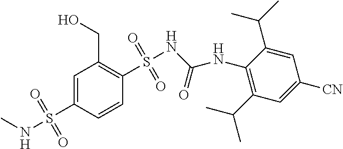 [SMILES: CNS(=O)(=O)c1ccc(S(=O)(=O)NC(=O)Nc2c(C(C)C)cc(C#N)cc2C(C)C)c(CO)c1] |
| 117 | 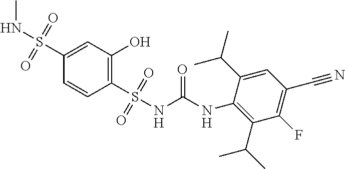 [SMILES: CNS(=O)(=O)c1ccc(S(=O)(=O)NC(=O)Nc2c(C(C)C)cc(C#N)c(F)c2C(C)C)c(O)c1] |
| 118 | 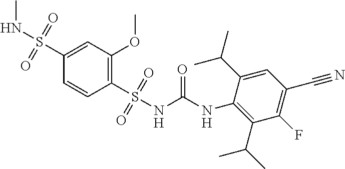 [SMILES: CNS(=O)(=O)c1ccc(S(=O)(=O)NC(=O)Nc2c(C(C)C)cc(C#N)c(F)c2C(C)C)c(OC)c1] |
| 119 | 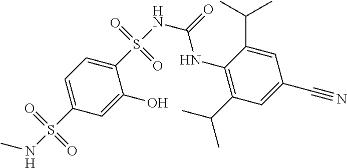 [SMILES: CNS(=O)(=O)c1ccc(S(=O)(=O)NC(=O)Nc2c(C(C)C)cc(C#N)cc2C(C)C)c(O)c1] |
| 120 | 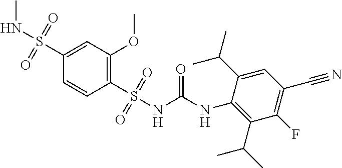 [SMILES: CNS(=O)(=O)c1ccc(S(=O)(=O)NC(=O)Nc2c(C(C)C)cc(C#N)c(F)c2C(C)C)c(OC)c1] |
| 121 | 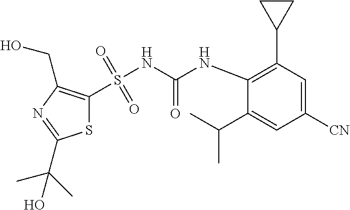 [SMILES: CC(C)c1cc(C#N)cc(C2CC2)c1NC(=O)NS(=O)(=O)c1sc(C(C)(C)O)nc1CO] |
| 122 | 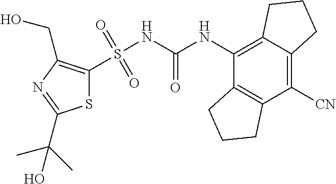 [SMILES: CC(C)(O)c1nc(CO)c(S(=O)(=O)NC(=O)Nc2c3c(c(C#N)c4c2CCC4)CCC3)s1] |
| 123 | 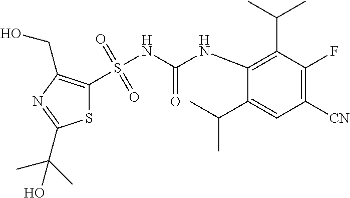 [SMILES: CC(C)c1cc(C#N)c(F)c(C(C)C)c1NC(=O)NS(=O)(=O)c1sc(C(C)(C)O)nc1CO] |
| 124 | 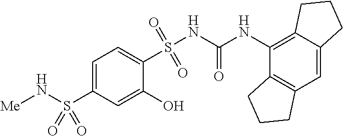 [SMILES: CNS(=O)(=O)c1ccc(S(=O)(=O)NC(=O)Nc2c3c(cc4c2CCC4)CCC3)c(O)c1] |

and pharmaceutically acceptable salts thereof.

44. A pharmaceutical composition comprising a compound or salt as claimed in any one of claims 1-43 and one or more pharmaceutically acceptable excipients.

45. A method for modulating NRLP3 activity, the method comprising contacting NRLP3 with an effective amount of a compound as claimed in any one of claims 1-43 or a pharmaceutical composition as claimed in claim 44.

46. The method of claim 45, wherein the modulating comprises antagonizing NRLP3.

47. A method of treating a disease, disorder or condition that is a metabolic disorder, comprising administering to a subject in need of such treatment an effective amount of a compound as claimed in any one of claims 1-43 or a pharmaceutical composition as claimed in claim 44,
wherein the metabolic disorder is Type 2 diabetes, atherosclerosis, obesity or gout.

48. A method of treating a disease, disorder or condition that is a disease of the central nervous system, comprising administering to a subject in need of such treatment an effective amount of a compound as claimed in any one of claims 1-43 or a pharmaceutical composition as claimed in claim 44,
wherein the disease of the central nervous system is Alzheimer's disease, multiple sclerosis, Amyotrophic Lateral Sclerosis or Parkinson's disease.

49. A method of treating a disease, disorder or condition that is lung disease, comprising administering to a subject in need of such treatment an effective amount of a compound as claimed in any one of claims 1-43 or a pharmaceutical composition as claimed in claim 44,
wherein the lung disease is asthma, COPD or pulmonary idiopathic fibrosis.

50. A method of treating a disease, disorder or condition that is liver disease, comprising administering to a subject in need of such treatment an effective amount of a compound as claimed in any one of claims 1-43 or a pharmaceutical composition as claimed in claim 44,
wherein the liver disease is NASH syndrome, viral hepatitis or cirrhosis.

51. A method of treating a disease, disorder or condition that is pancreatic disease, comprising administering to a subject in need of such treatment an effective amount of a compound as claimed in any one of claims 1-43 or a pharmaceutical composition as claimed in claim 44,
wherein the pancreatic disease is acute pancreatitis or chronic pancreatitis.

52. A method of treating a disease, disorder or condition that is kidney disease, comprising administering to a subject in need of such treatment an effective amount of a compound as claimed in any one of claims 1-43 or a pharmaceutical composition as claimed in claim 44, wherein the kidney disease is acute kidney injury or chronic kidney injury.

53. A method of treating a disease, disorder or condition that is intestinal disease, comprising administering to a subject in need of such treatment an effective amount of a compound as claimed in any one of claims 1-43 or a pharmaceutical composition as claimed in claim 44, wherein the intestinal disease is Crohn's disease or Ulcerative Colitis.

54. A method of treating a disease, disorder or condition that is skin disease, comprising administering to a subject in need of such treatment an effective amount of a compound as claimed in any one of claims 1-43 or a pharmaceutical composition as claimed in claim 44, wherein the skin disease is psoriasis.

55. A method of treating a disease, disorder or condition that is musculoskeletal disease, comprising administering to a subject in need of such treatment an effective amount of a compound as claimed in any one of claims 1-43 or a pharmaceutical composition as claimed in claim 44, wherein the musculoskeletal disease is scleroderma.

56. A method of treating a disease, disorder or condition that is a vessel disorder, comprising administering to a subject in need of such treatment an effective amount of a compound as cl aimed in any one of claims 1-43 or a pharmaceutical composition as claimed in claim 44, wherein the vessel disorder is giant cell arteritis.

57. A method of treating a disease, disorder or condition that is a disorder of the bones, comprising administering to a subject in need of such treatment an effective amount of a compound as claimed in any one of claims 1-43 or a pharmaceutical composition as claimed in claim 44, wherein the disorder of the bones is osteoarthritis, osteoporosis or osteopetrosis disorders.

58. A method of treating a disease, disorder or condition that is eye disease, comprising administering to a subject in need of such treatment an effective amount of a compound as cl aimed in any one of claims 1-43 or a pharmaceutical composition as claimed in claim 44, wherein the eye disease is glaucoma or macular degeneration.

59. A method of treating a disease, disorder or condition that is a disease caused by viral infection, comprising administering to a subject in need of such treatment an effective amount of a compound as claimed in any one of claims 1-43 or a pharmaceutical composition as claimed in claim 44, wherein the diseases caused by viral infection is HIV or AIDS.

60. A method of treating a disease, disorder or condition that is an autoimmune disease, comprising administering to a subject in need of such treatment an effective amount of a compound as cl aimed in any one of claims 1-43 or a pharmaceutical composition as claimed in claim 44, wherein the autoimmune disease is Rheumatoid Arthritis, Systemic Lupus Erythematosus, Autoimmune Thyroiditis.

61. A method of treating a disease, disorder or condition that is cancer or aging, comprising administering to a subject in need of such treatment an effective amount of a compound as claimed in any one of claims 1-43 or a pharmaceutical composition as claimed in claim 44.

62. A method of treating a disease, disorder or condition that is a cancer selected from: myeiodysplastic syndromes (AIDS): non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis, comprising administering to a subject in need of such treatment an effective amount of a compound as claimed in any one of claims 1-43 or a pharmaceutical composition as claimed in claim 44.

63. The method of any one of claims 47-62, further comprising administering a therapeutically effective amount of an anti-TNFα agent to the subject.


SEQUENCE LISTING (37 sequences)

SEQ ID NO: 1 - DNA, 702 bp, Homo sapiens
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 2 - DNA, 1368 bp, Homo sapiens
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatga

SEQ ID NO: 3 - DNA, 281 bp, Homo sapiens
attcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 4 - DNA, 939 bp, Homo sapiens
atggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctag

SEQ ID NO: 5 - DNA, 1506 bp, Homo sapiens
atggctgcagctagcgtgaccccccctggctccctggagttgctacagcccggcttctccaagaccctcctggggaccaagctggaagccaagtacctgtgctccgcctgcagaaacgtcctccgcaggcccttccaggcgcagtgtggccaccggtactgctccttctgcctggccagcatcctcagctctgggcctcagaactgtgctgcctgtgttcacgagggcatatatgaagaaggcatttctattttagaaagcagttcggccttcccagataatgctgcccgcagggaggtggagagcctgccggccgtctgtcccagtgatggatgcacctggaaggggaccctgaaagaatacgagagctgccacgaaggccgctgcccgctcatgctgaccgaatgtcccgcgtgcaaaggcctggtccgccttggtgaaaaggagcgccacctggagcacgagtgcccggagagaagcctgagctgccggcattgccgggcaccctgctgcggagcagacgtgaaggcgcaccacgaggtctgccccaagttccccttaacttgtgacggctgcggcaagaagaagatcccccgggagaagtttcaggaccacgtcaagacttgtggcaagtgtcgagtcccttgcagattccacgccatcggctgcctcgagacggtagagggtgagaaacagcaggagcacgaggtgcagtggctgcgggagcacctggccatgctactgagctcggtgctggaggcaaagcccctcttgggagaccagagccacgcggggtcagagctcctgcagaggtgcgagagcctggagaagaagacggccacttttgagaacattgtctgcgtcctgaaccgggaggtggagagggtggccatgactgccgaggcctgcagccggcagcaccggctggaccaagacaagattgaagccctgagtagcaaggtgcagcagctggagaggagcattggcctcaaggacctggcgatggctgacttggagcagaaggtcttggagatggaggcatccacctacgatggggtcttcatctggaagatctcagacttcgccaggaagcgccaggaagctgtggctggccgcatacccgccatcttctccccagccttctacaccagcaggtacggctacaagatgtgtctgcgtatctacctgaacggcgacggcaccgggcgaggaacacacctgtccctcttctttgtggtgatgaagggcccgaatgacgccctgctgcggtggcccttcaaccagaaggtgaccttaatgctgctcgaccagaataaccgggagcacgtgattgacgccttcaggcccgacgtgacttcatcctcttttcagaggccagtcaacgacatgaacatcgcaagcggctgccccctcttctgccccgtctccaagatggaggcaaagaattcctacgtgcgggacgatgccatcttcatcaaggccattgtggacctgacagggctctaa

SEQ ID NO: 6 - DNA, 1044 bp, Homo sapiens
atggaaacacccttctacggcgatgaggcgctgagcggcctgggcggcggcgccagtggcagcggcggcagcttcgcgtccccgggccgcttgttccccggggcgcccccgacggccgcggccggcagcatgatgaagaaggacgcgctgacgctgagcctgagtgagcaggtggcggcagcgctcaagcctgcggccgcgccgcctcctacccccctgcgcgccgacggcgcccccagcgcggcaccccccgacggcctgctcgcctctcccgacctggggctgctgaagctggcctcccccgagctcgagcgcctcatcatccagtccaacgggctggtcaccaccacgccgacgagctcacagttcctctaccccaaggtggcggccagcgaggagcaggagttcgccgagggcttcgtcaaggccctggaggatttacacaagcagaaccagctcggcgcgggcgcggccgctgccgccgccgccgccgccgccggggggccctcgggcacggccacgggctccgcgccccccggcgagctggccccggcggcggccgcgcccgaagcgcctgtctacgcgaacctgagcagctacgcgggcggcgccgggggcgcggggggcgccgcgacggtcgccttcgctgccgaacctgtgcccttcccgccgccgccacccccaggcgcgttggggccgccgcgcctggctgcgctcaaggacgagccacagacggtgcccgacgtgccgagcttcggcgagagcccgccgttgtcgcccatcgacatggacacgcaggagcgcatcaaggcggagcgcaagcggctgcgcaaccgcatcgccgcctccaagtgccgcaagcgcaagctggagcgcatctcgcgcctggaagagaaagtgaagaccctcaagagtcagaacacggagctggcgtccacggcgagcctgctgcgcgagcaggtggcgcagctcaagcagaaagtcctcagccacgtcaacagcggctgccagctgctgccccagcaccaggtgcccgcgtactga

SEQ ID NO: 7 - DNA, 4124 bp, Homo sapiens
atgagcacggaggcggacgagggcatcactttctctgtgccacccttcgccccctcgggcttctgcaccatccccgagggcggcatctgcaggaggggaggagcggcggcggtgggcgagggcgaggagcaccagctgccaccgccgccgccgggcagtttctggaacgtggagagcgccgctgcccctggcatcggttgtccggcggccacctcctcgagcagtgccacccgaggccggggcagctctgttggcgggggcagccgacggaccacggtggcatatgtgatcaacgaagcgagccaagggcaactggtggtggccgagagcgaggccctgcagagcttgcgggaggcgtgcgagacagtgggcgccaccctggaaccctgcattttgggaaactcgactttggagaaaccaccgtgctggaccgcttttacaatgcagatattgcggtggtggagatgagcgatgccttccggcagccgtccttgttttaccaccttggggtgagagaaagtttcagcatggccaacaacatcatcctctactgcgatactaactcggactctctgcagtcactgaaggaaatcatttgccagaagaatactatgtgcactgggaactacacctttgttccttacatgataactccacataacaaagtctactgctgtgacagcagcttcatgaaggggttgacagagctcatgcaaccgaacttcgagctgcttcttggacccatctgcttacctcttgtggatcgttttattcaacttttgaaggtggcacaagcaagttctagccagtacttccgggaatctatactcaatgacatcaggaaagctcgtaatttatacactggtaaagaattggcagctgagttggcaagaattcggcagcgagtagataatatcgaagtcttgacagcagatattgtcataaatctgttactttcctacagagatatccaggactatgattctattgtgaagctggtagagactttagaaaaactgccaacctttgatttggcctcccatcaccatgtgaagtttcattatgcatttgcactgaataggagaaatctccctggtgacagagcaaaagctcttgatattatgattcccatggtgcaaagcgaaggacaagttgcttcagatatgtattgcctagttggtcgaatctacaaagatatgtttttggactctaatttcacggacactgaaagcagagaccatggagcttcttggttcaaaaaggcatttgaatctgagccaacactacagtcaggaattaattatgcggtcctcctcctggcagctggacaccagtttgaatcttcctttgagctccggaaagttggggtgaagctaagtagtcttcttggtaaaaagggaaacttggaaaaactccagagctactgggaagttggattttttctgggggccagcgtcctagccaatgaccacatgagagtcattcaagcatctgaaaagctttttaaactgaagacaccagcatggtacctcaagtctattgtagagacaattttgatatataagcattttgtgaaactgaccacagaacagcctgtggccaagcaagaacttgtggacttttggatggatttcctggtcgaggccacaaagacagatgttactgtggttaggtttccagtattaatattagaaccaaccaaaatctatcaaccttcttatttgtctatcaacaatgaagttgaggaaaagacaatctctatttggcacgtgcttcctgatgacaagaaaggtatacatgagtggaattttagtgcctcttctgtcaggggagtgagtatttctaaatttgaagaaagatgctgctttctttatgtgcttcacaattctgatgatttccaaatctatttctgtacagaacttcattgtaaaaagttttttgagatggtgaacaccattaccgaagagaaggggagaagcacagaggaaggagactgtgaaagtgacttgctggagtatgactatgaatatgatgaaaatggtgacagagtcgttttaggaaaaggcacttatgggatagtctacgcaggtcgggacttgagcaaccaagtcagaattgctattaaggaaatcccagagagagacagcagatactctcagcccctgcatgaagaaatagcattgcataaacacctgaagcacaaaaatattgtccagtatctgggctctttcagtgagaatggtttcattaaaatcttcatggagcaggtccctggaggaagtctttctgctctccttcgttccaaatggggtccattaaaagacaatgagcaaacaattggcttttatacaaagcaaatactggaaggattaaaatatctccatgacaatcagatagttcaccgggacataaagggtgacaatgtgttgattaatacctacagtggtgttctcaagatctctgacttcggaacatcaaagaggcttgctggcataaacccctgtactgaaacttttactggtaccctccagtatatggcaccagaaataatagataaaggaccaagaggctacggaaaagcagcagacatctggtctctgggctgtacaatcattgaaatggccacaggaaaacccccattttatgaactgggagaaccacaagcagctatgttcaaggtgggaatgtttaaagtccaccctgagatcccagagtccatgtctgcagaggccaaggcattcatactgaaatgttttgaaccagatcctgacaagagagcctgtgctaacgacttgcttgttgatgagtttttaaaagtttcaagcaaaaagaaaaagacacaacctaagctttcagctctttcagctggatcaaatgaatatctcaggagtatatccttgccggtacctgtgctggtggaggacaccagcagcagcagtgagtacggctcagtttcacccgacacggagttgaaagtggaccccttctctttcaaaacaagagccaagtcctgcggagaaagagatgtcaagggaattcggacactctttttgggcattccagatgagaattttgaagatcacagtgctcctccttcccctgaagaaaaagattctggattcttcatgctgaggaaggacagtgagaggcgagctacccttcacaggatcctgacggaagaccaagacaaaattgtgagaaacctaatggaatctttagctcagggggctgaagaaccgaaactaaaatgggaacacatcacaaccctcattgcaagcctcagagaatttgtgagatccactgaccgaaaaatcatagccaccacactgtcaaagctgaaactggagctggacttcgacagccatggcattagccaagtccaggtggtactctttggttttcaagatgctgtcaataaagttcttcggaatcataacatcaagccgcactggatgtttgccttagacagtatcattcggaaggcggtacagacagccattaccatcctggttccagaactaaggccacatttcagccttgcatctgagagtgatactgctgatcaagaagacttggatgtagaagatgaccatgaggaacagccttcaaatcaaactgtccgaagacctcaggctgtcattgaagatgctgtggctacctcaggcgtgagcacgctcagttctactgtgtctcatgattcccagagtgctcaccggtcactgaatgtacagcttggaaggatgaaaatagaaaccaatagattactggaagaattggttcggaaagagaaagaattacaagcactccttcatcgagctattgaagaaaaagaccaagaaattaaacacctgaagcttaagtcccaacccatagaaattcctgaattgcctgtatttcatctaaattcttctggcacaaatactgaagattctgaacttaccgactggctgagagtgaatggagctgatgaagacactataagccggtttttggctgaagattatacactattggatgttctctactatgttacacgtgatgacttaaaatgcttgagactaaggggagggatgctgtgcacactgtggaaggctatcattgactttcgaaacaaacagacttga

SEQ ID NO: 8 - DNA, 1128 bp, Homo sapiens
atggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaa

SEQ ID NO: 9 - DNA, 1140 bp, Homo sapiens
atggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctag

SEQ ID NO: 10 - DNA, 1083 bp, Homo sapiens
atggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaa

SEQ ID NO: 11 - DNA, 2265 bp, Homo sapiens
atgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 12 - DNA, 954 bp, Homo sapiens
atgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatga

SEQ ID NO: 13 - DNA, 2139 bp, Homo sapiens
atggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctga

SEQ ID NO: 14 - DNA, 1284 bp, Homo sapiens
atgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgttgacatttggtcagttgggtgcatcatgggagaaatgatcaaaggtggtgttttgttcccaggtacagatcatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatga

SEQ ID NO: 15 - DNA, 1446 bp, Homo sapiens
atgggggccttggccagagccctgccgtccatactgctggcattgctgcttacgtccaccccagaggctctgggtgccaaccccggcttggtcgccaggatcaccgacaagggactgcagtatgcggcccaggaggggctattagctctgcagagtgagctgctcaggatcacgctgcctgacttcaccggggacttgaggatcccccacgtcggccgtgggcgctatgagttccacagcctgaacatccacagctgtgagctgcttcactctgcgctgaggcctgtccctggccagggcctgagtctcagcatctccgactcctccatccgggtccagggcaggtggaaggtgcgcaagtcattcttcaaactacagggctcctttgatgtcagtgtcaagggcatcagcatttcggtcaacctcctgttgggcagcgagtcctccgggaggcccacagttactgcctccagctgcagcagtgacatcgctgacgtggaggtggacatgtcgggagacttggggtggctgttgaacctcttccacaaccagattgagtccaagttccagaaagtactggagagcaggatttgcgaaatgatccagaaatcggtgtcctccgatctacagccttatctccaaactctgccagttacaacagagattgacagtttcgccgacattgattatagcttagtggaagcccctcgggcaacagcccagatgctggaggtgatgtttaagggtgaaatctttcatcgtaaccaccgttctccagttaccctccttgctgcagtcatgagccttcctgaggaacacaacaaaatggtctactttgccatctcggattatgtcttcaacacggccagcctggtttatcatgaggaaggatatctgaacttctccatcacagatgacatgataccgcctgactctaatatccgactgaccaccaagtccttccgacccttcgtcccacggttagccaggctctaccccaacatgaacctggaactccagggatcagtgccctctgctccgctcctgaacttcagccctgggaatctgtctgtggacccctatatggagatagatgcctttgtgctcctgcccagctccagcaaggagcctgtcttccggctcagtgtggccactaatgtgtccgccaccttgaccttcaataccagcaagatcactgggttcctgaagccaggaaaggtaaaagtggaactgaaagaatccaaagttggactattcaatgcagagctgttggaagcgctcctcaactattacatccttaacaccctctaccccaagttcaatgataagttggccgaaggcttcccccttcctctgctgaagcgtgttcagctctacgaccttgggctgcagatccataaggacttcctgttcttgggtgccaatgtccaatacatgagagtttga

SEQ ID NO: 16 - DNA, 1182 bp, Homo sapiens
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 17 - DNA, 1203 bp, Homo sapiens
atgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtga

SEQ ID NO: 18 - DNA, 957 bp, Homo sapiens
atgtccaagccacccgcacccaaccccacacccccccggaacctggactcccggaccttcatcaccattggagacagaaactttgaggtggaggctgatgacttggtgaccatctcagaactgggccgtggagcctatggggtggtagagaaggtgcggcacgcccagagcggcaccatcatggccgtgaagcggatccgggccaccgtgaactcacaggagcagaagcggctgctcatggacctggacatcaacatgcgcacggtcgactgtttctacactgtcaccttctacggggcactattcagagagggagacgtgtggatctgcatggagctcatggacacatccttggacaagttctaccggaaggtgctggataaaaacatgacaattccagaggacatccttggggagattgctgtgtctatcgtgcgggccctggagcatctgcacagcaagctgtcggtgatccacagagatgtgaagccctccaatgtccttatcaacaaggagggccatgtgaagatgtgtgactttggcatcagtggctacttggtggactctgtggccaagacgatggatgccggctgcaagccctacatggcccctgagaggatcaacccagagctgaaccagaagggctacaatgtcaagtccgacgtctggagcctgggcatcaccatgattgagatggccatcctgcggttcccttacgagtcctgggggaccccgttccagcagctgaagcaggtggtggaggagccgtccccccagctcccagccgaccgtttctcccccgagtttgtggacttcactgctcagtgcctgaggaagaaccccgcagagcgtatgagctacctggagctgatggagcaccccttcttcaccttgcacaaaaccaagaagacggacattgctgccttcgtgaaggagatcctgggagaagactcatag

SEQ ID NO: 19 - DNA, 1005 bp, Homo sapiens
atgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaa

SEQ ID NO: 20 - DNA, 4539 bp, Homo sapiens
atggcggcggcggcggggaatcgcgcctcgtcgtcgggattcccgggcgccagggctacgagccctgaggcaggcggcggcggaggagccctcaaggcgagcagcgcgcccgcggctgccgcgggactgctgcgggaggcgggcagcgggggccgcgagcgggcggactggcggcggcggcagctgcgcaaagtgcggagtgtggagctggaccagctgcctgagcagccgctcttccttgccgcctcaccgccggcctcctcgacttccccgtcgccggagcccgcggacgcagcggggagtgggaccggcttccagcctgtggcggtgccgccgccccacggagccgcgagccgcggcggcgcccaccttaccgagtcggtggcggcgccggacagcggcgcctcgagtcccgcagcggccgagcccggggagaagcgggcgcccgccgccgagccgtctcctgcagcggcccccgccggtcgtgagatggagaataaagaaactctcaaagggttgcacaagatggatgatcgtccagaggaacgaatgatcagggagaaactgaaggcaacctgtatgccagcctggaagcacgaatggttggaaaggagaaataggcgagggcctgtggtggtaaaaccaatcccagttaaaggagatggatctgaaatgaatcacttagcagctgagtctccaggagaggtccaggcaagtgcggcttcaccagcttccaaaggccgacgcagtccttctcctggcaactccccatcaggtcgcacagtgaaatcagaatctccaggagtaaggagaaaaagagtttccccagtgccttttcagagtggcagaatcacaccaccccgaagagccccttcaccagatggcttctcaccatatagccctgaggaaacaaaccgccgtgttaacaaagtgatgcgggccagactgtacttactgcagcagatagggcctaactctttcctgattggaggagacagcccagacaataaataccgggtgtttattgggcctcagaactgcagctgtgcacgtggaacattctgtattcatctgctatttgtgatgctccgggtgtttcaactagaaccttcagacccaatgttatggagaaaaactttaaagaattttgaggttgagagtttgttccagaaatatcacagtaggcgtagctcaaggatcaaagctccatctcgtaacaccatccagaagtttgtttcacgcatgtcaaattctcatacattgtcatcatctagtacttctacgtctagttcagaaaacagcataaaggatgaagaggaacagatgtgtcctatttgcttgttgggcatgcttgatgaagaaagtcttacagtgtgtgaagacggctgcaggaacaagctgcaccaccactgcatgtcaatttgggcagaagagtgtagaagaaatagagaacctttaatatgtcccctttgtagatctaagtggagatctcatgatttctacagccacgagttgtcaagtcctgtggattccccttcttccctcagagctgcacagcagcaaaccgtacagcagcagcctttggctggatcacgaaggaatcaagagagcaattttaaccttactcattatggaactcagcaaatccctcctgcttacaaagatttagctgagccatggattcaggtgtttggaatggaactcgttggctgcttattttctagaaactggaatgtgagagagatggccctcaggcgtctttcccatgatgtcagtggggccctgctgttggcaaatggggagagcactggaaattctgggggcagcagtggaagcagcccgagtgggggagccaccagtgggtcttcccagaccagtatctcaggagatgtggtggaggcatgctgcagcgttctgtcaatggtctgtgctgaccctgtctacaaagtgtacgttgctgctttaaaaacattgagagccatgctggtatatactccttgccacagtttagcggaaagaatcaaacttcagagacttctccagccagttgtagacaccatcctagtcaaatgtgcagatgccaatagccgcacaagtcagctgtccatatcaacactgttggaactgtgcaaaggccaagcaggagagttggcagttggcagagaaatactaaaagctggatccattggtattggtggtgttgattatgtcttaaattgtattcttggaaaccaaactgaatcaaacaattggcaagaacttcttggccgcctttgtcttatagatagactgttgttggaatttcctgctgaattttatcctcatattgtcagtactgatgtttcacaagctgagcctgttgaaatcaggtataagaagctgctgtccctcttaacctttgctttgcagtccattgataattcccactcaatggttggcaaactttccagaaggatctacttgagttctgcaagaatggttactacagtaccccatgtgttttcaaaactgttagaaatgctgagtgtttccagttccactcacttcaccaggatgcgtcgccgtttgatggctattgcagatgaggtggaaattgccgaagccatccagttgggcgtagaagacactttggatggtcaacaggacagcttcttgcaggcatctgttcccaacaactatctggaaaccacagagaacagttcccctgagtgcacagtccatttagagaaaactggaaaaggattatgtgctacaaaattgagtgccagttcagaggacatttctgagagactggccagcatttcagtaggaccttctagttcaacaacaacaacaacaacaacaacagagcaaccaaagccaatggttcaaacaaaaggcagaccccacagtcagtgtttgaactcctctcctttatctcatcattcccaattaatgtttccagccttgtcaaccccttcttcttctaccccatctgtaccagctggcactgcaacagatgtctctaagcatagacttcagggattcattccctgcagaataccttctgcatctcctcaaacacagcgcaagttttctctacaattccacagaaactgtcctgaaaacaaagactcagataaactttccccagtctttactcagtcaagacccttgccctccagtaacatacacaggccaaagccatctagacctaccccaggtaatacaagtaaacagggagatccctcaaaaaatagcatgacacttgatctgaacagtagttccaaatgtgatgacagctttggctgtagcagcaatagtagtaatgctgttatacccagtgacgagacagtgttcaccccagtagaggagaaatgcagattagatgtcaatacagagctcaactccagtattgaggaccttcttgaagcatctatgccttcaagtgatacaacagtaacttttaagtcagaagttgctgtcctgtctcctgaaaaggctgaaaatgatgatacctacaaagatgatgtgaatcataatcaaaagtgcaaagagaagatggaagctgaagaagaagaagctttagcaattgccatggcaatgtcagcgtctcaggatgccctccccatagttcctcagctgcaggttgaaaatggagaagatatcatcattattcaacaggatacaccagagactctaccaggacataccaaagcaaaacaaccgtatagagaagacactgaatggctgaaaggtcaacagataggccttggagcattttcttcttgttatcaggctcaagatgtgggaactggaactttaatggctgttaaacaggtgacttatgtcagaaacacatcttctgagcaagaagaagtagtagaagcactaagagaagagataagaatgatgagccatctgaatcatccaaacatcattaggatgttgggagccacgtgtgagaagagcaattacaatctcttcattgaatggatggcagggggatcggtggctcatttgctgagtaaatatggagccttcaaagaatcagtagttattaactacactgaacagttactccgtggcctttcgtatctccatgaaaaccaaatcattcacagagatgtcaaaggtgccaatttgctaattgacagcactggtcagagactaagaattgcagattttggagctgcagccaggttggcatcaaaaggaactggtgcaggagagtttcagggacaattactggggacaattgcatttatggcacctgaggtactaagaggtcaacagtatggaaggagctgtgatgtatggagtgttggctgtgctattatagaaatggcttgtgcaaaaccaccatggaatgcagaaaaacactccaatcatcttgctttgatatttaagattgctagtgcaactactgctccatcgatcccttcacatttgtctcctggtttacgagatgtggctcttcgttgtttagaacttcaacctcaggacagacctccatcaagagagctactgaagcatccagtctttcgtactacatggtag

SEQ ID NO: 21 - DNA, 1974 bp, Homo sapiens
atggacgaacaggaggcattgaactcaatcatgaacgatctggtggccctccagatgaaccgacgtcaccggatgcctggatatgagaccatgaagaacaaagacacaggtcactcaaataggcagaaaaaacacaacagcagcagctcagcccttctgaacagccccacagtaacaacaagctcatgtgcaggggccagtgagaaaaagaaatttttgagtgacgtcagaatcaagttcgagcacaacggggagaggcgaattatagcgttcagccggcctgtgaaatatgaagatgtggagcacaaggtgacaacagtatttggacaacctcttgatctacattacatgaacaatgagctctccatcctgctgaaaaaccaagatgatcttgataaagcaattgacattttagatagaagctcaagcatgaaaagccttaggatattgctgttgtcccaggacagaaaccataacagttcctctccccactctggggtgtccagacaggtgcggatcaaggcttcccagtccgcaggggatataaatactatctaccagccccccgagcccagaagcaggcacctctctgtcagctcccagaaccctggccgaagctcacctccccctggctatgttcctgagcggcagcagcacattgcccggcaggggtcctacaccagcatcaacagtgagggggagttcatcccagagaccagcgagcagtgcatgctggatcccctgagcagtgcagaaaattccttgtctggaagctgccaatccttggacaggtcagcagacagcccatccttccggaaatcacgaatgtcccgtgcccagagcttccctgacaacagacaggaatactcagatcgggaaactcagctttatgacaaaggggtcaaaggtggaacctacccccggcgctaccacgtgtctgtgcaccacaaggactacagtgatggcagaagaacatttccccgaatacggcgtcatcaaggcaacttgttcaccctggtgccctccagccgctccctgagcacaaatggcgagaacatgggtctggctgtgcaatacctggacccccgtgggcgcctgcggagtgcggacagcgagaatgccctctctgtgcaggagaggaatgtgccaaccaagtctcccagtgcccccatcaactggcgccggggaaagctcctgggccagggtgccttcggcagggtctatttgtgctatgacgtggacacgggacgtgaacttgcttccaagcaggtccaatttgatccagacagtcctgagacaagcaaggaggtgagtgctctggagtgcgagatccagttgctaaagaacttgcagcatgagcgcatcgtgcagtactatggctgtctgcgggaccgcgctgagaagaccctgaccatcttcatggagtacatgccagggggctcggtgaaagaccagttgaaggcttacggtgctctgacagagagcgtgacccgaaagtacacgcggcagatcctggagggcatgtcctacctgcacagcaacatgattgttcaccgggacattaagggagccaacatcctccgagactctgctgggaatgtaaagctgggggactttggggccagcaaacgcctgcagacgatctgtatgtcggggacgggcatgcgctccgtcactggcacaccctactggatgagccctgaggtgatcagcggcgagggctatggaaggaaagcagacgtgtggagcctgggctgcactgtggtggagatgctgacagagaaaccaccgtgggcagagtatgaagctatggccgccatcttcaagattgccacccagcccaccaatcctcagctgccctcccacatctctgaacatggccgggacttcctgaggcgcatttttgtggaggctcgccagagaccttcagctgaggagctgctcacacaccactttgcacagctcatgtactga

SEQ ID NO: 22 - DNA, 4826 bp, Homo sapiens
atgagagaagccgctgccgcgctggtccctcctcccgcctttgccgtcacgcctgccgccgccatggaggagccgccgccaccgccgccgccgccaccaccgccaccggaacccgagaccgagtcagaacccgagtgctgcttggcggcgaggcaagagggcacattgggagattcagcttgcaagagtcctgaatctgatctagaagacttctccgatgaaacaaatacagagaatctttatggtacctctccccccagcacacctcgacagatgaaacgcatgtcaaccaaacatcagaggaataatgtggggaggccagccagtcggtctaatttgaaagaaaaaatgaatgcaccaaatcagcctccacataaagacactggaaaaacagtggagaatgtggaagaatacagctataagcaggagaaaaagatccgagcagctcttagaacaacagagcgtgatcataaaaaaaatgtacagtgctcattcatgttagactcagtgggtggatctttgccaaaaaaatcaattccagatgtggatctcaataagccttacctcagccttggctgtagcaatgctaagcttccagtatctgtgcccatgcctatagccagacctgcacgccagacttctaggactgactgtccagcagatcgtttaaagttttttgaaactttacgacttttgctaaagcttacctcagtctcaaagaaaaaagacagggagcaaagaggacaagaaaatacgtctggtttctggcttaaccgatctaacgaactgatctggttagagctacaagcctggcatgcaggacggacaattaacgaccaggacttctttttatatacagcccgtcaagccatcccagatattattaatgaaatccttactttcaaagtcgactatgggagcttcgcctttgttagagatagagctggttttaatggtacttcagtagaagggcagtgcaaagccactcctggaacaaagattgtaggttactcaacacatcatgagcatctccaacgccagagggtctcatttgagcaggtaaaacggataatggagctgctagagtacatagaagcactttatccatcattgcaggctcttcagaaggactatgaaaaatatgctgcaaaagacttccaggacagggtgcaggcactctgtttgtggttaaacatcacaaaagacttaaatcagaaattaaggattatgggcactgttttgggcatcaagaatttatcagacattggctggccagtgtttgaaatcccttcccctcgaccatccaaaggtaatgagccggagtatgagggtgatgacacagaaggagaattaaaggagttggaaagtagtacggatgagagtgaagaagaacaaatctctgatcctagggtaccggaaatcagacagcccatagataacagcttcgacatccagtcgcgggactgcatatccaagaagcttgagaggctcgaatctgaggatgattctcttggctggggagcaccagactggagcacagaagcaggctttagtagacattgtctgacttctatttatagaccatttgtagacaaagcactgaagcagatggggttaagaaagttaattttaagacttcacaagctaatggatggttccttgcaaagggcacgtatagcattggtaaagaacgatcgtccagtggagttttctgaatttccagatcccatgtggggttcagattatgtgcagttgtcaaggacaccaccttcatctgaggagaaatgcagtgctgtgtcgtgggaggagctgaaggccatggatttaccttcattcgaacctgccttcctagttctctgccgagtccttctgaatgtcatacatgagtgtctgaagttaagattggagcagagacctgctggagaaccatctctcttgagtattaagcagctggtgagagagtgtaaggaggtcctgaagggcggcctgctgatgaagcagtactaccagttcatgctgcaggaggttctggaggacttggagaagcccgactgcaacattgacgcttttgaagaggatctacataaaatgcttatggtgtattttgattacatgagaagctggatccaaatgctacagcaattacctcaagcatcgcatagtttaaaaaatctgttagaagaagaatggaatttcaccaaagaaataactcattacatacggggaggagaagcacaggccgggaagcttttctgtgacattgcaggaatgctgctgaaatctacaggaagttttttagaatttggcttacaggagagctgtgctgaattttggactagtgcggatgacagcagtgcttccgacgaaatcaggaggtctgttatagagatcagtcgagccctgaaggagctcttccatgaagccagagaaagggcttccaaagcacttggatttgctaaaatgttgagaaaggacctggaaatagcagcagaattcaggctttcagccccagttagagacctcctggatgttctgaaatcaaaacagtatgtcaaggtgcaaattcctgggttagaaaacttgcaaatgtttgttccagacactcttgctgaggagaagagtattattttgcagttactcaatgcagctgcaggaaaggactgttcaaaagattcagatgacgtactcatcgatgcctatctgcttctgaccaagcacggtgatcgagcccgtgattcagaggacagctggggcacctgggaggcacagcctgtcaaagtcgtgcctcaggtggagactgttgacaccctgagaagcatgcaggtggataatcttttactagttgtcatgcagtctgcgcatctcacaattcagagaaaagctttccagcagtccattgagggacttatgactctgtgccaggagcagacatccagtcagccggtcatcgccaaagctttgcagcagctgaagaatgatgcattggagctatgcaacaggataagcaatgccattgaccgcgtggaccacatgttcacatcagaatttgatgctgaggttgatgaatctgaatctgtcaccttgcaacagtactaccgagaagcaatgattcaggggtacaattttggatttgagtatcataaagaagttgttcgtttgatgtctggggagtttagacagaagataggagacaaatatataagctttgcccggaagtggatgaattatgtcctgactaaatgtgagagtggtagaggtacaagacccaggtgggcgactcaaggatttgattttctacaagcaattgaacctgcctttatttcagctttaccagaagatgacttcttgagtttacaagccttgatgaatgaatgcattggccatgtcataggaaaaccacacagtcctgttacaggtttgtaccttgccattcatcggaacagcccccgtcctatgaaggtacctcgatgccatagtgaccctcctaacccacacctcattatccccactccagagggattcagcactcggagcatgccttccgacgcgcggagccatggcagccctgctgctgctgctgctgctgctgctgctgctgttgctgccagtcggcccagcccctctggtggtgactctgtgctgcccaaatccatcagcagtgcccatgataccaggggttccagcgttcctgaaaatgatcgattggcttccatagctgctgaattgcagtttaggtccctgagtcgtcactcaagccccacggaggagcgagatgaaccagcatatccaagaggagattcaagtgggtccacaagaagaagttgggaacttcggacactaatcagccagagtaaagatactgcttctaaactaggacccatagaagctatccagaagtcagtccgattgtttgaagaaaagaggtaccgagaaatgaggagaaagaatatcattggtcaagtttgtgatacgcctaagtcctatgataatgttatgcacgttggcttgaggaaggtgaccttcaaatggcaaagaggaaacaaaattggagaaggccagtatgggaaggtgtacacctgcatcagcgtcgacaccggggagctgatggccatgaaagagattcgatttcaacctaatgaccataagactatcaaggaaactgcagacgaattgaaaatattcgaaggcatcaaacaccccaatctggttcggtattttggtgtggagctccatagagaagaaatgtacatcttcatggagtactgcgatgaggggactttagaagaggtgtcaaggctgggacttcaggaacatgtgattaggctgtattcaaagcagatcaccattgcgatcaacgtcctccatgagcatggcatagtccaccgtgacattaaaggtgccaatatcttccttacctcatctggattaatcaaactgggagattttggatgttcagtaaagctcaaaaacaatgcccagaccatgcctggtgaagtgaacagcaccctggggacagcagcatacatggcacctgaagtcatcactcgtgccaaaggagagggccatgggcgtgcggccgacatctggagtctggggtgtgttgtcatagagatggtgactggcaagaggccttggcatgagtatgagcacaactttcaaattatgtataaagtggggatgggacataagccaccaatccctgaaagattaagccctgaaggaaaggacttcctttctcactgccttgagagtgacccaaagatgagatggaccgccagccagctcctcgaccattcgtttgtcaaggtttgcacagatgaagaatg

SEQ ID NO: 23 - DNA, 3867 bp, Homo sapiens
atggcggggccgtgtccccggtccggggcggagcgcgccggcagctgctggcaggacccgctggccgtggcgctgagccggggccggcagctcgcggcgcccccgggccggggctgcgcgcggagccggccgctcagcgtggtctacgtgctgacccgggagccgcagcccgggctcgagcctcgggagggaaccgaggcggagccgctgcccctgcgctgcctgcgcgaggcttgcgcgcaggtcccccggccgcggccgcccccgcagctgcgcagcctgcccttcgggacgctggagctaggcgacaccgcggctctggatgccttctacaacgcggatgtggtggtgctggaggtgagcagctcgctggtacagccctccctgttctaccaccttggtgtgcgtgagagcttcagcatgaccaacaatgtgctcctctgctcccaggccgacctccctgacctgcaggccctgcgggaggatgttttccagaagaactcggattgcgttggcagctacacactgatcccctatgtggtgacggccactggtcgggtgctgtgtggtgatgcaggccttctgcggggcctggctgatgggctggtacaggctggagtggggaccgaggccctgctcactcccctggtgggccggcttgcccgcctgctggaggccacacccacagactcttgtggctatttccgggagaccattcggcgggacatccggcaggcgcgggagcggttcagtgggccacagctgcggcaggagctggctcgcctgcagcggagactggacagcgtggagctgctgagccccgacatcatcatgaacttgctgctctcctaccgcgatgtgcaggactactcggccatcattgagctggtggagacgctgcaggccttgcccacctgtgatgtggccgagcagcataatgtctgcttccactacacttttgccctcaaccggaggaacaggcctggggaccgggcgaaggccctgtctgtgctgctgccgctggtacagcttgagggctctgtggcgcccgatctgtactgcatgtgtggccgtatctacaaggacatgttcttcagctcgggtttccaggatgctgggcaccgggagcaggcctatcactggtatcgcaaggcttttgacgtagagcccagccttcactcaggcatcaatgcagctgtgctcctcattgctgccgggcagcactttgaggattccaaagagctccggctaataggcatgaagctgggctgcctgctggcccgcaaaggctgcgtggagaagatgcagtattactgggatgtgggtttctacctgggagcccagatcctcgccaatgaccccacccaggtggtgctggctgcagagcagctgtataagctcaatgcccccatatggtacctggtgtccgtgatggagaccttcctgctctaccagcacttcaggcccacgccagagccccctggagggccaccacgccgtgcccacttctggctccacttcttgctacagtcctgccaaccattcaagacagcctgtgcccagggcgaccagtgcttggtgctggtcctggagatgaacaaggtgctgctgcctgcaaagctcgaggttcggggtactgacccagtaagcacagtgaccctgagcctgctggagcctgagacccaggacattccctccagctggaccttcccagtcgcctccatatgcggagtcagcgcctcaaagcgcgacgagcgctgctgcttcctctatgcactccccccggctcaggacgtccagctgtgcttccccagcgtagggcactgccagtggttctgcggcctgatccaggcctgggtgacgaacccggattccacggcgcccgcggaggaggcggagggcgcgggggagatgttggagtttgattatgagtacacggagacgggcgagcggctggtgctgggcaagggcacgtatggggtggtgtacgcgggccgcgatcgccacacgagggtgcgcatcgccatcaaggagatcccggagcgggacagcaggttctctcagcccctgcatgaagagatcgctcttcacagacgcctgcgccacaagaacatagtgcgctatctgggctcagctagccagggcggctaccttaagatcttcatggaggaagtgcctggaggcagcctgtcctccttgctgcggtcggtgtggggacccctgaaggacaacgagagcaccatcagtttctacacccgccagatcctgcagggacttggctacttgcacgacaaccacatcgtgcacagggacataaaaggggacaatgtgctgatcaacaccttcagtgggctgctcaagatttctgacttcggcacctccaagcggctggcaggcatcacaccttgcactgagaccttcacaggaactctgcagtatatggccccagaaatcattgaccagggcccacgcgggtatgggaaagcagctgacatctggtcactgggctgcactgtcattgagatggccacaggtcgcccccccttccacgagctcgggagcccacaggctgccatgtttcaggtgggtatgtacaaggtccatccgccaatgcccagctctctgtcggccgaggcccaagcctttctcctccgaacttttgagccagacccccgcctccgagccagcgcccagacactgctgggggaccccttcctgcagcctgggaaaaggagccgcagccccagctccccacgacatgctccacggccctcagatgccccttctgccagtcccactccttcagccaactcaaccacccagtctcagacattcccgtgccctcaggcaccctctcagcacccacccagccccccgaagcgctgcctcagttatgggggcaccagccagctccgggtgcccgaggagcctgcggccgaggagcctgcgtctccggaggagagttcggggctgagcctgctgcaccaggagagcaagcgtcgggccatgctggccgcagtattggagcaggagctgccagcgctggcggagaatctgcaccaggagcagaagcaagagcagggggcccgtctgggcagaaaccatgtggaagagctgctgcgctgcctcggggcacacatccacactcccaaccgccggcagctcgcccaggagctgcgggcgctgcaaggacggctgagggcccagggccttgggcctgcgcttctgcacagaccgctgtttgccttcccggatgcggtgaagcagatcctccgcaagcgccagatccgtccacactggatgttcgttctggactcactgctcagccgtgctgtgcgggcagccctgggtgtgctaggaccggaggtggagaaggaggcggtctcaccgaggtcagaggagctgagtaatgaaggggactcccagcagagcccaggccagcagagcccgcttccggtggagcccgagcagggccccgctcctctgatggtgcagctgagcctcttgagggcagagactgatcggctgcgcgaaatcctggcggggaaggaacgggagtaccaggccctggtgcagcgggctctacagcggctgaatgaggaagcccggacctatgtcctggccccagagcctccaactgctctttcaacggaccagggcctggtgcagtggctacaggaactgaatgtggattcaggcaccatccaaatgctgttgaaccatagcttcaccctccacactctgctcacctatgccactcgagatgacctcatctacacccgcatcaggggagggatggtatgccgcatctggagggccatcttggcacagcgagcaggatccacaccagtcacctctggaccctga

SEQ ID NO: 24 - DNA, 1740 bp, Homo sapiens
atgtctacagcctctgccgcctcctcctcctcctcgtcttcggccggtgagatgatcgaagccccttcccaggtcctcaactttgaagagatcgactacaaggagatcgaggtggaagaggttgttggaagaggagcctttggagttgtttgcaaagctaagtggagagcaaaagatgttgctattaaacaaatagaaagtgaatctgagaggaaagcgtttattgtagagcttcggcagttatcccgtgtgaaccatcctaatattgtaaagctttatggagcctgcttgaatccagtgtgtcttgtgatggaatatgctgaagggggctctttatataatgtgctgcatggtgctgaaccattgccatattatactgctgcccacgcaatgagttggtgtttacagtgttcccaaggagtggcttatcttcacagcatgcaacccaaagcgctaattcacagggacctgaaaccaccaaacttactgctggttgcaggggggacagttctaaaaatttgtgattttggtacagcctgtgacattcagacacacatgaccaataacaaggggagtgctgcttggatggcacctgaagtttttgaaggtagtaattacagtgaaaaatgtgacgtcttcagctggggtattattctttgggaagtgataacgcgtcggaaaccctttgatgagattggtggcccagctttccgaatcatgtgggctgttcataatggtactcgaccaccactgataaaaaatttacctaagcccattgagagcctgatgactcgttgttggtctaaagatccttcccagcgcccttcaatggaggaaattgtgaaaataatgactcacttgatgcggtactttccaggagcagatgagccattacagtatccttgtcagtattcagatgaaggacagagcaactctgccaccagtacaggctcattcatggacattgcttctacaaatacgagtaacaaaagtgacactaatatggagcaagttcctgccacaaatgatactattaagcgcttagaatcaaaattgttgaaaaatcaggcaaagcaacagagtgaatctggacgtttaagcttgggagcctcccgtgggagcagtgtggagagcttgcccccaacctctgagggcaagaggatgagtgctgacatgtctgaaatagaagctaggatcgccgcaaccacaggcaacggacagccaagacgtagatccatccaagacttgactgtaactggaacagaacctggtcaggtgagcagtaggtcatccagtcccagtgtcagaatgattactacctcaggaccaacctcagaaaagccaactcgaagtcatccatggacccctgatgattccacagataccaatggatcagataactccatcccaatggcttatcttacactggatcaccaactacagcctctagcaccgtgcccaaactccaaagaatctatggcagtgtttgaacagcattgtaaaatggcacaagaatatatgaaagttcaaacagaaattgcattgttattacagagaaagcaagaactagttgcagaactggaccaggatgaaaaggaccagcaaaatacatctcgcctggtacaggaacataaaaagcttttagatgaaaacaaaagcctttctacttactaccagcaatgcaaaaaacaactagaggtcatcagaagtcagcagcagaaacgacaaggcacttcatga

SEQ ID NO: 25 - DNA, 1113 bp, Homo sapiens
atgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtga

SEQ ID NO: 26 - DNA, 954 bp, Homo sapiens
atgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggttggctagaaggccacggggtgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 27 - DNA, 2907 bp, Homo sapiens
atggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 28 - DNA, 2844 bp, Homo sapiens
atggcagtgatggaaatggcctgcccaggtgcccctggctcagcagtggggcagcagaaggaactccccaaagccaaggagaagacgccgccactggggaagaaacagagctccgtctacaagcttgaggccgtggagaagagccctgtgttctgcggaaagtgggagatcctgaatgacgtgattaccaagggcacagccaaggaaggctccgaggcagggccagctgccatctctatcatcgcccaggctgagtgtgagaatagccaagagttcagccccaccttttcagaacgcattttcatcgctgggtccaaacagtacagccagtccgagagtcttgatcagatccccaacaatgtggcccatgctacagagggcaaaatggcccgtgtgtgttggaagggaaagcgtcgcagcaaagcccggaagaaacggaagaagaagagctcaaagtccctggctcatgcaggagtggccttggccaaacccctccccaggacccctgagcaggagagctgcaccatcccagtgcaggaggatgagtctccactcggcgccccatatgttagaaacaccccgcagttcaccaagcctctgaaggaaccaggccttgggcaactctgttttaagcagcttggcgagggcctacggccggctctgcctcgatcagaactccacaaactgatcagccccttgcaatgtctgaaccacgtgtggaaactgcaccacccccaggacggaggccccctgcccctgcccacgcaccccttcccctatagcagactgcctcatcccttcccattccaccctctccagccctggaaacctcaccctctggagtccttcctgggcaaactggcctgtgtagacagccagaaacccttgcctgacccacacctgagcaaactggcctgtgtagacagtccaaagcccctgcctggcccacacctggagcccagctgcctgtctcgtggtgcccatgagaagttttctgtggaggaatacctagtgcatgctctgcaaggcagcgtgagctcaggccaggcccacagcctgaccagcctggccaagacctgggcagcaaggggctccagatcccgggagcccagccccaaaactgaggacaacgagggtgtcctgctcactgagaaactcaagccagtggattatgagtaccgagaagaagtccactgggccacgcaccagctccgcctgggcagaggctccttcggagaggtgcacaggatggaggacaagcagactggcttccagtgcgctgtcaaaaaggtgcggctggaagtatttcgggcagaggagctgatggcatgtgcaggattgacctcacccagaattgtccctttgtatggagctgtgagagaagggccttgggtcaacatcttcatggagctgctggaaggtggctccctgggccagctggtcaaggagcagggctgtctcccagaggaccgggccctgtactacctgggccaggccctggagggtctggaatacctccactcacgaaggattctgcatggggacgtcaaagctgacaacgtgctcctgtccagcgatgggagccacgcagccctctgtgactttggccatgctgtgtgtcttcaacctgatggcctgggaaagtccttgctcacaggggactacatccctggcacagagacccacatggctccggaggtggtgctgggcaggagctgcgacgccaaggtggatgtctggagcagctgctgtatgatgctgcacatgctcaacggctgccacccctggactcagttcttccgagggccgctctgcctcaagattgccagcgagcctccgcctgtgagggagatcccaccctcctgcgcccctctcacagcccaggccatccaagaggggctgaggaaagagcccatccaccgcgtgtctgcagcggagctgggagggaaggtgaaccgggcactacagcaagtgggaggtctgaagagcccttggaggggagaatataaagaaccaagacatccaccgccaaatcaagccaattaccaccagaccctccatgcccagccgagagagctttcgccaagggccccagggccccggccagctgaggagacaacaggcagagcccctaagctccagcctcctctcccaccagagcccccagagccaaacaagtctcctcccttgactttgagcaaggaggagtctgggatgtgggaacccttacctctgtcctccctggagccagcccctgccagaaaccccagctcaccagagcggaaagcaaccgtcccggagcaggaactgcagcagctggaaatagaattattcctcaacagcctgtcccagccattttctctggaggagcaggagcaaattctctcgtgcctcagcatcgacagcctctccctgtcggatgacagtgagaagaacccatcaaaggcctctcaaagctcgcgggacaccctgagctcaggcgtacactcctggagcagccaggccgaggctcgaagctccagctggaacatggtgctggcccgggggcggcccaccgacaccccaagctatttcaatggtgtgaaagtccaaatacagtctcttaatggtgaacacctgcacatccgggagttccaccgggtcaaagtgggagacatcgccactggcatcagcagccagatcccagctgcagccttcagcttggtcaccaaagacgggcagcctgttcgctacgacatggaggtgccagactcgggcatcgacctgcagtgcacactggcccctgatggcagcttcgcctggagctggagggtcaagcatggccagctggagaacaggccctaa

SEQ ID NO: 29 - DNA, 1083 bp, Homo sapiens
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctga

SEQ ID NO: 30 - DNA, 1656 bp, Homo sapiens
atggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttag

SEQ ID NO: 31 - DNA, 1443 bp, Homo sapiens
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 32 - DNA, 2514 bp, Homo sapiens
atggctagcaaacgaaaatctacaactccatgcatggttcggacatcacaagtagtagaacaagatgtgcccgaggaagtagacagggccaaagagaaaggaatcggcacaccacagcctgacgtggccaaggacagttgggcagcagaacttgaaaactcttccaaagaaaacgaagtgatagaggtgaaatctatgggggaaagccagtccaaaaaactccaaggtggttatgagtgcaaatactgcccctactccacgcaaaacctgaacgagttcacggagcatgtcgacatgcagcatcccaacgtgattctcaaccccctctacgtgtgtgcagaatgtaacttcacaaccaaaaagtacgactccctatccgaccacaactccaagttccatcccggggaggccaacttcaagctgaagttaattaaacgcaataatcaaactgtcttggaacagtccatcgaaaccaccaaccatgtcgtgtccatcaccaccagtggccctggaactggtgacagtgattctgggatctcggtgagtaaaacccccatcatgaagcctggaaaaccaaaagcggatgccaagaaggtgcccaagaagcccgaggagatcacccccgagaaccacgtggaagggaccgcccgcctggtgacagacacagctgagatcctctcgagactcggcggggtggagctcctccaagacacattaggacacgtcatgccttctgtacagctgccaccaaatatcaaccttgtgcccaaggtccctgtcccactaaatactaccaaatacaactctgccctggatacaaatgccacgatgatcaactctttcaacaagtttccttacccgacccaggctgagttgtcctggctgacagctgcctccaaacacccagaggagcacatcagaatctggtttgccacccagcgcttaaagcatggcatcagctggtccccagaagaggtggaggaggcccggaagaagatgttcaacggcaccatccagtcagtacccccgaccatcactgtgctgcccgcccagttggcccccacaaaggtgacgcagcccatcctccagacggctctaccgtgccagatcctcggccagactagcctggtgctgactcaggtgaccagcgggtcaacaaccgtctcttgctcccccatcacacttgccgtggcaggagtcaccaaccatggccagaagagacccttggtgactccccaagctgcccccgaacccaagcgtccacacatcgctcaggtgccagagcccccacccaaggtggccaaccccccgctcacaccagccagtgaccgcaagaagacaaaggagcagatagcacatctcaaggccagctttctccagagccagttccctgacgatgccgaggtttaccggctcatcgaggtgactggccttgccaggagcgagatcaagaagtggttcagtgaccaccgatatcggtgtcaaaggggcatcgtccacatcaccagcgaatcccttgccaaagaccagttggccatcgcggcctcccgacacggtcgcacgtatcatgcgtacccagactttgccccccagaagttcaaagagaaaacacagggtcaggttaaaatcttggaagacagctttttgaaaagttcttttcctacccaagcagaactggatcggctaagggtggagaccaagctgagcaggagagagatcgactcctggttctcggagaggcggaagcttcgagacagcatggaacaagctgtcttggattccatggggtctggcaaaaaaggccaagatgtgggagcccccaatggtgctctgtctcgactcgaccagctctccggtgcccagttaacaagttctctgcccagcccttcgccagcaattgcaaaaagtcaagaacaggttcatctcctgaggagcacgtttgcaagaacccagtggcctactccccaggagtacgaccagttagcggccaagactggcctggtccgaactgagattgtgcgttggttcaaggagaacagatgcttgctgaaaacgggaaccgtgaagtggatggagcagtaccagcaccagcccatggcagatgatcacggctacgatgccgtagcaaggaaagcaacaaaacccatggccgagagcccaaagaacgggggtgatgtggttccacaatattacaaggaccccaaaaagctctgcgaagaggacttggagaagttggtgaccagggtaaaagtaggcagcgagccagcaaaagactgtttgccagcaaagccctcagaggccacctcagaccggtcagagggcagcagccgggacggccagggtagcgacgagaacgaggagtcgagcgttgtggattacgtggaggtgacggtcggggaggaggatgcgatctcagatagatcagatagctggagtcaggctgcggcagaaggtgtgtcggaactggctgaatcagactccgactgcgtccctgcagaggctggccaggcctag

SEQ ID NO: 33 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 34 - DNA, 570 bp, Homo sapiens
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 35 - DNA, 2016 bp, Homo sapiens
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaa

SEQ ID NO: 36 - DNA, 1569 bp, Homo sapiens
atgagtctgctaaactgtgaaaacagctgtggatccagccagtctgaaagtgactgctgtgtggccatggccagctcctgtagcgctgtaacaaaagatgatagtgtgggtggaactgccagcacggggaacctctccagctcatttatggaggagatccagggatatgatgtagagtttgacccacccctggaaagcaagtatgaatgccccatctgcttgatggcattacgagaagcagtgcaaacgccatgcggccataggttctgcaaagcctgcatcataaaatcaataagggatgcaggtcacaaatgtccagttgacaatgaaatactgctggaaaatcaactatttccagacaattttgcaaaacgtgagattctttctctgatggtgaaatgtccaaatgaaggttgtttgcacaagatggaactgagacatcttgaggatcatcaagcacattgtgagtttgctcttatggattgtccccaatgccagcgtcccttccaaaaattccatattaatattcacattctgaaggattgtccaaggagacaggtttcttgtgacaactgtgctgcatcaatggcatttgaagataaagagatccatgaccagaactgtcctttggcaaatgtcatctgtgaatactgcaatactatactcatcagagaacagatgcctaatcattatgatctagactgccctacagccccaattccatgcacattcagtacttttggttgccatgaaaagatgcagaggaatcacttggcacgccacctacaagagaacacccagtcacacatgagaatgttggcccaggctgttcatagtttgagcgttatacccgactctgggtatatctcagaggtccggaatttccaggaaactattcaccagttagagggtcgccttgtaagacaagaccatcaaatccgggagctgactgctaaaatggaaactcagagtatgtatgtaagtgagctcaaacgaaccattcgaacccttgaggacaaagttgctgaaatcgaagcacagcagtgcaatggaatttatatttggaagattggcaactttggaatgcatttgaaatgtcaagaagaggagaaacctgttgtgattcatagccctggattctacactggcaaacccgggtacaaactgtgcatgcgcttgcaccttcagttaccgactgctcagcgctgtgcaaactatatatccctttttgtccacacaatgcaaggagaatatgacagccacctcccttggcccttccagggtacaatacgccttacaattcttgatcagtctgaagcacctgtaaggcaaaaccacgaagagataatggatgccaaaccagagctgcttgctttccagcgacccacaatcccacggaacccaaaaggttttggctatgtaacttttatgcatctggaagccctaagacaaagaactttcattaaggatgacacattattagtgcgctgtgaggtctccacccgctttgacatgggtagccttcggagggagggttttcagccacgaagtactgatgcaggggtatag

SEQ ID NO: 37 - DNA, 999 bp, Homo sapiens
atggccaaccgttacaccatggatctgactgccatctacgagagcctcctgtcgctgagccctgacgtgcccgtgccatccgaccatggagggactgagtccagcccaggctggggctcctcgggaccctggagcctgagcccctccgactccagcccgtctggggtcacctcccgcctgcctggccgctccaccagcctagtggagggccgcagctgtggctgggtgcccccaccccctggcttcgcaccgctggctccccgcctgggccctgagctgtcaccctcacccacttcgcccactgcaacctccaccaccccctcgcgctacaagactgagctatgtcggaccttctcagagagtgggcgctgccgctacggggccaagtgccagtttgcccatggcctgggcgagctgcgccaggccaatcgccaccccaaatacaagacggaactctgtcacaagttctacctccagggccgctgcccctacggctctcgctgccacttcatccacaaccctagcgaagacctggcggccccgggccaccctcctgtgcttcgccagagcatcagcttctccggcctgccctctggccgccggacctcaccaccaccaccaggcctggccggcccttccctgtcctccagctccttctcgccctccagctccccaccaccacctggggaccttccactgtcaccctctgccttctctgctgcccctggcacccccctggctcgaagagaccccaccccagtctgttgcccctcctgccgaagggccactcctatcagcgtctgggggcccttgggtggcctggttcggaccccctctgtacagtccctgggatccgaccctgatgaatatgccagcagcggcagcagcctggggggctctgactctcccgtcttcgaggcgggagtttttgcaccaccccagcccgtggcagccccccggcgactccccatcttcaatcgcatctctgtttctgagtga